(19) United States
(12) Patent Application Publication
(10) Pub. No.: US 2025/0108064 A1
(43) Pub. Date: Apr. 3, 2025

(54) PHOSPHATE PRODRUGS OF CANNABINOIDS

(71) Applicant: Emory University, Atlanta, GA (US)

(72) Inventors: Luke E. Hodson, Atlanta, GA (US); Nicole Pribut, Atlanta, GA (US); Stephen F. Traynelis, Decatur, GA (US); Dennis C. Liotta, Atlanta, GA (US)

(73) Assignee: Emory University, Atlanta, GA (US)

(21) Appl. No.: 18/291,630

(22) PCT Filed: Jul. 29, 2022
(86) PCT No.: PCT/US2022/038876

§ 371 Date: Jan. 24, 2024

Related U.S. Application Data
(60) Provisional application No. 63/227,296, filed on Jul. 29, 2021.

(51) Int. Cl. A61K 31/661; A61P 25/08; C07F 9/117

(52) U.S. Cl. CPC A61K 31/661; A61P 25/08; C07F 9/117

(57) ABSTRACT

Phosphate prodrugs of cannabinoids are disclosed herein as are pharmaceutical compositions comprising one or more of the same. Such compounds and compositions are useful for the treatment of diseases, disorders, and/or conditions, including neurological disorders and neuropathic pain.

This application claims the benefit under 35 U.S.C. § 119(e) to U.S. Provisional Application No. 63/227,296 filed Jul. 29, 2021, which application is incorporated herein by reference in its entirety.

The present disclosure relates to prodrugs of cannabinoids (CBs), pharmaceutical compositions comprising same, and uses related thereto.

Cannabinoids refer to a heterogenous family of molecules extracted from Cannabis sativa, which has been shown to exhibit pharmacological properties by interacting with specific receptors, such as membrane receptors coupled to G proteins. The pharmacology of these compounds is rapidly expanding, and there is a growing body of pharmacological effects and therapeutic properties of CB receptor agonists. These include analgesia, muscle relaxation, immunosuppressant, anti-inflammatory and anti-allergic effects, anxiolytic effects, improvement of mood, stimulation of appetite, anti-emesis, lowering of intraocular pressure, bronchodilation, neuroprotection anti-convulsant effects, and antineoplastic effects.

Cannabidiol (CBD), the main non-psychotropic component of this family, constitutes up to 40% of the Cannabis extract. CBD is a pharmacologically promiscuous compound, and the complete mechanism of action for all its biological effects is yet undetermined. CBD acts as an allosteric antagonist of cannabinoid 1 receptors (CB₁Rs) and cannabinoid 2 receptors (CB₂Rs). Evidence suggests that CBD produces many of its effects in vivo via facilitatory interactions with serotonin 1A (5-HT_{1A}) receptors. This activity extends to the and 6 opioid receptors and transient receptor potential vanilloid type-1 (TRPV1) cation channels.

In the past two decades, comprehensive clinical and preclinical studies have revealed several positive physiological and behavioural effects of CBD. CBD possesses anxiolytic, analgesic, antiemetic, and antipsychotic properties, allowing for the potential treatment of a variety of conditions ranging from psychiatric disorders such as dementia and schizophrenia, to addiction and nausea. Additionally, CBD exhibits physiological effects that promote and maintain health, including anti-inflammatory, anti-oxidative and neuroprotective effects. These attractive properties have resulted in CBD being utilized as an emerging therapeutic strategy for pain relief and the treatment of diabetes, epilepsy, and cancer.

However, CBD therapy is not as accessible as it should be due to its complicated pharmacokinetic properties that hinders the achievement of therapeutically effective systemic concentrations. The oral bioavailability of CBD has been shown to be very low (around 13-19%). It undergoes extensive first-pass metabolism and its metabolites are mostly excreted via the kidneys. Plasma and brain concentrations are dose-dependent. Systemic bioavailability of inhaled CBD ranges from 11-45%, and daily oral doses of 10 mg/kg chronically administered resulted in a mean plasma concentration of 5.9-11.2 ng/ml. CBD is highly fat-soluble and rapidly redistributed into fatty tissues throughout the body, where it may be stored for as long as four weeks and released slowly into the blood at sub-therapeutic concentrations. A large portion of CBD is excreted intact or as its glucuronide metabolite.

Most of the clinical trials have been carried out with the two commercially available preparations EPIDIOLEX® and SATIVEX®. SATIVEX®, which contains CBD and Δ⁹-tetrahydrocannabinol (Δ⁹-THC), is administered by oromucosal spray administration and has been approved for the treatment of multiple sclerosis symptoms. It has been shown that co-administration of CBD and Δ⁹-THC may alter the pharmacological effects of the latter, potentiating some putative benefits while attenuating some of its negative effects. GW Pharmaceuticals has developed an oral sesame seed oil base formulation of pure CBD (EPIDIOLEX®) for the treatment of severe, orphan, early-onset, treatment-resistant epilepsy syndromes, showing significant reductions in seizure frequency compared to placebo in several trials. The recommended daily dose requires at least 400 mg (see U.S. Pat. Nos. 9,066,920 and 9,522,123), which suggests that a high amount needs to be administered for effective results.

Thus, there is a need for CB drugs with enhanced physicochemical, pharmacological, and/or pharmacokinetic properties, such as solubility and oral bioavailability. For example, there is a need for improved prodrug strategies that can overcome one or more of the physicochemical, pharmacological, and/or pharmacokinetic liabilities of CBs.

Disclosed are cannabinoid prodrugs of Formula (I):

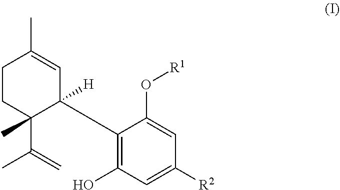

and pharmaceutically acceptable salts thereof, wherein R¹ and R² are defined herein.

As used herein, “compound of Formula (I)” includes cannabinoid prodrugs of Formula (I) and pharmaceutically acceptable salts thereof.

In some embodiments, pharmaceutical compositions comprising at least one compound of Formula (I) and at least one additional pharmaceutically acceptable ingredient are presented.

In some embodiments, a method for treatment and/or prevention of at least one disease, disorder, and/or condition where treatment with a cannabinoid is useful is disclosed, the method comprising administering to a subject in need thereof an effective amount of at least one compound of Formula (I) and/or a pharmaceutical composition comprising at least one compound of Formula (I).

### BRIEF DESCRIPTION OF THE DRAWINGS

FIG. 1 is a diagram illustrating the synthesis of compounds 7a-7f.

FIG. 2 is a diagram illustrating the synthesis of compounds 8 and 14a-14k.

FIG. 3 is a diagram illustrating the synthesis of compound 24.

FIG. 4 is a diagram illustrating the synthesis of compounds 29a-29c.

FIG. 5 is a diagram illustrating the synthesis of compound 32.

FIG. 6 is a diagram illustrating the general synthesis of compounds 38a-38c.

FIG. 7 is a diagram illustrating a prophetic synthesis of compound 43.

FIG. 8 is a graph illustrating the hepatocyte cellular uptake of compound 7b showing metabolism of compound 7b and formation of CBD.

FIG. 9 is a graph illustrating the hepatocyte cellular uptake of compound 8 showing metabolism of compound 8 and formation of both compound 7b and CBD.

FIG. 10 is a graph illustrating the hepatocyte cellular uptake of compound 14a showing metabolism of compound 14a and formation of both compound 7b and CBD.

FIG. 11 is a graph illustrating the brain homogenate stability of compound 7b, showing metabolism of compound 7b and formation of CBD.

FIG. 12 is a graph illustrating CYP inhibition of compound 7b and CBD at 10 μM concentration.

FIG. 13 is a graph illustrating CBD released from compounds 7c, 29a, 32 and 38c in plasma and the brain when administered PO dose of 50 mg/kg equiv.

FIG. 14 is a graph illustrating 6 Hz 22 mA electroshock studies of compounds 7a, 7b, and CBD, 2 hours post IP dose.

Disclosed herein are cannabinoid prodrugs and pharmaceutical compositions comprising the same. The compounds and compositions of the present disclosure may be useful for treating and/or preventing at least one disease, disorder, and/or condition that is treatable or preventable by administering a cannabinoid.

The compounds of the present disclosure may have at least one improved physicochemical, pharmacological, and/or pharmacokinetic property.

In some embodiments, presented are cannabinoid prodrugs of Formula (I):

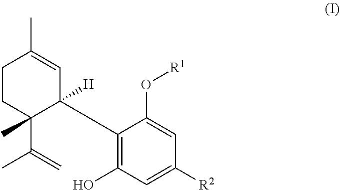

and pharmaceutically acceptable salts thereof, wherein
R¹ is chosen from

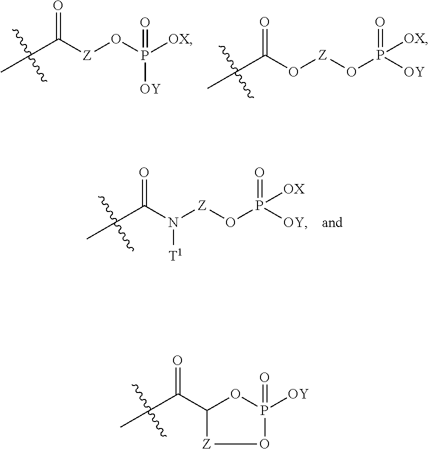

groups, wherein
Z is chosen from divalent C₁₋₁₈ alkyl and divalent C₁₋₁₈ haloalkyl groups, wherein the divalent C₁₋₁₈ alkyl and divalent C₁₋₁₈ haloalkyl groups are optionally substituted with one or more groups independently chosen from C₆₋₁₈ aryl, C₁₋₁₃ heteroaryl, C₂₋₁₂ heterocyclyl, —OC₁₋₁₈ alkyl, —SC₁₋₁₈ alkyl, —N(T²)C₁₋₁₈ alkyl, and —N(T²)AA groups, wherein T² is chosen from H and C₁₋₈ alkyl groups and AA is chosen from amino acid residues and is attached to the nitrogen via its C-terminus carbonyl group (i.e., forming an amide bond);
X and Y, which may be identical or different, are independently chosen from H, Q, C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

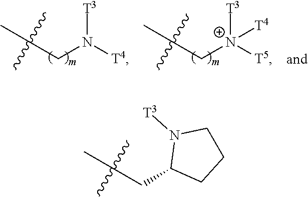

groups, wherein
   each T³, T⁴, and T⁵, which may be the same or different, are independently chosen from H and C₁₋₈ alkyl groups,
   each Q is independently chosen from pharmaceutically acceptable cations,
   each n is independently chosen from integers ranging from 1 to 12, and
   each m is independently chosen from integers ranging from 1 to 8; and
T¹ is chosen from H, C₁₋₁₈ alkyl, and C₁₋₁₈ haloalkyl groups; and
R² is chosen from C₁₋₈ alkyl groups.

In some embodiments, R¹ is chosen from

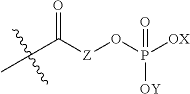

groups. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

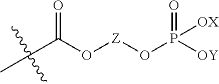

groups.

In some embodiments, R¹ is chosen from

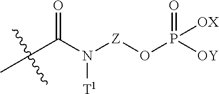

groups. In some embodiments, T¹ is H. In some embodiments, T¹ is methyl

In some embodiments, R¹ is chosen from

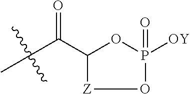

groups.

In some embodiments, Z is chosen from divalent C₁₋₁₈ alkyl groups. In some embodiments, Z is chosen from divalent C₁₋₁₂ alkyl groups. In some embodiments, Z is chosen from divalent C₁₋₈ alkyl groups. In some embodiments, Z is chosen from divalent C₁₋₆ alkyl groups. In some embodiments, Z is chosen from divalent C₁₋₄ alkyl groups. In some embodiments, Z is chosen from divalent C₁₋₃ alkyl groups. In some embodiments, Z is chosen from divalent C₁₋₂ alkyl groups. In some embodiments, Z is a divalent C₁ alkyl group, i.e., divalent methylene.

In some embodiments, Z is chosen from —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—,

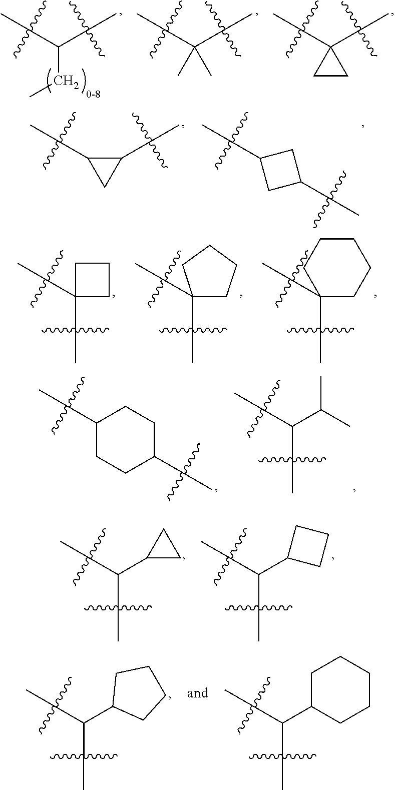

In some embodiments, Z is —CH₂—.

In some embodiments, Z is —(CH₂)₂—.

In some embodiments, Z is —(CH₂)₃—.

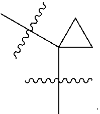 [SMILES: *C1(*)CC1]

In some embodiments, Z is

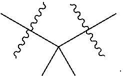 [SMILES: *C(*)(C)C]

In some embodiments, Z is

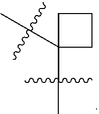 [SMILES: *C1(*)CCC1]

In some embodiments, Z is

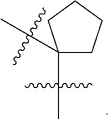 [SMILES: *C1(*)CCCC1]

In some embodiments, Z is

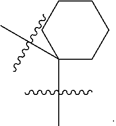 [SMILES: *C1(*)CCCCC1]

In some embodiments, Z is

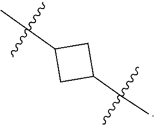 [SMILES: *C1CC(*)C1]

In some embodiments, Z is

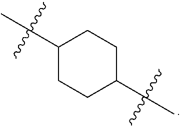 [SMILES: *C1CCC(*)CC1]

In some embodiments, Z is chosen from divalent C₁₋₁₈ haloalkyl groups. In some embodiments, Z is chosen from divalent C₁₋₁₂ haloalkyl groups. In some embodiments, Z is chosen from divalent C₁₋₈ haloalkyl groups. In some embodiments, Z is chosen from divalent C₁₋₆ haloalkyl groups. In some embodiments, Z is chosen from divalent C₁₋₄ haloalkyl groups. In some embodiments, Z is chosen from divalent C₁₋₃ haloalkyl groups. In some embodiments, Z is chosen from divalent C₁₋₂ haloalkyl groups.

In some embodiments, Z is

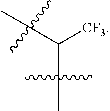 [SMILES: *C(*)C(F)(F)F]

In some embodiments, Z is

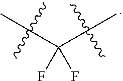 [SMILES: *C(*)(F)F]

In some embodiments, Z is chosen from divalent C₁₋₁₈ alkyl groups substituted with at least one group chosen from C₆₋₁₈ aryl groups. In some embodiments, Z is chosen from divalent C₁₋₁₆ alkyl groups substituted with at least one group chosen from C₆₋₁₈ aryl groups. In some embodiments, Z is chosen from divalent C₁₋₁₄ alkyl groups substituted with at least one group chosen from C₆₋₁₈ aryl groups. In some embodiments, Z is chosen from divalent C₁₋₁₂ alkyl groups substituted with at least one group chosen from C₆₋₁₈ aryl groups. In some embodiments, Z is chosen from divalent C₁₋₁₀ alkyl groups substituted with at least one group chosen from C₆₋₁₈ aryl groups. In some embodiments, Z is chosen from divalent C₁₋₈ alkyl groups substituted with at least one group chosen from C₆₋₁₈ aryl groups. In some embodiments, Z is chosen from divalent C₁₋₆ alkyl groups substituted with at least one group chosen from C₆₋₁₈ aryl groups. In some embodiments, Z is chosen from divalent C₁₋₄ alkyl groups substituted with at least one group chosen from C₆₋₁₈ aryl groups. In some embodiments, Z is chosen from divalent C₂ alkyl groups substituted with at least one group chosen from C₆₋₁₈ aryl groups. In some embodiments, Z is a divalent C₁ alkyl group substituted with at least one group chosen from C₆₋₁₈ aryl groups.

In some embodiments, Z is chosen from divalent C₁₋₁₈ alkyl groups substituted with at least one group chosen from C₆₋₁₀ aryl groups. In some embodiments, Z is chosen from divalent C₁₋₁₆ alkyl groups substituted with at least one group chosen from C₆₋₁₀ aryl groups. In some embodiments, Z is chosen from divalent C₁₋₁₄ alkyl groups substituted with at least one group chosen from C₆₋₁₀ aryl groups. In some embodiments, Z is chosen from divalent C₁₋₁₂ alkyl groups substituted with at least one group chosen from C₆₋₁₀ aryl groups. In some embodiments, Z is chosen from divalent C₁₋₁₀ alkyl groups substituted with at least one group chosen from C₆₋₁₀ aryl groups. In some embodiments, Z is chosen from divalent C₁₋₈ alkyl groups substituted with at least one group chosen from C₆₋₁₀ aryl groups. In some embodiments, Z is chosen from divalent C₁₋₆ alkyl groups substituted with at least one group chosen from C₆₋₁₀ aryl groups. In some embodiments, Z is chosen from divalent C₁₋₄ alkyl groups substituted with at least one group chosen from C₆₋₁₀ aryl groups. In some embodiments, Z is chosen from divalent C₂ alkyl groups substituted with at least one group chosen from C₆₋₁₀ aryl groups. In some embodiments, Z is a divalent C₁ alkyl group substituted with at least one group chosen from C₆₋₁₀ aryl groups. In some embodiments, the at least one group chosen from C₆₋₁₀ aryl groups is phenyl.

In some embodiments, Z is

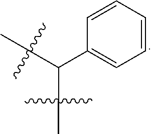 [SMILES: *C(*)c1ccccc1]

In some embodiments, Z is

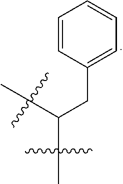 [SMILES: *C(*)Cc1ccccc1]

In some embodiments, Z is chosen from divalent C₁₋₁₈ alkyl groups substituted with at least one group chosen from C₁₋₁₃ heteroaryl groups. In some embodiments, Z is chosen from divalent C₁₋₁₆ alkyl groups substituted with at least one group chosen from C₁₋₁₃ heteroaryl groups. In some embodiments, Z is chosen from divalent C₁₋₁₄ alkyl groups substituted with at least one group chosen from C₁₋₁₃ heteroaryl groups. In some embodiments, Z is chosen from divalent C₁₋₁₂ alkyl groups substituted with at least one group chosen from C₁₋₁₃ heteroaryl groups. In some embodiments, Z is chosen from divalent C₁₋₁₀ alkyl groups substituted with at least one group chosen from C₁₋₁₃ heteroaryl groups. In some embodiments, Z is chosen from divalent C₁₋₈ alkyl groups substituted with at least one group chosen from C₁₋₁₃ heteroaryl groups. In some embodiments, Z is chosen from divalent C₁₋₆ alkyl groups substituted with at least one group chosen from C₁₋₁₃ heteroaryl groups. In some embodiments, Z is chosen from divalent C₁₋₄ alkyl groups substituted with at least one group chosen from C₁₋₁₃ heteroaryl groups. In some embodiments, Z is chosen from divalent C₂ alkyl groups substituted with at least one group chosen from C₁₋₁₃ heteroaryl groups. In some embodiments, Z is a divalent C₁ alkyl group substituted with at least one group chosen from C₁₋₁₃ heteroaryl groups.

In some embodiments, Z is chosen from divalent C₁₋₁₈ alkyl groups substituted with at least one group chosen from C₁₋₈ heteroaryl groups. In some embodiments, Z is chosen from divalent C₁₋₁₆ alkyl groups substituted with at least one group chosen from C₁₋₈ heteroaryl groups. In some embodiments, Z is chosen from divalent C₁₋₁₄ alkyl groups substituted with at least one group chosen from C₁₋₈ heteroaryl groups. In some embodiments, Z is chosen from divalent C₁₋₁₂ alkyl groups substituted with at least one group chosen from C₁₋₈ heteroaryl groups. In some embodiments, Z is chosen from divalent C₁₋₁₀ alkyl groups substituted with at least one group chosen from C₁₋₈ heteroaryl groups. In some embodiments, Z is chosen from divalent C₁₋₈ alkyl groups substituted with at least one group chosen from C₁₋₈ heteroaryl groups. In some embodiments, Z is chosen from divalent C₁₋₆ alkyl groups substituted with at least one group chosen from C₁₋₈ heteroaryl groups. In some embodiments, Z is chosen from divalent C₁₋₄ alkyl groups substituted with at least one group chosen from C₁₋₈ heteroaryl groups. In some embodiments, Z is chosen from divalent C₂ alkyl groups substituted with at least one group chosen from C₁₋₈ heteroaryl groups. In some embodiments, Z is a divalent C₁ alkyl group substituted with at least one group chosen from C₁₋₈ heteroaryl groups.

In some embodiments, Z is chosen from divalent C₁₋₁₈ alkyl groups substituted with at least one group chosen from C₁₋₅ heteroaryl groups. In some embodiments, Z is chosen from divalent C₁₋₁₆ alkyl groups substituted with at least one group chosen from C₁₋₅ heteroaryl groups. In some embodiments, Z is chosen from divalent C₁₋₁₄ alkyl groups substituted with at least one group chosen from C₁₋₅ heteroaryl groups. In some embodiments, Z is chosen from divalent C₁₋₁₂ alkyl groups substituted with at least one group chosen from C₁₋₅ heteroaryl groups. In some embodiments, Z is chosen from divalent C₁₋₁₀ alkyl groups substituted with at least one group chosen from C₁₋₅ heteroaryl groups. In some embodiments, Z is chosen from divalent C₁₋₈ alkyl groups substituted with at least one group chosen from C₁₋₅ heteroaryl groups. In some embodiments, Z is chosen from divalent C₁₋₆ alkyl groups substituted with at least one group chosen from C₁₋₅ heteroaryl groups. In some embodiments, Z is chosen from divalent C₁₋₄ alkyl groups substituted with at least one group chosen from C₁₋₅ heteroaryl groups. In some embodiments, Z is chosen from divalent C₂ alkyl groups substituted with at least one group chosen from C₁₋₅ heteroaryl groups. In some embodiments Z is a divalent C₁ alkyl group substituted with at least one group chosen from C₁₋₅ heteroaryl groups. In some embodiments, the at least one group chosen from C₁₋₅ heteroaryl groups is chosen from

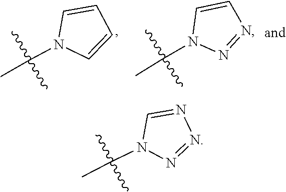 [SMILES: *n1cccc1 ; *n1ccnn1 ; *n1cnnn1]

In some embodiments, Z is chosen from

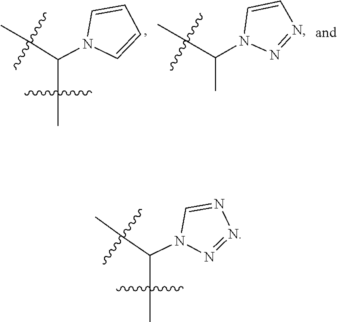

In some embodiments, Z is chosen from divalent C₁₋₁₈ alkyl groups substituted with at least one group chosen from C₂₋₁₂ heterocyclyl groups. In some embodiments, Z is chosen from divalent C₁₋₁₆ alkyl groups substituted with at least one group chosen from C₂₋₁₂ heterocyclyl groups. In some embodiments, Z is chosen from divalent C₁₋₁₄ alkyl groups substituted with at least one group chosen from C₂₋₁₂ heterocyclyl groups. In some embodiments, Z is chosen from divalent C₁₋₁₂ alkyl groups substituted with at least one group chosen from C₂₋₁₂ heterocyclyl groups. In some embodiments, Z is chosen from divalent C₁₋₁₀ alkyl groups substituted with at least one group chosen from C₂₋₁₂ heterocyclyl groups. In some embodiments, Z is chosen from divalent C₁₋₈ alkyl groups substituted with at least one group chosen from C₂₋₁₂ heterocyclyl groups. In some embodiments, Z is chosen from divalent C₁₋₆ alkyl groups substituted with at least one group chosen from C₂₋₁₂ heterocyclyl groups. In some embodiments, Z is chosen from divalent C₁₋₄ alkyl groups substituted with at least one group chosen from C₂₋₁₂ heterocyclyl groups. In some embodiments, Z is chosen from divalent C₂ alkyl groups substituted with at least one group chosen from C₂₋₁₂ heterocyclyl groups. In some embodiments, Z is a divalent C₁ alkyl group substituted with at least one group chosen from C₂₋₁₂ heterocyclyl groups.

In some embodiments, Z is chosen from divalent C₁₋₁₈ alkyl groups substituted with at least one group chosen from C₂₋₅ heterocyclyl groups. In some embodiments, Z is chosen from divalent C₁₋₁₆ alkyl groups substituted with at least one group chosen from C₂₋₅ heterocyclyl groups. In some embodiments, Z is chosen from divalent C₁₋₁₄ alkyl groups substituted with at least one group chosen from C₂₋₅ heterocyclyl groups. In some embodiments, Z is chosen from divalent C₁₋₁₂ alkyl groups substituted with at least one group chosen from C₂₋₅ heterocyclyl groups. In some embodiments, Z is chosen from divalent C₁₋₁₀ alkyl groups substituted with at least one group chosen from C₂₋₅ heterocyclyl groups. In some embodiments, Z is chosen from divalent C₁₋₈ alkyl groups substituted with at least one group chosen from C₂₋₅ heterocyclyl groups. In some embodiments, Z is chosen from divalent C₁₋₆ alkyl groups substituted with at least one group chosen from C₂₋₅ heterocyclyl groups. In some embodiments, Z is chosen from divalent C₁₋₄ alkyl groups substituted with at least one group chosen from C₂₋₅ heterocyclyl groups. In some embodiments, Z is chosen from divalent C₂ alkyl groups substituted with at least one group chosen from C₂₋₅ heterocyclyl groups. In some embodiments, Z is a divalent C₁ alkyl group substituted with at least one group chosen from C₂₋₅ heterocyclyl groups. In some embodiments, the at least one group chosen from C₂₋₅ heterocyclyl groups is chosen from

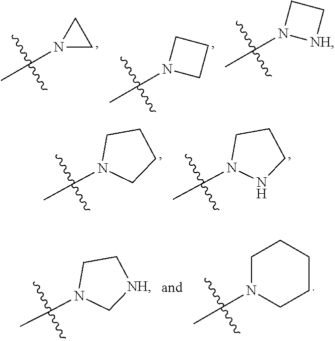

In some embodiments, Z is chosen from

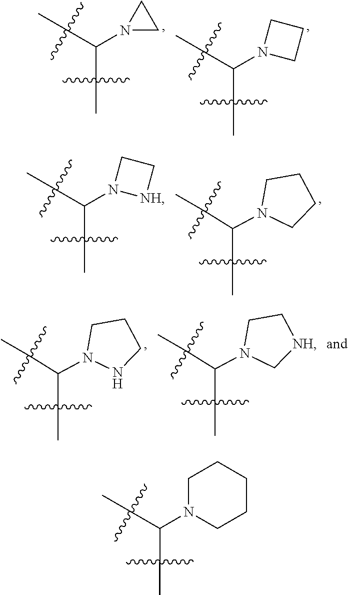

In some embodiments, Z is chosen from divalent C₁₋₁₈ alkyl groups substituted with at least one group chosen from —OC₁₋₁₈ alkyl groups. In some embodiments, Z is chosen from divalent C₁₋₁₆ alkyl groups substituted with at least one group chosen from —OC₁₋₁₈ alkyl groups. In some embodiments, Z is chosen from divalent C₁₋₁₄ alkyl groups substituted with at least one group chosen from —OC₁₋₁₈ alkyl groups. In some embodiments, Z is chosen from divalent C₁₋₁₂ alkyl groups substituted with at least one group chosen from —OC₁₋₁₈ alkyl groups. In some embodiments, Z is chosen from divalent C₁₋₁₀ alkyl groups substituted with at least one group chosen from —OC₁₋₁₈ alkyl groups. In some embodiments, Z is chosen from divalent C₁₋₈ alkyl groups substituted with at least one group chosen from —OC₁₋₁₈ alkyl groups. In some embodiments, Z is chosen from divalent C₁₋₆ alkyl groups substituted with at least one group chosen from —OC₁₋₁₈ alkyl groups. In some embodiments, Z is chosen from divalent C₁₋₄ alkyl groups substituted with at least one group chosen from —OC₁₋₁₈ alkyl groups. In some embodiments, Z is chosen from divalent C₂ alkyl groups substituted with at least one group chosen from —OC₁₋₁₈ alkyl groups. In some embodiments, Z is a divalent C₁ alkyl group substituted with at least one group chosen from —OC₁₋₁₈ alkyl groups.

In some embodiments, Z is chosen from divalent C₁₋₁₈ alkyl groups substituted with at least one group chosen from —OC₁₋₁₀ alkyl groups. In some embodiments, Z is chosen from divalent C₁₋₁₆ alkyl groups substituted with at least one group chosen from —OC₁₋₁₀ alkyl groups. In some embodiments, Z is chosen from divalent C₁₋₁₄ alkyl groups substituted with at least one group chosen from —OC₁₋₁₀ alkyl groups. In some embodiments, Z is chosen from divalent C₁₋₁₂ alkyl groups substituted with at least one group chosen from —OC₁₋₁₀ alkyl groups. In some embodiments, Z is chosen from divalent C₁₋₁₀ alkyl groups substituted with at least one group chosen from —OC₁₋₁₀ alkyl groups. In some embodiments, Z is chosen from divalent C₁₋₈ alkyl groups substituted with at least one group chosen from —OC₁₋₁₀ alkyl groups. In some embodiments, Z is chosen from divalent C₁₋₆ alkyl groups substituted with at least one group chosen from —OC₁₋₁₀ alkyl groups. In some embodiments, Z is chosen from divalent C₁₋₄ alkyl groups substituted with at least one group chosen from —OC₁₋₁₀ alkyl groups. In some embodiments, Z is chosen from divalent C₂ alkyl groups substituted with at least one group chosen from —OC₁₋₁₀ alkyl groups. In some embodiments, Z is a divalent C₁ alkyl group substituted with at least one group chosen from —OC₁₋₁₀ alkyl groups. In some embodiments, the at least one group chosen from —OC₁₋₁₀ alkyl groups is chosen from

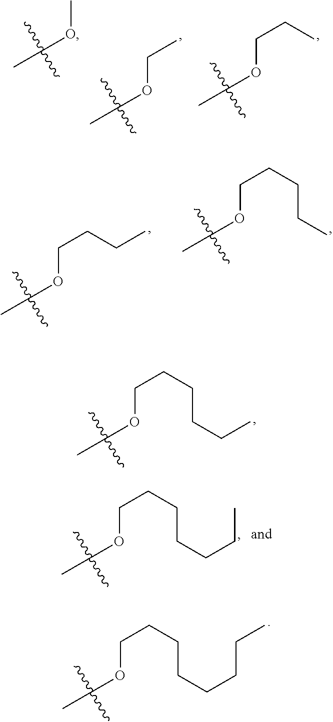

In some embodiments, Z is chosen from

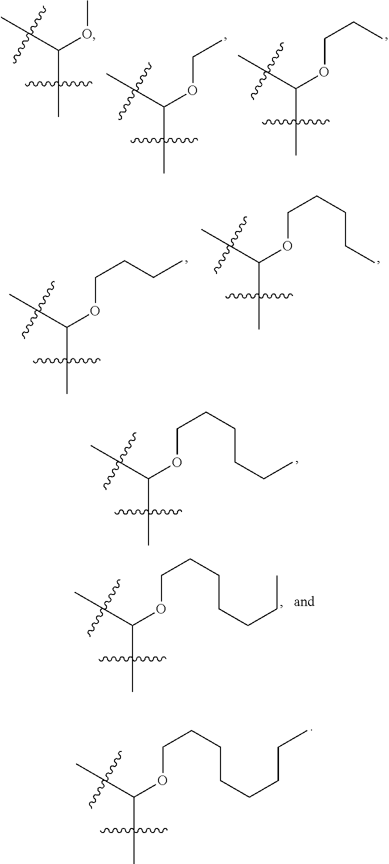

In some embodiments, Z is chosen from divalent C₁₋₁₈ alkyl groups substituted with at least one group chosen from —SC₁₋₁₈ alkyl groups. In some embodiments, Z is chosen from divalent C₁₋₁₆ alkyl groups substituted with at least one group chosen from —SC₁₋₁₈ alkyl groups. In some embodiments, Z is chosen from divalent C₁₋₁₄ alkyl groups substituted with at least one group chosen from —SC₁₋₁₈ alkyl groups. In some embodiments, Z is chosen from divalent C₁₋₁₂ alkyl groups substituted with at least one group chosen from —SC₁₋₁₈ alkyl groups. In some embodiments, Z is chosen from divalent C₁₋₁₀ alkyl groups substituted with at least one group chosen from —SC₁₋₁₈ alkyl groups. In some embodiments, Z is chosen from divalent C₁₋₈ alkyl groups substituted with at least one group chosen from —SC₁₋₁₈ alkyl groups. In some embodiments, Z is chosen from divalent C₁₋₆ alkyl groups substituted with at least one group chosen from —SC₁₋₁₈ alkyl groups. In some embodiments, Z is chosen from divalent C₁₋₄ alkyl groups substituted with at least one group chosen from —SC₁₋₁₈ alkyl groups. In some embodiments, Z is chosen from divalent C₂ alkyl groups substituted with at least one group chosen from —SC₁₋₁₈ alkyl groups. In some embodiments, Z is a divalent C₁ alkyl group substituted with at least one group chosen from —SC₁₋₁₈ alkyl groups.

In some embodiments, Z is chosen from divalent C₁₋₁₈ alkyl groups substituted with at least one group chosen from —SC₁₋₁₀ alkyl groups. In some embodiments, Z is chosen from divalent C₁₋₁₆ alkyl groups substituted with at least one group chosen from —SC₁₋₁₀ alkyl groups. In some embodiments, Z is chosen from divalent C₁₋₁₄ alkyl groups substituted with at least one group chosen from —SC₁₋₁₀ alkyl groups. In some embodiments, Z is chosen from divalent C₁₋₁₂ alkyl groups substituted with at least one group chosen from —SC₁₋₁₀ alkyl groups. In some embodiments, Z is chosen from divalent C₁₋₁₀ alkyl groups substituted with at least one group chosen from —SC₁₋₁₀ alkyl groups. In some embodiments, Z is chosen from divalent C₁₋₁₂ alkyl groups substituted with at least one group chosen from —SC₁₋₁₀ alkyl groups. In some embodiments, Z is chosen from divalent C₁₋₆ alkyl groups substituted with at least one group chosen from —SC₁₋₁₀ alkyl groups. In some embodiments, Z is chosen from divalent C₁₋₄ alkyl groups substituted with at least one group chosen from —SC₁₋₁₀ alkyl groups. In some embodiments, Z is chosen from divalent C₂ alkyl groups substituted with at least one group chosen from —SC₁₋₁₀ alkyl groups. In some embodiments, Z is a divalent C₁ alkyl group substituted with at least one group chosen from —SC₁₋₁₀ alkyl groups. In some embodiments, the at least one group chosen from —SC₁₋₁₀ alkyl groups is chosen from

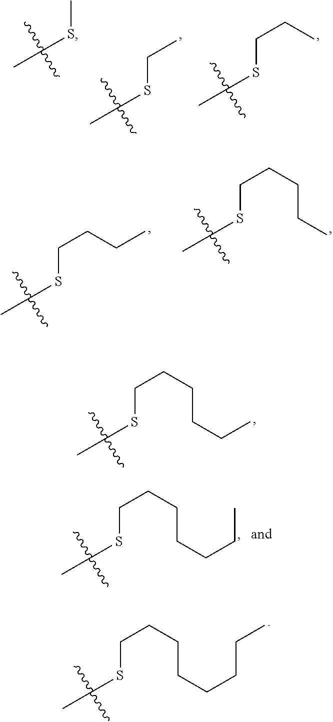

In some embodiments, Z is chosen from

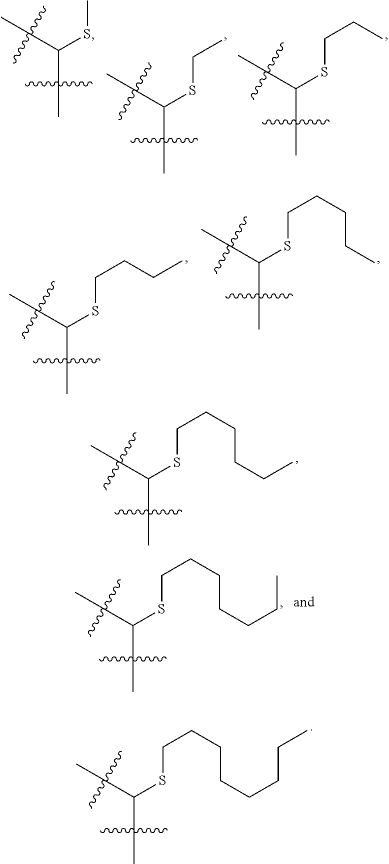

In some embodiments, Z is chosen from divalent C₁₋₁₈ alkyl groups substituted with at least one group chosen from —N(T²)C₁₋₁₈ alkyl groups. In some embodiments, Z is chosen from divalent C₁₋₁₆ alkyl groups substituted with at least one group chosen from —N(T²)C₁₋₁₈ alkyl groups. In some embodiments, Z is chosen from divalent C₁₋₁₄ alkyl groups substituted with at least one group chosen from —N(T²)C₁₋₁₈ alkyl groups. In some embodiments, Z is chosen from divalent C₁₋₁₂ alkyl groups substituted with at least one group chosen from —N(T²)C₁₋₁₈ alkyl groups. In some embodiments, Z is chosen from divalent C₁₋₁₀ alkyl groups substituted with at least one group chosen from —N(T²)C₁₋₁₈ alkyl groups. In some embodiments, Z is chosen from divalent C₁₋₈ alkyl groups substituted with at least one group chosen from —N(T²)C₁₋₁₈ alkyl groups. In some embodiments, Z is chosen from divalent C₁₋₆ alkyl groups substituted with at least one group chosen from —N(T²)C₁₋₁₈ alkyl groups. In some embodiments, Z is chosen from divalent C₁₋₄ alkyl groups substituted with at least one group chosen from —N(T²)C₁₋₁₈ alkyl groups. In some embodiments, Z is chosen from divalent C₂ alkyl groups substituted with at least one group chosen from —N(T²)C₁₋₁₈ alkyl groups. In some embodiments, Z is a divalent C₁ alkyl group substituted with at least one group chosen from —N(T²)C₁₋₁₈ alkyl groups. In some embodiments, the at least one group chosen from —N(T²)C₁₋₁₈ alkyl groups is chosen from —NHC₁₋₁₈ alkyl groups, i.e., T² is H. In some embodiments, the at least one group chosen from —N(T²)C₁₋₁₈ alkyl groups is chosen from —N(C₁₋₈ alkyl groups)C₁₋₁₈ alkyl groups, i.e., T² is C₁₋₈ alkyl groups.

In some embodiments, Z is chosen from divalent C₁₋₁₈ alkyl groups substituted with at least one group chosen from —N(T²)C₁₋₁₀ alkyl groups. In some embodiments, Z is chosen from divalent C₁₋₁₆ alkyl groups substituted with at least one group chosen from —N(T²)C₁₋₁₀ alkyl groups. In some embodiments, Z is chosen from divalent C₁₋₁₄ alkyl groups substituted with at least one group chosen from —N(T²)C₁₋₁₀ alkyl groups. In some embodiments, Z is chosen from divalent C₁₋₁₂ alkyl groups substituted with at least one group chosen from —N(T²)C₁₋₁₀ alkyl groups. In some embodiments, Z is chosen from divalent C₁₋₁₀ alkyl groups substituted with at least one group chosen from —N(T²)C₁₋₁₀ alkyl groups. In some embodiments, Z is chosen from divalent C₁₋₈ alkyl groups substituted with at least one group chosen from —N(T²)C₁₋₁₀ alkyl groups. In some embodiments, Z is chosen from divalent C₁₋₆ alkyl groups substituted with at least one group chosen from —N(T²)C₁₋₁₀ alkyl groups. In some embodiments, Z is chosen from divalent C₁₋₄ alkyl groups substituted with at least one group chosen from —N(T²)C₁₋₁₀ alkyl groups. In some embodiments, Z is chosen from divalent C₂ alkyl groups substituted with at least one group chosen from —N(T²)C₁₋₁₀ alkyl groups. In some embodiments, Z is a divalent C₁ alkyl group substituted with at least one group chosen from —N(T²)C₁₋₁₀ alkyl groups. In some embodiments, the at least one group chosen from —N(T²)C₁₋₁₀ alkyl groups is chosen from —NHC₁₋₁₀ alkyl groups. In some embodiments, the at least one group chosen from —N(T²)C₁₋₁₀ alkyl groups is chosen from —N(C₁₋₈ alkyl groups)C₁₋₁₀ alkyl groups. In some embodiments, the at least one group chosen from —N(T²)C₁₋₁₀ alkyl groups is chosen from

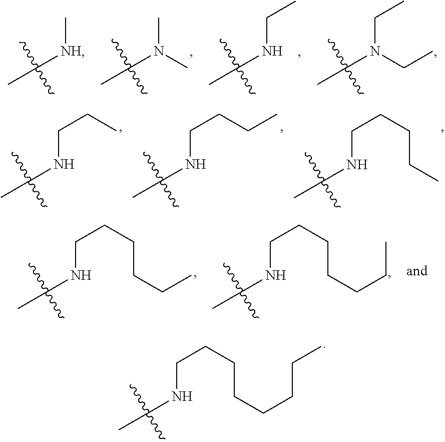

In some embodiments, Z is chosen from

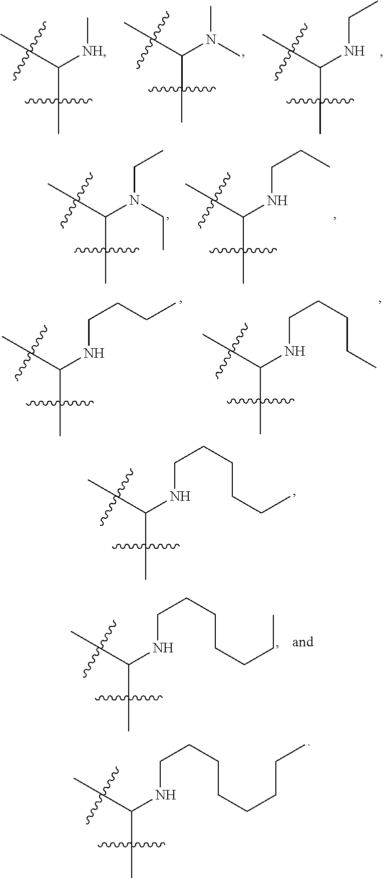

In some embodiments, Z is chosen from divalent C₁₋₁₈ alkyl groups substituted with at least one group chosen from —N(T²)AA groups. In some embodiments, Z is chosen from divalent C₁₋₁₆ alkyl groups substituted with at least one group chosen from —N(T²)AA groups. In some embodiments, Z is chosen from divalent C₁₋₁₄ alkyl groups substituted with at least one group chosen from —N(T²)AA groups. In some embodiments, Z is chosen from divalent C₁₋₁₂ alkyl groups substituted with at least one group chosen from —N(T²)AA groups. In some embodiments, Z is chosen from divalent C₁₋₁₀ alkyl groups substituted with at least one group chosen from —N(T²)AA groups. In some embodiments, Z is chosen from divalent C₁₋₈ alkyl groups substituted with at least one group chosen from —N(T²)AA groups. In some embodiments, Z is chosen from divalent C₁₋₆ alkyl groups substituted with at least one group chosen from —N(T²)AA groups. In some embodiments, Z is chosen from divalent C₁₋₄ alkyl groups substituted with at least one group chosen from —N(T²)AA groups. In some embodiments, Z is chosen from divalent C₂ alkyl groups substituted with at least one group chosen from —N(T²)AA groups. In some embodiments, Z is a divalent C₁ alkyl group substituted with at least one group chosen from —N(T²)AA groups. In some embodiments, the at least one group chosen from —N(T²)AA groups is chosen from —NHAA groups. In some embodiments, the at least one group chosen from —N(T²)AA groups is chosen from —N(C₁₋₈ alkyl groups)AA groups.

In some embodiments, Z is chosen from

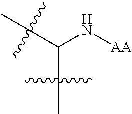

groups.

In some embodiments, AA is chosen from natural amino acid residues. In some embodiments, AA is chosen from unnatural amino acid residues. Exemplary unnatural amino acids can be found in (1) Unnatural Amino Acid: Tools for Drug Discovery, ChemFiles (by Sigma-Aldrich), Vol. 4, No. 5; and (2) Schultz et al., J. Bio., Chem, 2010, 285(15), 11039-11044. In some embodiments, AA is chosen from L-amino acid residues. In some embodiments, AA is chosen from natural L-amino acid residues.

In some embodiments, X is chosen from C₁₋₁₈ alkyl groups. In some embodiments, X is chosen from C₁₋₁₄ alkyl groups. In some embodiments, X is chosen from C₁₋₁₂ alkyl groups. In some embodiments, X is chosen from C₁₋₁₀ alkyl groups. In some embodiments, X is chosen from C₁₋₈ alkyl groups. In some embodiments, X is chosen from C₁₋₄ alkyl groups. In some embodiments, X is chosen from C₃₋₁₀ alkyl groups. In some embodiments, X is chosen from —CH₃, —(CH₂)CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —(CH₂)₈CH₃, and —(CH₂)₉CH₃.

In some embodiments, X is chosen from

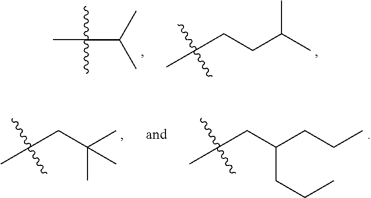

In some embodiments, X is chosen from C₁₋₁₈ haloalkyl groups. In some embodiments, X is chosen from C₁₋₁₄ haloalkyl groups. In some embodiments, X is chosen from C₁₋₁₂ haloalkyl groups. In some embodiments, X is chosen from C₁₋₁₀ haloalkyl groups. In some embodiments, X is chosen from C₁₋₈ haloalkyl groups. In some embodiments, X is chosen from C₁₋₄ haloalkyl groups.

In some embodiments, X is chosen from C₆₋₁₈ aryl and C₇₋₁₉ arylalkyl groups. In some embodiments, X is chosen from C₆₋₁₂ aryl and C₇₋₁₃ arylalkyl groups. In some embodiments, X is chosen from C₆₋₁₈ aryl groups. In some embodiments, X is chosen from C₆₋₁₂ aryl groups. In some embodiments, X is chosen from C₇₋₁₉ arylalkyl groups. In some embodiments, X is chosen from C₇₋₁₃ arylalkyl groups.

In some embodiments, X is

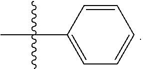 [SMILES: *c1ccccc1]

In some embodiments, X is

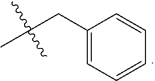 [SMILES: *Cc1ccccc1]

In some embodiments, X is chosen from C₁₋₁₃ heteroaryl and C₂₋₁₄ heteroarylalkyl groups. In some embodiments, X is chosen from C₁₋₁₃ heteroaryl groups. In some embodiments, X is chosen from C₁₋₁₀ heteroaryl groups. In some embodiments, X is chosen from C₁₋₈ heteroaryl groups. In some embodiments, X is chosen from C₁₋₆ heteroaryl groups. In some embodiments, X is chosen from C₂₋₁₄ heteroarylalkyl groups. In some embodiments, X is chosen from C₂₋₁₁ heteroarylalkyl groups. In some embodiments, X is chosen from C₂₋₉ heteroarylalkyl groups. In some embodiments, X is chosen from C₂₋₇ heteroarylalkyl groups.

In some embodiments, X is chosen from —(CH₂CH₂O)ₙCH₃ groups. In some embodiments, n is chosen from integers ranging from 1 to 10. In some embodiments, n is chosen from integers ranging from 1 to 8. In some embodiments, n is chosen from integers ranging from 1 to 6. In some embodiments, n is chosen from integers ranging from 1 to 4. In some embodiments, n is 1. In some embodiments, n is 2. In some embodiments, n is 3. In some embodiments, n is 4.

In some embodiments, X is

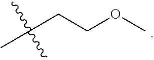 [SMILES: *CCOC]

In some embodiments, X is

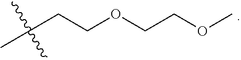 [SMILES: *CCOCCOC]

In some embodiments, X is chosen from

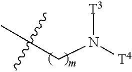

groups.

In some embodiments, X is chosen from

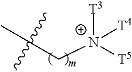

groups.

In some embodiments, X is chosen from

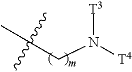

groups,
wherein m is chosen from integers ranging from 1 to 6. In some embodiments, m is chosen from integers ranging from 1 to 5. In some embodiments, m is chosen from integers ranging from 1 to 4. In some embodiments, m is chosen from integers ranging from 1 to 3. In some embodiments, m is 1. In some embodiments, m is 2. In some embodiments, m is 3. In some embodiments, m is 4.

In some embodiments, X is chosen from

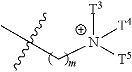

groups,
wherein m is chosen from integers ranging from 1 to 6. In some embodiments, m is chosen from integers ranging from 1 to 5. In some embodiments, m is chosen from integers ranging from 1 to 4. In some embodiments, m is chosen from integers ranging from 1 to 3. In some embodiments, m is 1. In some embodiments, m is 2. In some embodiments, m is 3. In some embodiments, m is 4.

In some embodiments, X is chosen from

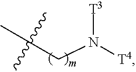

wherein T³ and T⁴, which may be the same or different, are independently chosen from H and C₁₋₅ alkyl groups. In some embodiments, T³ and T⁴ are each H. In some embodiments, T³ and T⁴, which may be the same or different, are independently chosen from C₁₋₅ alkyl groups. In some embodiments, T³ is H and T⁴ is chosen from C₁₋₅ alkyl groups.

In some embodiments, X is chosen from

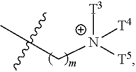

wherein T³, T⁴, and T⁵, which may be the same or different, are independently chosen from H and C₁₋₅ alkyl groups. In some embodiments, T³, T⁴, and T⁵ are each H. In some embodiments, T³, T⁴, and T⁵, which may be the same or different, are independently chosen from C₁₋₅ alkyl groups. In some embodiments, T³ is H, and T⁴ and T⁵, which may be the same or different, are independently chosen from C₁₋₅ alkyl groups. In some embodiments, T³ and T₄ are each H, and T⁵ is chosen from C₁₋₅ alkyl groups.

In some embodiments, X is chosen from

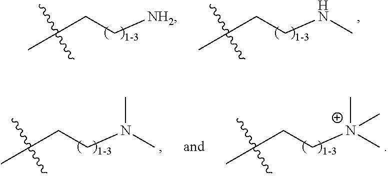

In some embodiments, X is chosen from

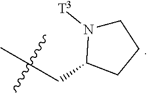

In some embodiments, X is

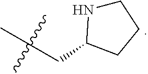 [SMILES: *C[C@H]1CCCN1]

In some embodiments, Y is chosen from C₁₋₁₈ alkyl groups. In some embodiments, Y is chosen from C₁₋₁₄ alkyl groups. In some embodiments, Y is chosen from C₁₋₁₂ alkyl groups. In some embodiments, Y is chosen from C₁₋₁₀ alkyl groups. In some embodiments, Y is chosen from C₁₋₈ alkyl groups. In some embodiments, Y is chosen from C₁₋₄ alkyl groups. In some embodiments, Y is chosen from C₃₋₁₀ alkyl groups. In some embodiments, Y is chosen from —CH₃, —(CH₂)CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —(CH₂)₈CH₃, and —(CH₂)₉CH₃.

In some embodiments, Y is chosen from

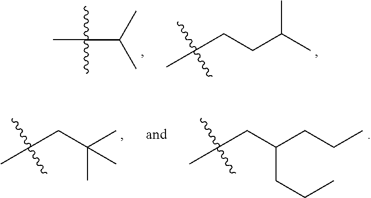

In some embodiments, Y is chosen from C₁₋₁₈ haloalkyl groups. In some embodiments, Y is chosen from C₁₋₁₄ haloalkyl groups. In some embodiments, Y is chosen from C₁₋₁₂ haloalkyl groups. In some embodiments, Y is chosen from C₁₋₁₀ haloalkyl groups. In some embodiments, Y is chosen from C₁₋₈ haloalkyl groups. In some embodiments, Y is chosen from C₁₋₄ haloalkyl groups.

In some embodiments, Y is chosen from C₆₋₈ aryl and C₇₋₁₉ arylalkyl groups. In some embodiments, Y is chosen from C₆₋₁₂ aryl and C₇₋₁₃ arylalkyl groups. In some embodiments, Y is chosen from C₆₋₁₈ aryl groups. In some embodiments, Y is chosen from C₆₋₁₂ aryl groups. In some embodiments, Y is chosen from C₇₋₁₉ arylalkyl groups. In some embodiments, Y is chosen from C₇₋₁₃ arylalkyl groups.

In some embodiments, Y is

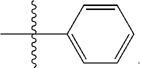 [SMILES: *c1ccccc1]

In some embodiments, Y is

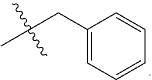 [SMILES: *Cc1ccccc1]

In some embodiments, Y is chosen from C₁₋₁₃ heteroaryl and C₂₋₁₄ heteroarylalkyl groups. In some embodiments, Y is chosen from C₁₋₁₃ heteroaryl groups. In some embodiments, Y is chosen from C₁₋₁₀ heteroaryl groups. In some embodiments, Y is chosen from C₁₋₈ heteroaryl groups. In some embodiments, Y is chosen from C₁₋₆ heteroaryl groups. In some embodiments, Y is chosen from C₂₋₁₄ heteroarylalkyl groups. In some embodiments, Y is chosen from C₂₋₁₁ heteroarylalkyl groups. In some embodiments, Y is chosen from C₂₋₉ heteroarylalkyl groups. In some embodiments, Y is chosen from C₂₋₇ heteroarylalkyl groups.

In some embodiments, Y is chosen from —(CH₂CH₂O)ₙCH₃ groups. In some embodiments, n is chosen from integers ranging from 1 to 10. In some embodiments, n is chosen from integers ranging from 1 to 8. In some embodiments, n is chosen from integers ranging from 1 to 6. In some embodiments, n is chosen from integers ranging from 1 to 4. In some embodiments, n is 1. In some embodiments, n is 2. In some embodiments, n is 3. In some embodiments, n is 4.

In some embodiments, Y is

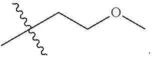 [SMILES: *CCOC]

In some embodiments, Y is

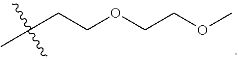 [SMILES: *CCOCCOC]

In some embodiments, Y is chosen from

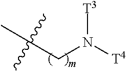

groups.

In some embodiments, Y is chosen from

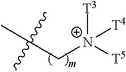

groups.

In some embodiments, Y is chosen from

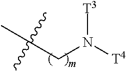

groups,
wherein m is chosen from integers ranging from 1 to 6. In some embodiments, m is chosen from integers ranging from 1 to 5. In some embodiments, m is chosen from integers ranging from 1 to 4. In some embodiments, m is chosen from integers ranging from 1 to 3. In some embodiments, m is 1. In some embodiments, m is 2. In some embodiments, m is 3. In some embodiments, m is 4.

In some embodiments, Y is chosen from

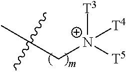

groups,
wherein m is chosen from integers ranging from 1 to 6. In some embodiments, m is chosen from integers ranging from 1 to 5. In some embodiments, m is chosen from integers ranging from 1 to 4. In some embodiments, m is chosen from integers ranging from 1 to 3. In some embodiments, m is 1. In some embodiments, m is 2. In some embodiments, m is 3. In some embodiments, m is 4.

In some embodiments, Y is chosen from

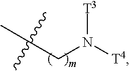

wherein T³ and T⁴, which may be the same or different, are independently chosen from H and C₁₋₅ alkyl groups. In some embodiments, T³ and T⁴ are each H. In some embodiments, T³ and T⁴, which may be the same or different, are independently chosen from C₁₋₅ alkyl groups. In some embodiments, T³ is H and T⁴ is chosen from C₁₋₅ alkyl groups.

In some embodiments, Y is chosen from

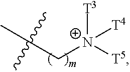

wherein T³, T⁴, and T⁵, which may be the same or different, are independently chosen from H and C₁₋₅ alkyl groups. In some embodiments, T³, T⁴, and T⁵ are each H. In some embodiments, T³, T⁴, and T⁵, which may be the same or different, are independently chosen from C₁₋₅ alkyl groups. In some embodiments, T³ is H, and T⁴ and T⁵, which may be the same or different, are independently chosen from C₁₋₅ alkyl groups. In some embodiments, T³ and T₄ are each H, and T⁵ is chosen from C₁₋₅ alkyl groups.

In some embodiments, Y is chosen from

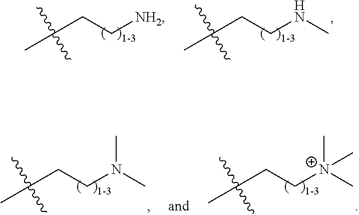

In some embodiments, Y is chosen from

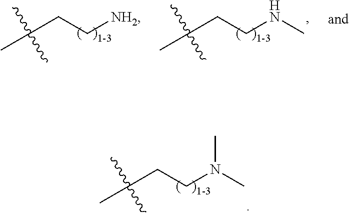

In some embodiments, Y is chosen from

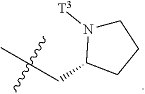

In some embodiments, Y is

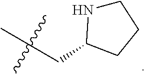 [SMILES: *C[C@H]1CCCN1]

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q.

In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

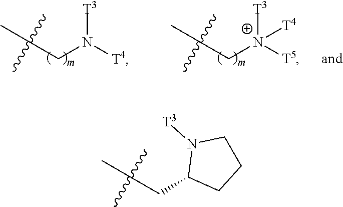

groups.

In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl and isopropyl.

Non-limiting examples of Q include cations of aluminium, arginine, benzathine, calcium, choline, copper, amines (substituted, e.g., diethylamine, and unsubstituted), diolamine, glycine, iron, lithium, lysine, magnesium, manganese, meglumine, olamine, potassium, sodium, tromethamine and zinc. In some embodiments, at least one Q is chosen from ammonium (substituted and unsubstituted) cations. In some embodiments, at least one Q is unsubstituted ammonium cation. In some embodiments, each Q is chosen from ammonium (substituted and unsubstituted) cations. In some embodiments, each Q is unsubstituted ammonium cation.

In some embodiments, T¹ is H. In some embodiments, T¹ is chosen from C₁₋₁₈ alkyl groups. In some embodiments, T¹ is chosen from C₁₋₁₂ alkyl groups. In some embodiments, T¹ is chosen from C₁₋₁₀ alkyl groups. In some embodiments, T¹ is chosen from C₁₋₈ alkyl groups. In some embodiments, T¹ is chosen from C₁₋₄ alkyl groups. In some embodiments, T¹ is chosen from Me, Et, n-Pr, iPr, n-Bu, s-Bu, i-Bu, t-Bu, cyclopropyl, and cyclobutyl. In some embodiments, T¹ is Me.

In some embodiments, T¹ is chosen from C₁₋₁₈ haloalkyl groups. In some embodiments, T¹ is chosen from C₁₋₁₂ haloalkyl groups. In some embodiments, T¹ is chosen from C₁₋₁₀ haloalkyl groups. In some embodiments, T¹ is chosen from C₁₋₈ haloalkyl groups. In some embodiments, T¹ is CF₂CF₃. In some embodiments, T¹ is CF₃.

In some embodiments, R² is chosen from C₂₋₆ alkyl groups. In some embodiments, R² is chosen from C₃ alkyl groups. In some embodiments, R² is chosen from C₄ alkyl groups. In some embodiments, R² is chosen from C₅ alkyl groups. In some embodiments, R² is n-propyl. In some embodiments, R² is n-pentyl.

In some embodiments, the compound of Formula (I) is chosen from compounds having the following formula:

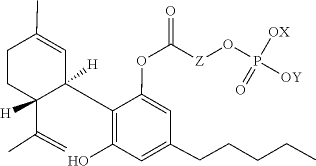

and pharmaceutically acceptable salts thereof. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃,

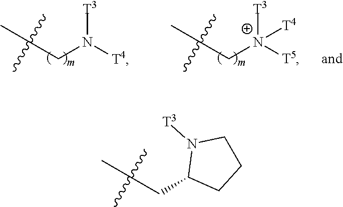

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

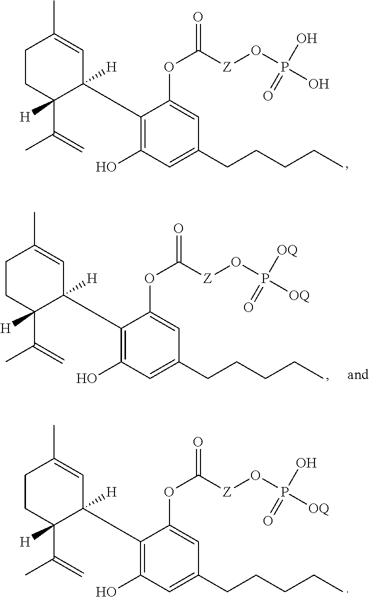

In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (I) is chosen from compounds having the following formulae:

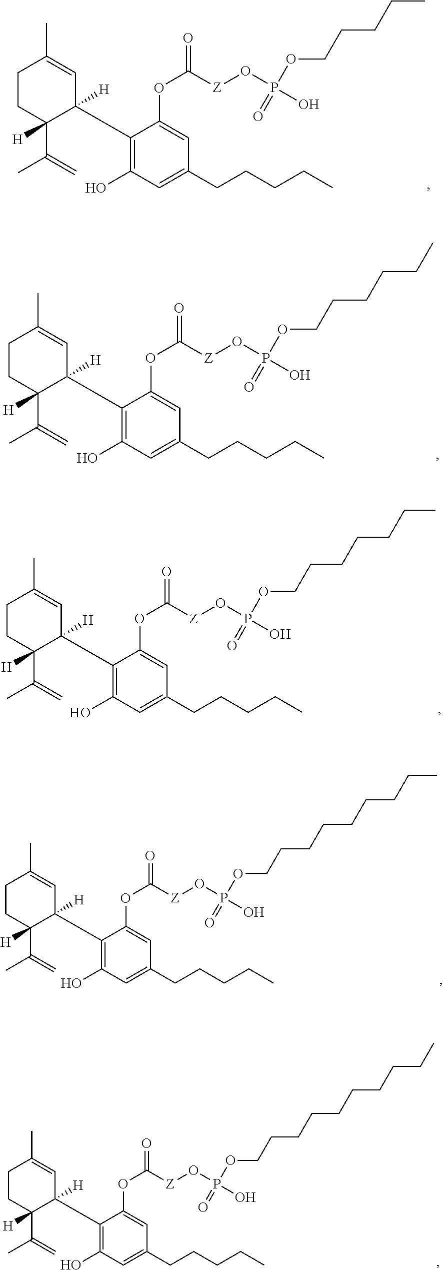

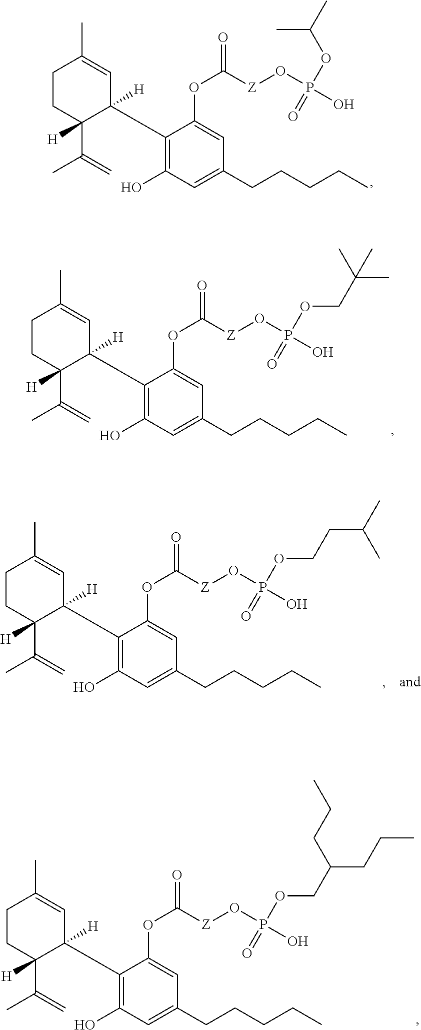

and pharmaceutically acceptable salts of any of the foregoing. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (I) is chosen from compounds having the following formulae:

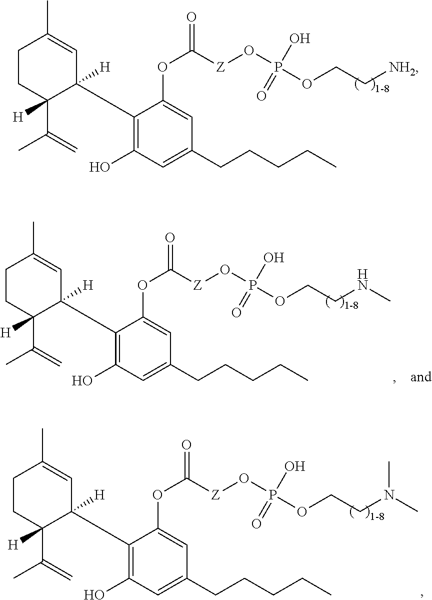

and pharmaceutically acceptable salts of any of the foregoing. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (I) is chosen from compounds having the following formulae:

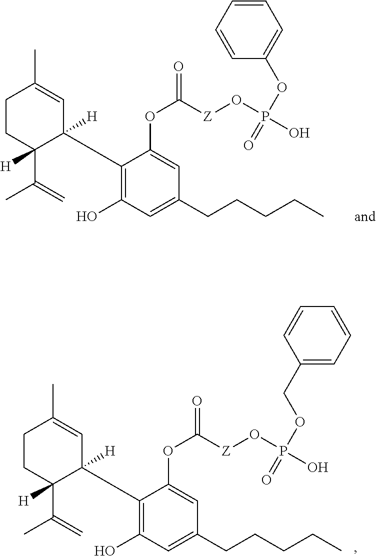

and pharmaceutically acceptable salts of any of the foregoing. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (I) is chosen from compounds having the following formulae:

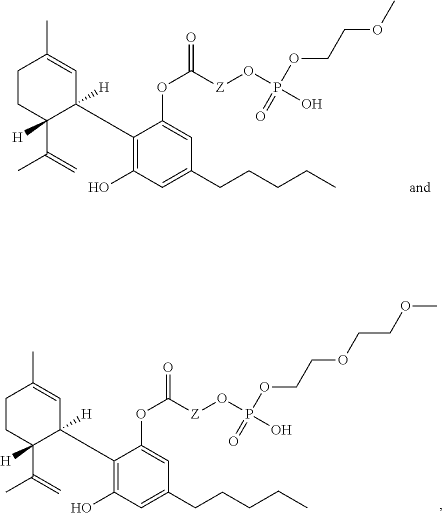

and pharmaceutically acceptable salts of any of the foregoing. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (I) is chosen from compounds having the following formula:

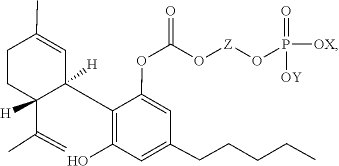

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃

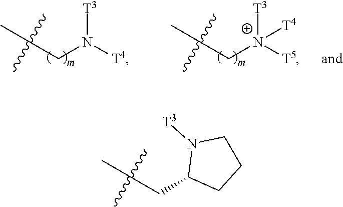

groups.

In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

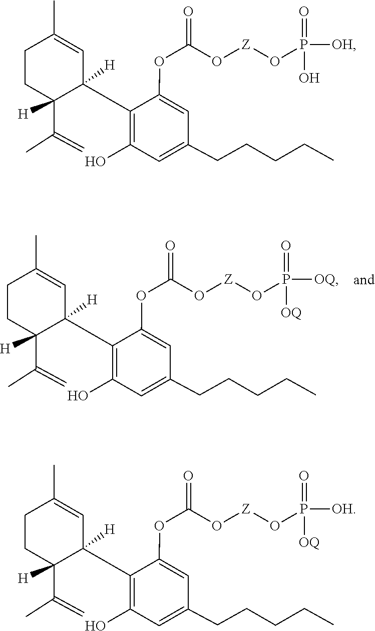

In some embodiments, the compound of Formula (I) is chosen from compounds

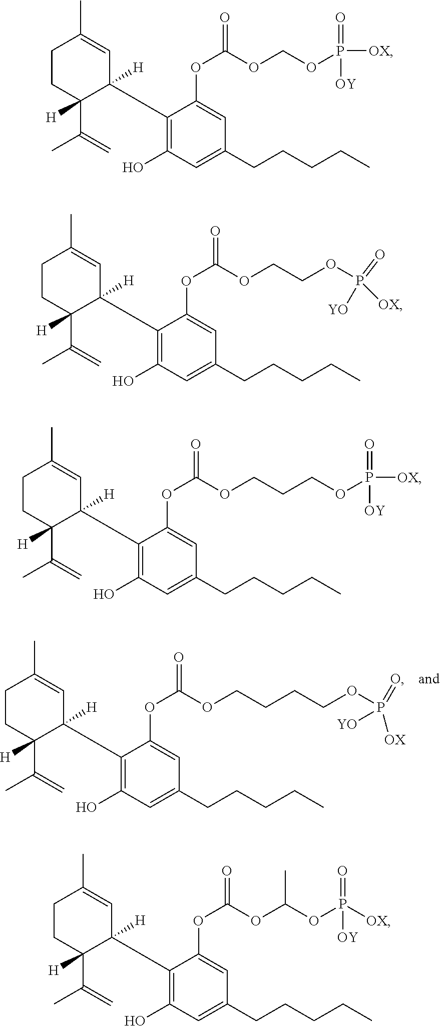

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃

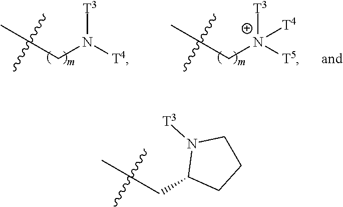

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

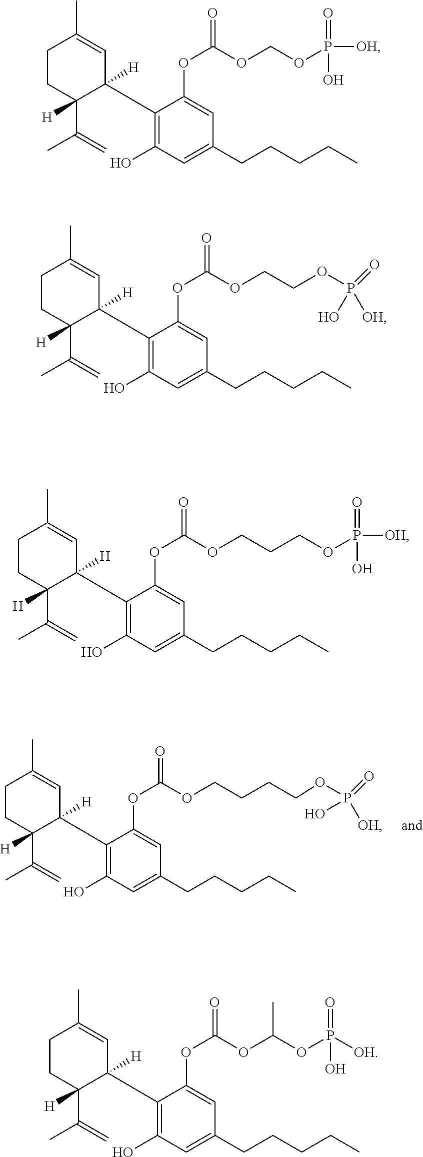 [SMILES: C=C(C)[C@@H]1CCC(C)=C[C@H]1c1c(O)cc(CCCCC)cc1OC(=O)OCOP(=O)(O)O ; C=C(C)[C@@H]1CCC(C)=C[C@H]1c1c(O)cc(CCCCC)cc1OC(=O)OCCOP(=O)(O)O ; C=C(C)[C@@H]1CCC(C)=C[C@H]1c1c(O)cc(CCCCC)cc1OC(=O)OCCCOP(=O)(O)O ; C=C(C)[C@@H]1CCC(C)=C[C@H]1c1c(O)cc(CCCCC)cc1OC(=O)OCCCCOP(=O)(O)O ; "and" ; C=C(C)[C@@H]1CCC(C)=C[C@H]1c1c(O)cc(CCCCC)cc1OC(=O)OC(C)OP(=O)(O)O]

In some embodiments, the compound of Formula (I) is chosen from compounds having the following formulae:

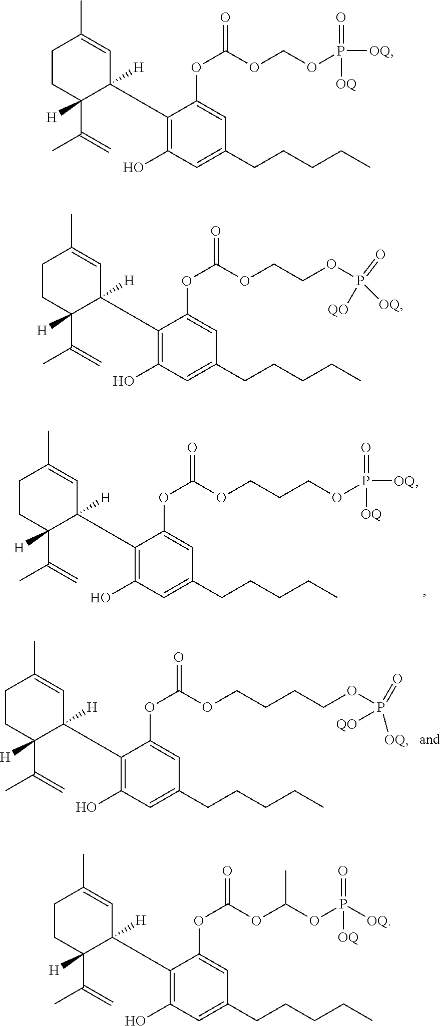

In some embodiments, the compound of Formula (I) is chosen from compounds

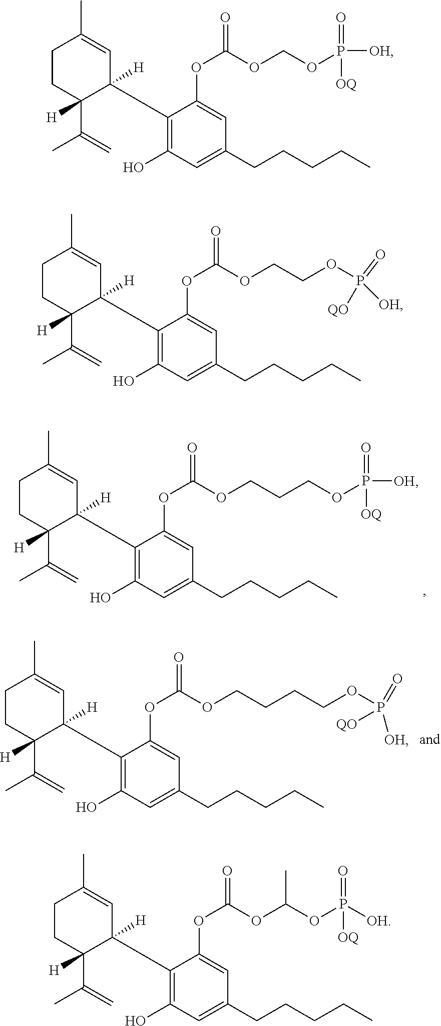

In some embodiments, the compound of Formula (I) is chosen from compounds having the following formula:

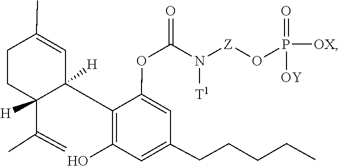

and pharmaceutically acceptable salts of any of the foregoing. In some embodiments, T¹ is H. In some embodiments, T¹ is methyl. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

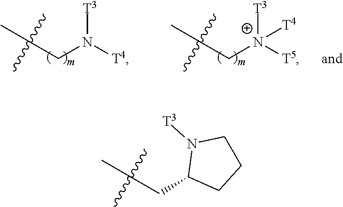

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

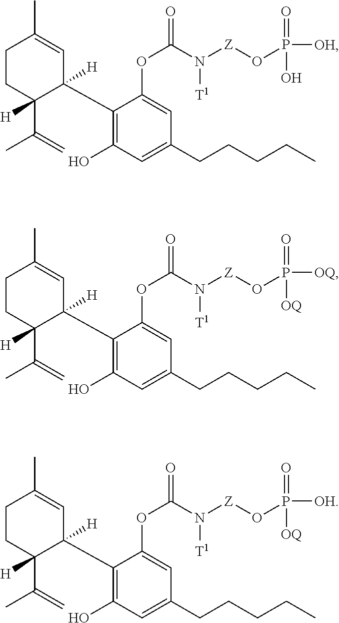

In some embodiments, T¹ is H. In some embodiments, T¹ is methyl

In some embodiments the compound of Formula (I) is chosen from compounds having the following formulae:

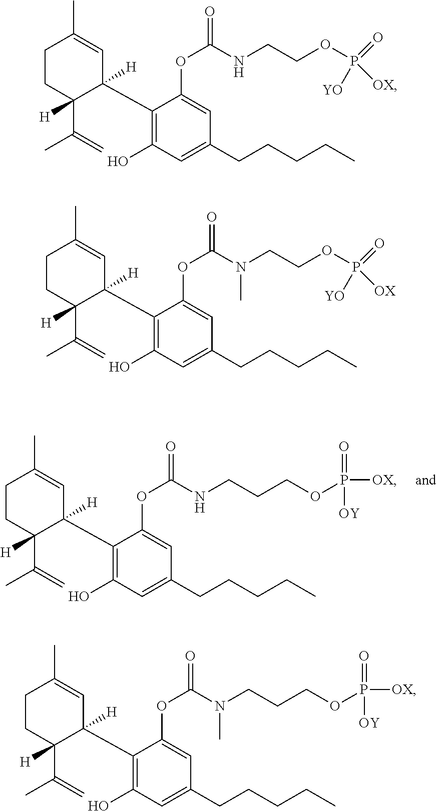

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, (CH₂CH₂O)ₙCH₃

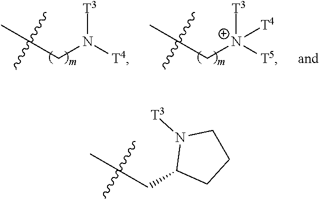

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

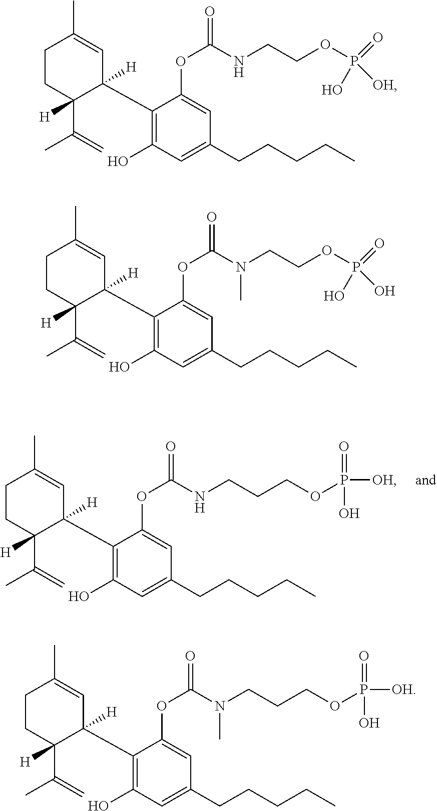

In some embodiments, the compound of Formula (I) is chosen from compounds having the following formulae:

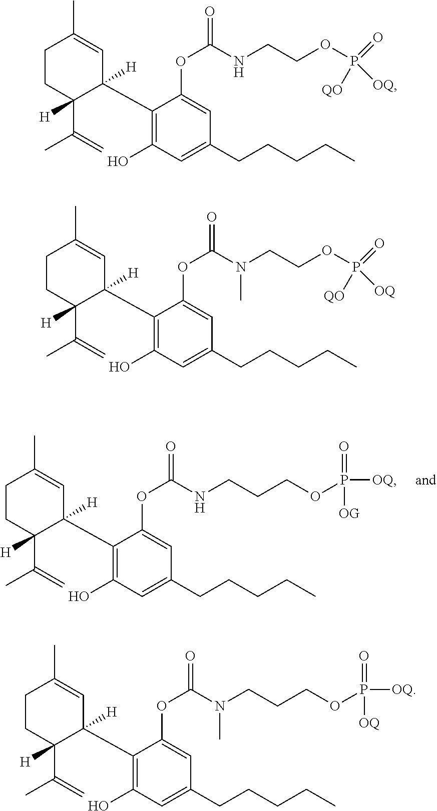

In some embodiments, the compound of Formula (I) is chosen from compounds having the following formulae:

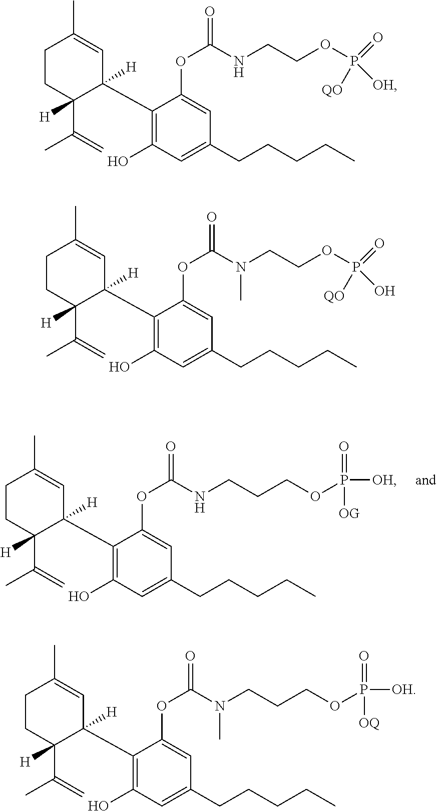

In some embodiments, the compound of Formula (I) is chosen from compounds having the following formula:

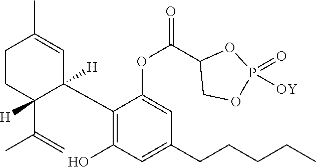

and pharmaceutically acceptable salts thereof.

In some embodiments, the compound of Formula (I) is chosen from compounds having the following formulae:

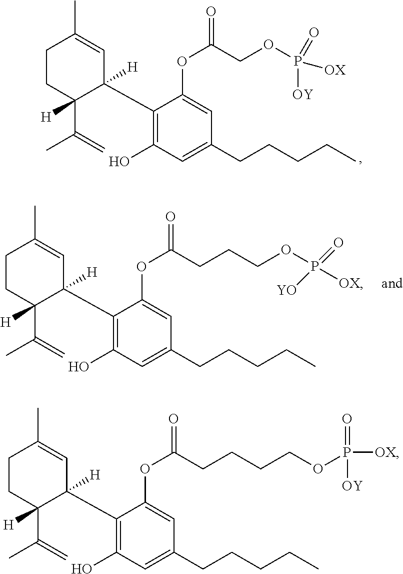

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃,

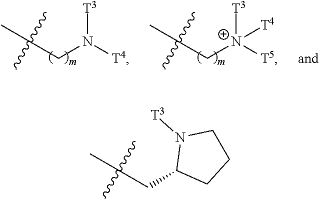

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

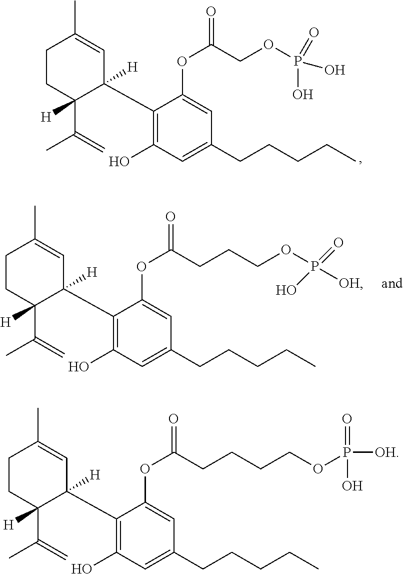

In some embodiments, the compound of Formula (I) is chosen from compounds having the following formulae.

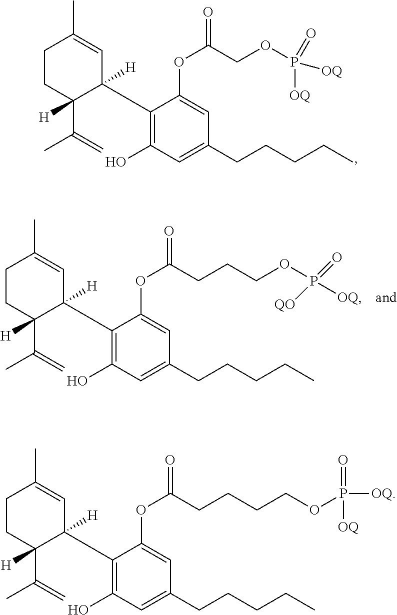

In some embodiments, the compound of Formula (I) is chosen from compounds having the following formulae:

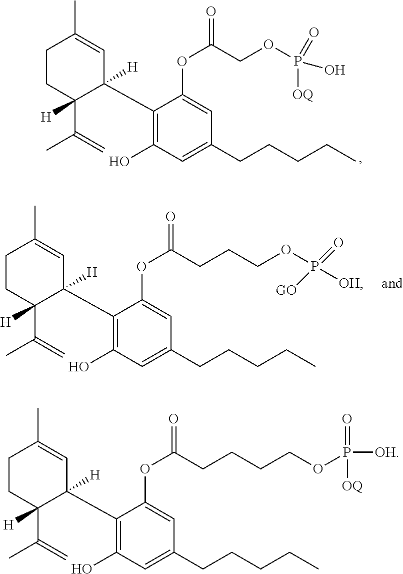

In some embodiments, the compound of Formula (I) is chosen from compounds having the following formulae:

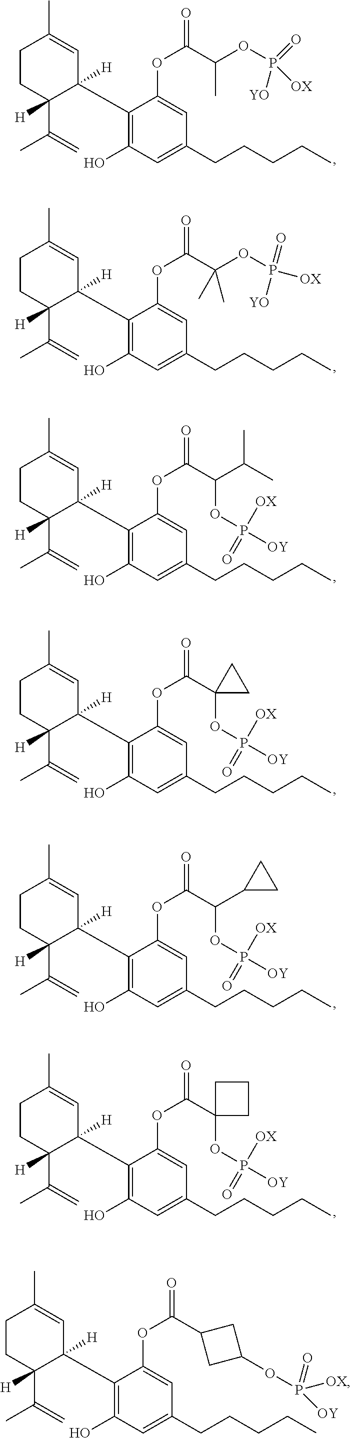

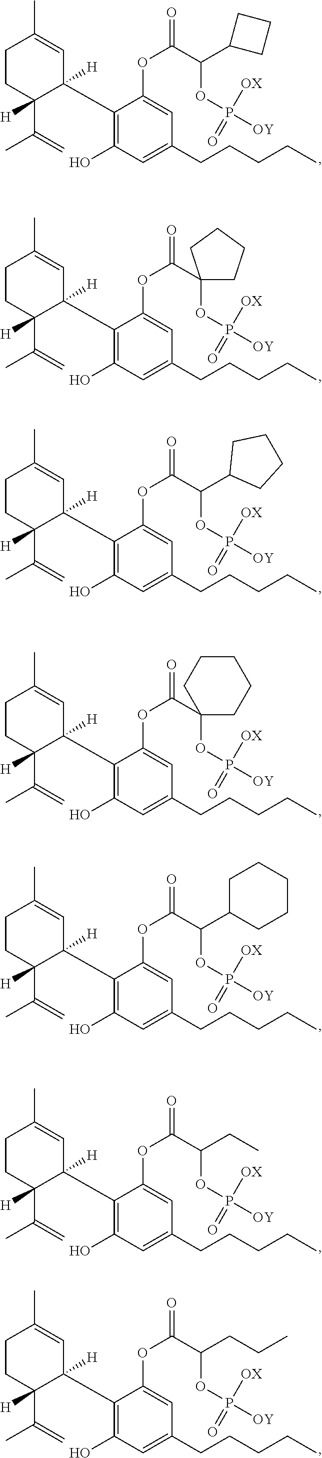

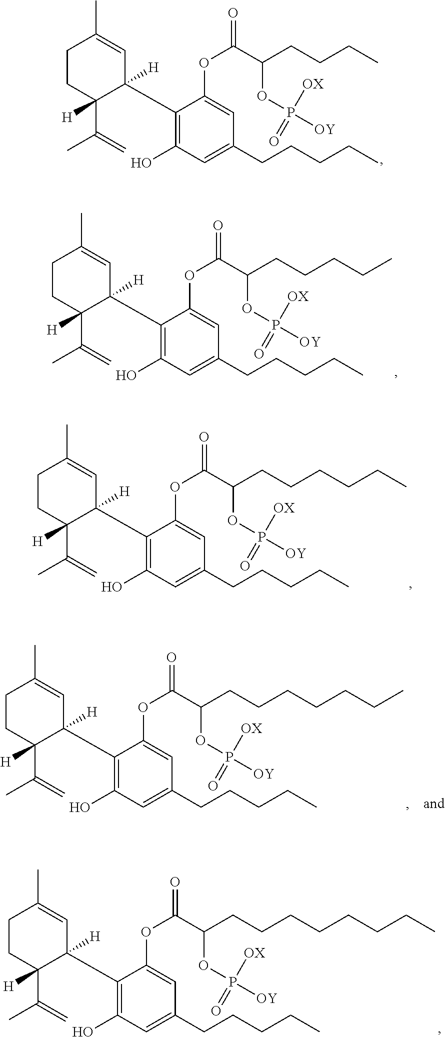

and pharmaceutically acceptable salts of any of the foregoing. In some embodiments, X is H. In some embodiments, X is Q. In some embodiments, Y is H. In some embodiments, Y is Q.

In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

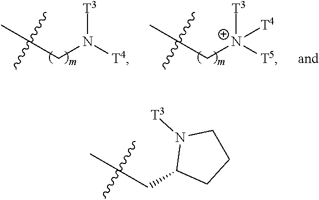

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

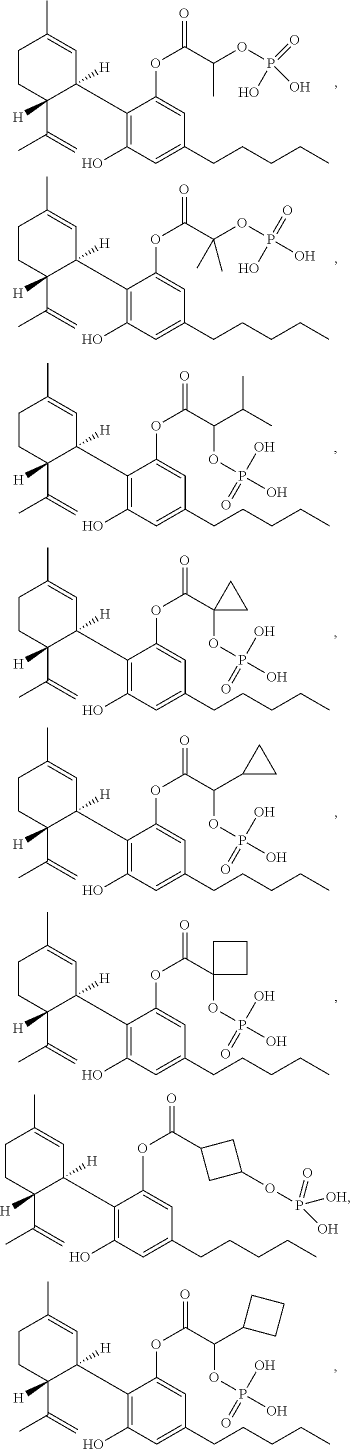

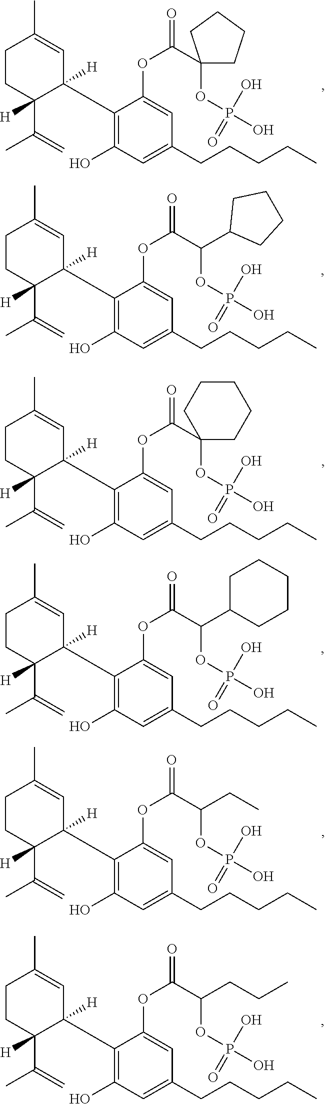

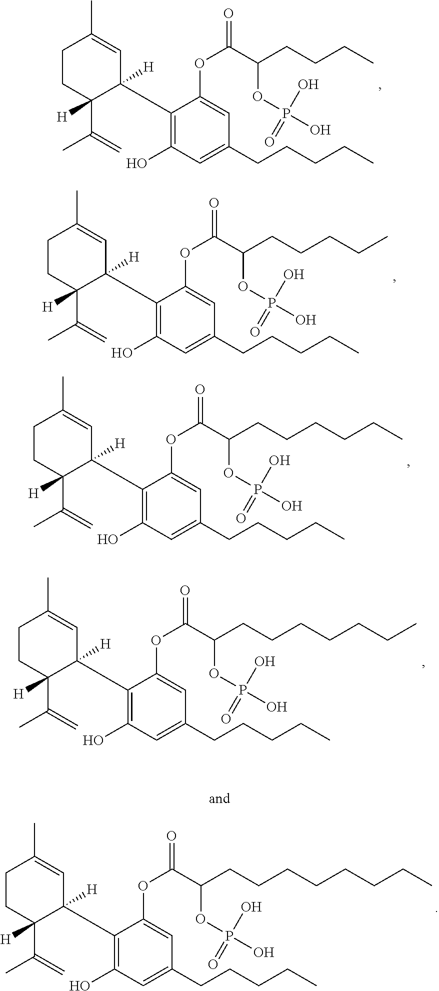

In some embodiments, the compound of Formula (I) is chosen from compounds having the following formulae:

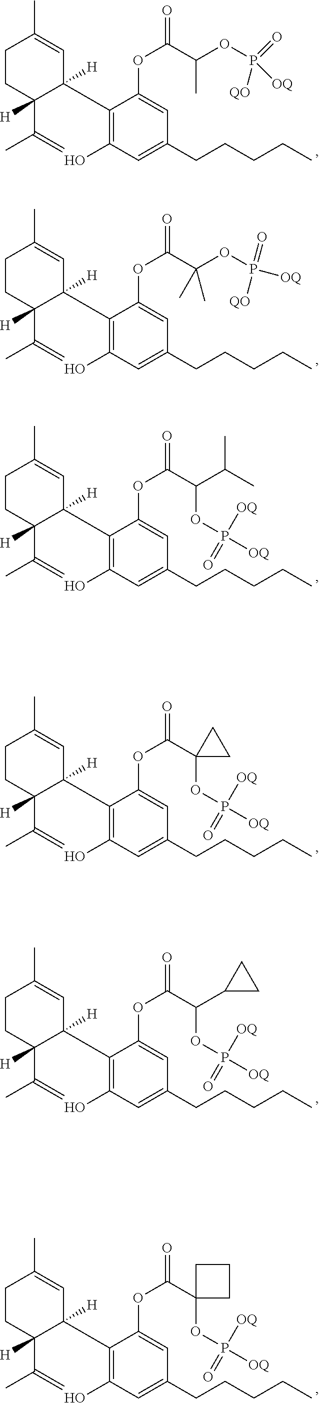

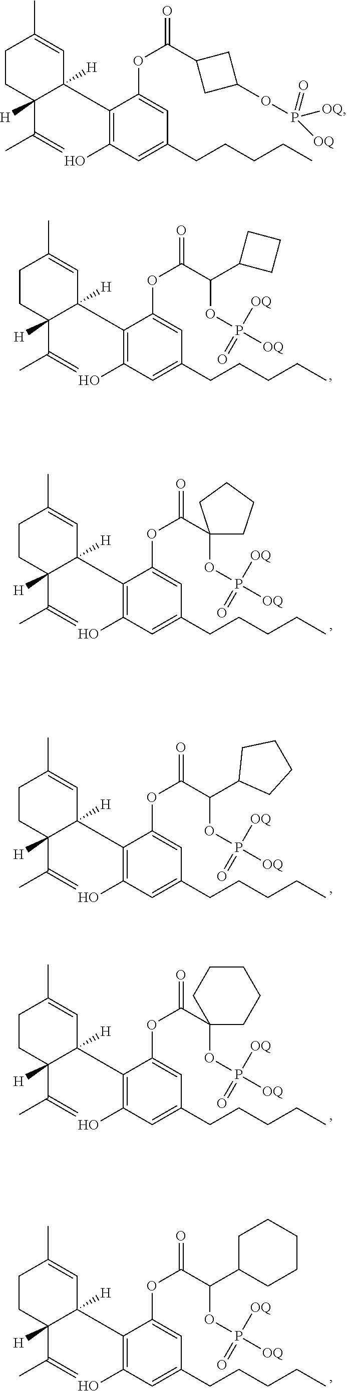

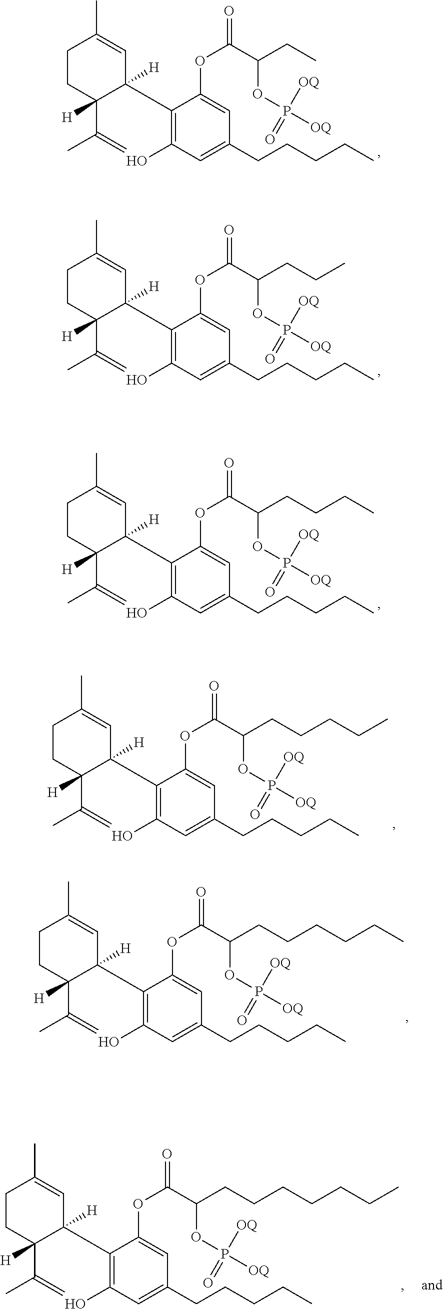

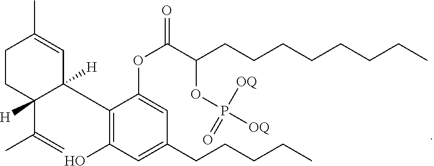

In some embodiments, the compound of Formula (I) is chosen from compounds having the following formulae:

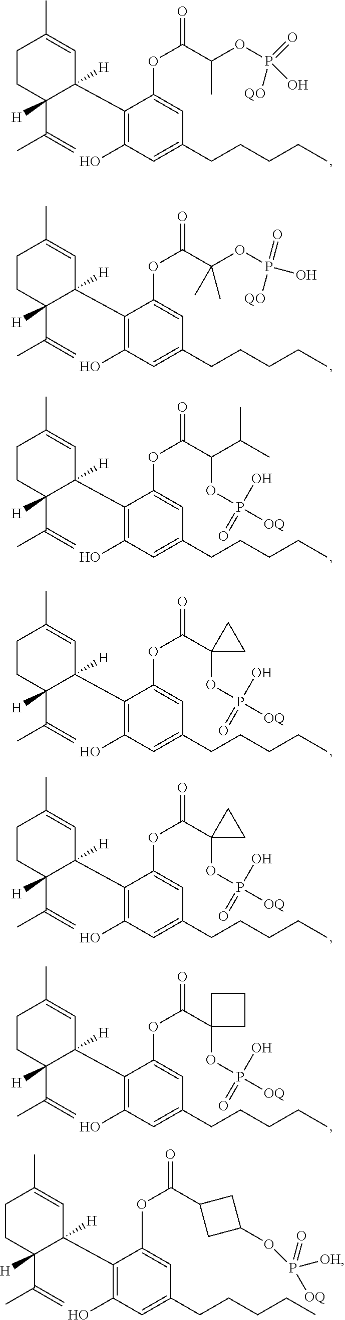

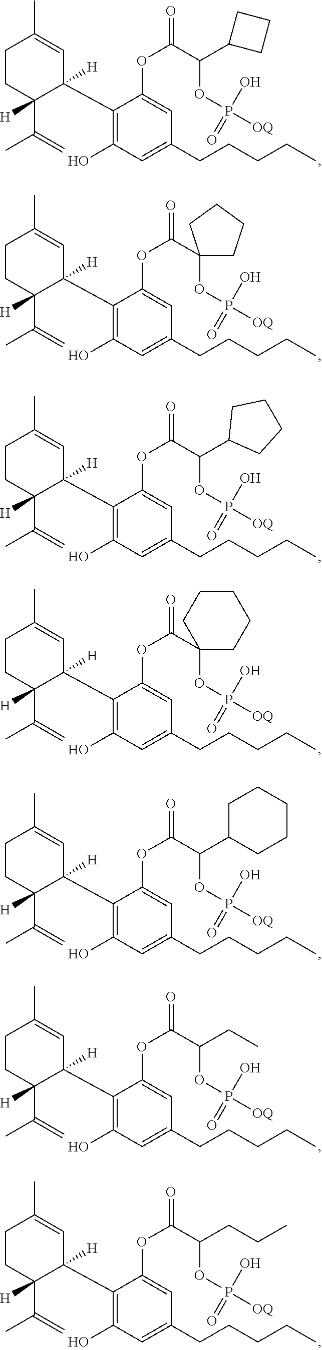

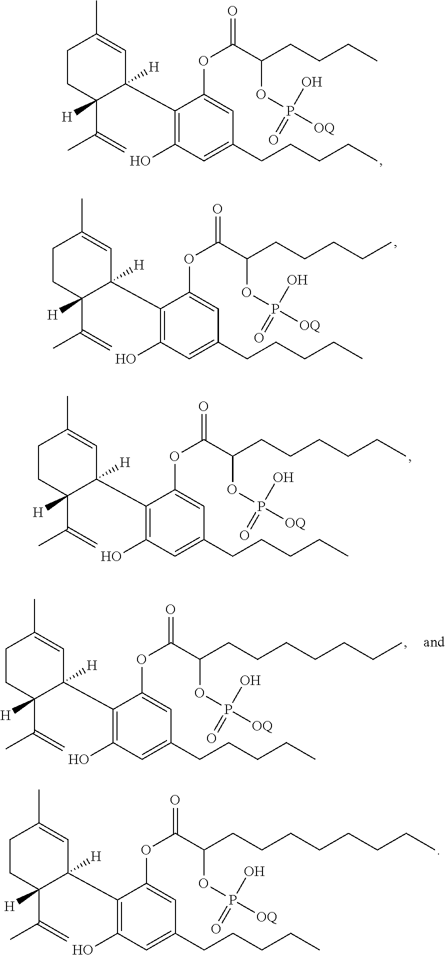

In some embodiments, the compound of Formula (I) is chosen from compounds having the following formulae:

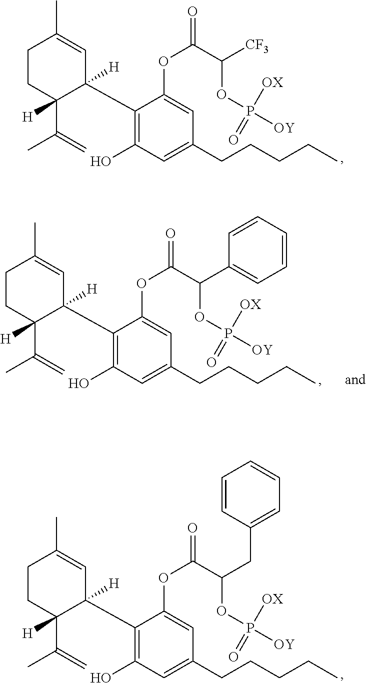

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)CH₃

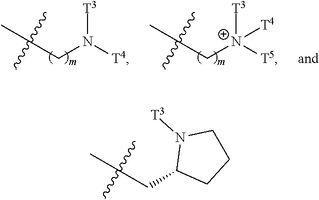

groups.

In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

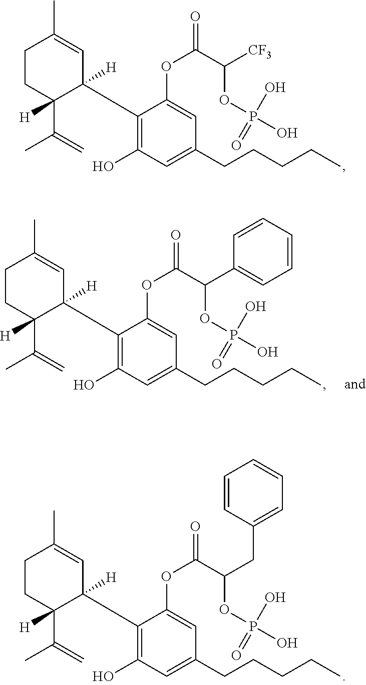 [SMILES: C=C(C)[C@@H]1CCC(C)=C[C@H]1c1c(O)cc(CCCCC)cc1OC(=O)C(OP(=O)(O)O)C(F)(F)F ; C=C(C)[C@@H]1CCC(C)=C[C@H]1c1c(O)cc(CCCCC)cc1OC(=O)C(OP(=O)(O)O)c1ccccc1 ; C=C(C)[C@@H]1CCC(C)=C[C@H]1c1c(O)cc(CCCCC)cc1OC(=O)C(Cc1ccccc1)OP(=O)(O)O]

In some embodiments, the compound of Formula (I) is chosen from compounds having the following formulae:

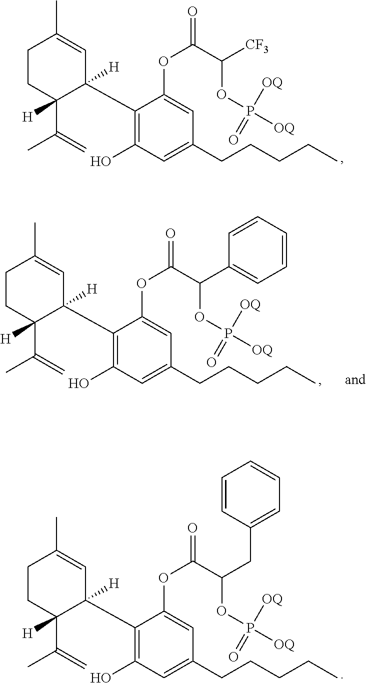

In some embodiments, the compound of Formula (I) is chosen from compounds having the following formulae:

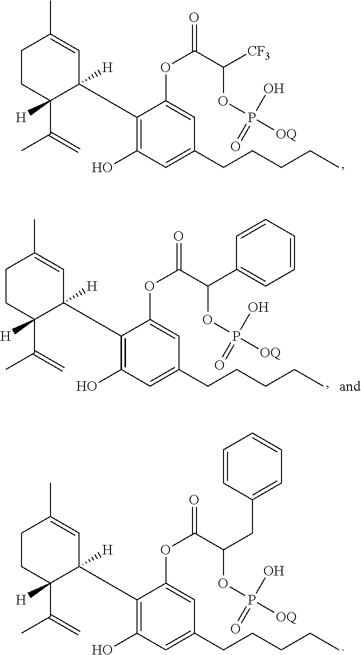

In some embodiments, the compound of Formula (I) is chosen from compounds having the following formulae:

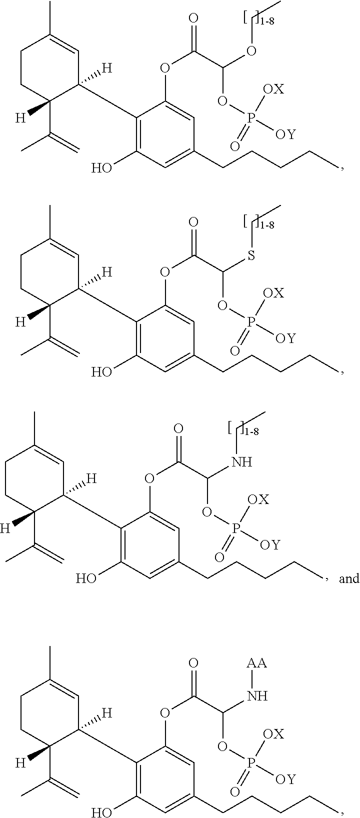

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl,
C₂₋₁₄ heteroarylalkyl, —(CH₂CH₂O)ₙCH₃,

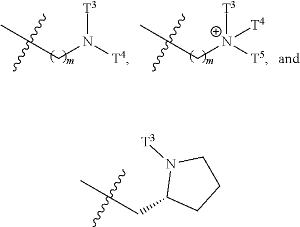

groups.

In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following forumlae:

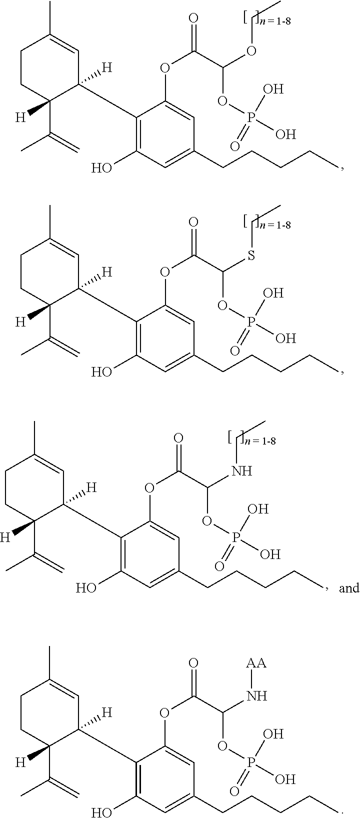

In some embodiments, the compound of Formula (I) is chosen from compounds having the following formulae:

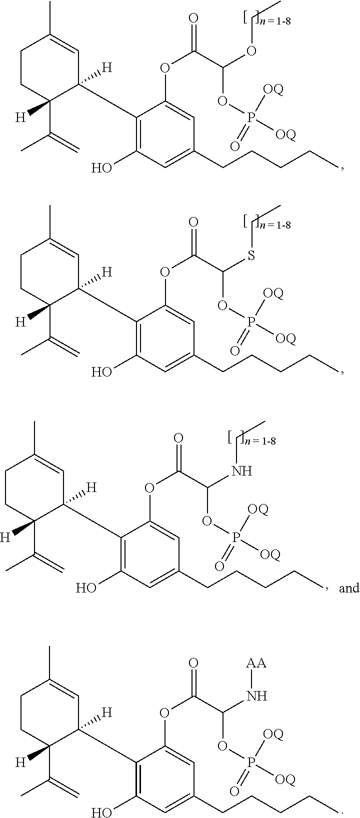

In some embodiments, the compound of Formula (I) is chosen from compounds having the following formulae:

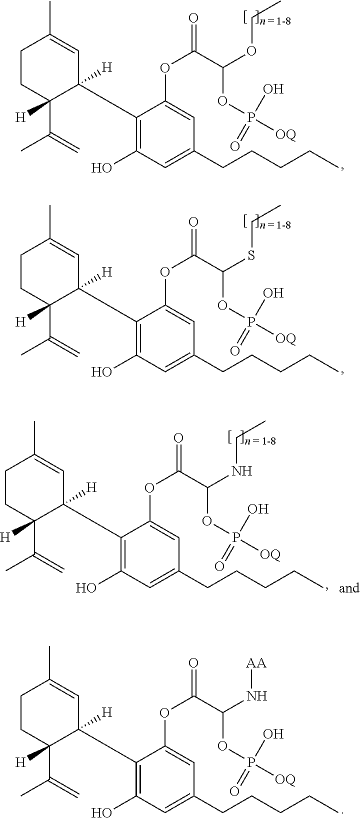

In some embodiments, the compound of Formula (I) is chosen from compounds having the following formulae:

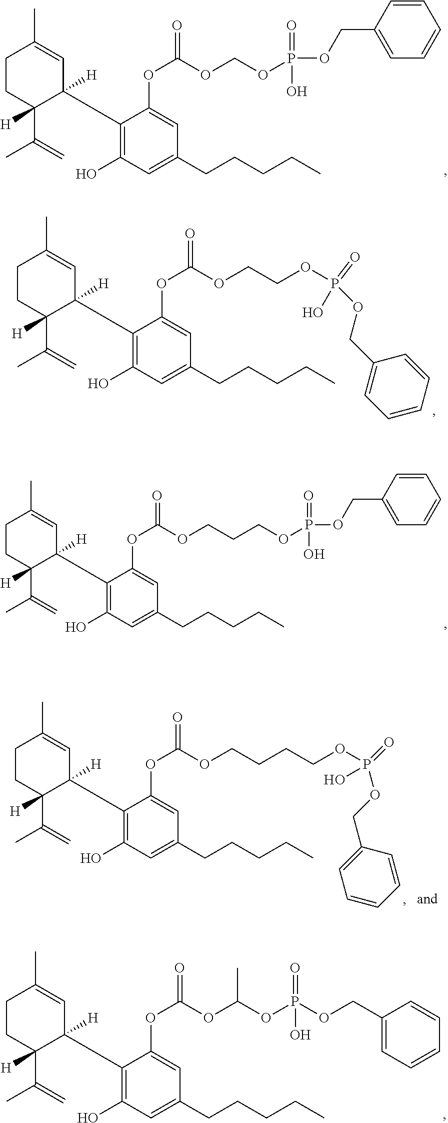 [SMILES: C=C(C)[C@@H]1CCC(C)=C[C@H]1c1c(O)cc(CCCCC)cc1OC(=O)OCOP(=O)(O)OCc1ccccc1 ; C=C(C)[C@@H]1CCC(C)=C[C@H]1c1c(O)cc(CCCCC)cc1OC(=O)OCCOP(=O)(O)OCc1ccccc1 ; C=C(C)[C@@H]1CCC(C)=C[C@H]1c1c(O)cc(CCCCC)cc1OC(=O)OCCCOP(=O)(O)OCc1ccccc1 ; C=C(C)[C@@H]1CCC(C)=C[C@H]1c1c(O)cc(CCCCC)cc1OC(=O)OCCCCOP(=O)(O)OCc1ccccc1 ; C=C(C)[C@@H]1CCC(C)=C[C@H]1c1c(O)cc(CCCCC)cc1OC(=O)OC(C)OP(=O)(O)OCc1ccccc1]

and pharmaceutically acceptable salts of any of the foregoing.

In some embodiments, the compound of Formula (I) is chosen form compounds having the following formulae:

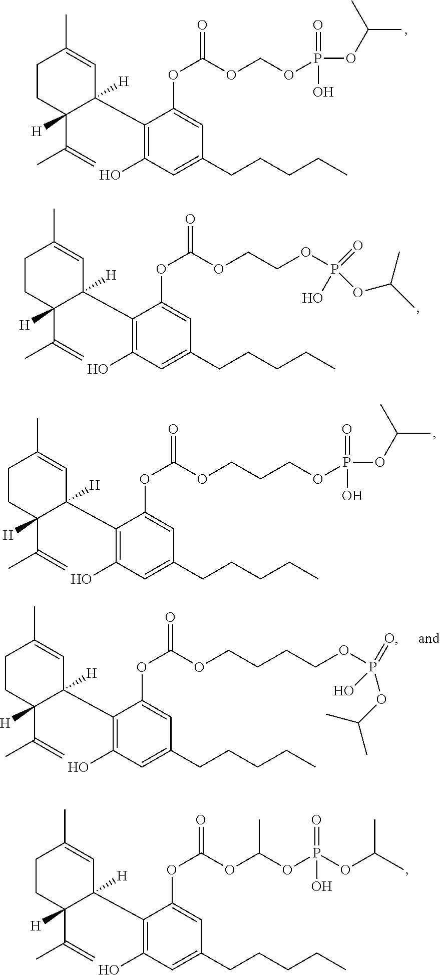

and pharmaceutically acceptable salts of any of the foregoing.

In some embodiments, the compound of Formula (I) is chosen from compounds having the following formulae:

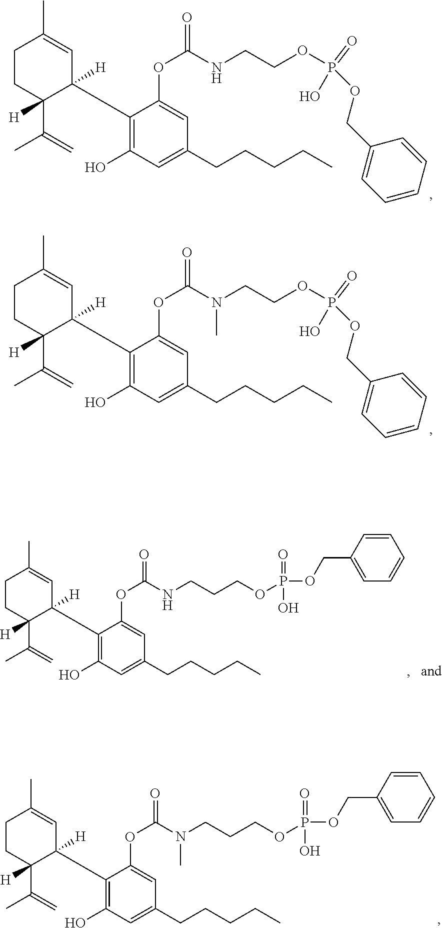 [SMILES: C=C(C)[C@@H]1CCC(C)=C[C@H]1c1c(O)cc(CCCCC)cc1OC(=O)NCCOP(=O)(O)OCc1ccccc1 ; C=C(C)[C@@H]1CCC(C)=C[C@H]1c1c(O)cc(CCCCC)cc1OC(=O)N(C)CCOP(=O)(O)OCc1ccccc1 ; C=C(C)[C@@H]1CCC(C)=C[C@H]1c1c(O)cc(CCCCC)cc1OC(=O)NCCCOP(=O)(O)OCc1ccccc1 ; C=C(C)[C@@H]1CCC(C)=C[C@H]1c1c(O)cc(CCCCC)cc1OC(=O)N(C)CCCOP(=O)(O)OCc1ccccc1]

and pharmaceutically acceptable salts of any of the foregoing.

In some embodiments, the compound of Formula (I) is chosen form compounds

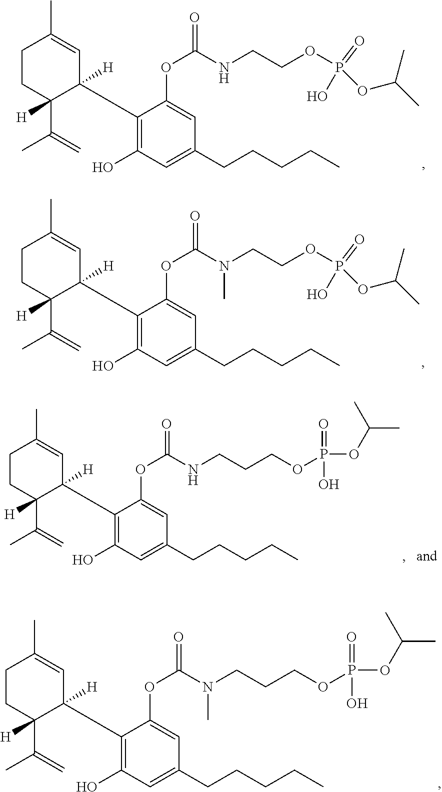

and pharmaceutically acceptable salts of any of the foregoing.

In some embodiments, the compound of Formula (I) is chosen from compounds having the following formula:

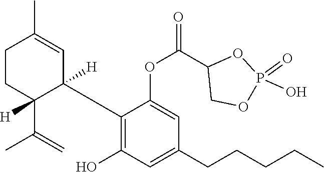 [SMILES: C=C(C)[C@@H]1CCC(C)=C[C@H]1c1c(O)cc(CCCCC)cc1OC(=O)C1COP(=O)(O)O1]

and pharmaceutically acceptable salts thereof.

In some embodiments, the compound of Formula (I) is chosen from compounds having the following formulae:

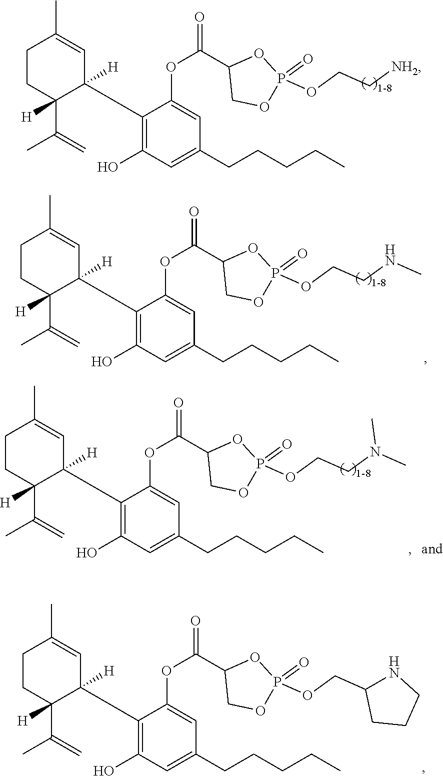

and pharmaceutically acceptable salts of any of the foregoing.

In some embodiments, the compound of Formula (I) is chosen from compounds having the following formulae:

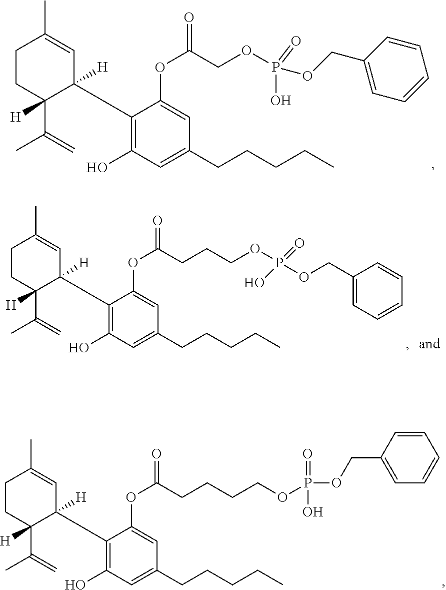 [SMILES: C=C(C)[C@@H]1CCC(C)=C[C@H]1c1c(O)cc(CCCCC)cc1OC(=O)COP(=O)(O)OCc1ccccc1 ; C=C(C)[C@@H]1CCC(C)=C[C@H]1c1c(O)cc(CCCCC)cc1OC(=O)CCCOP(=O)(O)OCc1ccccc1 ; C=C(C)[C@@H]1CCC(C)=C[C@H]1c1c(O)cc(CCCCC)cc1OC(=O)CCCCOP(=O)(O)OCc1ccccc1]

and pharmaceutically acceptable salts of any of the foregoing.

In some embodiments, the compound of Formula (I) is chosen form compounds having the following formulae:

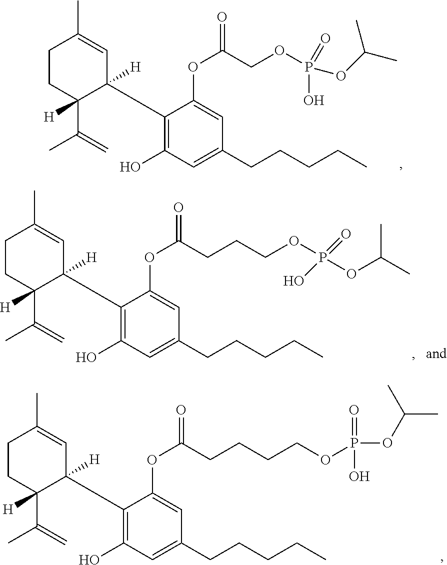

and pharmaceutically acceptable salts of any of the foregoing.

In some embodiments, the compound of Formula (I) is chosen from compounds

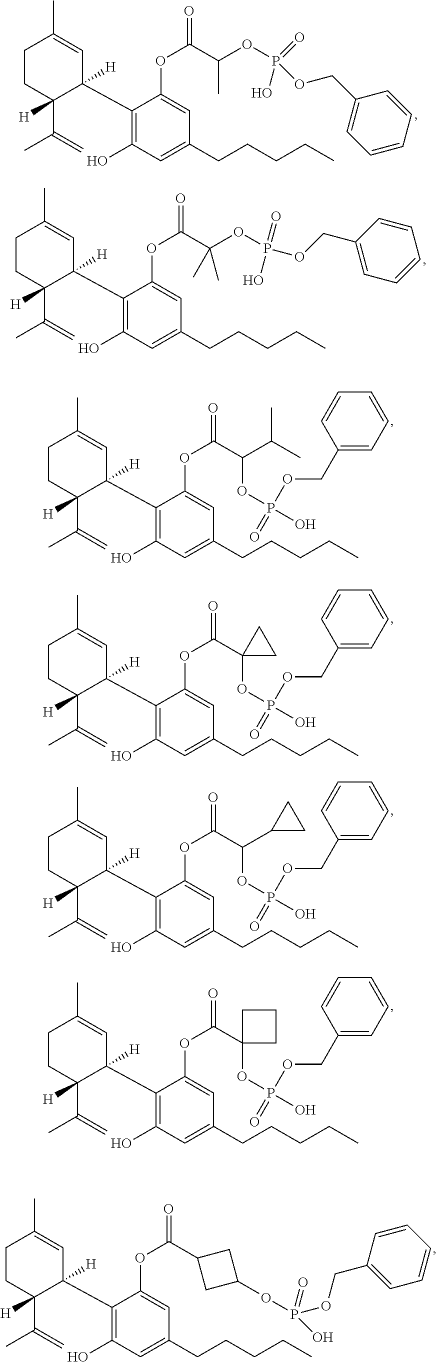

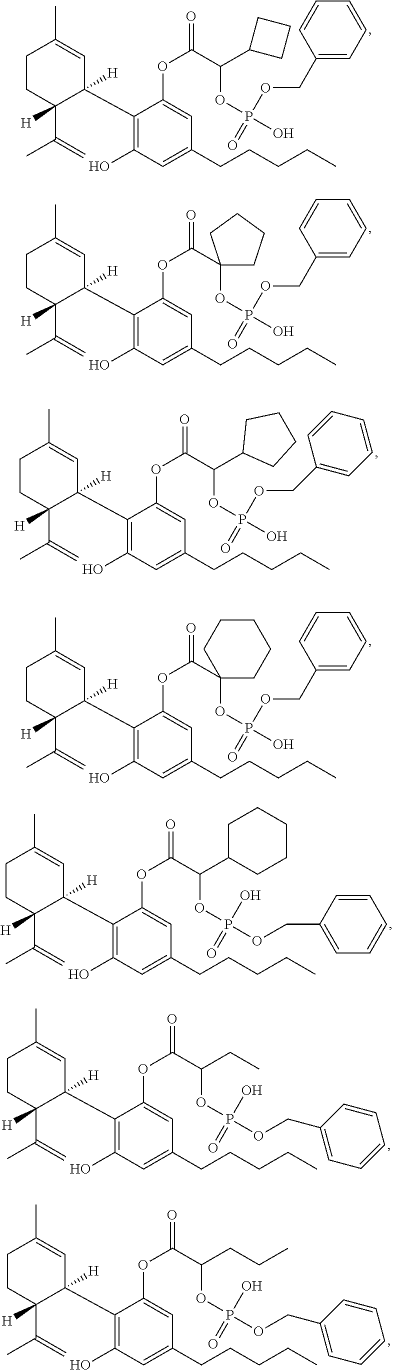

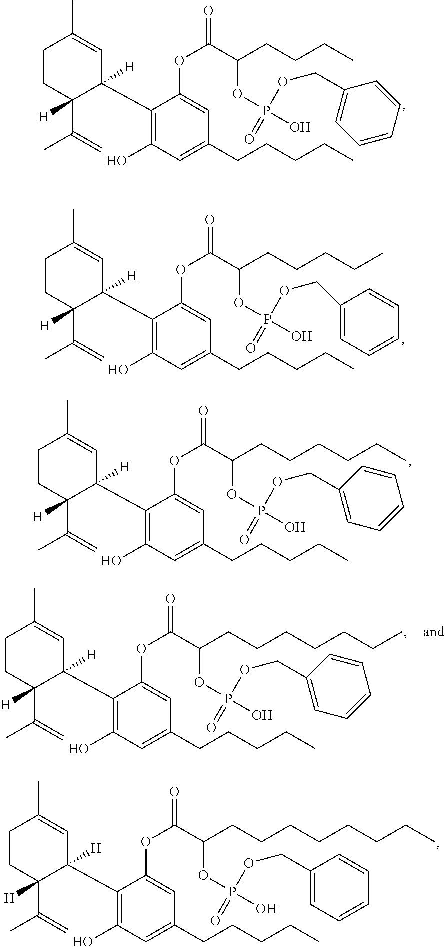

and pharmaceutically acceptable salts of any of the foregoing.

In some embodiments, the compound of Formula (I) is chosen form compounds having the following formulae:

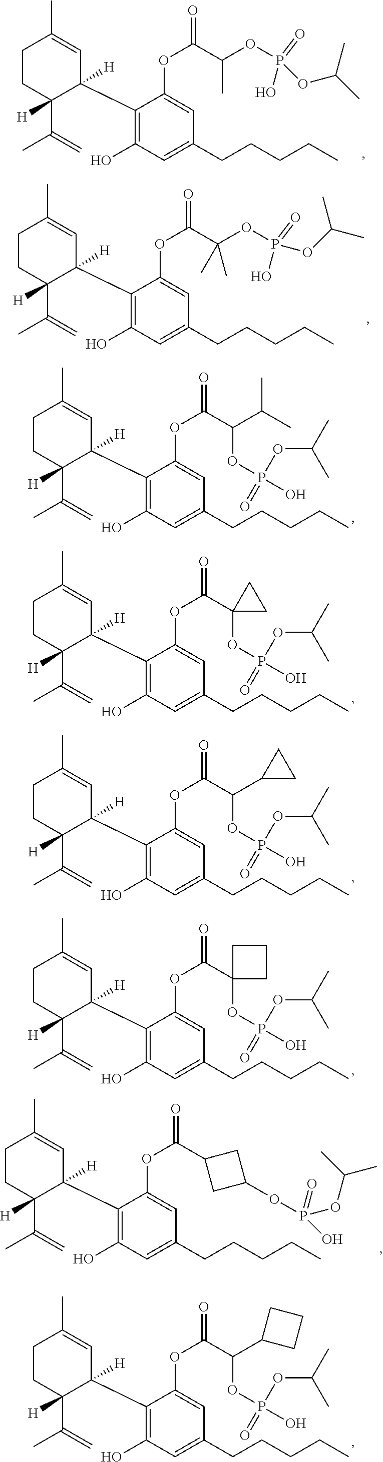

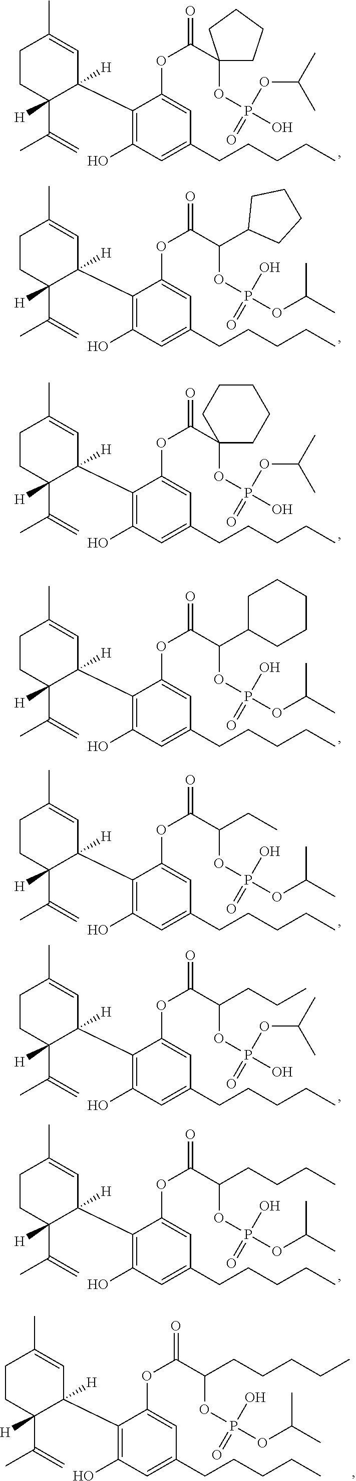

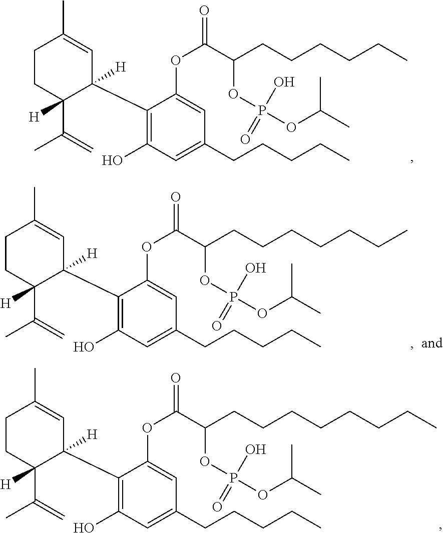

and pharmaceutically acceptable salts of any of the foregoing.

In some embodiments, the compound of Formula (I) is chosen from compounds having the following formulae:

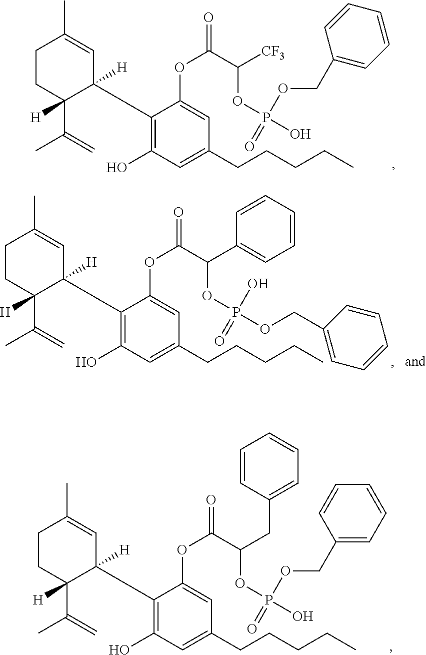

and pharmaceutically acceptable salts of any of the foregoing.

In some embodiments, the compound of Formula (I) is chosen form compounds having the following formulae:

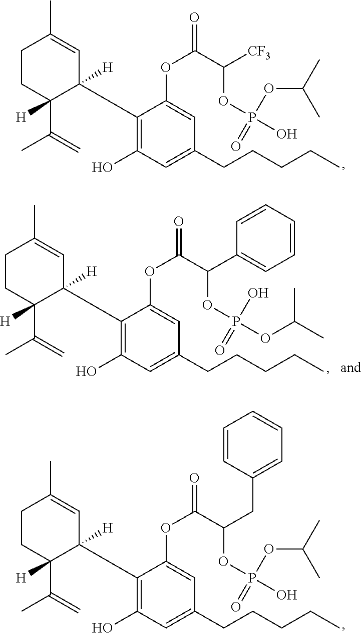

and pharmaceutically acceptable salts of any of the foregoing.

In some embodiments, the compound of Formula (I) is chosen from compounds

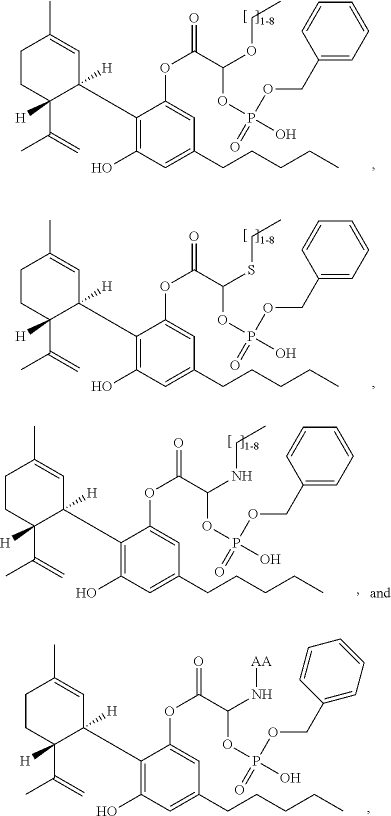

and pharmaceutically acceptable salts of any of the foregoing.

In some embodiments, the compound of Formula (I) is chosen form compounds having the following formulae:

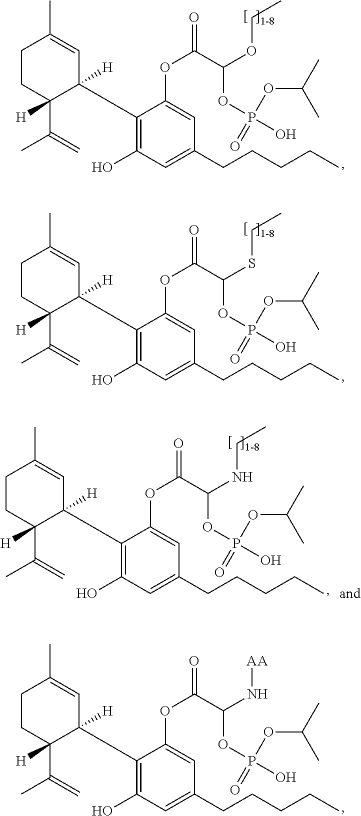

and pharmaceutically acceptable salts of any of the foregoing.

In some embodiments, the compound of Formula (I) is chosen from compounds having the following formula:

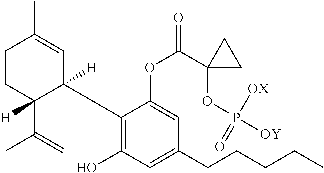

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl,
C₂₋₁₄ heteroarylalkyl, —(CH₂CH₂O)ₙCH₃,

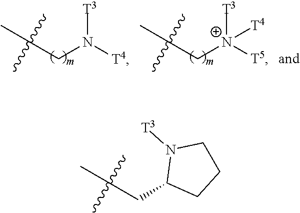

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formula:

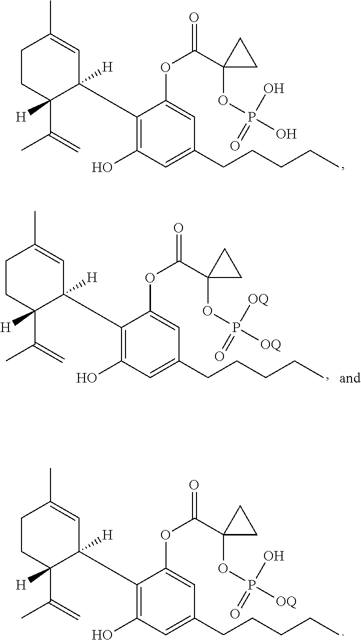

In some embodiments, the compound of Formula (I) is chosen from compounds having the following formula:

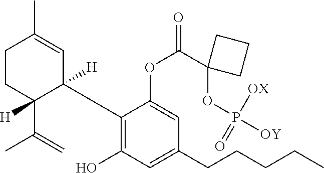

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl,
C₂₋₁₄ heteroarylalkyl, —(CH₂CH₂O)ₙCH₃

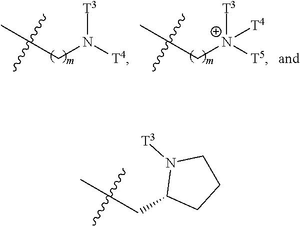

groups.

In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

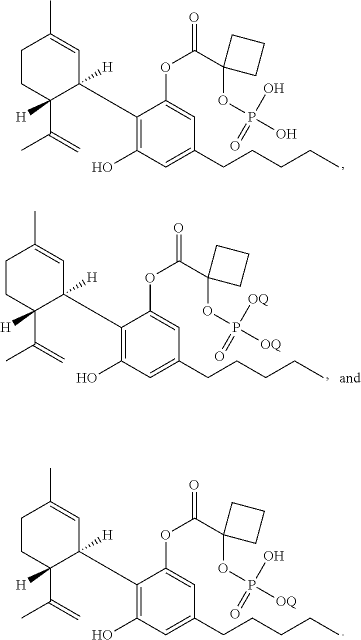

In some embodiments, the compound of Formula (I) is chosen from compounds having the following formula:

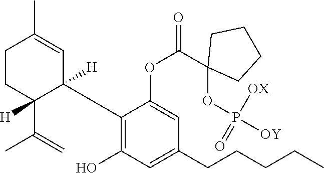

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃

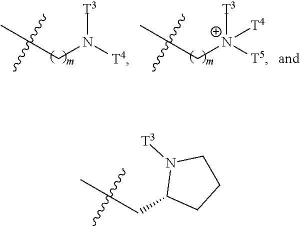

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

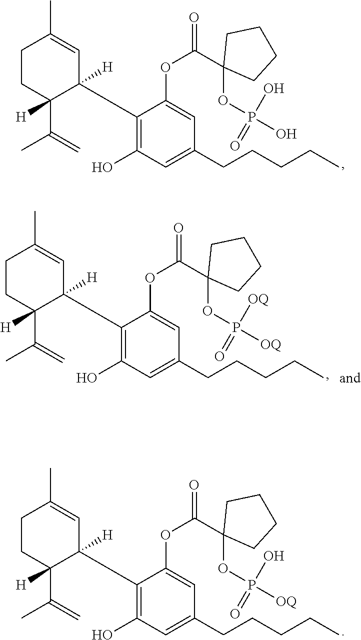

In some embodiments, the compound of Formula (I) is chosen from compounds having the following formula:

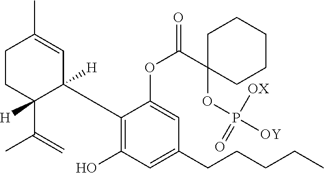

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃

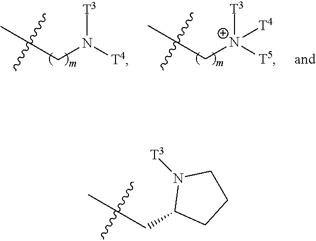

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

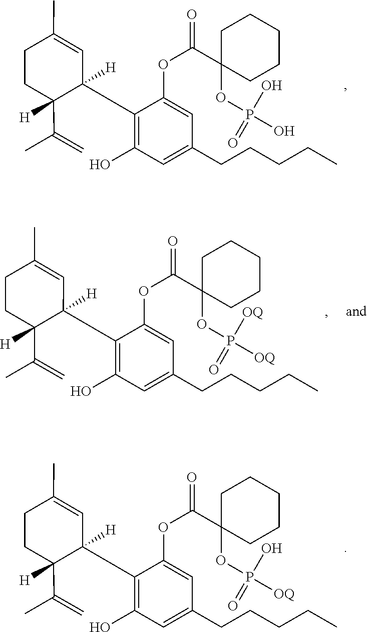

In some embodiments, the compound of Formula (I) is chosen from compounds having the following formulae:

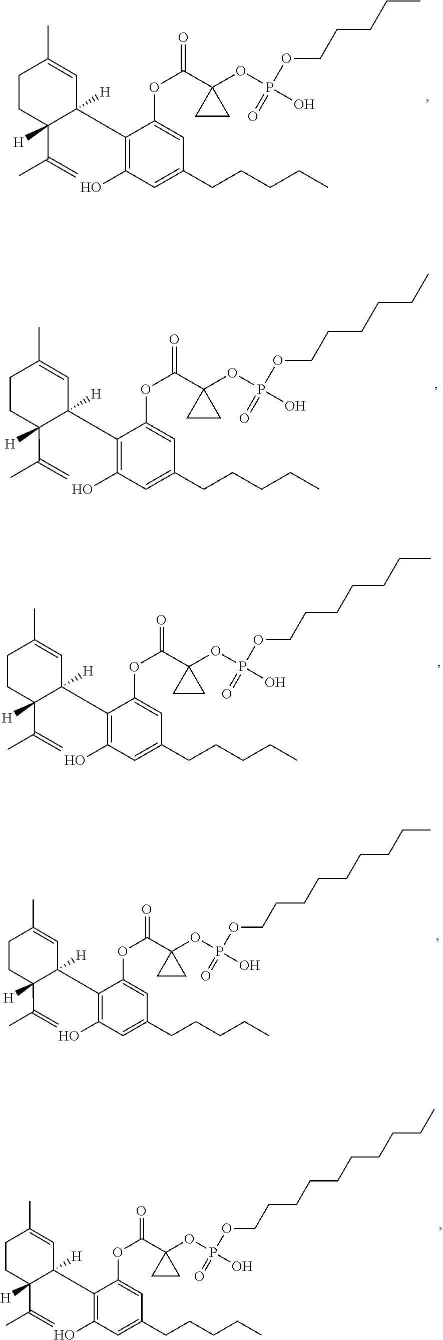 [SMILES: C=C(C)[C@@H]1CCC(C)=C[C@H]1c1c(O)cc(CCCCC)cc1OC(=O)C1(OP(=O)(O)OCCCCC)CC1 ; C=C(C)[C@@H]1CCC(C)=C[C@H]1c1c(O)cc(CCCCC)cc1OC(=O)C1(OP(=O)(O)OCCCCCC)CC1 ; C=C(C)[C@@H]1CCC(C)=C[C@H]1c1c(O)cc(CCCCC)cc1OC(=O)C1(OP(=O)(O)OCCCCCCC)CC1 ; C=C(C)[C@@H]1CCC(C)=C[C@H]1c1c(O)cc(CCCCC)cc1OC(=O)C1(OP(=O)(O)OCCCCCCCCC)CC1 ; C=C(C)[C@@H]1CCC(C)=C[C@H]1c1c(O)cc(CCCCC)cc1OC(=O)C1(OP(=O)(O)OCCCCCCCCCC)CC1]

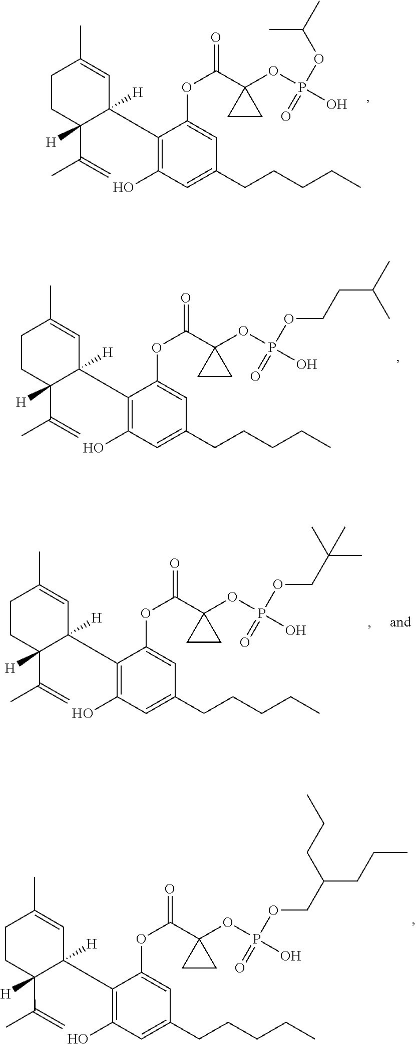 [SMILES: C=C(C)[C@@H]1CCC(C)=C[C@H]1c1c(O)cc(CCCCC)cc1OC(=O)C1(OP(=O)(O)OC(C)C)CC1 ; C=C(C)[C@@H]1CCC(C)=C[C@H]1c1c(O)cc(CCCCC)cc1OC(=O)C1(OP(=O)(O)OCCC(C)C)CC1 ; C=C(C)[C@@H]1CCC(C)=C[C@H]1c1c(O)cc(CCCCC)cc1OC(=O)C1(OP(=O)(O)OCC(C)(C)C)CC1 ; C=C(C)[C@@H]1CCC(C)=C[C@H]1c1c(O)cc(CCCCC)cc1OC(=O)C1(OP(=O)(O)OCC(CCC)CCC)CC1]

and pharmaceutically acceptable salts of any of the foregoing.

In some embodiments, the compound of Formula (I) is chosen from compounds having the following formulae:

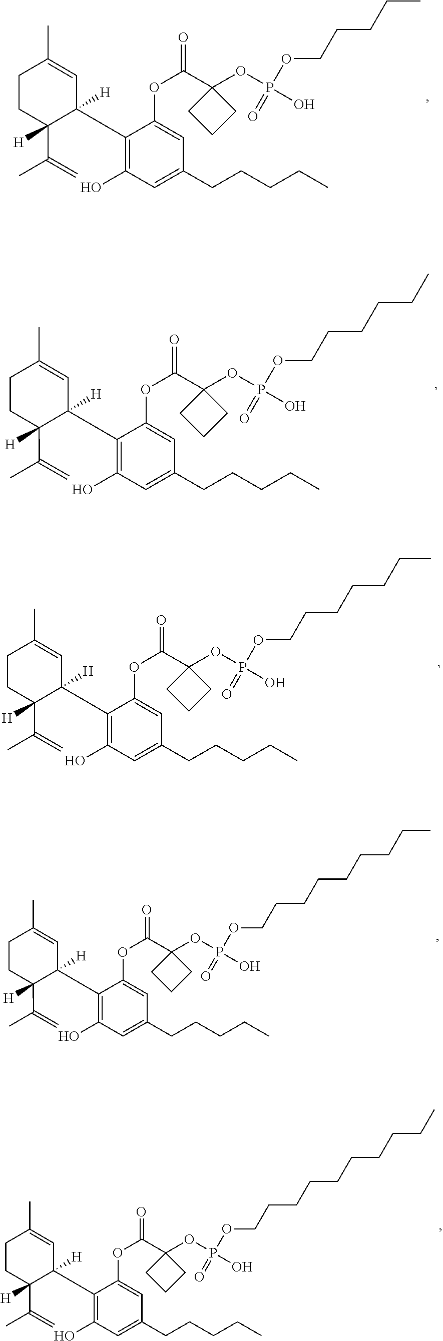 [SMILES: C=C(C)[C@@H]1CCC(C)=C[C@H]1c1c(O)cc(CCCCC)cc1OC(=O)C1(OP(=O)(O)OCCCCC)CCC1 ; C=C(C)[C@@H]1CCC(C)=C[C@H]1c1c(O)cc(CCCCC)cc1OC(=O)C1(OP(=O)(O)OCCCCCC)CCC1 ; C=C(C)[C@@H]1CCC(C)=C[C@H]1c1c(O)cc(CCCCC)cc1OC(=O)C1(OP(=O)(O)OCCCCCCC)CCC1 ; C=C(C)[C@@H]1CCC(C)=C[C@H]1c1c(O)cc(CCCCC)cc1OC(=O)C1(OP(=O)(O)OCCCCCCCCC)CCC1 ; C=C(C)[C@@H]1CCC(C)=C[C@H]1c1c(O)cc(CCCCC)cc1OC(=O)C1(OP(=O)(O)OCCCCCCCCCC)CCC1]

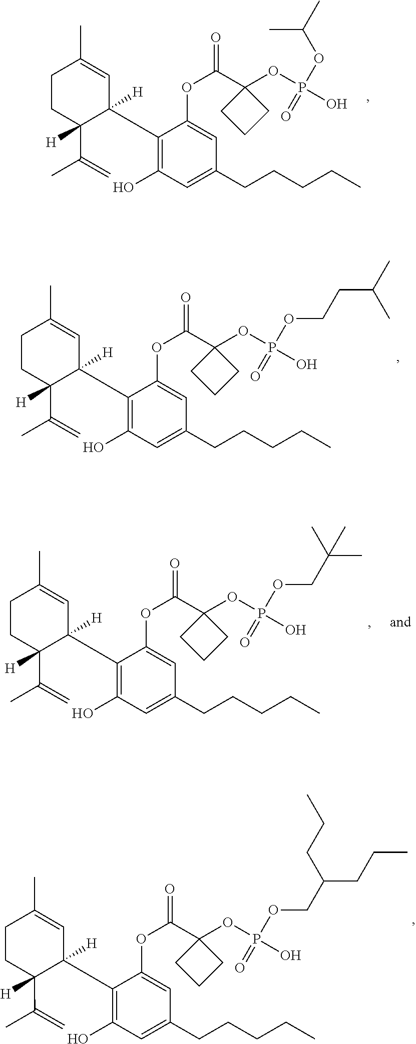 [SMILES: C=C(C)[C@@H]1CCC(C)=C[C@H]1c1c(O)cc(CCCCC)cc1OC(=O)C1(OP(=O)(O)OC(C)C)CCC1 ; C=C(C)[C@@H]1CCC(C)=C[C@H]1c1c(O)cc(CCCCC)cc1OC(=O)C1(OP(=O)(O)OCCC(C)C)CCC1 ; C=C(C)[C@@H]1CCC(C)=C[C@H]1c1c(O)cc(CCCCC)cc1OC(=O)C1(OP(=O)(O)OCC(C)(C)C)CCC1 ; C=C(C)[C@@H]1CCC(C)=C[C@H]1c1c(O)cc(CCCCC)cc1OC(=O)C1(OP(=O)(O)OCC(CCC)CCC)CCC1]

and pharmaceutically acceptable salts of any of the foregoing.

In some embodiments, the compound of Formula (I) is chosen from compounds having the following formulae:

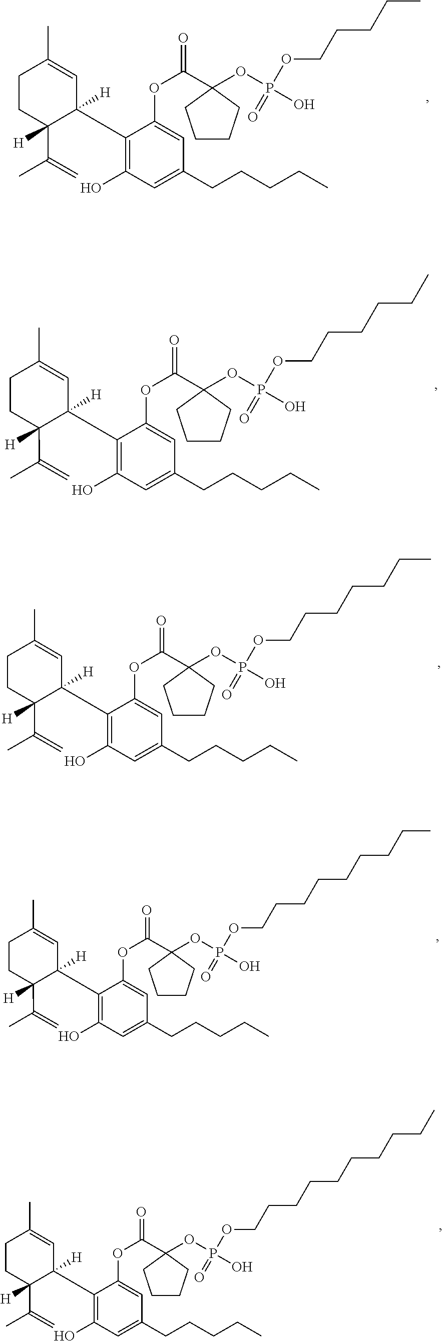 [SMILES: C=C(C)[C@@H]1CCC(C)=C[C@H]1c1c(O)cc(CCCCC)cc1OC(=O)C1(OP(=O)(O)OCCCCC)CCCC1 ; C=C(C)[C@@H]1CCC(C)=C[C@H]1c1c(O)cc(CCCCC)cc1OC(=O)C1(OP(=O)(O)OCCCCCC)CCCC1 ; C=C(C)[C@@H]1CCC(C)=C[C@H]1c1c(O)cc(CCCCC)cc1OC(=O)C1(OP(=O)(O)OCCCCCCC)CCCC1 ; C=C(C)[C@@H]1CCC(C)=C[C@H]1c1c(O)cc(CCCCC)cc1OC(=O)C1(OP(=O)(O)OCCCCCCCCC)CCCC1 ; C=C(C)[C@@H]1CCC(C)=C[C@H]1c1c(O)cc(CCCCC)cc1OC(=O)C1(OP(=O)(O)OCCCCCCCCCC)CCCC1]

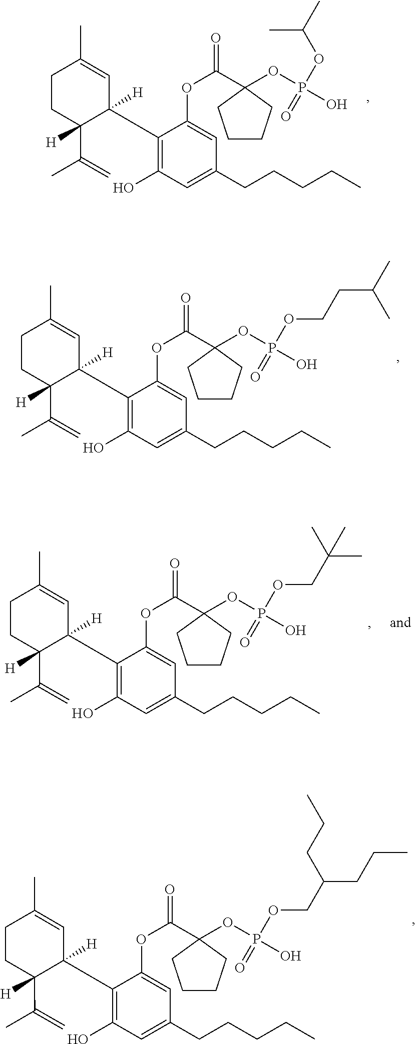 [SMILES: C=C(C)[C@@H]1CCC(C)=C[C@H]1c1c(O)cc(CCCCC)cc1OC(=O)C1(OP(=O)(O)OC(C)C)CCCC1 ; C=C(C)[C@@H]1CCC(C)=C[C@H]1c1c(O)cc(CCCCC)cc1OC(=O)C1(OP(=O)(O)OCCC(C)C)CCCC1 ; C=C(C)[C@@H]1CCC(C)=C[C@H]1c1c(O)cc(CCCCC)cc1OC(=O)C1(OP(=O)(O)OCC(C)(C)C)CCCC1 ; C=C(C)[C@@H]1CCC(C)=C[C@H]1c1c(O)cc(CCCCC)cc1OC(=O)C1(OP(=O)(O)OCC(CCC)CCC)CCCC1]

and pharmaceutically acceptable salts of any of the foregoing.

In some embodiments, the compound of Formula (I) is chosen from compounds having the following formulae:

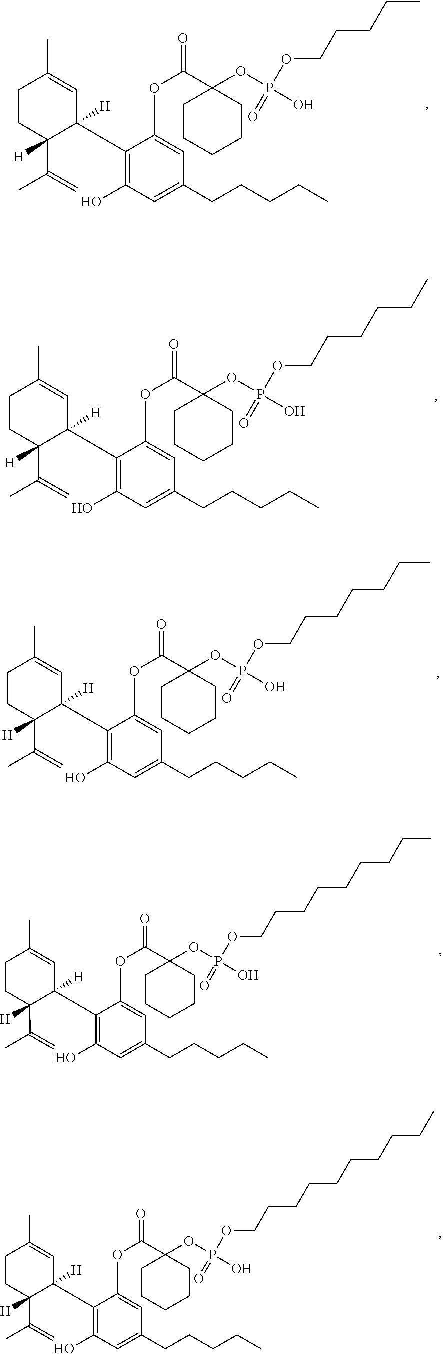 [SMILES: C=C(C)[C@@H]1CCC(C)=C[C@H]1c1c(O)cc(CCCCC)cc1OC(=O)C1(OP(=O)(O)OCCCCC)CCCCC1 ; C=C(C)[C@@H]1CCC(C)=C[C@H]1c1c(O)cc(CCCCC)cc1OC(=O)C1(OP(=O)(O)OCCCCCC)CCCCC1 ; C=C(C)[C@@H]1CCC(C)=C[C@H]1c1c(O)cc(CCCCC)cc1OC(=O)C1(OP(=O)(O)OCCCCCCC)CCCCC1 ; C=C(C)[C@@H]1CCC(C)=C[C@H]1c1c(O)cc(CCCCC)cc1OC(=O)C1(OP(=O)(O)OCCCCCCCCC)CCCCC1 ; C=C(C)[C@@H]1CCC(C)=C[C@H]1c1c(O)cc(CCCCC)cc1OC(=O)C1(OP(=O)(O)OCCCCCCCCCC)CCCCC1]

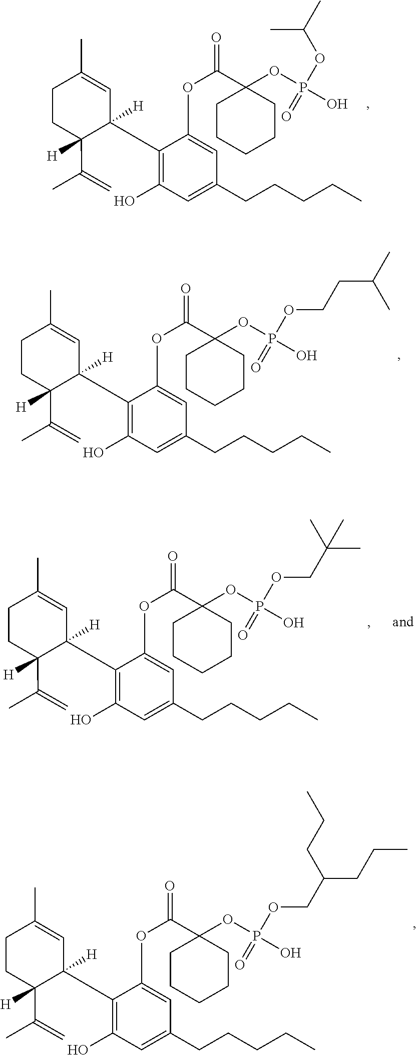 [SMILES: C=C(C)[C@@H]1CCC(C)=C[C@H]1c1c(O)cc(CCCCC)cc1OC(=O)C1(OP(=O)(O)OC(C)C)CCCCC1 ; C=C(C)[C@@H]1CCC(C)=C[C@H]1c1c(O)cc(CCCCC)cc1OC(=O)C1(OP(=O)(O)OCCC(C)C)CCCCC1 ; C=C(C)[C@@H]1CCC(C)=C[C@H]1c1c(O)cc(CCCCC)cc1OC(=O)C1(OP(=O)(O)OCC(C)(C)C)CCCCC1 ; C=C(C)[C@@H]1CCC(C)=C[C@H]1c1c(O)cc(CCCCC)cc1OC(=O)C1(OP(=O)(O)OCC(CCC)CCC)CCCCC1]

and pharmaceutically acceptable salts of any of the foregoing.

In some embodiments, the compound of Formula (I) is chosen from compounds having the following formulae:

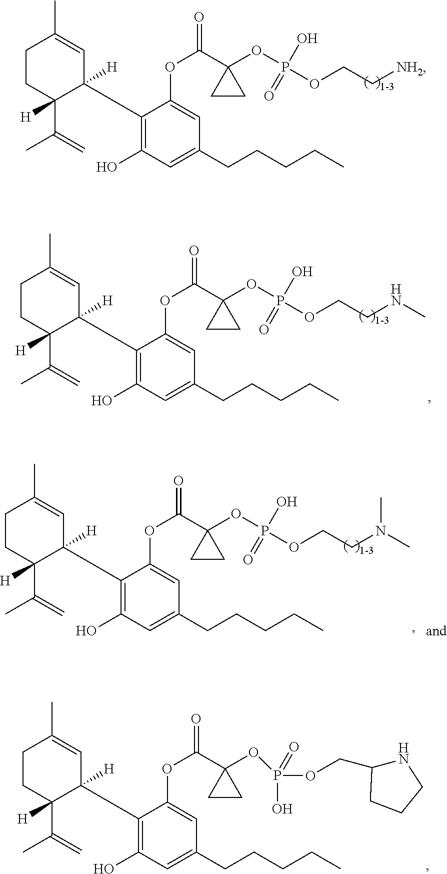

and pharmaceutically acceptable salts of any of the foregoing.

In some embodiments, the compound of Formula (I) is chosen from compounds having the following formulae:

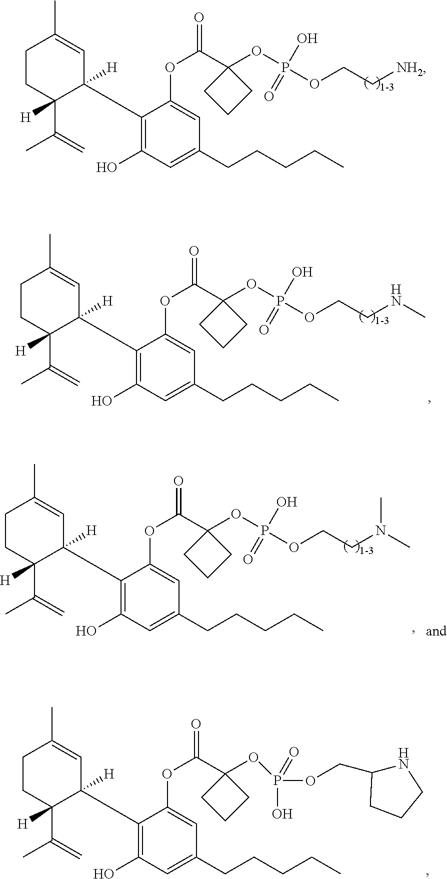

and pharmaceutically acceptable salts of any of the foregoing.

In some embodiments, the compound of Formula (I) is chosen from compounds

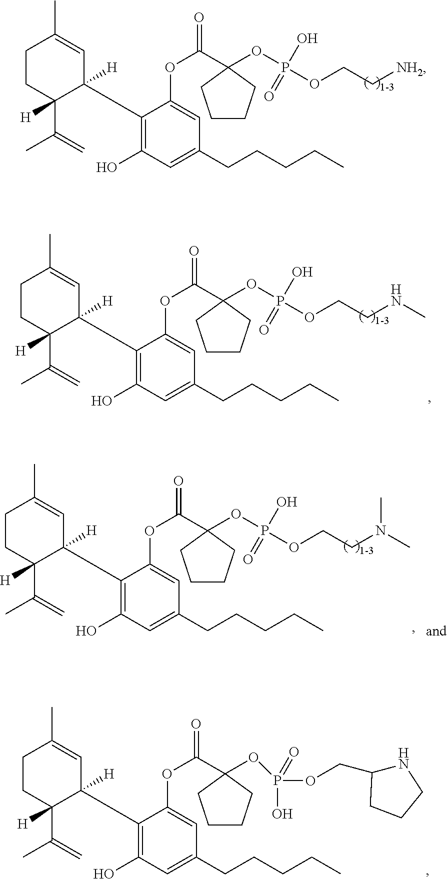

and pharmaceutically acceptable salts of any of the foregoing.

In some embodiments, the compound of Formula (I) is chosen from compounds having the following formulae:

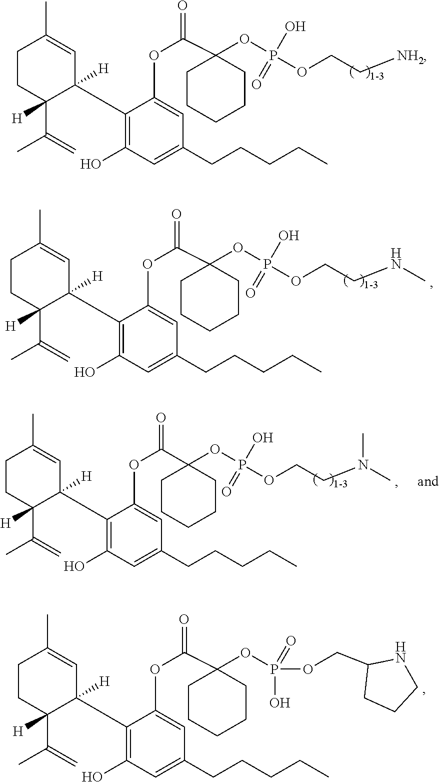

and pharmaceutically acceptable salts of any of the foregoing.

In some embodiments, the compound of Formula (I) is chosen from compounds having the following formulae:

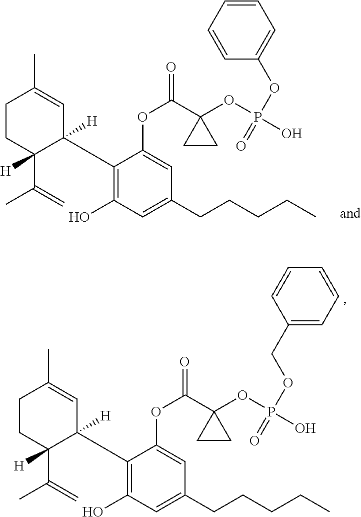 [SMILES: C=C(C)[C@@H]1CCC(C)=C[C@H]1c1c(O)cc(CCCCC)cc1OC(=O)C1(OP(=O)(O)Oc2ccccc2)CC1 ; C=C(C)[C@@H]1CCC(C)=C[C@H]1c1c(O)cc(CCCCC)cc1OC(=O)C1(OP(=O)(O)OCc2ccccc2)CC1]

and pharmaceutically acceptable salts of any of the foregoing.

In some embodiments, the compound of Formula (I) is chosen from compounds having the following formulae:

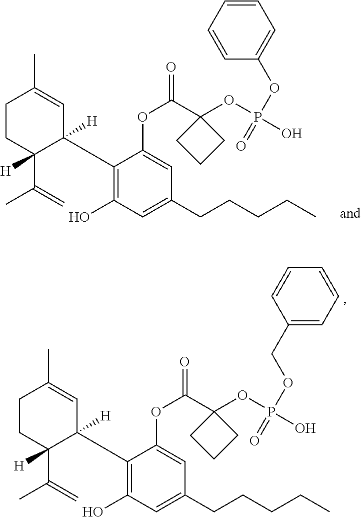 [SMILES: C=C(C)[C@@H]1CCC(C)=C[C@H]1c1c(O)cc(CCCCC)cc1OC(=O)C1(OP(=O)(O)Oc2ccccc2)CCC1 ; C=C(C)[C@@H]1CCC(C)=C[C@H]1c1c(O)cc(CCCCC)cc1OC(=O)C1(OP(=O)(O)OCc2ccccc2)CCC1]

and pharmaceutically acceptable salts of any of the foregoing.

In some embodiments, the compound of Formula (I) is chosen from compounds having the following formulae:

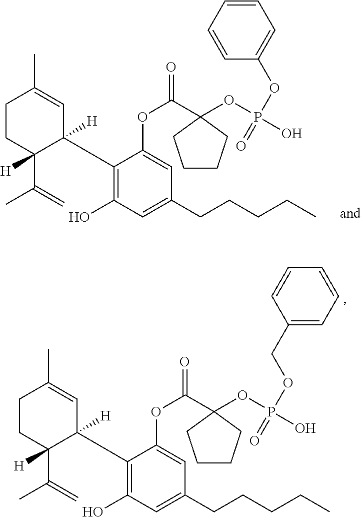 [SMILES: C=C(C)[C@@H]1CCC(C)=C[C@H]1c1c(O)cc(CCCCC)cc1OC(=O)C1(OP(=O)(O)Oc2ccccc2)CCCC1 ; C=C(C)[C@@H]1CCC(C)=C[C@H]1c1c(O)cc(CCCCC)cc1OC(=O)C1(OP(=O)(O)OCc2ccccc2)CCCC1]

and pharmaceutically acceptable salts of any of the foregoing.

In some embodiments, the compound of Formula (I) is chosen from compounds having the following formulae:

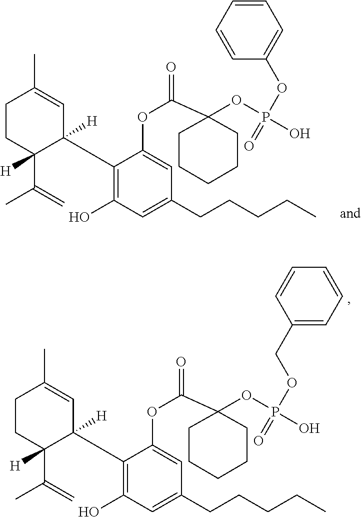 [SMILES: C=C(C)[C@@H]1CCC(C)=C[C@H]1c1c(O)cc(CCCCC)cc1OC(=O)C1(OP(=O)(O)Oc2ccccc2)CCCCC1 ; C=C(C)[C@@H]1CCC(C)=C[C@H]1c1c(O)cc(CCCCC)cc1OC(=O)C1(OP(=O)(O)OCc2ccccc2)CCCCC1]

and pharmaceutically acceptable salts of any of the foregoing.

In some embodiments, the compound of Formula (I) is chosen from compounds having the following formulae:

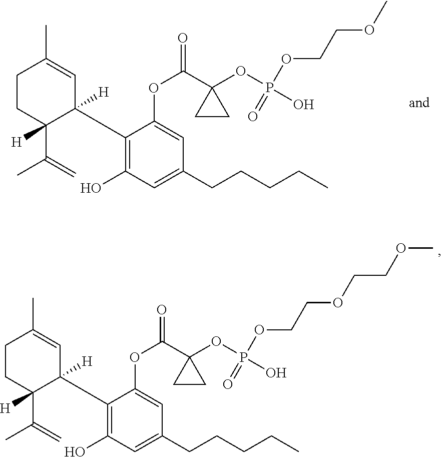 [SMILES: C=C(C)[C@@H]1CCC(C)=C[C@H]1c1c(O)cc(CCCCC)cc1OC(=O)C1(OP(=O)(O)OCCOC)CC1 ; C=C(C)[C@@H]1CCC(C)=C[C@H]1c1c(O)cc(CCCCC)cc1OC(=O)C1(OP(=O)(O)OCCOCCOC)CC1]

and pharmaceutically acceptable salts of any of the foregoing.

In some embodiments, the compound of Formula (I) is chosen from compounds having the following formulae:

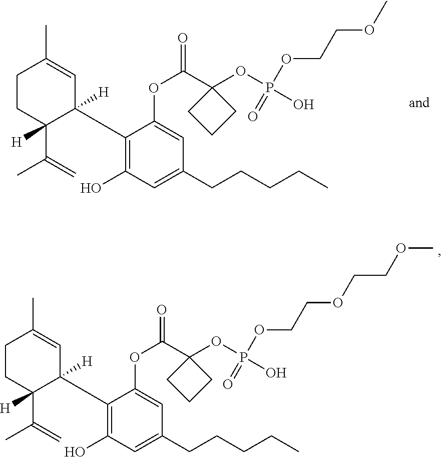 [SMILES: C=C(C)[C@@H]1CCC(C)=C[C@H]1c1c(O)cc(CCCCC)cc1OC(=O)C1(OP(=O)(O)OCCOC)CCC1 ; C=C(C)[C@@H]1CCC(C)=C[C@H]1c1c(O)cc(CCCCC)cc1OC(=O)C1(OP(=O)(O)OCCOCCOC)CCC1]

and pharmaceutically acceptable salts of any of the foregoing.

In some embodiments, the compound of Formula (I) is chosen from compounds having the following formulae:

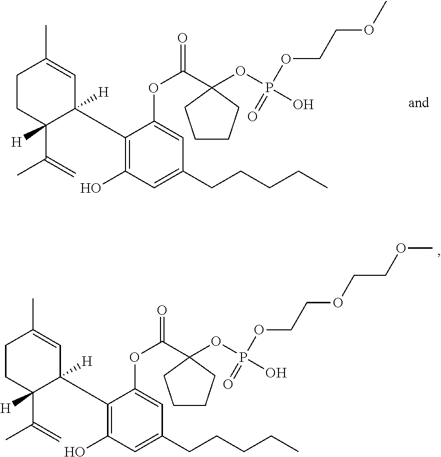 [SMILES: C=C(C)[C@@H]1CCC(C)=C[C@H]1c1c(O)cc(CCCCC)cc1OC(=O)C1(OP(=O)(O)OCCOC)CCCC1 ; C=C(C)[C@@H]1CCC(C)=C[C@H]1c1c(O)cc(CCCCC)cc1OC(=O)C1(OP(=O)(O)OCCOCCOC)CCCC1]

and pharmaceutically acceptable salts of any of the foregoing.

In some embodiments, the compound of Formula (I) is chosen from compounds

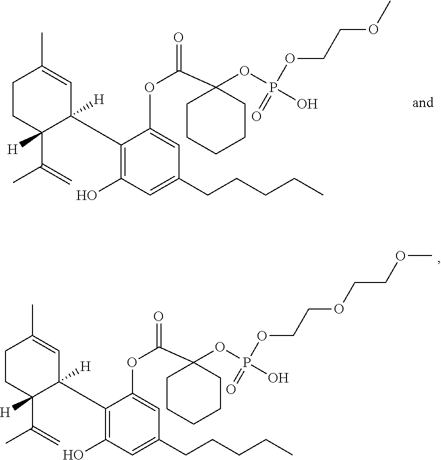 [SMILES: C=C(C)[C@@H]1CCC(C)=C[C@H]1c1c(O)cc(CCCCC)cc1OC(=O)C1(OP(=O)(O)OCCOC)CCCCC1 ; C=C(C)[C@@H]1CCC(C)=C[C@H]1c1c(O)cc(CCCCC)cc1OC(=O)C1(OP(=O)(O)OCCOCCOC)CCCCC1]

and pharmaceutically acceptable salts of any of the foregoing.

In some embodiments, the compound of Formula (I) is chosen from compounds having the following formulae:

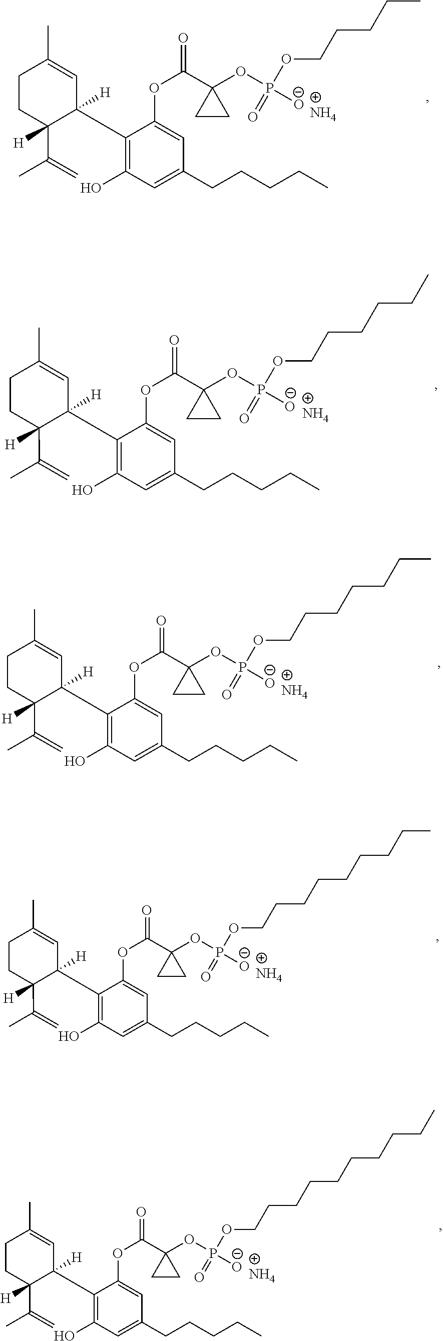

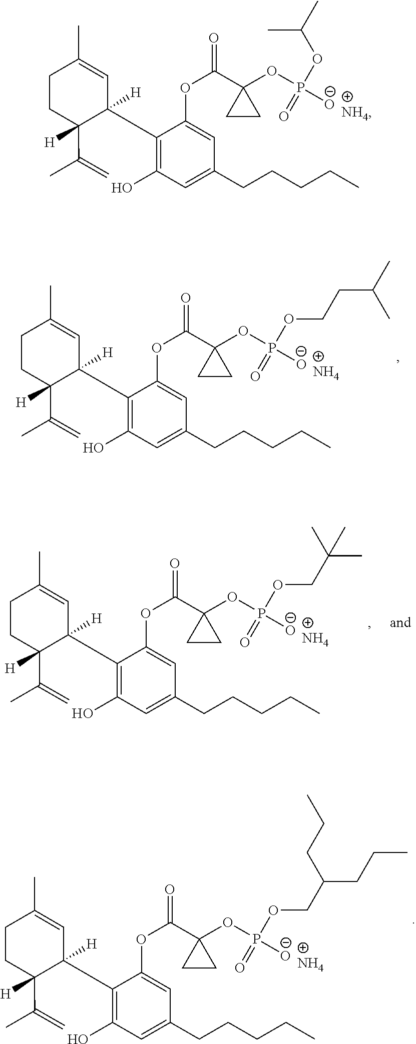

In some embodiments, the compound of Formula (I) is chosen from compounds having the following formulae:

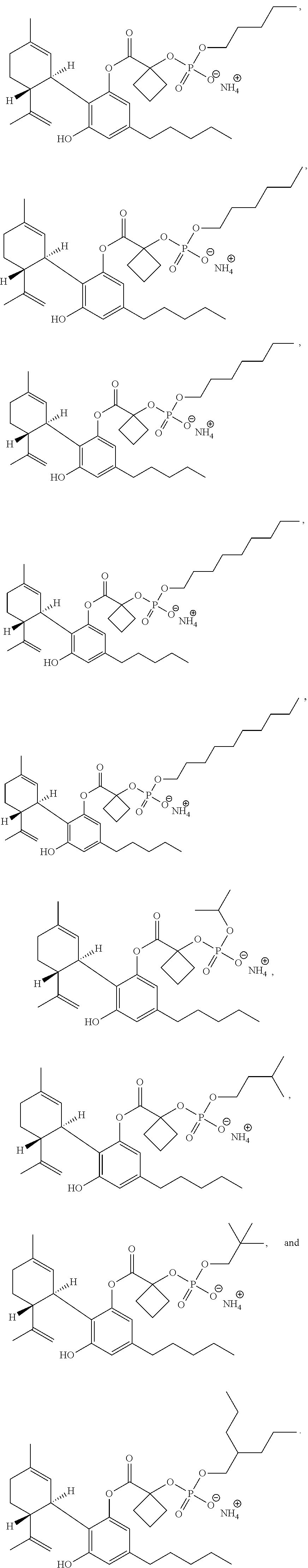

In some embodiments, the compound of Formula (I) is chosen from compounds

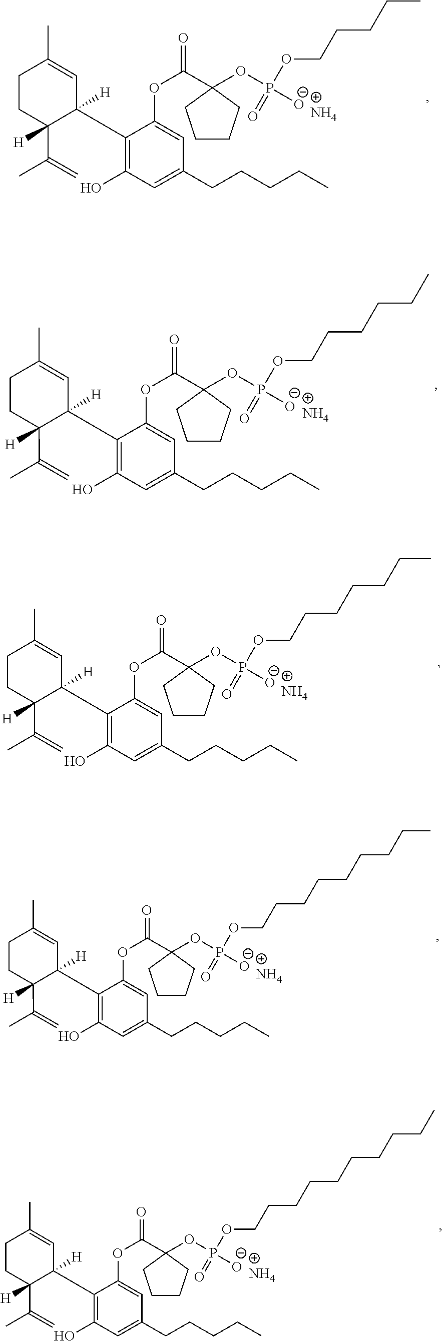

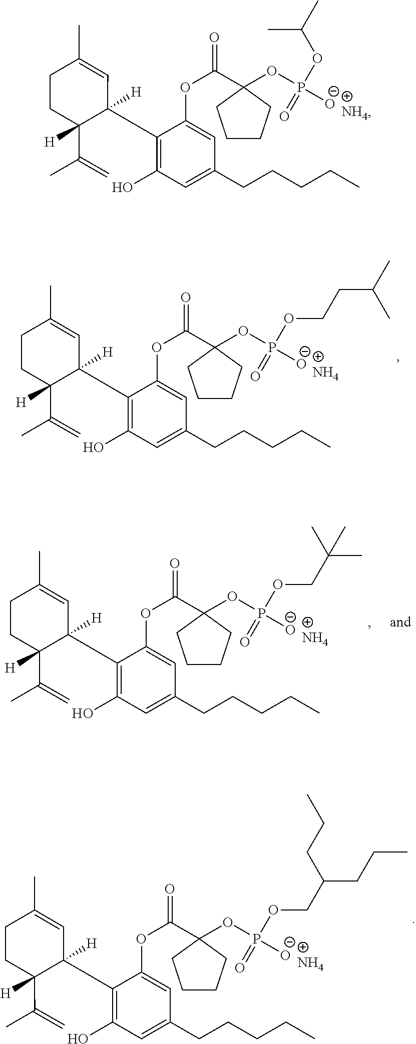

In some embodiments, the compound of Formula (I) is chosen from compounds having the following formulae:

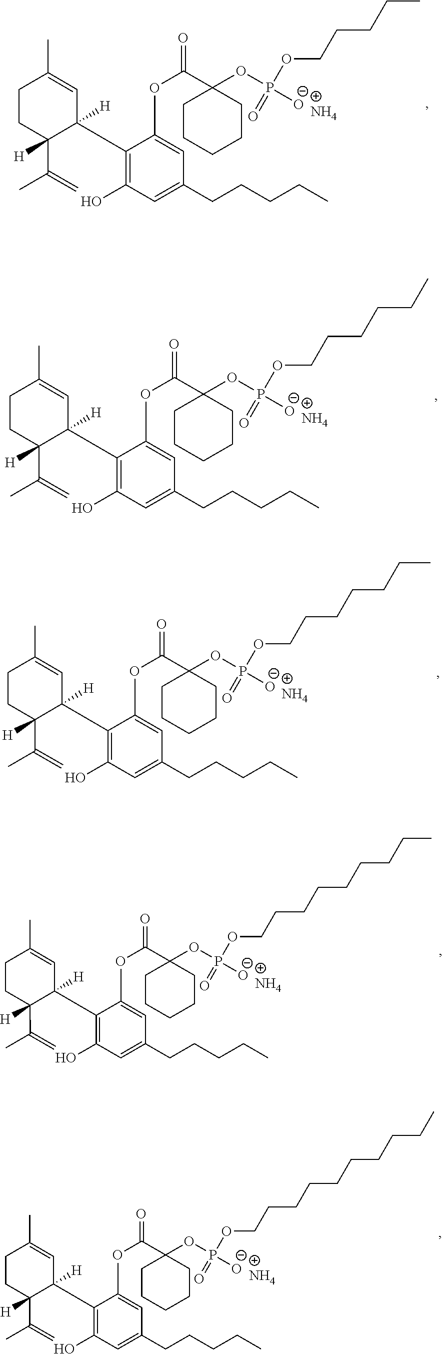

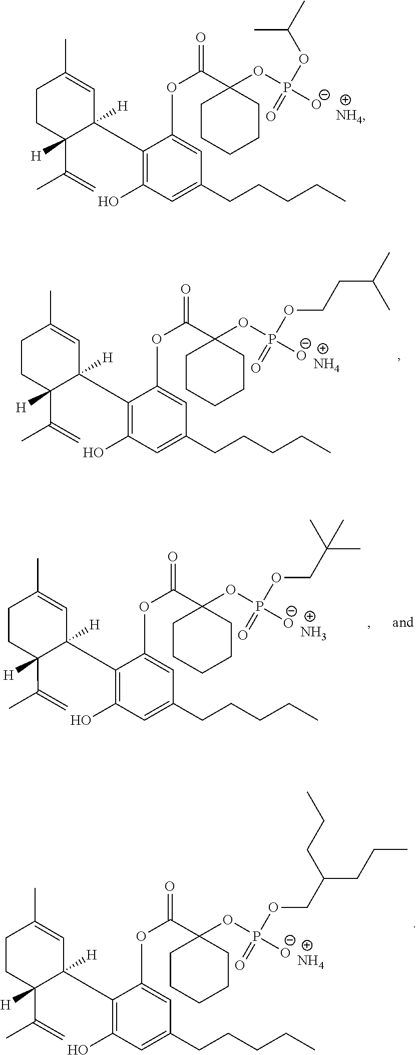

In some embodiments, the compound of Formula (I) is chosen from compounds having the following formulae:

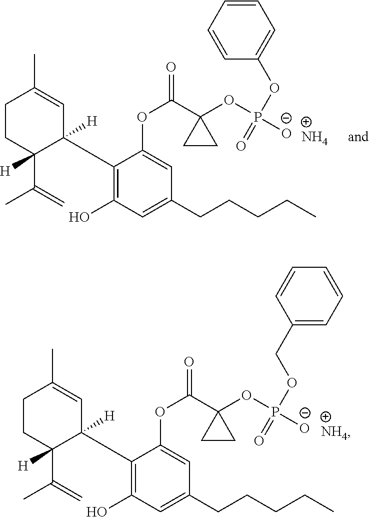

In some embodiments, the compound of Formula (I) is chosen from compounds having the following formulae:

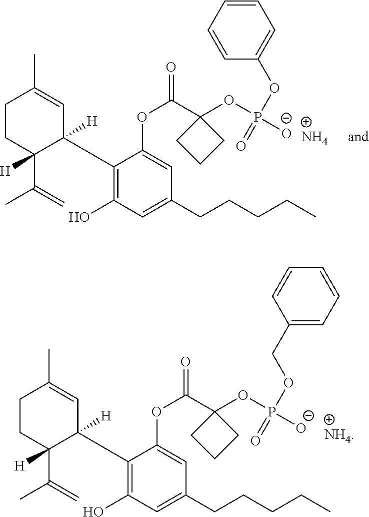

In some embodiments, the compound of Formula (I) is chosen from compounds having the following formulae:

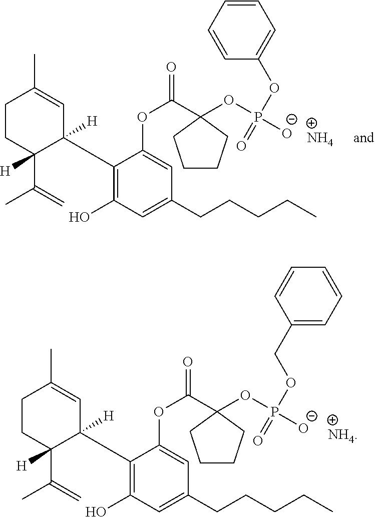

In some embodiments, the compound of Formula (I) is chosen from compounds having the following formulae:

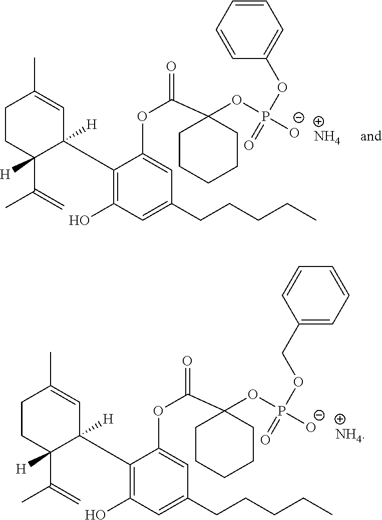

In some embodiments, the compound of Formula (I) is chosen from compounds having the following formulae:

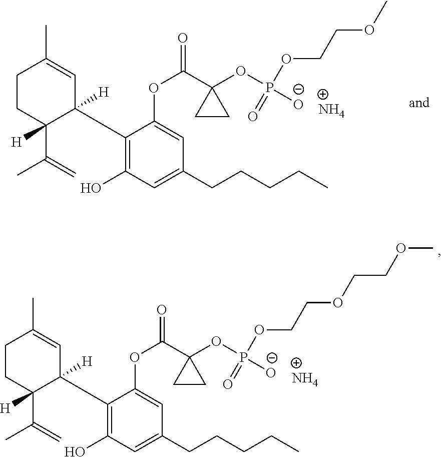 [SMILES: C=C(C)[C@@H]1CCC(C)=C[C@H]1c1c(O)cc(CCCCC)cc1OC(=O)C1(OP(=O)([O-])OCCOC)CC1 ; [NH4+] ; C=C(C)[C@@H]1CCC(C)=C[C@H]1c1c(O)cc(CCCCC)cc1OC(=O)C1(OP(=O)([O-])OCCOCCOC)CC1 ; [NH4+]]

In some embodiments, the compound of Formula (I) is chosen from compounds having the following formulae:

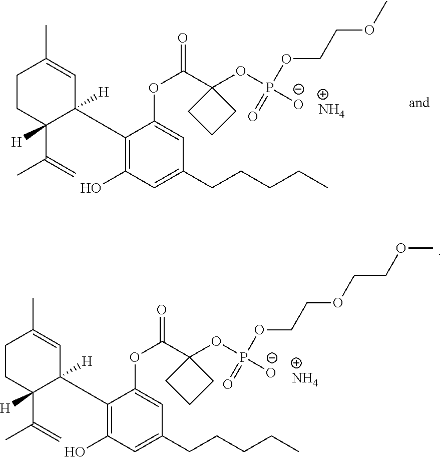 [SMILES: C=C(C)[C@@H]1CCC(C)=C[C@H]1c1c(O)cc(CCCCC)cc1OC(=O)C1(OP(=O)([O-])OCCOC)CCC1 ; [NH4+] ; C=C(C)[C@@H]1CCC(C)=C[C@H]1c1c(O)cc(CCCCC)cc1OC(=O)C1(OP(=O)([O-])OCCOCCOC)CCC1 ; [NH4+]]

In some embodiments, the compound of Formula (I) is chosen from compounds having the following formulae:

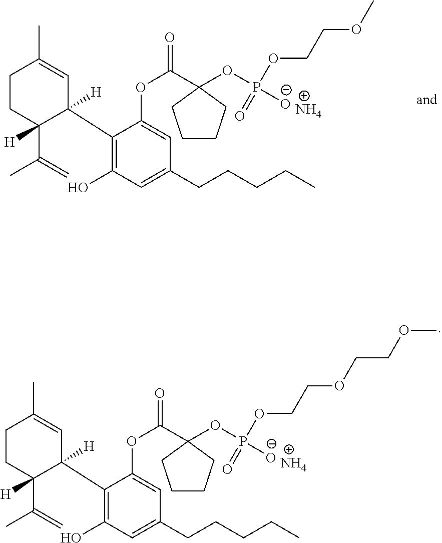 [SMILES: C=C(C)[C@@H]1CCC(C)=C[C@H]1c1c(O)cc(CCCCC)cc1OC(=O)C1(OP(=O)([O-])OCCOC)CCCC1 ; [NH4+] ; C=C(C)[C@@H]1CCC(C)=C[C@H]1c1c(O)cc(CCCCC)cc1OC(=O)C1(OP(=O)([O-])OCCOCCOC)CCCC1 ; [NH4+]]

In some embodiments, the compound of Formula (I) is chosen from compounds having the following formulae:

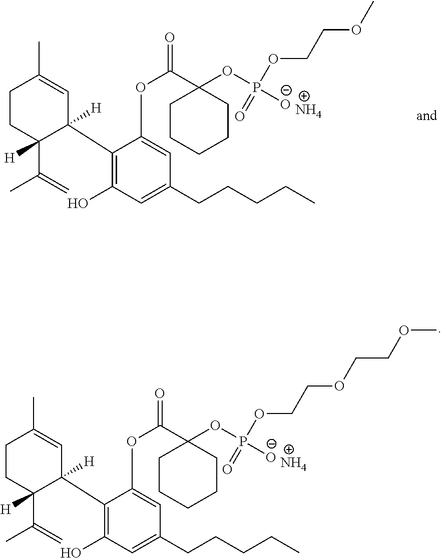 [SMILES: C=C(C)[C@@H]1CCC(C)=C[C@H]1c1c(O)cc(CCCCC)cc1OC(=O)C1(OP(=O)([O-])OCCOC)CCCCC1 ; [NH4+] ; C=C(C)[C@@H]1CCC(C)=C[C@H]1c1c(O)cc(CCCCC)cc1OC(=O)C1(OP(=O)([O-])OCCOCCOC)CCCCC1 ; [NH4+]]

In some embodiments, the compound of Formula (I) is chosen from compounds having the following formulae:

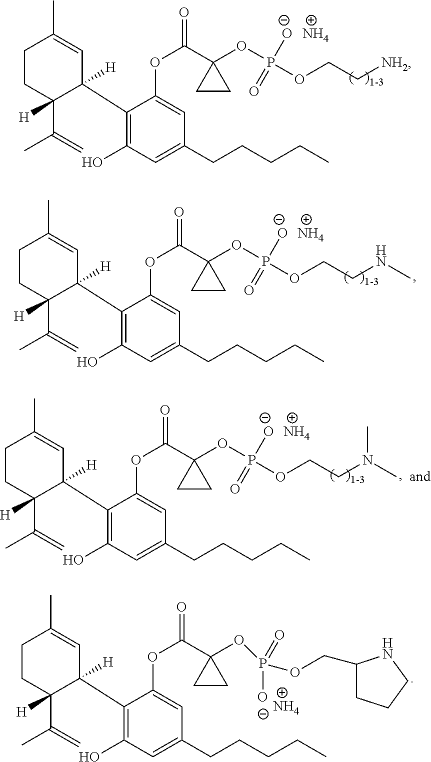

In some embodiments, the compound of Formula (I) is chosen from compounds

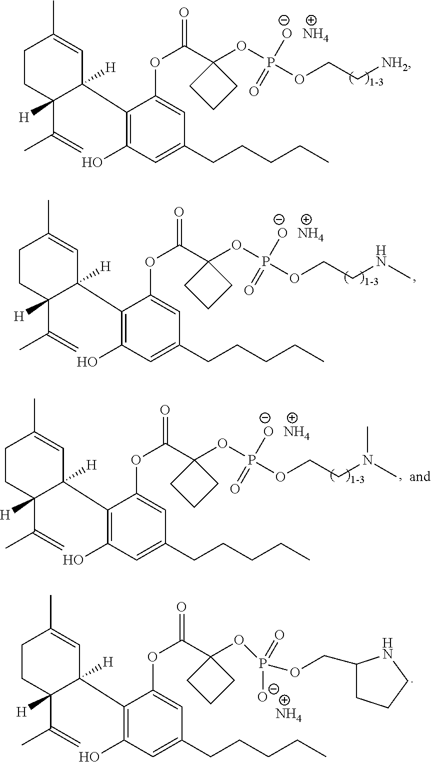

In some embodiments, the compound of Formula (I) is chosen from compounds having the following formulae:

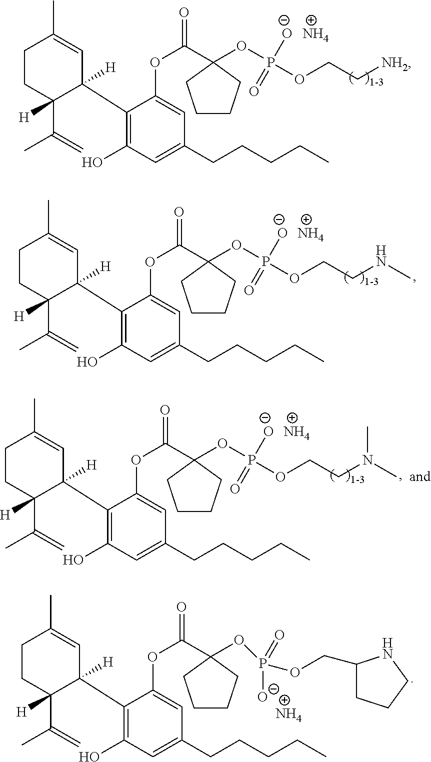

In some embodiments, the compound of Formula (I) is chosen from compounds having the following formulae:

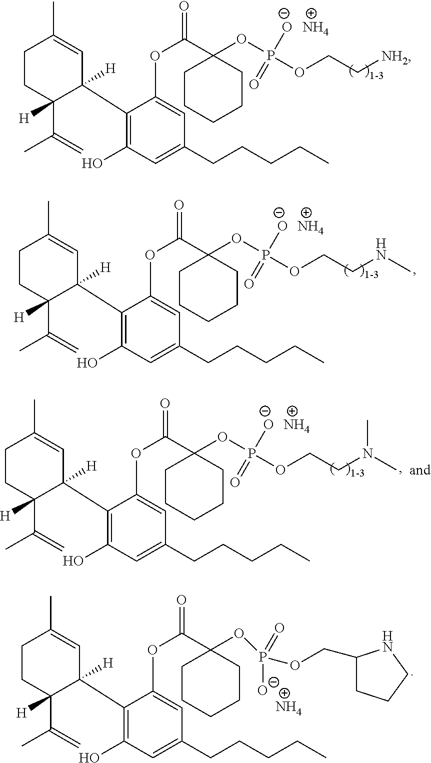

In some embodiments, the compound of Formula (I) is chosen from compounds having the following formula:

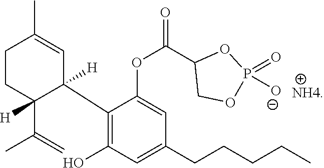

In some embodiments, the compound of Formula (I) is chosen from compounds having the following formula:

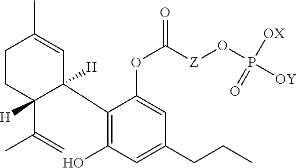

and pharmaceutically acceptable salts thereof. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —
(CH₂CH₂O)ₙCH₃,

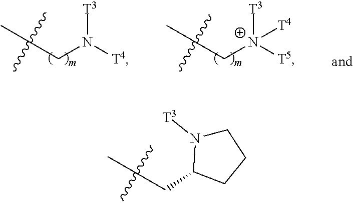

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

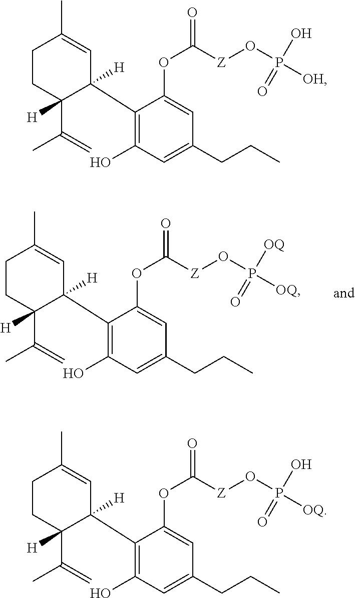

In some embodiments, the compound of Formula (I) is chosen from compounds having the following formulae:

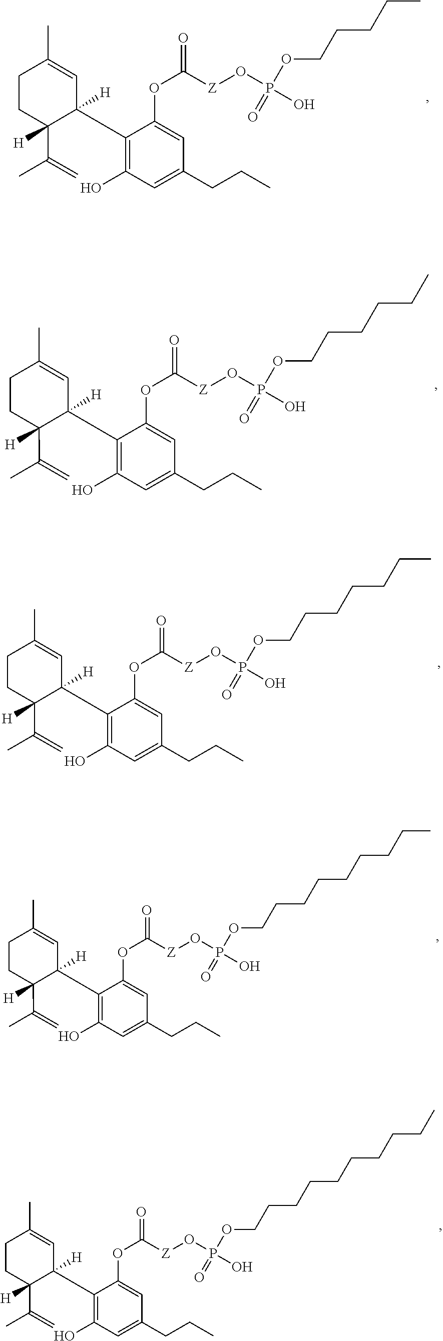

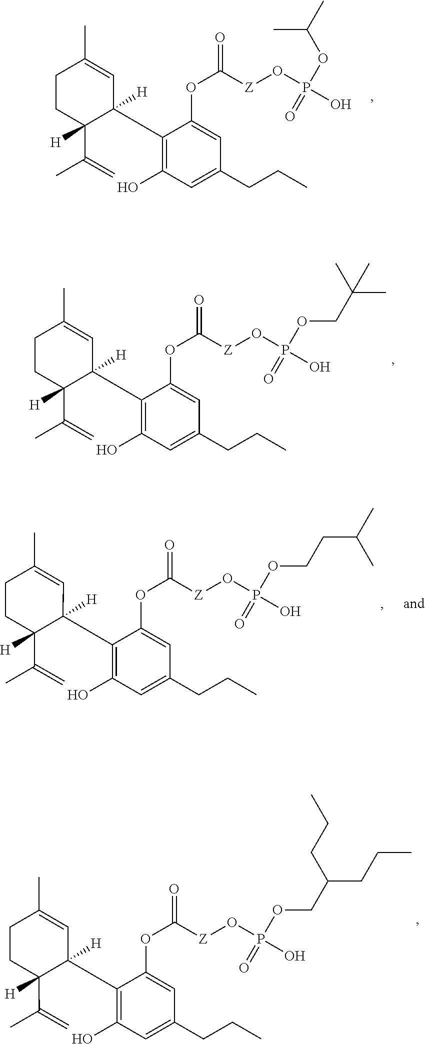

and pharmaceutically acceptable salts of any of the foregoing. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (I) is chosen from compounds having the following formulae:

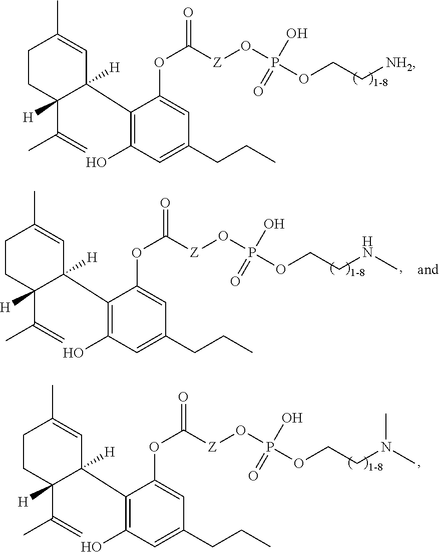

and pharmaceutically acceptable salts of any of the foregoing. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (I) is chosen from compounds having the following formulae:

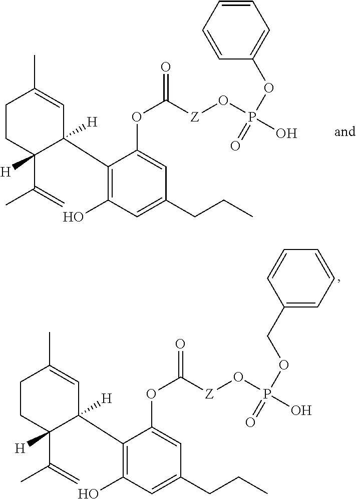

and pharmaceutically acceptable salts of any of the foregoing. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (I) is chosen from compounds having the following formulae:

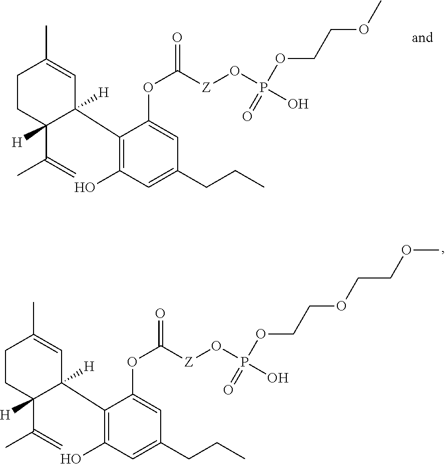

and pharmaceutically acceptable salts of any of the foregoing. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (I) is chosen from compounds having the following formula:

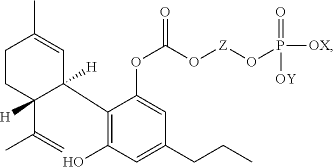

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃,

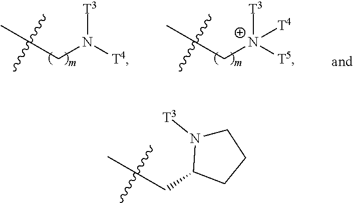

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

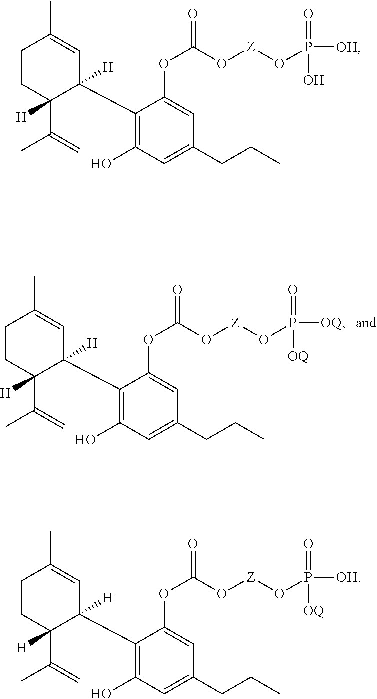

In some embodiments, the compound of Formula (I) is chosen from compounds having the following formulae:

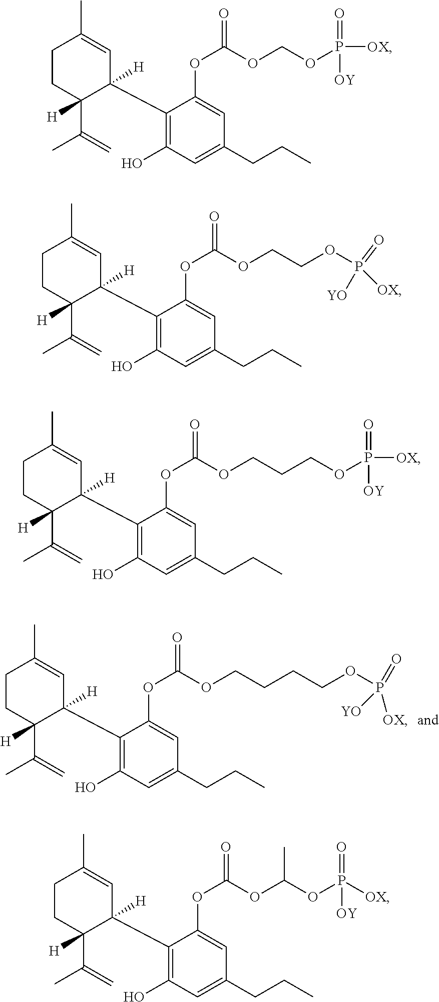

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

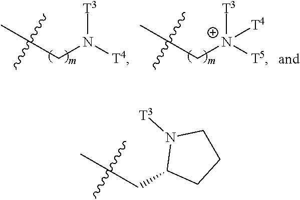

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds

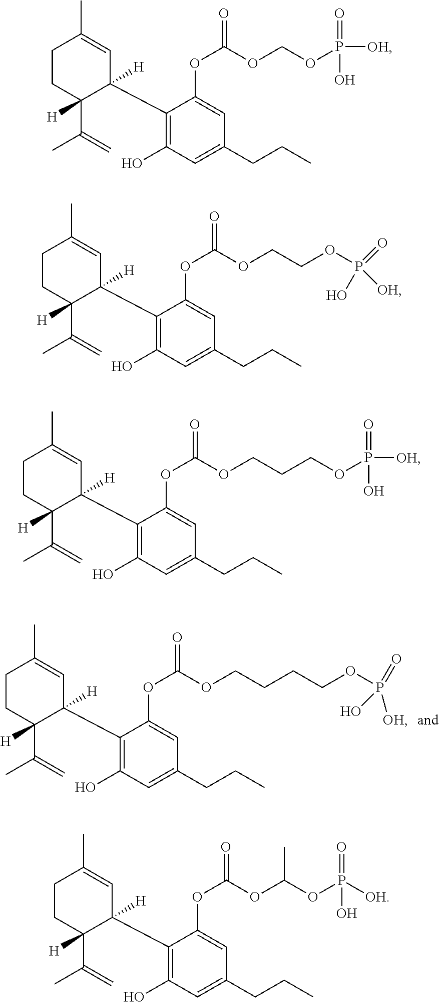 [SMILES: C=C(C)[C@@H]1CCC(C)=C[C@H]1c1c(O)cc(CCC)cc1OC(=O)OCOP(=O)(O)O ; C=C(C)[C@@H]1CCC(C)=C[C@H]1c1c(O)cc(CCC)cc1OC(=O)OCCOP(=O)(O)O ; C=C(C)[C@@H]1CCC(C)=C[C@H]1c1c(O)cc(CCC)cc1OC(=O)OCCCOP(=O)(O)O ; C=C(C)[C@@H]1CCC(C)=C[C@H]1c1c(O)cc(CCC)cc1OC(=O)OCCCCOP(=O)(O)O ; C=C(C)[C@@H]1CCC(C)=C[C@H]1c1c(O)cc(CCC)cc1OC(=O)OC(C)OP(=O)(O)O]

In some embodiments, the compound of Formula (I) is chosen from compounds having the following formulae:

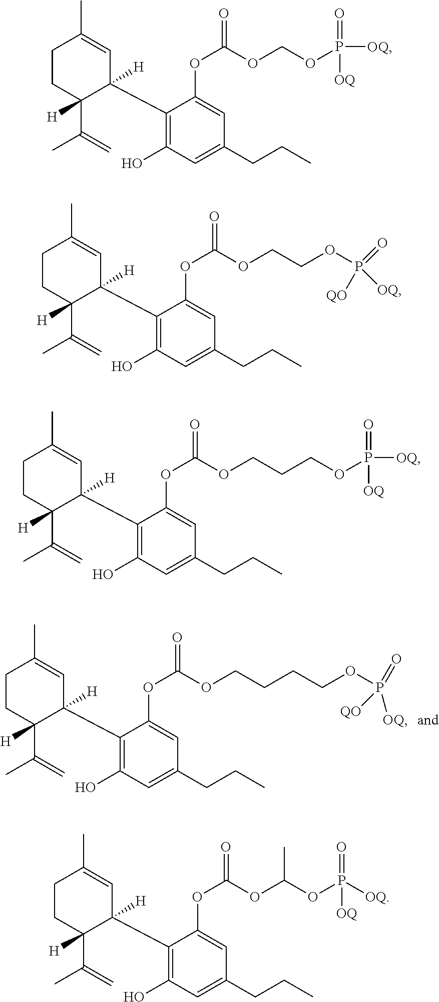

In some embodiments, the compound of Formula (I) is chosen from compounds having the following formulae:

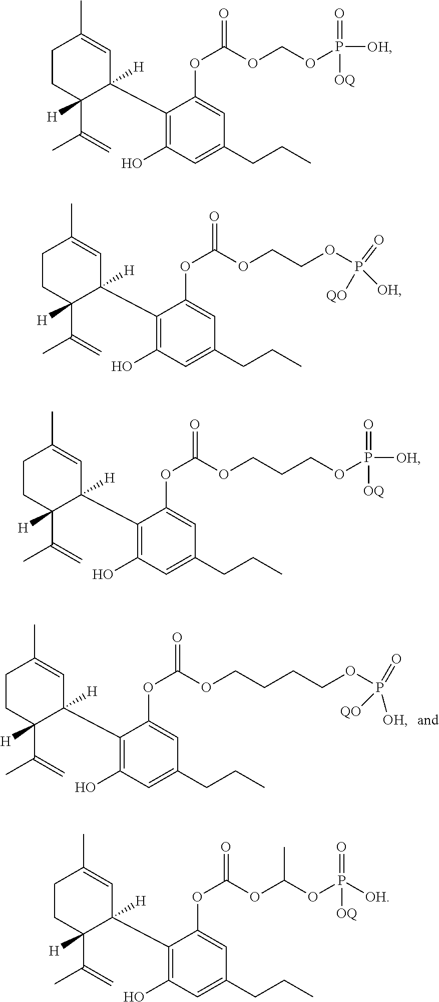

In some embodiments, the compound of Formula (I) is chosen from compounds having the following formula:

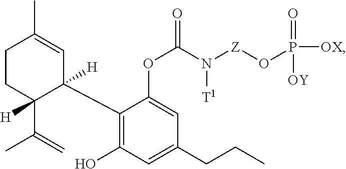

and pharmaceutically acceptable salts of any of the foregoing. In some embodiments, T¹ is H. In some embodiments, T¹ is methyl. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —
(CH₂CH₂O)ₙCH₃,

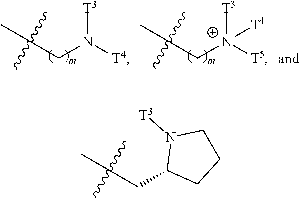

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

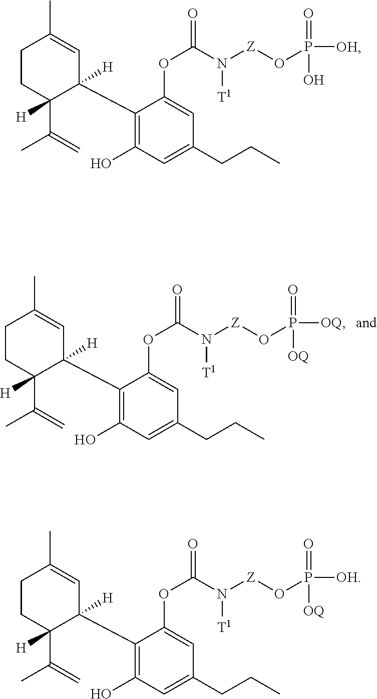

In some embodiments, the compound of Formula (I) is chosen from compounds having the following formulae:

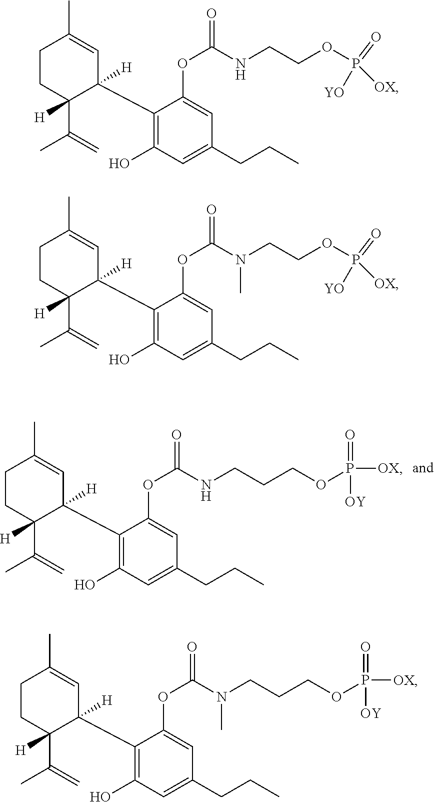

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

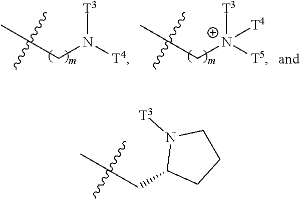

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

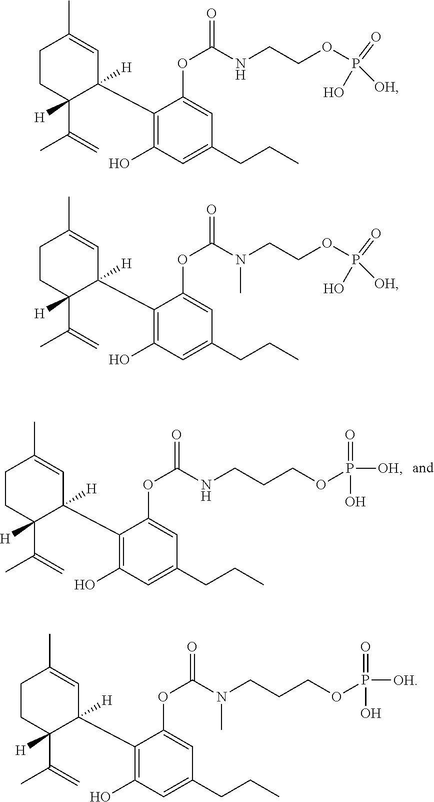 [SMILES: C=C(C)[C@@H]1CCC(C)=C[C@H]1c1c(O)cc(CCC)cc1OC(=O)NCCOP(=O)(O)O ; C=C(C)[C@@H]1CCC(C)=C[C@H]1c1c(O)cc(CCC)cc1OC(=O)N(C)CCOP(=O)(O)O ; C=C(C)[C@@H]1CCC(C)=C[C@H]1c1c(O)cc(CCC)cc1OC(=O)NCCCOP(=O)(O)O ; C=C(C)[C@@H]1CCC(C)=C[C@H]1c1c(O)cc(CCC)cc1OC(=O)N(C)CCCOP(=O)(O)O]

In some embodiments, the compound of Formula (I) is chosen from compounds having the following formulae:

In some embodiments, the compound of Formula (I) is chosen from compounds having the following formulae:

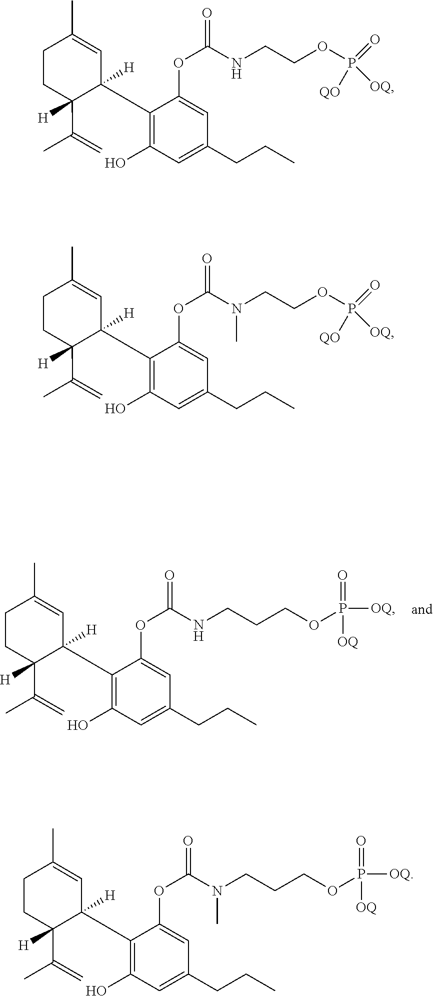

In some embodiments, the compound of Formula (I) is chosen from compounds having the following formulae:

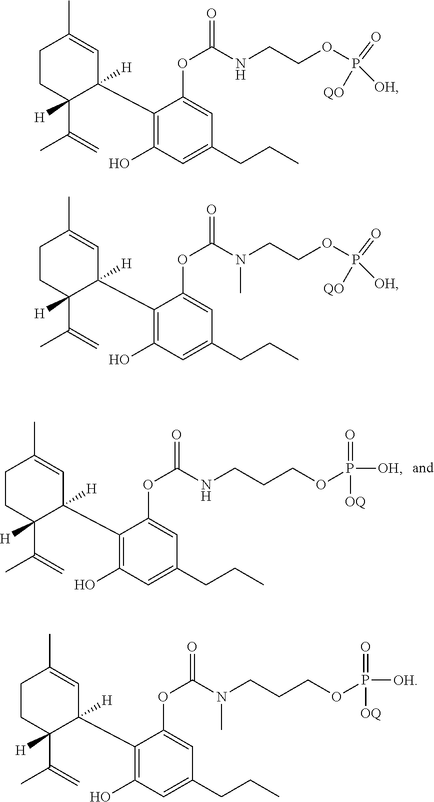

In some embodiments, the compound of Formula (I) is chosen from compounds having the following formula:

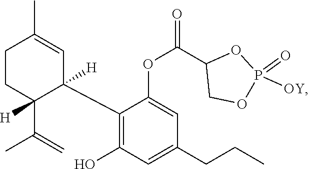

and pharmaceutically acceptable salts of any of the foregoing.

In some embodiments, the compound of Formula (I) is chosen from compounds having the following formulae:

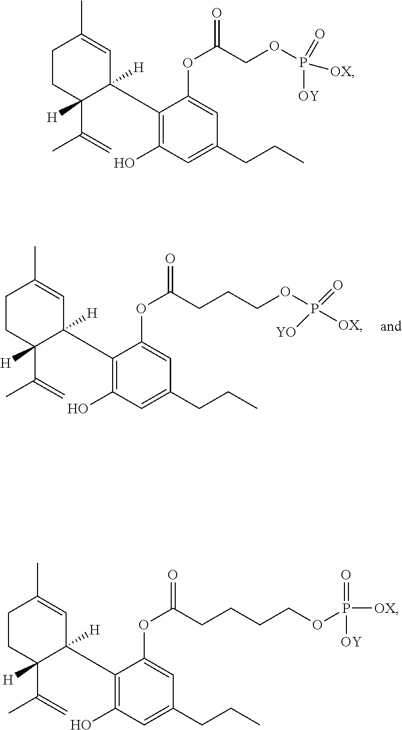

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃,

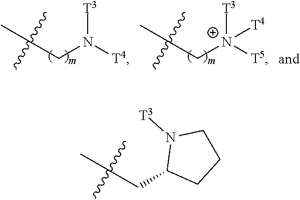

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

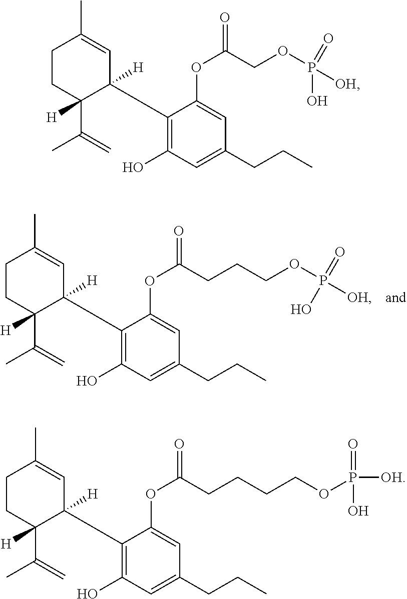 [SMILES: C=C(C)[C@@H]1CCC(C)=C[C@H]1c1c(O)cc(CCC)cc1OC(=O)COP(=O)(O)O ; C=C(C)[C@@H]1CCC(C)=C[C@H]1c1c(O)cc(CCC)cc1OC(=O)CCCOP(=O)(O)O ; C=C(C)[C@@H]1CCC(C)=C[C@H]1c1c(O)cc(CCC)cc1OC(=O)CCCCOP(=O)(O)O]

In some embodiments, the compound of Formula (I) is chosen from compounds having the following formulae:

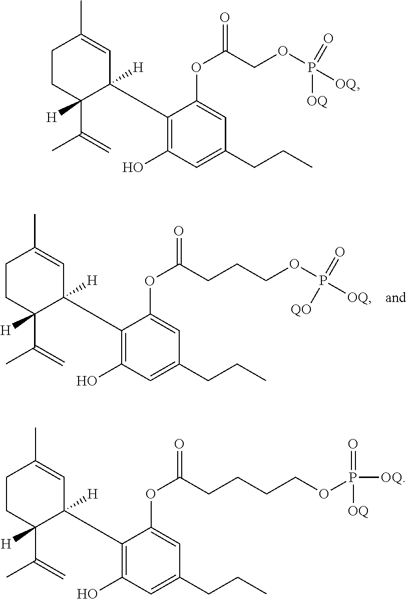

In some embodiments, the compound of Formula (I) is chosen from compounds having the following formulae:

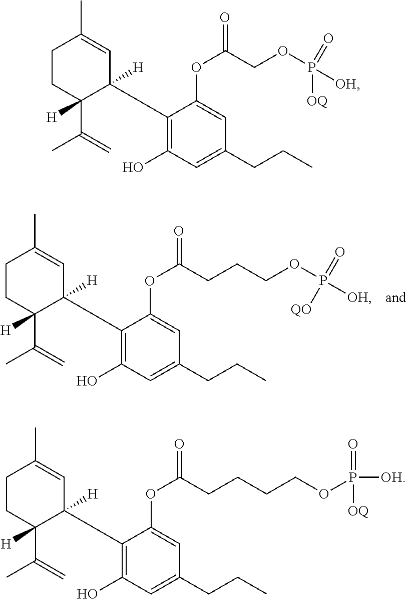

In some embodiments, the compound of Formula (I) is chosen from compounds having the following formulae:

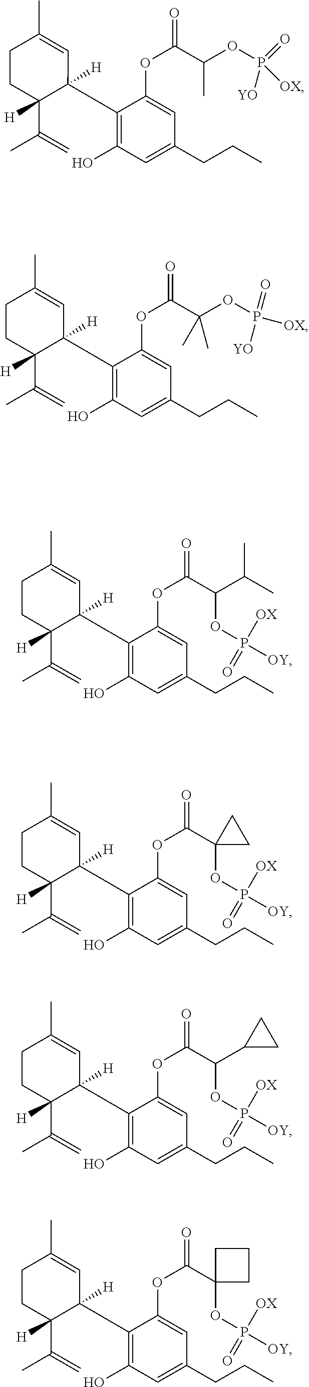

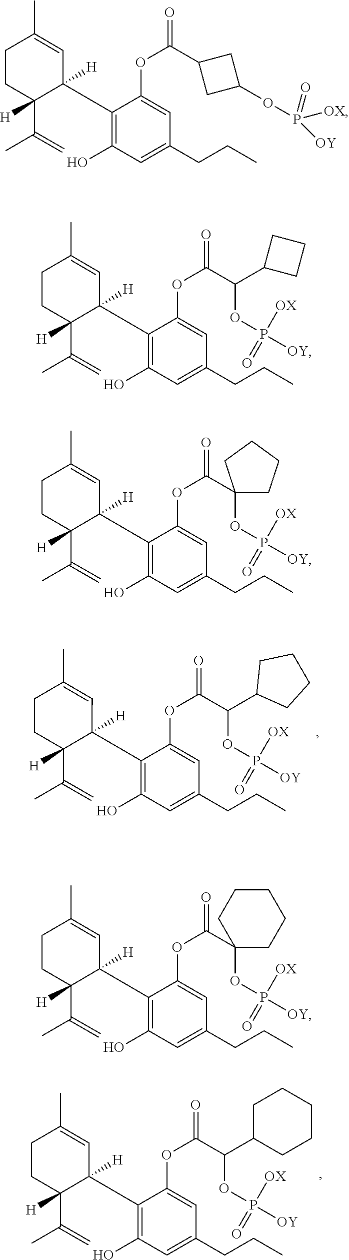

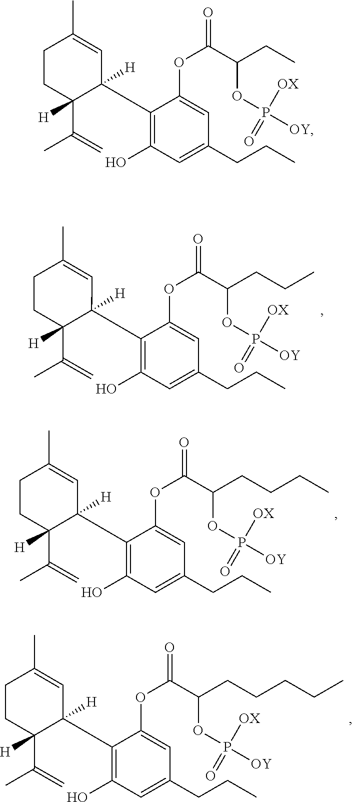

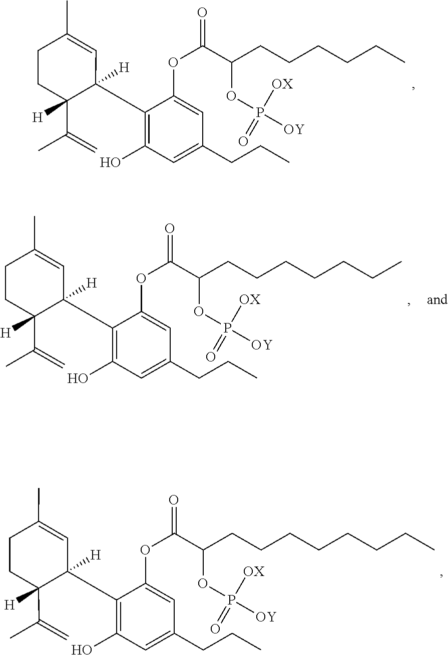

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃,

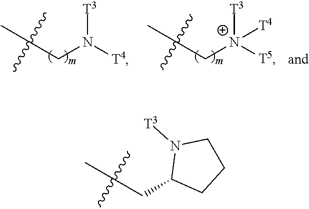

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

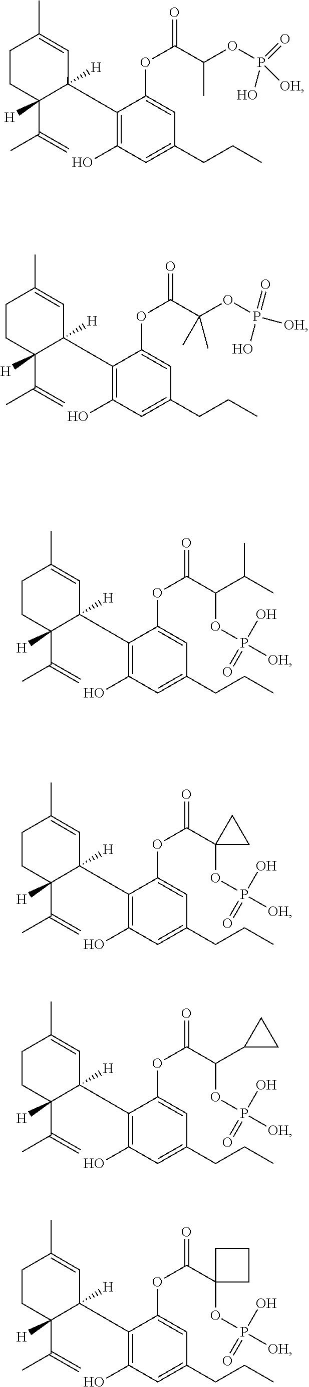 [SMILES: C=C(C)[C@@H]1CCC(C)=C[C@H]1c1c(O)cc(CCC)cc1OC(=O)C(C)OP(=O)(O)O ; C=C(C)[C@@H]1CCC(C)=C[C@H]1c1c(O)cc(CCC)cc1OC(=O)C(C)(C)OP(=O)(O)O ; C=C(C)[C@@H]1CCC(C)=C[C@H]1c1c(O)cc(CCC)cc1OC(=O)C(OP(=O)(O)O)C(C)C ; C=C(C)[C@@H]1CCC(C)=C[C@H]1c1c(O)cc(CCC)cc1OC(=O)C1(OP(=O)(O)O)CC1 ; C=C(C)[C@@H]1CCC(C)=C[C@H]1c1c(O)cc(CCC)cc1OC(=O)C(OP(=O)(O)O)C1CC1 ; C=C(C)[C@@H]1CCC(C)=C[C@H]1c1c(O)cc(CCC)cc1OC(=O)C1(OP(=O)(O)O)CCC1]

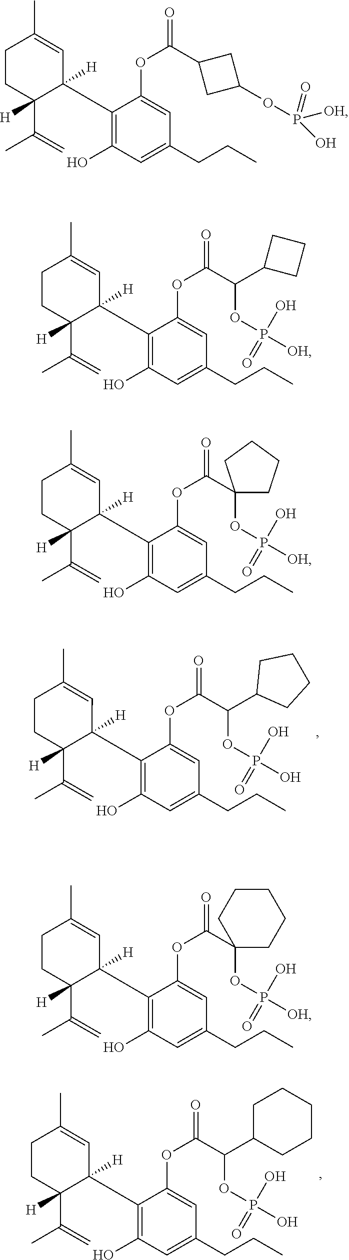

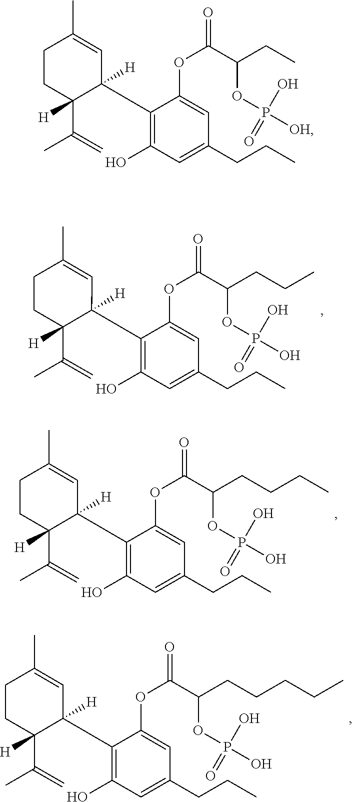 [SMILES: C=C(C)[C@@H]1CCC(C)=C[C@H]1c1c(O)cc(CCC)cc1OC(=O)C(CC)OP(=O)(O)O ; C=C(C)[C@@H]1CCC(C)=C[C@H]1c1c(O)cc(CCC)cc1OC(=O)C(CCC)OP(=O)(O)O ; C=C(C)[C@@H]1CCC(C)=C[C@H]1c1c(O)cc(CCC)cc1OC(=O)C(CCCC)OP(=O)(O)O ; C=C(C)[C@@H]1CCC(C)=C[C@H]1c1c(O)cc(CCC)cc1OC(=O)C(CCCCC)OP(=O)(O)O]

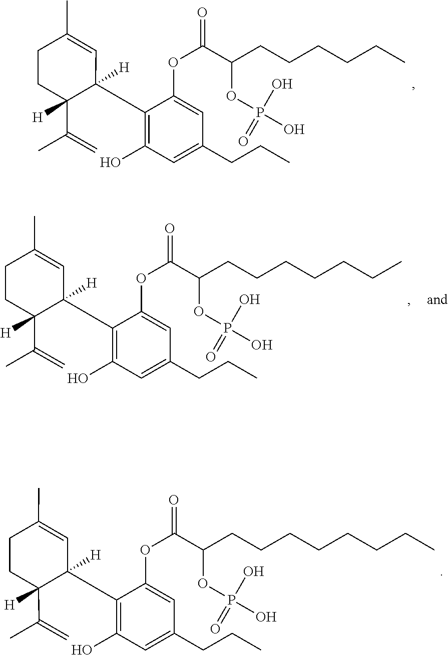 [SMILES: C=C(C)[C@@H]1CCC(C)=C[C@H]1c1c(O)cc(CCC)cc1OC(=O)C(CCCCCC)OP(=O)(O)O ; C=C(C)[C@@H]1CCC(C)=C[C@H]1c1c(O)cc(CCC)cc1OC(=O)C(CCCCCCC)OP(=O)(O)O ; C=C(C)[C@@H]1CCC(C)=C[C@H]1c1c(O)cc(CCC)cc1OC(=O)C(CCCCCCCC)OP(=O)(O)O]

In some embodiments, the compound of Formula (I) is chosen from compounds having the following formulae:

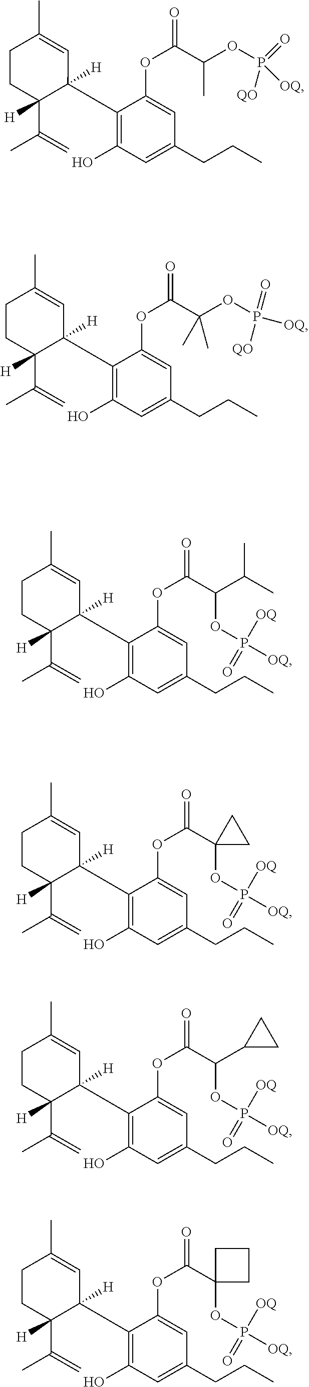

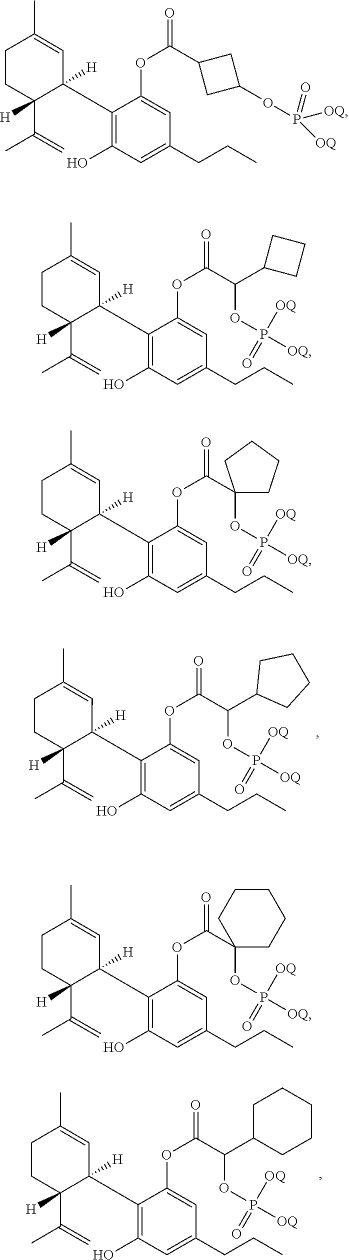

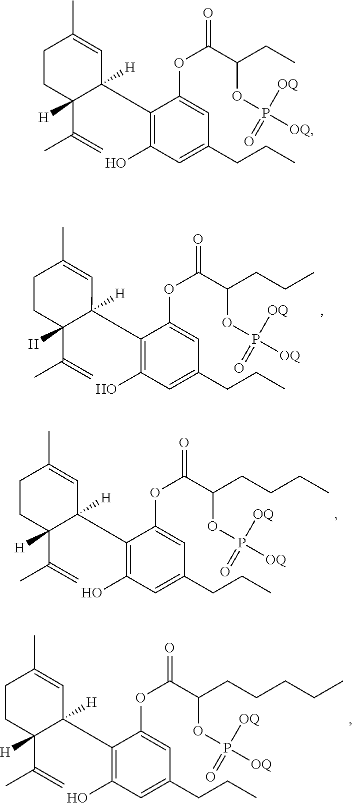

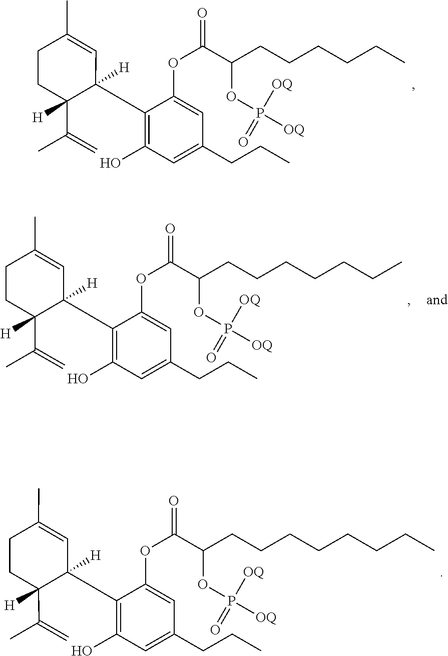

In some embodiments, the compound of Formula (I) is chosen from compounds having the following formulae:

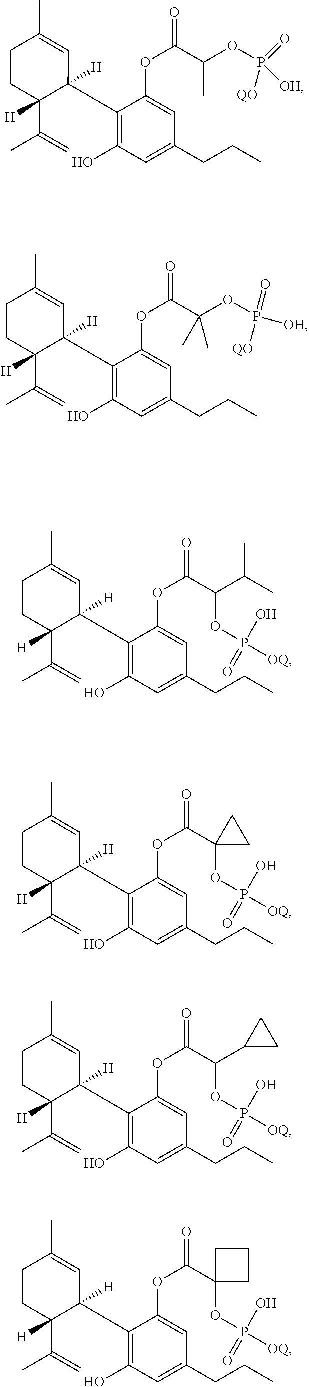

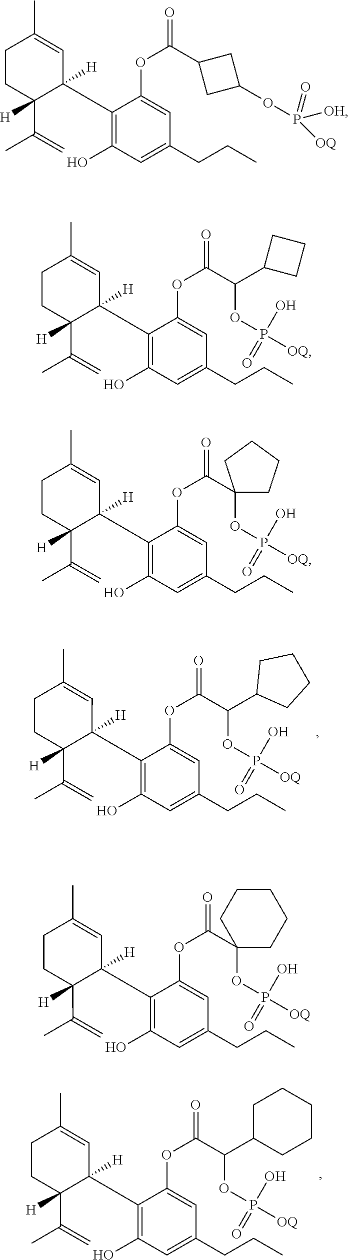

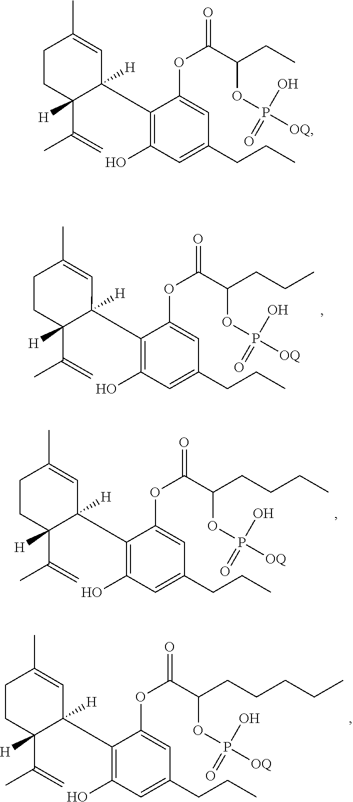

In some embodiments, the compound of Formula (I) is chosen from compounds having the following formulae:

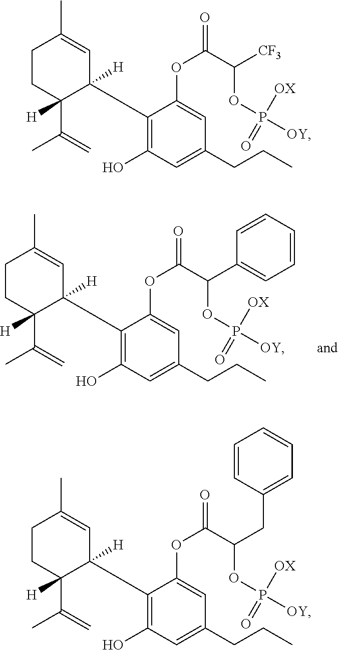

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃,

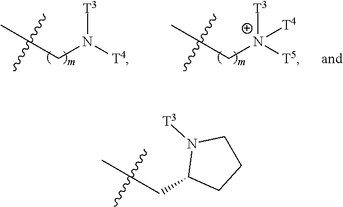

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

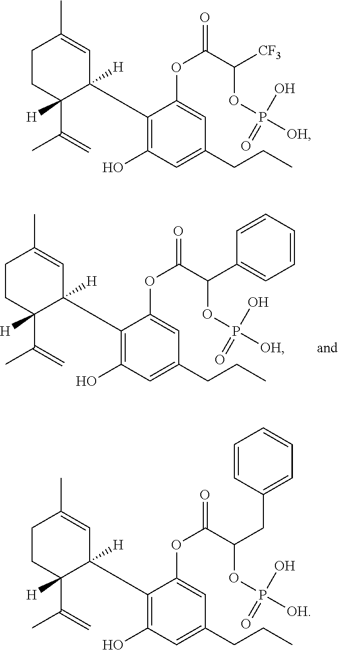 [SMILES: C=C(C)[C@@H]1CCC(C)=C[C@H]1c1c(O)cc(CCC)cc1OC(=O)C(OP(=O)(O)O)C(F)(F)F ; C=C(C)[C@@H]1CCC(C)=C[C@H]1c1c(O)cc(CCC)cc1OC(=O)C(OP(=O)(O)O)c1ccccc1 ; C=C(C)[C@@H]1CCC(C)=C[C@H]1c1c(O)cc(CCC)cc1OC(=O)C(Cc1ccccc1)OP(=O)(O)O]

In some embodiments, the compound of Formula (I) is chosen from compounds having the following formulae:

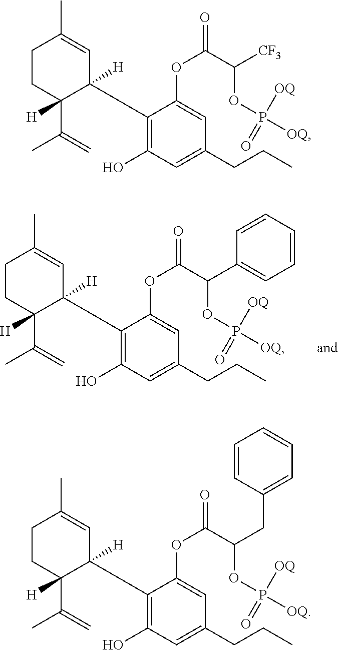

In some embodiments, the compound of Formula (I) is chosen from compounds having the following formulae:

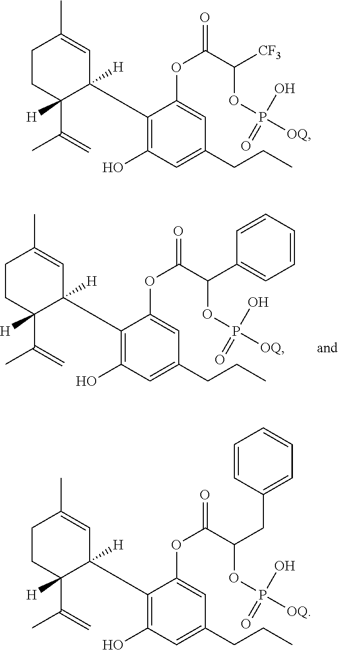

In some embodiments, the compound of Formula (I) is chosen from compounds having the following formulae:

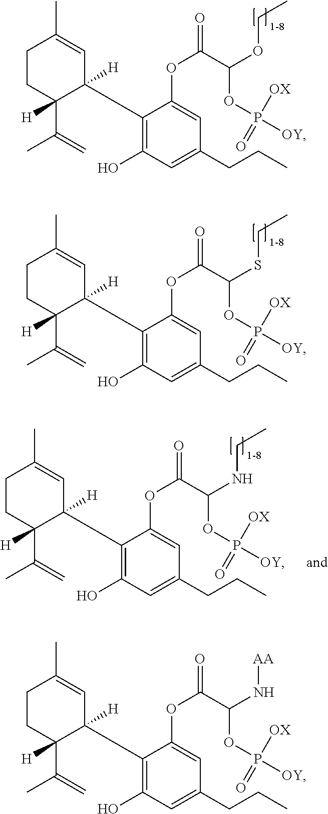

and pharmaceutically acceptable salts of any of the foregoing. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃,

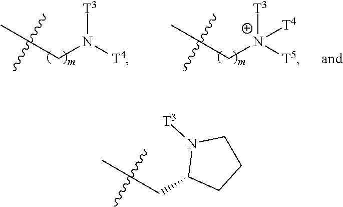

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formulae:

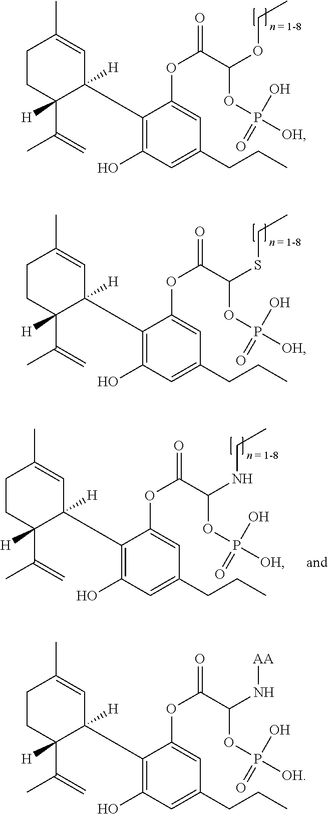

In some embodiments, the compound of Formula (I) is chosen from compounds having the following formulae:

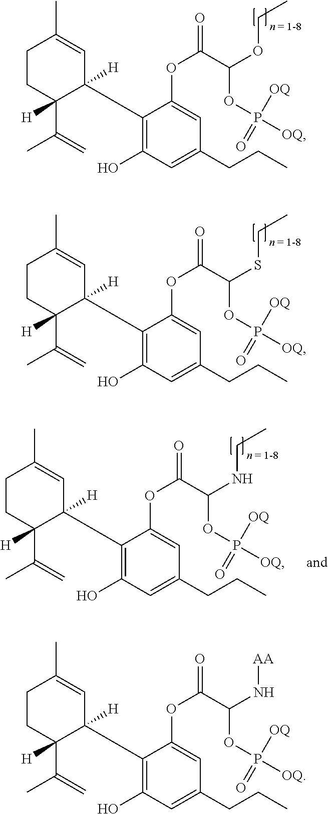

In some embodiments, the compound of Formula (I) is chosen from compounds having the following formulae:

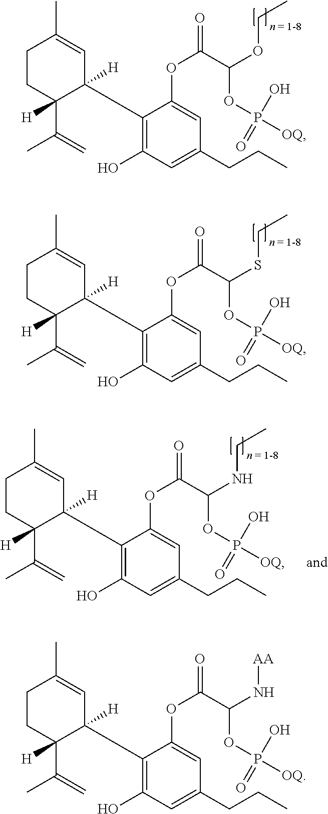

In some embodiments, the compound of Formula (I) is chosen from compounds having the following formulae:

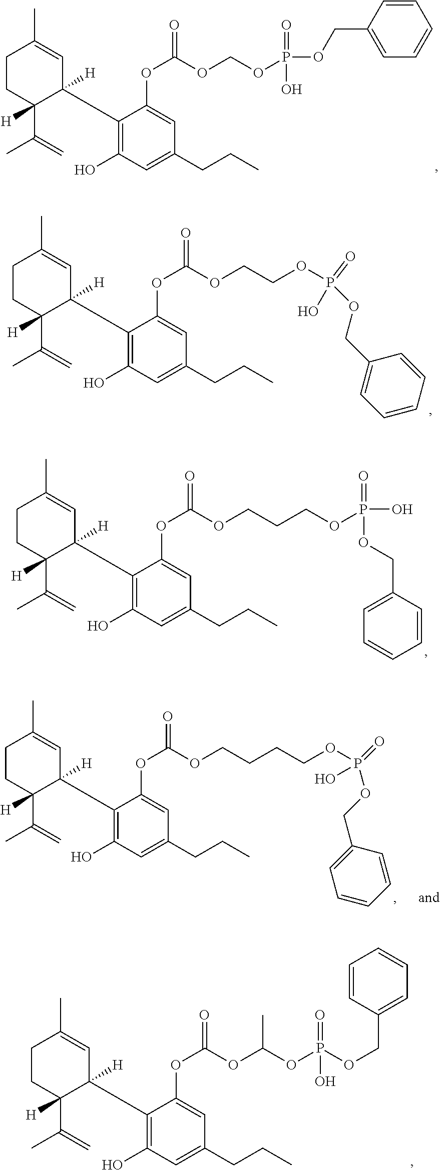

and pharmaceutically acceptable salts of any of the foregoing.

In some embodiments, the compound of Formula (I) is chosen form compounds having the following formulae:

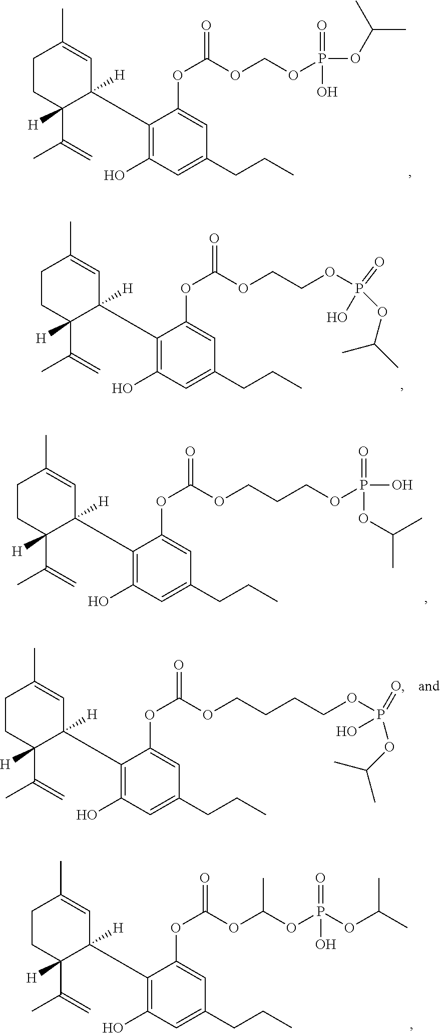 [SMILES: C=C(C)[C@@H]1CCC(C)=C[C@H]1c1c(O)cc(CCC)cc1OC(=O)OCOP(=O)(O)OC(C)C ; C=C(C)[C@@H]1CCC(C)=C[C@H]1c1c(O)cc(CCC)cc1OC(=O)OCCOP(=O)(O)OC(C)C ; C=C(C)[C@@H]1CCC(C)=C[C@H]1c1c(O)cc(CCC)cc1OC(=O)OCCCOP(=O)(O)OC(C)C ; C=C(C)[C@@H]1CCC(C)=C[C@H]1c1c(O)cc(CCC)cc1OC(=O)OCCCCOP(=O)(O)OC(C)C ; "and" ; C=C(C)[C@@H]1CCC(C)=C[C@H]1c1c(O)cc(CCC)cc1OC(=O)OC(C)OP(=O)(O)OC(C)C]

and pharmaceutically acceptable salts of any of the foregoing.

In some embodiments, the compound of Formula (I) is chosen from compounds having the following formulae:

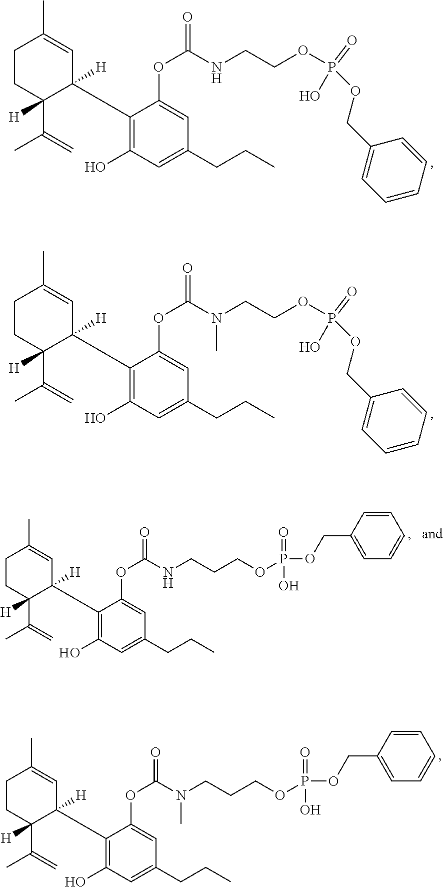 [SMILES: C=C(C)[C@@H]1CCC(C)=C[C@H]1c1c(O)cc(CCC)cc1OC(=O)NCCOP(=O)(O)OCc1ccccc1 ; C=C(C)[C@@H]1CCC(C)=C[C@H]1c1c(O)cc(CCC)cc1OC(=O)N(C)CCOP(=O)(O)OCc1ccccc1 ; C=C(C)[C@@H]1CCC(C)=C[C@H]1c1c(O)cc(CCC)cc1OC(=O)NCCCOP(=O)(O)OCc1ccccc1 ; C=C(C)[C@@H]1CCC(C)=C[C@H]1c1c(O)cc(CCC)cc1OC(=O)N(C)CCCOP(=O)(O)OCc1ccccc1]

and pharmaceutically acceptable salts of any of the foregoing.

In some embodiments, the compound of Formula (I) is chosen form compounds having the following formulae:

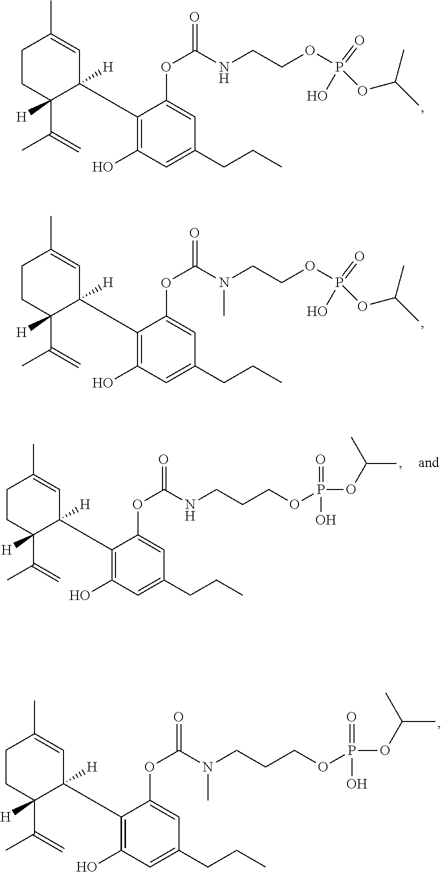 [SMILES: C=C(C)[C@@H]1CCC(C)=C[C@H]1c1c(O)cc(CCC)cc1OC(=O)NCCOP(=O)(O)OC(C)C ; C=C(C)[C@@H]1CCC(C)=C[C@H]1c1c(O)cc(CCC)cc1OC(=O)N(C)CCOP(=O)(O)OC(C)C ; C=C(C)[C@@H]1CCC(C)=C[C@H]1c1c(O)cc(CCC)cc1OC(=O)NCCCOP(=O)(O)OC(C)C ; C=C(C)[C@@H]1CCC(C)=C[C@H]1c1c(O)cc(CCC)cc1OC(=O)N(C)CCCOP(=O)(O)OC(C)C]

and pharmaceutically acceptable salts of any of the foregoing.

In some embodiments, the compound of Formula (I) is chosen from compounds having the following formula:

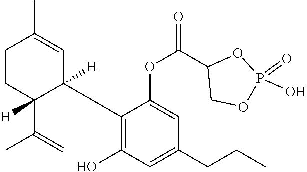 [SMILES: C=C(C)[C@@H]1CCC(C)=C[C@H]1c1c(O)cc(CCC)cc1OC(=O)C1COP(=O)(O)O1]

and pharmaceutically acceptable salts thereof.

In some embodiments, the compound of Formula (I) is chosen from compounds having the following formulae:

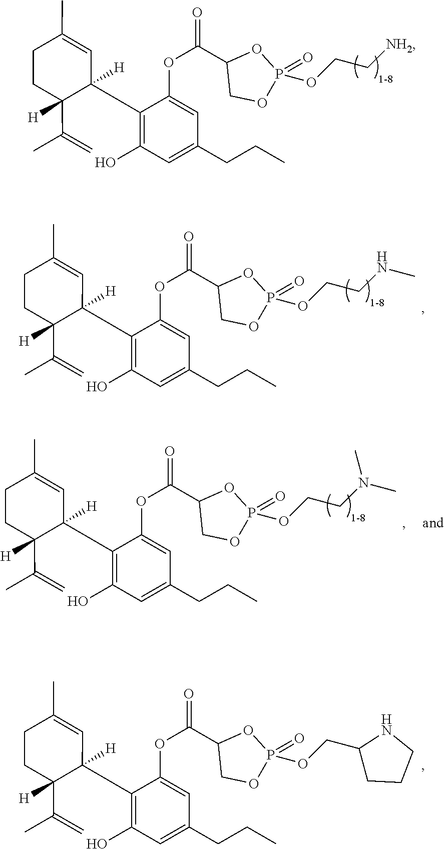

and pharmaceutically acceptable salts of any of the foregoing.

In some embodiments, the compound of Formula (I) is chosen from compounds having the following formulae:

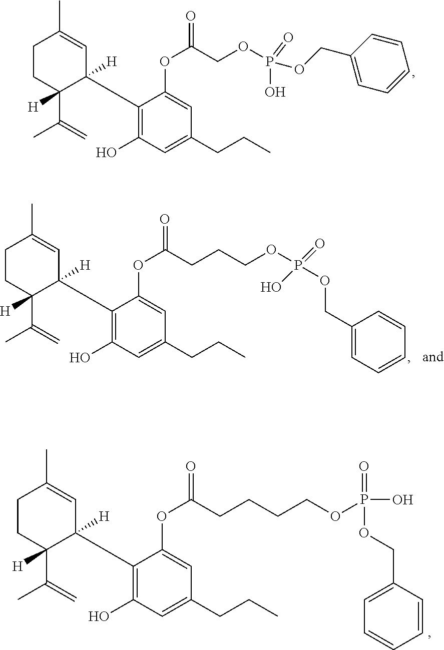 [SMILES: C=C(C)[C@@H]1CCC(C)=C[C@H]1c1c(O)cc(CCC)cc1OC(=O)COP(=O)(O)OCc1ccccc1 ; C=C(C)[C@@H]1CCC(C)=C[C@H]1c1c(O)cc(CCC)cc1OC(=O)CCCOP(=O)(O)OCc1ccccc1 ; C=C(C)[C@@H]1CCC(C)=C[C@H]1c1c(O)cc(CCC)cc1OC(=O)CCCCOP(=O)(O)OCc1ccccc1]

and pharmaceutically acceptable salts of any of the foregoing.

In some embodiments, the compound of Formula (I) is chosen form compounds having the following formulae:

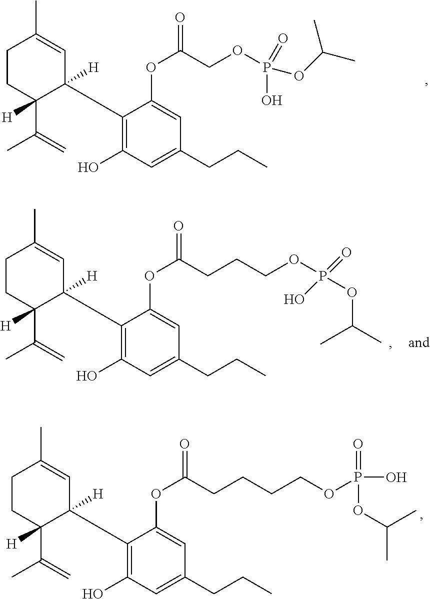 [SMILES: C=C(C)[C@@H]1CCC(C)=C[C@H]1c1c(O)cc(CCC)cc1OC(=O)COP(=O)(O)OC(C)C ; C=C(C)[C@@H]1CCC(C)=C[C@H]1c1c(O)cc(CCC)cc1OC(=O)CCCOP(=O)(O)OC(C)C ; C=C(C)[C@@H]1CCC(C)=C[C@H]1c1c(O)cc(CCC)cc1OC(=O)CCCCOP(=O)(O)OC(C)C]

and pharmaceutically acceptable salts of any of the foregoing.

In some embodiments, the compound of Formula (I) is chosen from compounds having the following formulae:

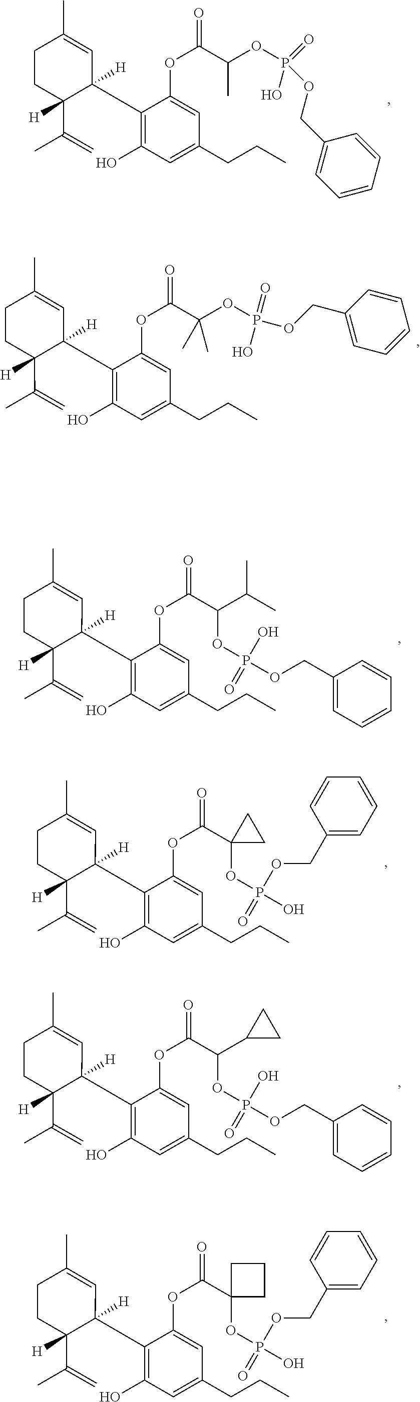 [SMILES: C=C(C)[C@@H]1CCC(C)=C[C@H]1c1c(O)cc(CCC)cc1OC(=O)C(C)OP(=O)(O)OCc1ccccc1 ; C=C(C)[C@@H]1CCC(C)=C[C@H]1c1c(O)cc(CCC)cc1OC(=O)C(C)(C)OP(=O)(O)OCc1ccccc1 ; C=C(C)[C@@H]1CCC(C)=C[C@H]1c1c(O)cc(CCC)cc1OC(=O)C(OP(=O)(O)OCc1ccccc1)C(C)C ; C=C(C)[C@@H]1CCC(C)=C[C@H]1c1c(O)cc(CCC)cc1OC(=O)C1(OP(=O)(O)OCc2ccccc2)CC1 ; C=C(C)[C@@H]1CCC(C)=C[C@H]1c1c(O)cc(CCC)cc1OC(=O)C(OP(=O)(O)OCc1ccccc1)C1CC1 ; C=C(C)[C@@H]1CCC(C)=C[C@H]1c1c(O)cc(CCC)cc1OC(=O)C1(OP(=O)(O)OCc2ccccc2)CCC1]

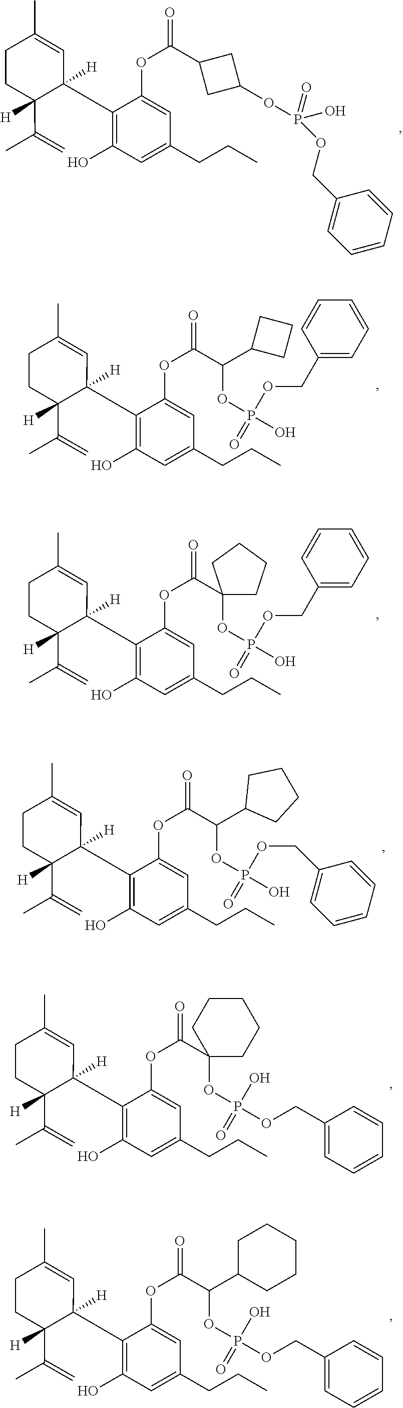

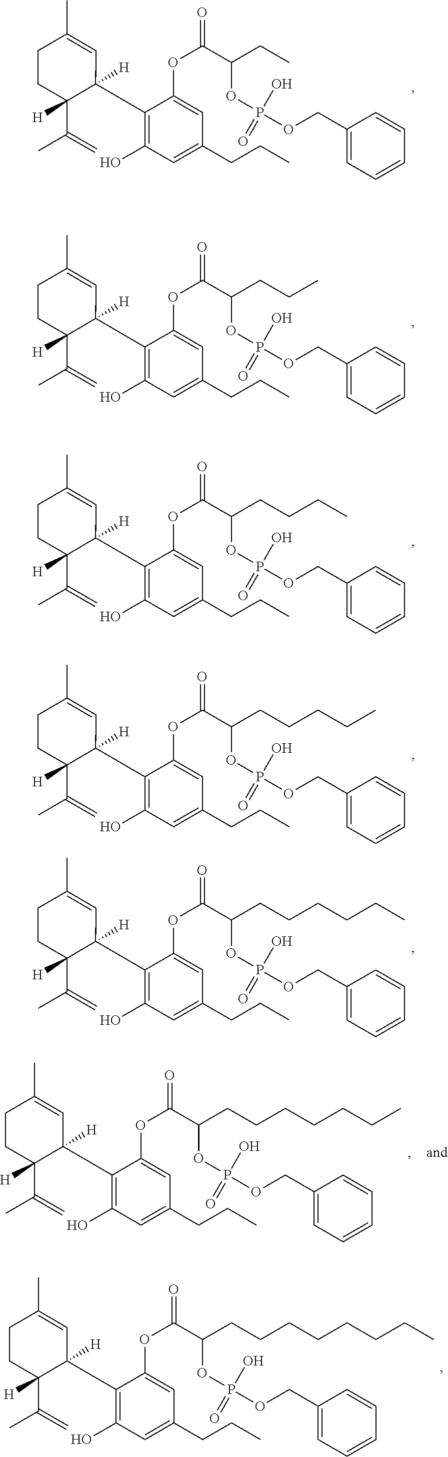

and pharmaceutically acceptable salts of any of the foregoing.

In some embodiments, the compound of Formula (I) is chosen form compounds having the following formulae:

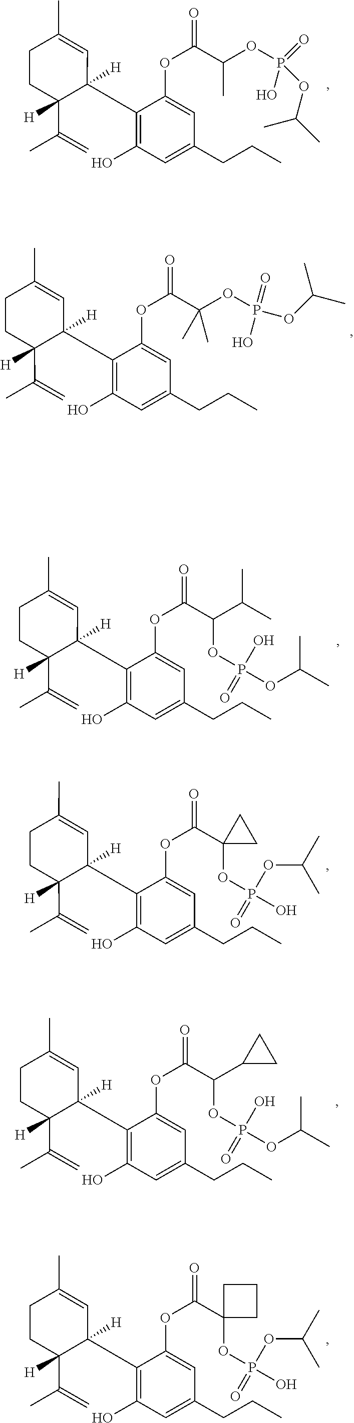 [SMILES: C=C(C)[C@@H]1CCC(C)=C[C@H]1c1c(O)cc(CCC)cc1OC(=O)C(C)OP(=O)(O)OC(C)C ; C=C(C)[C@@H]1CCC(C)=C[C@H]1c1c(O)cc(CCC)cc1OC(=O)C(C)(C)OP(=O)(O)OC(C)C ; C=C(C)[C@@H]1CCC(C)=C[C@H]1c1c(O)cc(CCC)cc1OC(=O)C(OP(=O)(O)OC(C)C)C(C)C ; C=C(C)[C@@H]1CCC(C)=C[C@H]1c1c(O)cc(CCC)cc1OC(=O)C1(OP(=O)(O)OC(C)C)CC1 ; C=C(C)[C@@H]1CCC(C)=C[C@H]1c1c(O)cc(CCC)cc1OC(=O)C(OP(=O)(O)OC(C)C)C1CC1 ; C=C(C)[C@@H]1CCC(C)=C[C@H]1c1c(O)cc(CCC)cc1OC(=O)C1(OP(=O)(O)OC(C)C)CCC1]

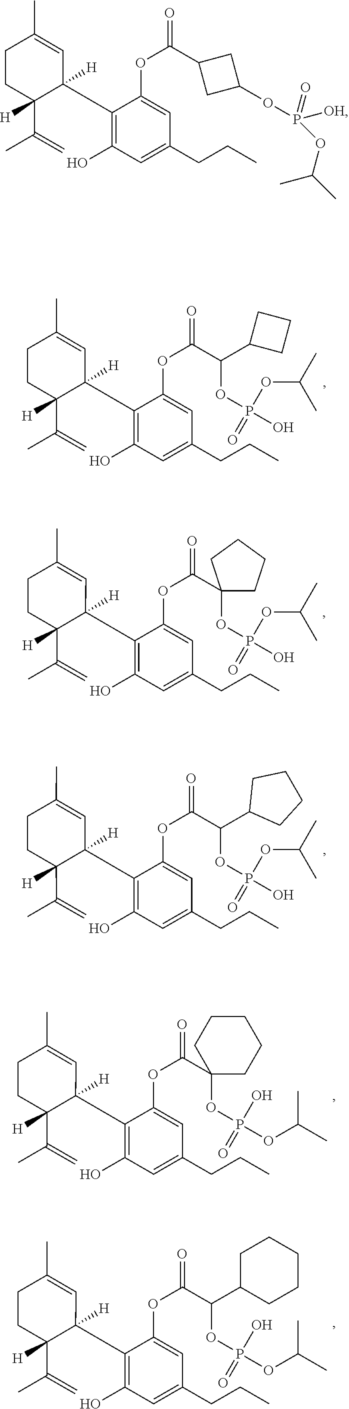 [SMILES: C=C(C)[C@@H]1CCC(C)=C[C@H]1c1c(O)cc(CCC)cc1OC(=O)C1CC(OP(=O)(O)OC(C)C)C1 ; C=C(C)[C@@H]1CCC(C)=C[C@H]1c1c(O)cc(CCC)cc1OC(=O)C(OP(=O)(O)OC(C)C)C1CCC1 ; C=C(C)[C@@H]1CCC(C)=C[C@H]1c1c(O)cc(CCC)cc1OC(=O)C1(OP(=O)(O)OC(C)C)CCCC1 ; C=C(C)[C@@H]1CCC(C)=C[C@H]1c1c(O)cc(CCC)cc1OC(=O)C(OP(=O)(O)OC(C)C)C1CCCC1 ; C=C(C)[C@@H]1CCC(C)=C[C@H]1c1c(O)cc(CCC)cc1OC(=O)C1(OP(=O)(O)OC(C)C)CCCCC1 ; C=C(C)[C@@H]1CCC(C)=C[C@H]1c1c(O)cc(CCC)cc1OC(=O)C(OP(=O)(O)OC(C)C)C1CCCCC1]

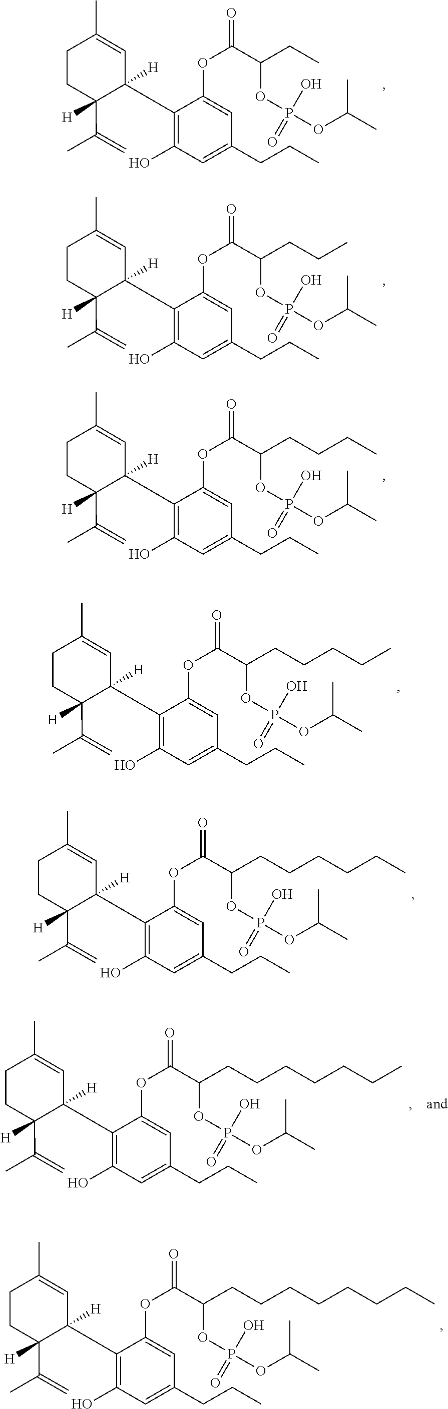

and pharmaceutically acceptable salts of any of the foregoing.

In some embodiments, the compound of Formula (I) is chosen from compounds having the following formulae:

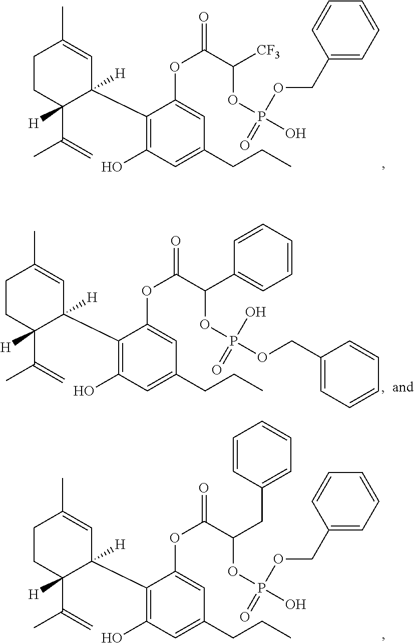

and pharmaceutically acceptable salts of any of the foregoing.

In some embodiments, the compound of Formula (I) is chosen form compounds having the following formulae:

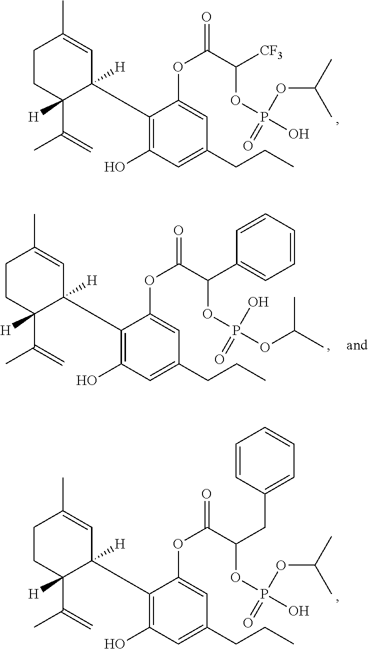 [SMILES: C=C(C)[C@@H]1CCC(C)=C[C@H]1c1c(O)cc(CCC)cc1OC(=O)C(OP(=O)(O)OC(C)C)C(F)(F)F ; C=C(C)[C@@H]1CCC(C)=C[C@H]1c1c(O)cc(CCC)cc1OC(=O)C(OP(=O)(O)OC(C)C)c1ccccc1 ; C=C(C)[C@@H]1CCC(C)=C[C@H]1c1c(O)cc(CCC)cc1OC(=O)C(Cc1ccccc1)OP(=O)(O)OC(C)C]

and pharmaceutically acceptable salts of any of the foregoing.

In some embodiments, the compound of Formula (I) is chosen from compounds having the following formulae:

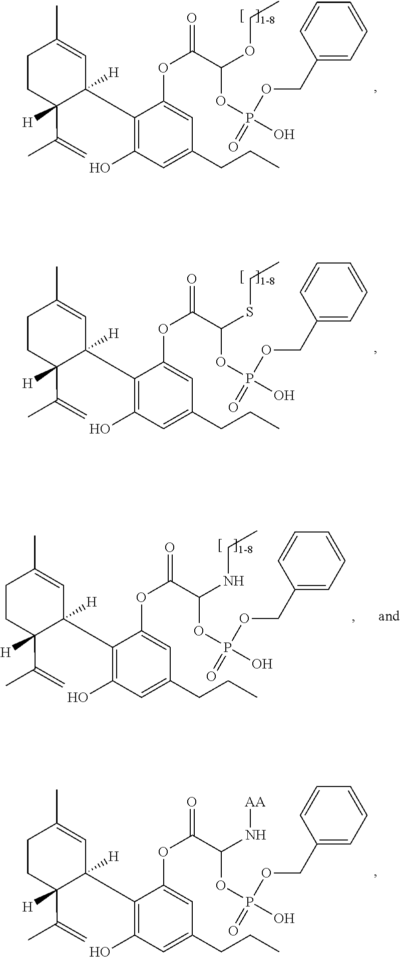

and pharmaceutically acceptable salts of any of the foregoing.

In some embodiments, the compound of Formula (I) is chosen form compounds having the following formulae:

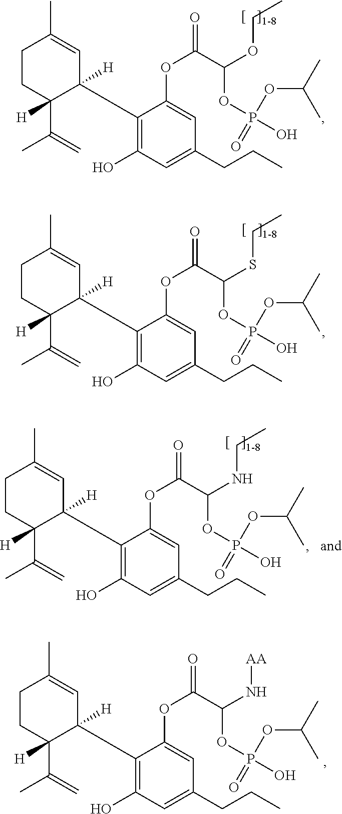

and pharmaceutically acceptable salts of any of the foregoing.

In some embodiments, the compound of Formula (I) is chosen from compounds having the following formula:

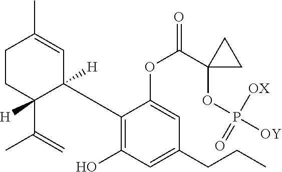

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)CH₃

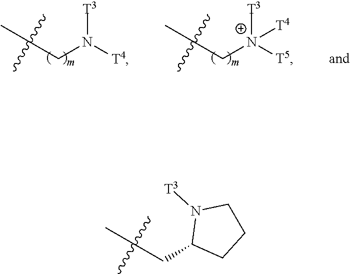

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formula:

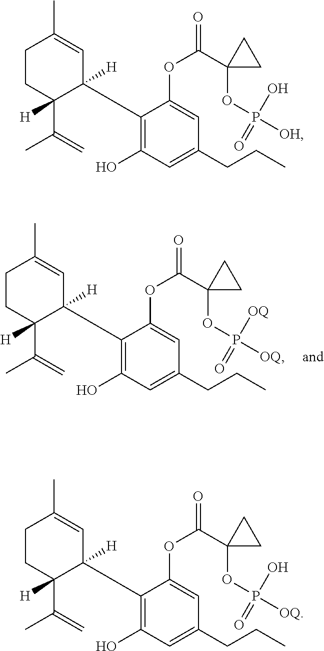

In some embodiments, the compound of Formula (I) is chosen from compounds having the following formula:

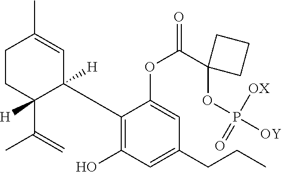

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃

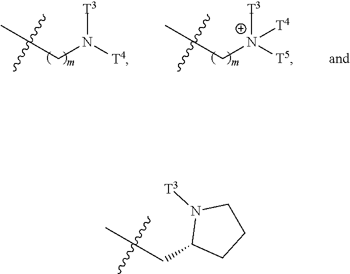

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formula:

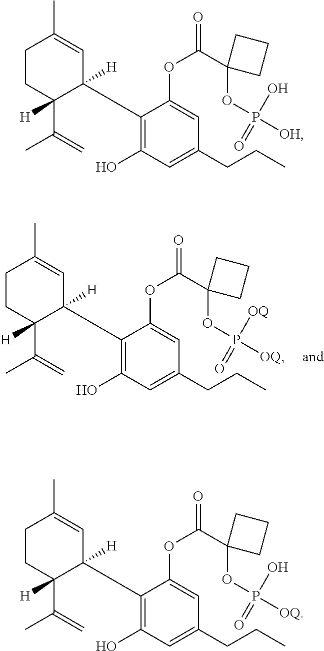

In some embodiments, the compound of Formula (I) is chosen from compounds having the following formula:

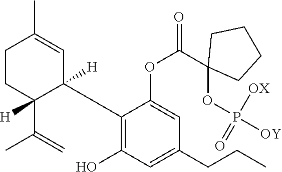

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃,

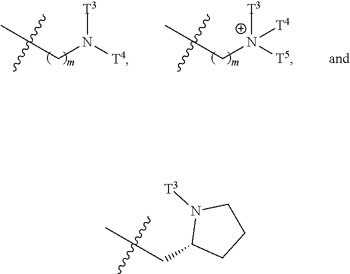

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formula:

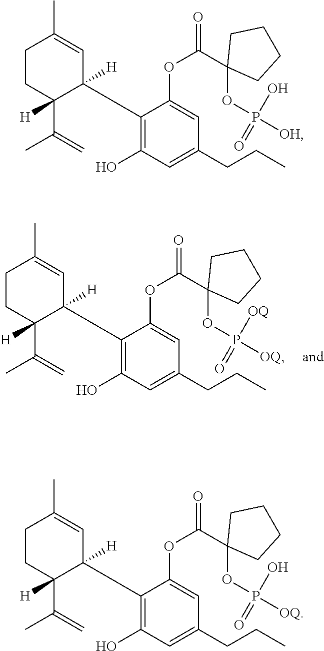

In some embodiments, the compound of Formula (I) is chosen from compounds having the following formula:

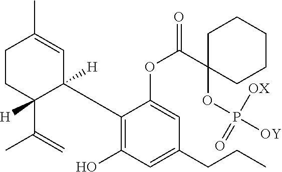

and pharmaceutically acceptable salts thereof. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃,

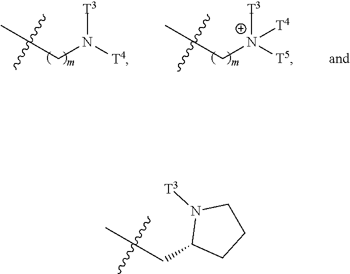

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (I) is chosen from compounds having the following formula:

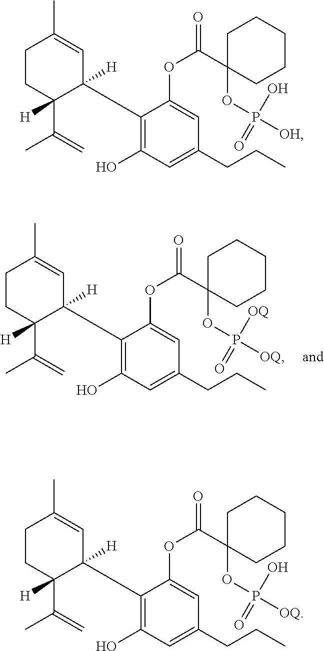

In some embodiments, the compound of Formula (I) is chosen from compounds having the following formulae:

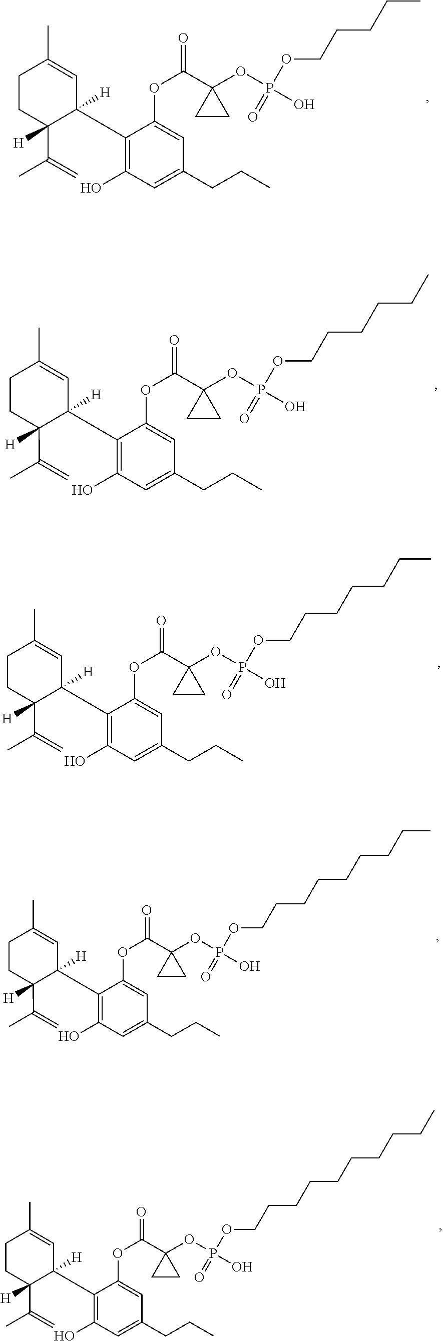 [SMILES: C=C(C)[C@@H]1CCC(C)=C[C@H]1c1c(O)cc(CCC)cc1OC(=O)C1(OP(=O)(O)OCCCCC)CC1 ; C=C(C)[C@@H]1CCC(C)=C[C@H]1c1c(O)cc(CCC)cc1OC(=O)C1(OP(=O)(O)OCCCCCC)CC1 ; C=C(C)[C@@H]1CCC(C)=C[C@H]1c1c(O)cc(CCC)cc1OC(=O)C1(OP(=O)(O)OCCCCCCC)CC1 ; C=C(C)[C@@H]1CCC(C)=C[C@H]1c1c(O)cc(CCC)cc1OC(=O)C1(OP(=O)(O)OCCCCCCCCC)CC1 ; C=C(C)[C@@H]1CCC(C)=C[C@H]1c1c(O)cc(CCC)cc1OC(=O)C1(OP(=O)(O)OCCCCCCCCCC)CC1]

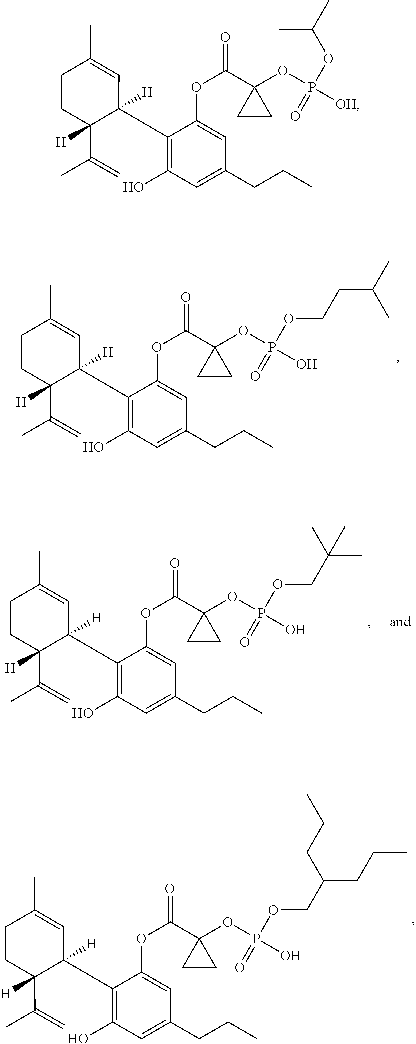 [SMILES: C=C(C)[C@@H]1CCC(C)=C[C@H]1c1c(O)cc(CCC)cc1OC(=O)C1(OP(=O)(O)OC(C)C)CC1 ; C=C(C)[C@@H]1CCC(C)=C[C@H]1c1c(O)cc(CCC)cc1OC(=O)C1(OP(=O)(O)OCCC(C)C)CC1 ; C=C(C)[C@@H]1CCC(C)=C[C@H]1c1c(O)cc(CCC)cc1OC(=O)C1(OP(=O)(O)OCC(C)(C)C)CC1 ; C=C(C)[C@@H]1CCC(C)=C[C@H]1c1c(O)cc(CCC)cc1OC(=O)C1(OP(=O)(O)OCC(CCC)CCC)CC1]

and pharmaceutically acceptable salts of any of the foregoing.

In some embodiments, the compound of Formula (I) is chosen from compounds having the following formulae:

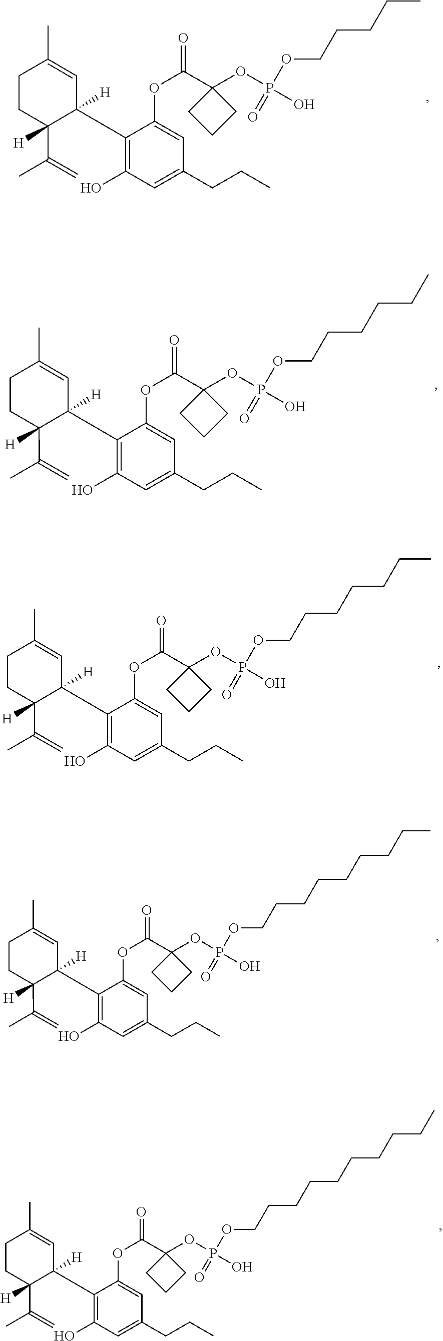 [SMILES: C=C(C)[C@@H]1CCC(C)=C[C@H]1c1c(O)cc(CCC)cc1OC(=O)C1(OP(=O)(O)OCCCCC)CCC1 ; C=C(C)[C@@H]1CCC(C)=C[C@H]1c1c(O)cc(CCC)cc1OC(=O)C1(OP(=O)(O)OCCCCCC)CCC1 ; C=C(C)[C@@H]1CCC(C)=C[C@H]1c1c(O)cc(CCC)cc1OC(=O)C1(OP(=O)(O)OCCCCCCC)CCC1 ; C=C(C)[C@@H]1CCC(C)=C[C@H]1c1c(O)cc(CCC)cc1OC(=O)C1(OP(=O)(O)OCCCCCCCCC)CCC1 ; C=C(C)[C@@H]1CCC(C)=C[C@H]1c1c(O)cc(CCC)cc1OC(=O)C1(OP(=O)(O)OCCCCCCCCCC)CCC1]

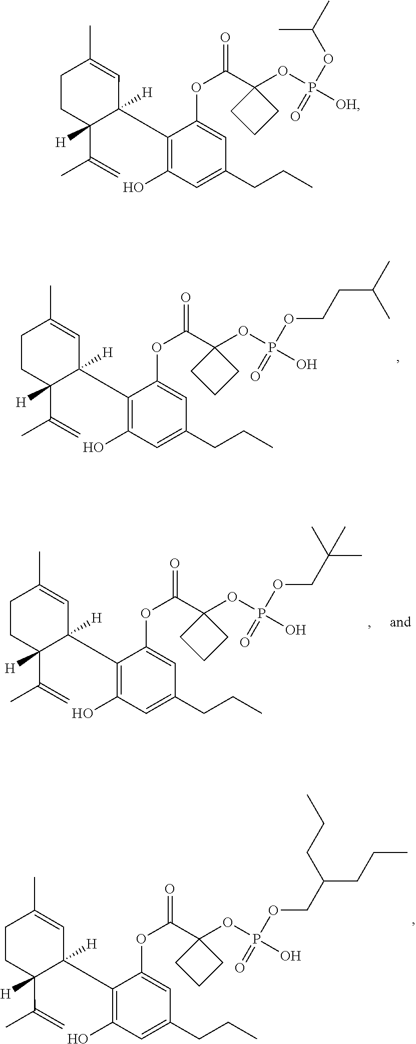 [SMILES: C=C(C)[C@@H]1CCC(C)=C[C@H]1c1c(O)cc(CCC)cc1OC(=O)C1(OP(=O)(O)OC(C)C)CCC1 ; C=C(C)[C@@H]1CCC(C)=C[C@H]1c1c(O)cc(CCC)cc1OC(=O)C1(OP(=O)(O)OCCC(C)C)CCC1 ; C=C(C)[C@@H]1CCC(C)=C[C@H]1c1c(O)cc(CCC)cc1OC(=O)C1(OP(=O)(O)OCC(C)(C)C)CCC1 ; C=C(C)[C@@H]1CCC(C)=C[C@H]1c1c(O)cc(CCC)cc1OC(=O)C1(OP(=O)(O)OCC(CCC)CCC)CCC1]

and pharmaceutically acceptable salts of any of the foregoing.

In some embodiments, the compound of Formula (I) is chosen from compounds having the following formulae:

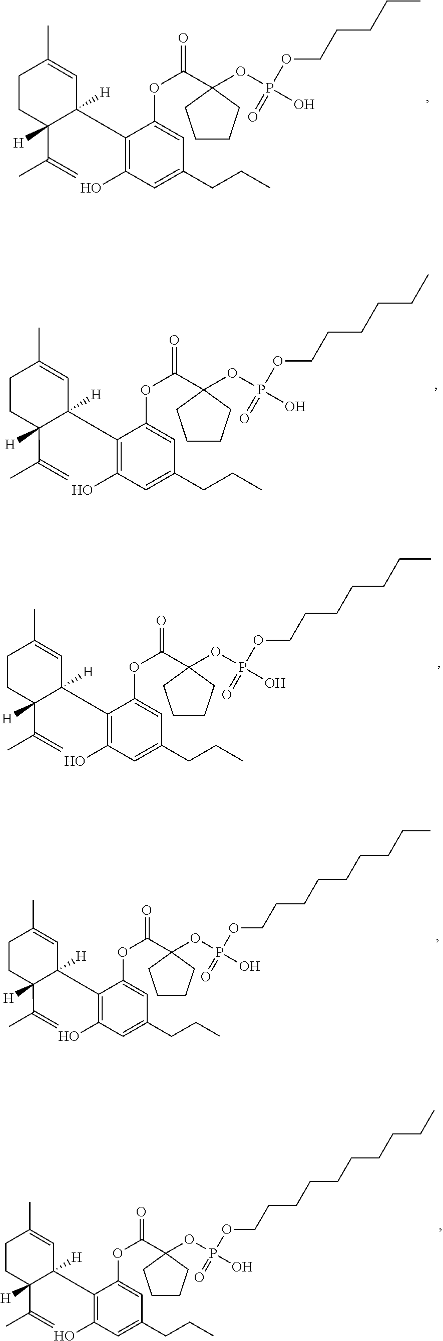 [SMILES: C=C(C)[C@@H]1CCC(C)=C[C@H]1c1c(O)cc(CCC)cc1OC(=O)C1(OP(=O)(O)OCCCCC)CCCC1 ; C=C(C)[C@@H]1CCC(C)=C[C@H]1c1c(O)cc(CCC)cc1OC(=O)C1(OP(=O)(O)OCCCCCC)CCCC1 ; C=C(C)[C@@H]1CCC(C)=C[C@H]1c1c(O)cc(CCC)cc1OC(=O)C1(OP(=O)(O)OCCCCCCC)CCCC1 ; C=C(C)[C@@H]1CCC(C)=C[C@H]1c1c(O)cc(CCC)cc1OC(=O)C1(OP(=O)(O)OCCCCCCCCC)CCCC1 ; C=C(C)[C@@H]1CCC(C)=C[C@H]1c1c(O)cc(CCC)cc1OC(=O)C1(OP(=O)(O)OCCCCCCCCCC)CCCC1]

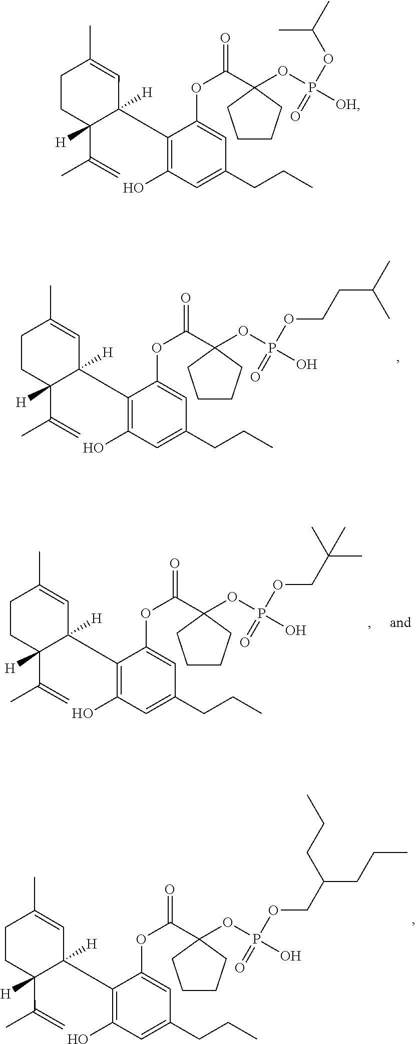 [SMILES: C=C(C)[C@@H]1CCC(C)=C[C@H]1c1c(O)cc(CCC)cc1OC(=O)C1(OP(=O)(O)OC(C)C)CCCC1 ; C=C(C)[C@@H]1CCC(C)=C[C@H]1c1c(O)cc(CCC)cc1OC(=O)C1(OP(=O)(O)OCCC(C)C)CCCC1 ; C=C(C)[C@@H]1CCC(C)=C[C@H]1c1c(O)cc(CCC)cc1OC(=O)C1(OP(=O)(O)OCC(C)(C)C)CCCC1 ; C=C(C)[C@@H]1CCC(C)=C[C@H]1c1c(O)cc(CCC)cc1OC(=O)C1(OP(=O)(O)OCC(CCC)CCC)CCCC1]

and pharmaceutically acceptable salts of any of the foregoing.

In some embodiments, the compound of Formula (I) is chosen from compounds having the following formulae:

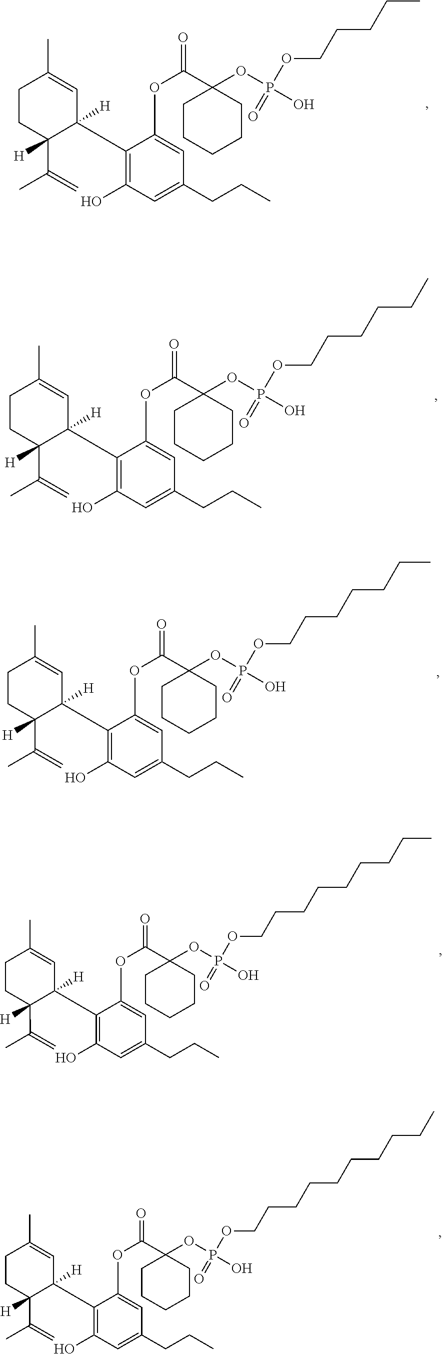 [SMILES: C=C(C)[C@@H]1CCC(C)=C[C@H]1c1c(O)cc(CCC)cc1OC(=O)C1(OP(=O)(O)OCCCCC)CCCCC1 ; C=C(C)[C@@H]1CCC(C)=C[C@H]1c1c(O)cc(CCC)cc1OC(=O)C1(OP(=O)(O)OCCCCCC)CCCCC1 ; C=C(C)[C@@H]1CCC(C)=C[C@H]1c1c(O)cc(CCC)cc1OC(=O)C1(OP(=O)(O)OCCCCCCC)CCCCC1 ; C=C(C)[C@@H]1CCC(C)=C[C@H]1c1c(O)cc(CCC)cc1OC(=O)C1(OP(=O)(O)OCCCCCCCCC)CCCCC1 ; C=C(C)[C@@H]1CCC(C)=C[C@H]1c1c(O)cc(CCC)cc1OC(=O)C1(OP(=O)(O)OCCCCCCCCCC)CCCCC1]

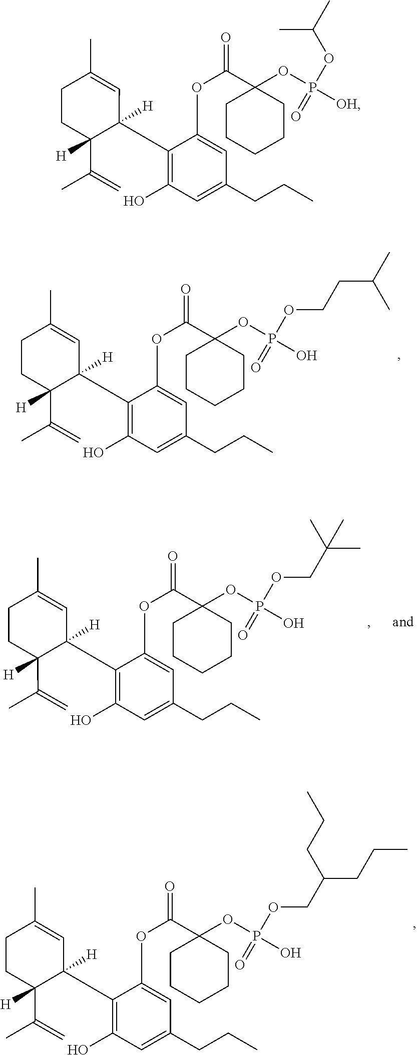 [SMILES: C=C(C)[C@@H]1CCC(C)=C[C@H]1c1c(O)cc(CCC)cc1OC(=O)C1(OP(=O)(O)OC(C)C)CCCCC1 ; C=C(C)[C@@H]1CCC(C)=C[C@H]1c1c(O)cc(CCC)cc1OC(=O)C1(OP(=O)(O)OCCC(C)C)CCCCC1 ; C=C(C)[C@@H]1CCC(C)=C[C@H]1c1c(O)cc(CCC)cc1OC(=O)C1(OP(=O)(O)OCC(C)(C)C)CCCCC1 ; C=C(C)[C@@H]1CCC(C)=C[C@H]1c1c(O)cc(CCC)cc1OC(=O)C1(OP(=O)(O)OCC(CCC)CCC)CCCCC1]

and pharmaceutically acceptable salts of any of the foregoing.

In some embodiments, the compound of Formula (I) is chosen from compounds having the following formulae:

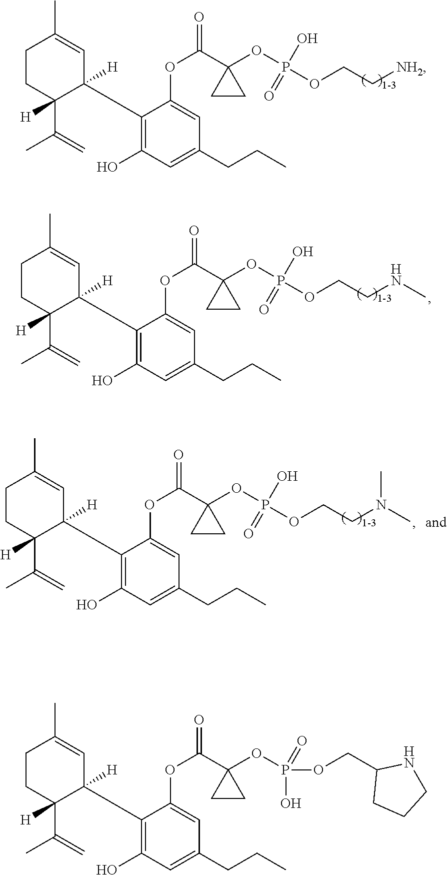

and pharmaceutically acceptable salts of any of the foregoing.

In some embodiments, the compound of Formula (I) is chosen from compounds having the following formulae:

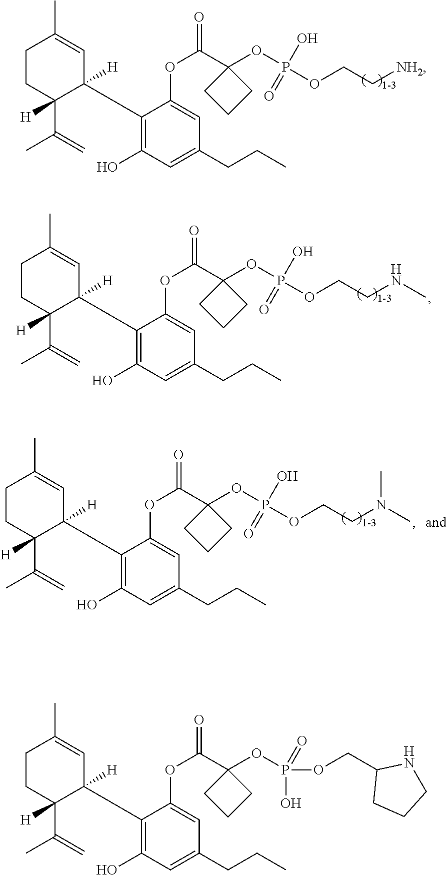

and pharmaceutically acceptable salts of any of the foregoing.

In some embodiments, the compound of Formula (I) is chosen from compounds

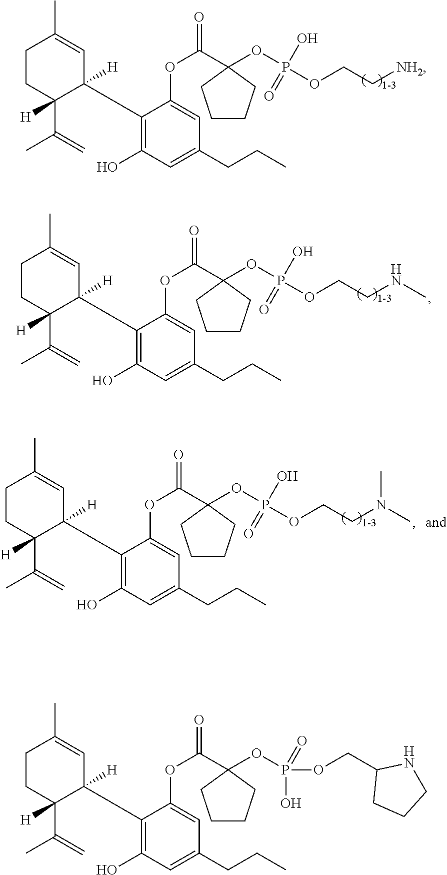

and pharmaceutically acceptable salts of any of the foregoing.

In some embodiments, the compound of Formula (I) is chosen from compounds having the following formulae:

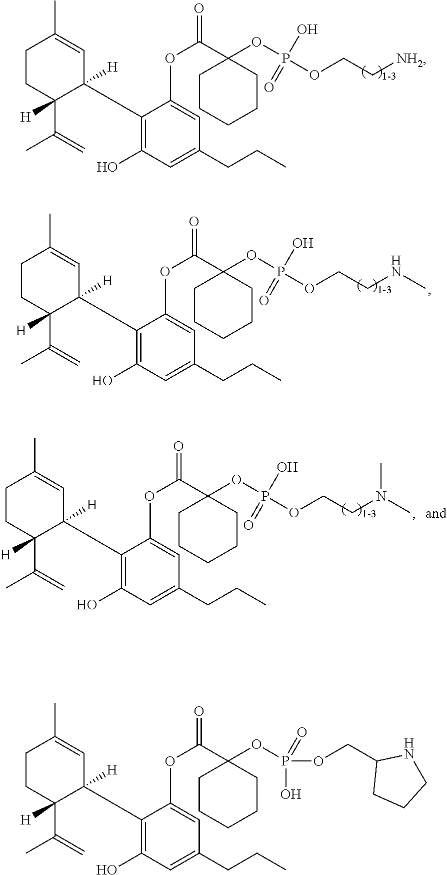

and pharmaceutically acceptable salts of any of the foregoing.

In some embodiments, the compound of Formula (I) is chosen from compounds having the following formulae:

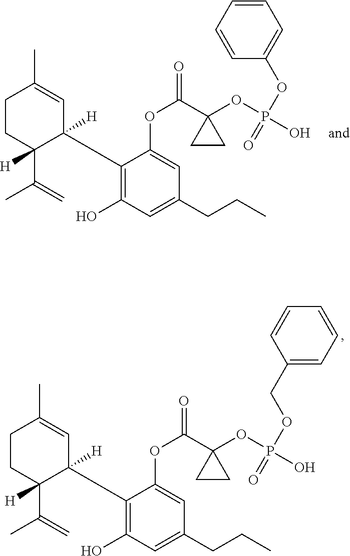 [SMILES: C=C(C)[C@@H]1CCC(C)=C[C@H]1c1c(O)cc(CCC)cc1OC(=O)C1(OP(=O)(O)Oc2ccccc2)CC1 ; C=C(C)[C@@H]1CCC(C)=C[C@H]1c1c(O)cc(CCC)cc1OC(=O)C1(OP(=O)(O)OCc2ccccc2)CC1]

and pharmaceutically acceptable salts of any of the foregoing.

In some embodiments, the compound of Formula (I) is chosen from compounds having the following formulae:

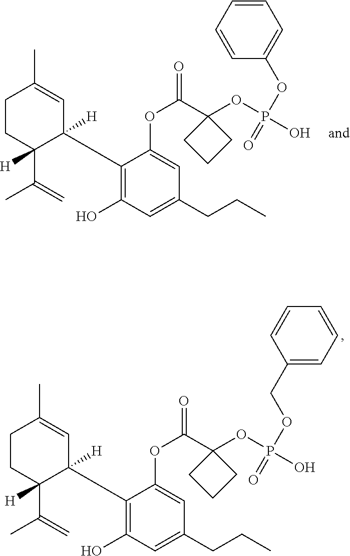 [SMILES: C=C(C)[C@@H]1CCC(C)=C[C@H]1c1c(O)cc(CCC)cc1OC(=O)C1(OP(=O)(O)Oc2ccccc2)CCC1 ; C=C(C)[C@@H]1CCC(C)=C[C@H]1c1c(O)cc(CCC)cc1OC(=O)C1(OP(=O)(O)OCc2ccccc2)CCC1]

and pharmaceutically acceptable salts of any of the foregoing.

In some embodiments, the compound of Formula (I) is chosen from compounds having the following formulae:

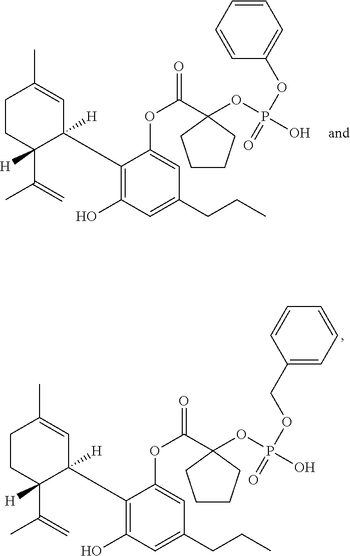 [SMILES: C=C(C)[C@@H]1CCC(C)=C[C@H]1c1c(O)cc(CCC)cc1OC(=O)C1(OP(=O)(O)Oc2ccccc2)CCCC1 ; C=C(C)[C@@H]1CCC(C)=C[C@H]1c1c(O)cc(CCC)cc1OC(=O)C1(OP(=O)(O)OCc2ccccc2)CCCC1]

and pharmaceutically acceptable salts of any of the foregoing.

In some embodiments, the compound of Formula (I) is chosen from compounds having the following formulae:

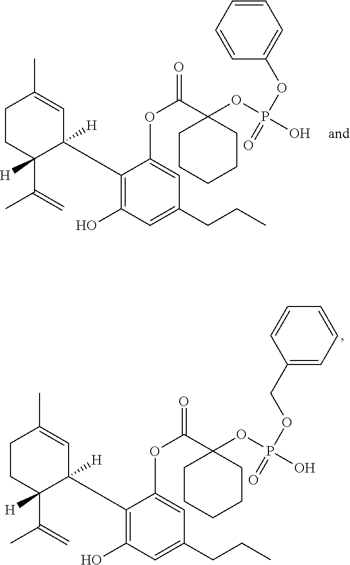 [SMILES: C=C(C)[C@@H]1CCC(C)=C[C@H]1c1c(O)cc(CCC)cc1OC(=O)C1(OP(=O)(O)Oc2ccccc2)CCCCC1 ; C=C(C)[C@@H]1CCC(C)=C[C@H]1c1c(O)cc(CCC)cc1OC(=O)C1(OP(=O)(O)OCc2ccccc2)CCCCC1]

and pharmaceutically acceptable salts of any of the foregoing.

In some embodiments, the compound of Formula (I) is chosen from compounds having the following formulae:

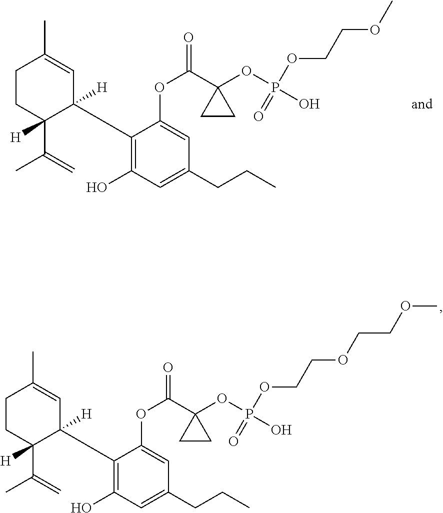 [SMILES: C=C(C)[C@@H]1CCC(C)=C[C@H]1c1c(O)cc(CCC)cc1OC(=O)C1(OP(=O)(O)OCCOC)CC1 ; C=C(C)[C@@H]1CCC(C)=C[C@H]1c1c(O)cc(CCC)cc1OC(=O)C1(OP(=O)(O)OCCOCCOC)CC1]

and pharmaceutically acceptable salts ofany of the foregoing.

In some embodiments, the compound of Formula (I) is chosen from compounds having the following formulae:

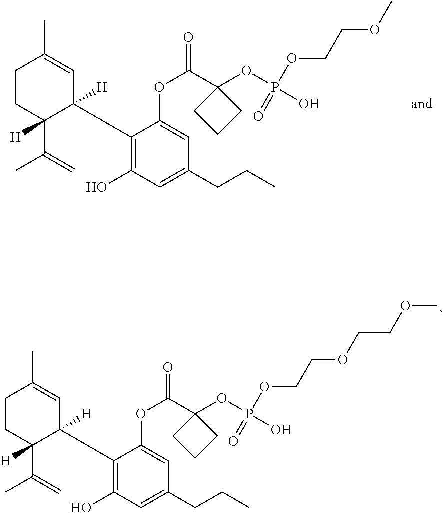 [SMILES: C=C(C)[C@@H]1CCC(C)=C[C@H]1c1c(O)cc(CCC)cc1OC(=O)C1(OP(=O)(O)OCCOC)CCC1 ; C=C(C)[C@@H]1CCC(C)=C[C@H]1c1c(O)cc(CCC)cc1OC(=O)C1(OP(=O)(O)OCCOCCOC)CCC1]

and pharmaceutically acceptable salts of any of the foregoing.

In some embodiments, the compound of Formula (I) is chosen from compounds having the following formulae:

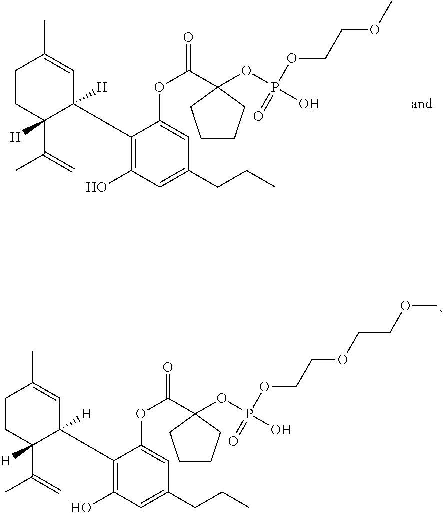 [SMILES: C=C(C)[C@@H]1CCC(C)=C[C@H]1c1c(O)cc(CCC)cc1OC(=O)C1(OP(=O)(O)OCCOC)CCCC1 ; C=C(C)[C@@H]1CCC(C)=C[C@H]1c1c(O)cc(CCC)cc1OC(=O)C1(OP(=O)(O)OCCOCCOC)CCCC1]

and pharmaceutically acceptable salts of any of the foregoing.

In some embodiments, the compound of Formula (I) is chosen from compounds

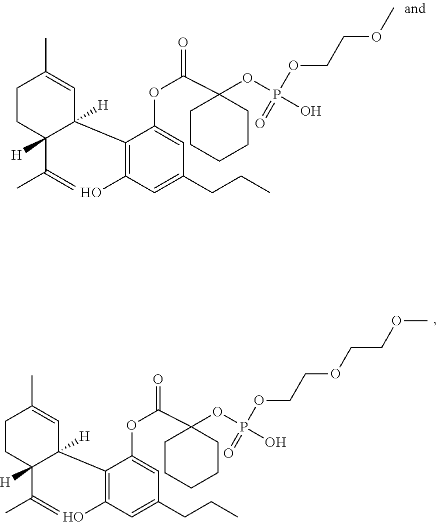 [SMILES: C=C(C)[C@@H]1CCC(C)=C[C@H]1c1c(O)cc(CCC)cc1OC(=O)C1(OP(=O)(O)OCCOC)CCCCC1 ; C=C(C)[C@@H]1CCC(C)=C[C@H]1c1c(O)cc(CCC)cc1OC(=O)C1(OP(=O)(O)OCCOCCOC)CCCCC1]

and pharmaceutically acceptable salts of any of the foregoing.

In some embodiments, the compound of Formula (I) is chosen from compounds having the following formulae:

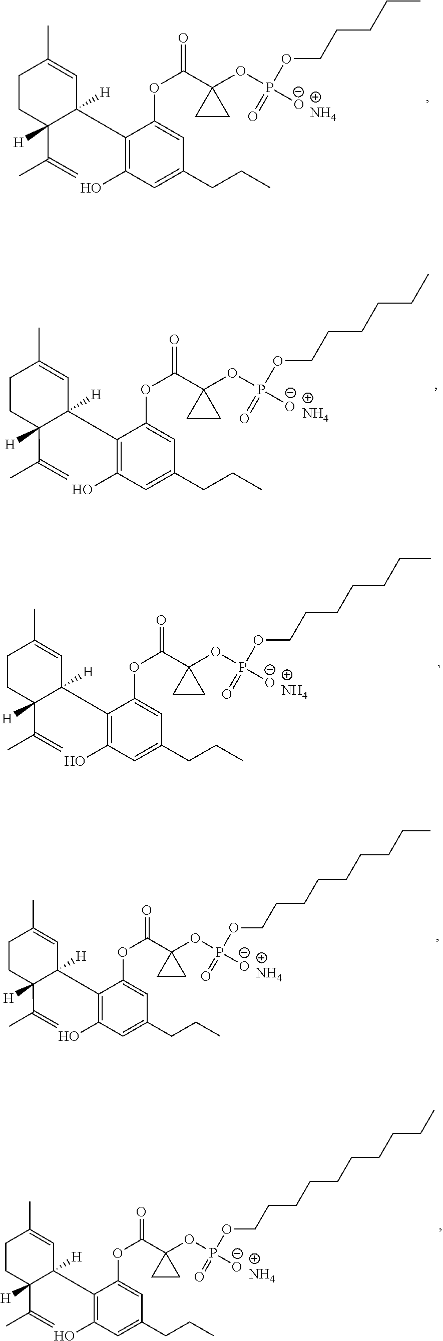

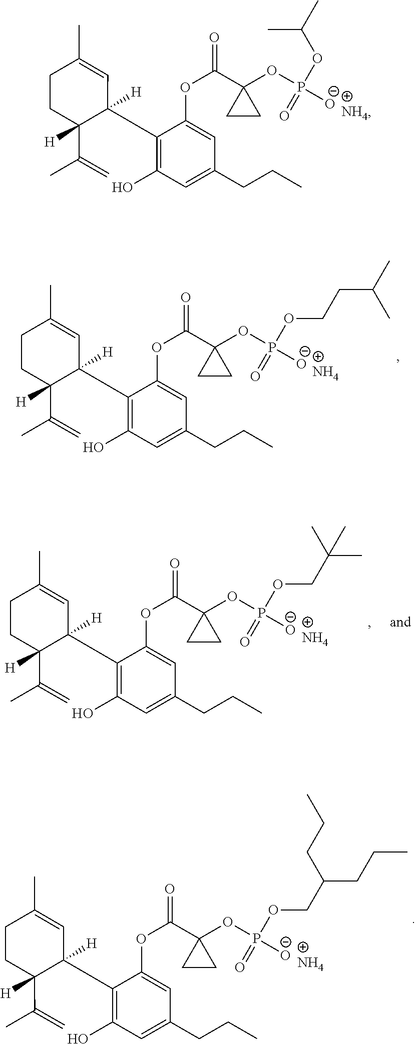

In some embodiments, the compound of Formula (I) is chosen from compounds having the following formulae:

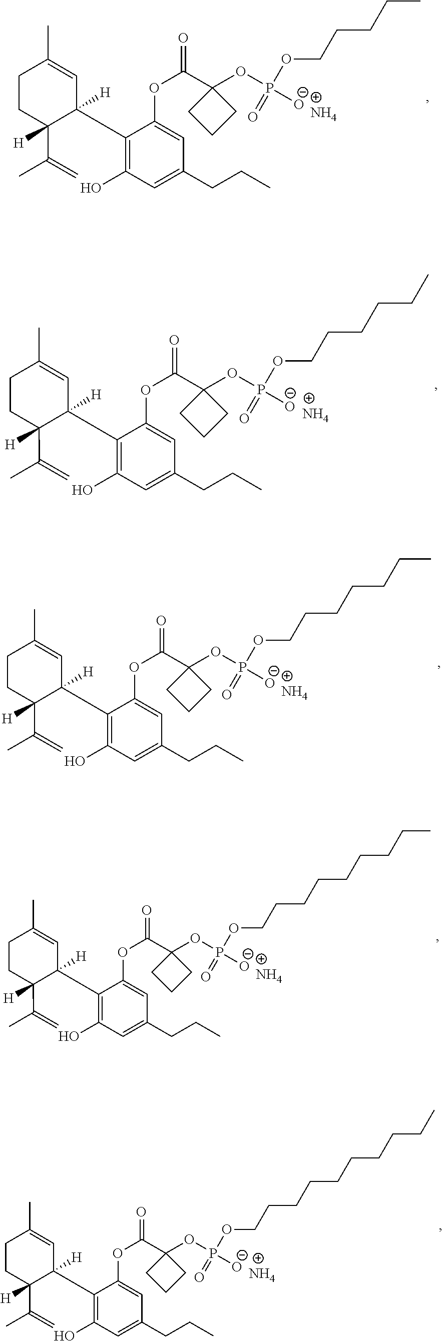

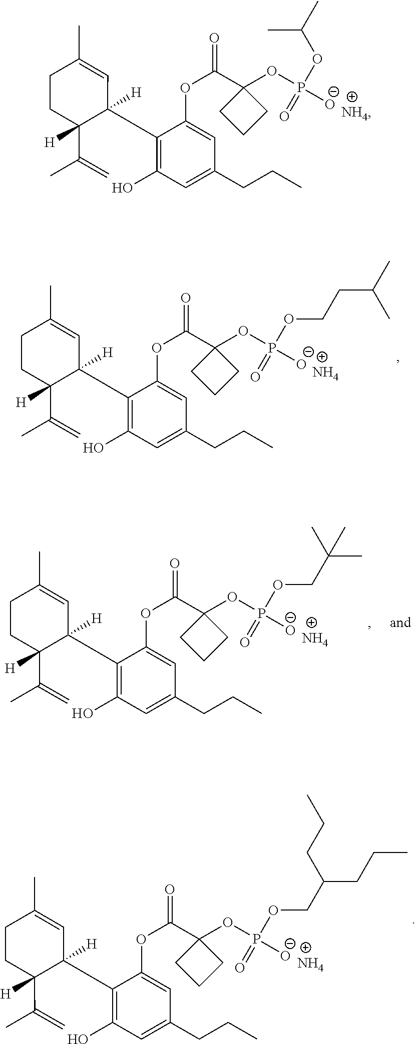

In some embodiments, the compound of Formula (I) is chosen from compounds having the following formulae:

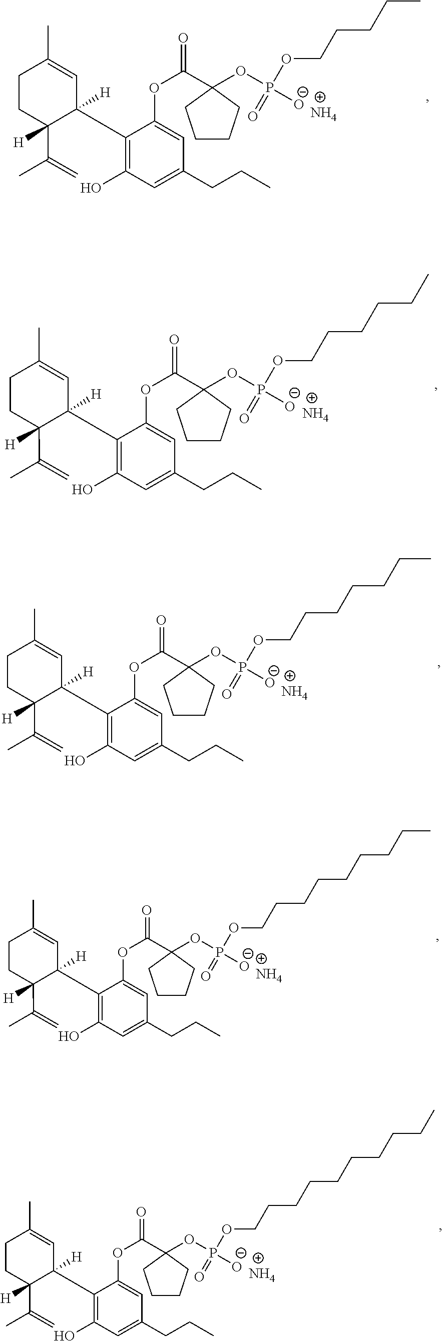

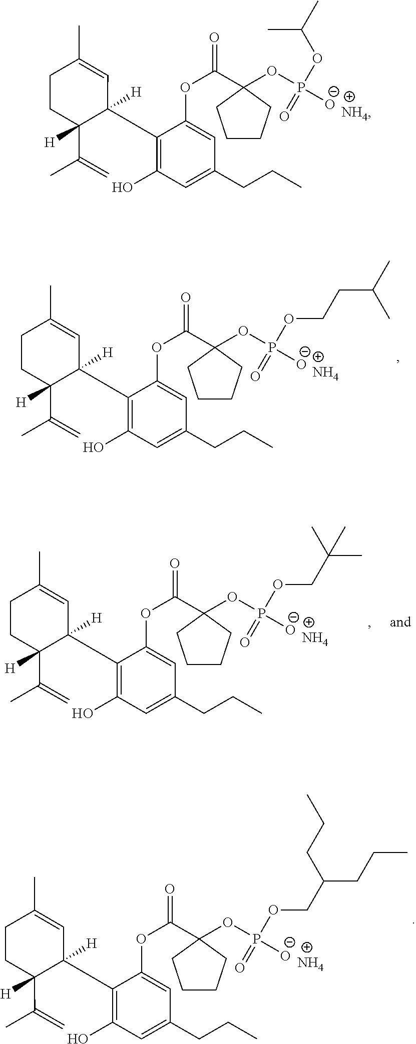

In some embodiments, the compound of Formula (I) is chosen from compounds having the following formulae:

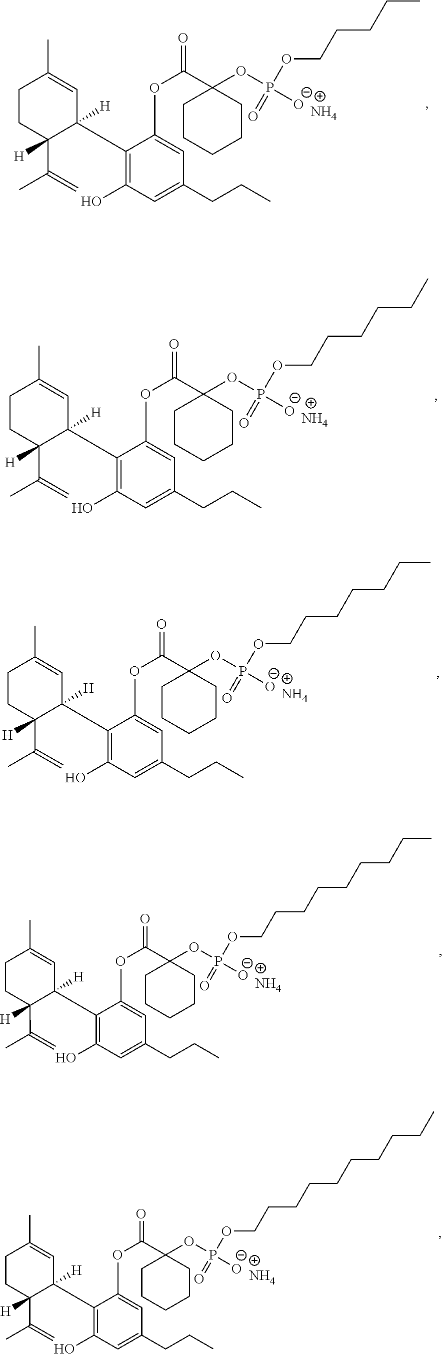

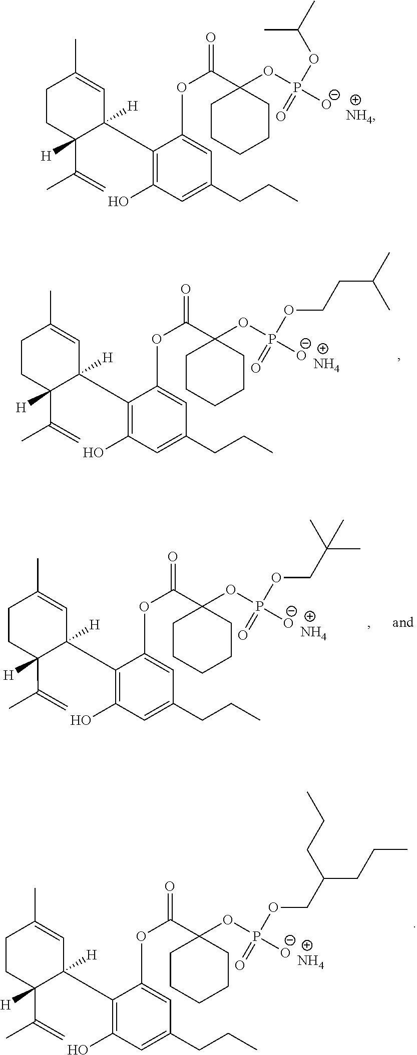

In some embodiments, the compound of Formula (I) is chosen from compounds having the following formulae:

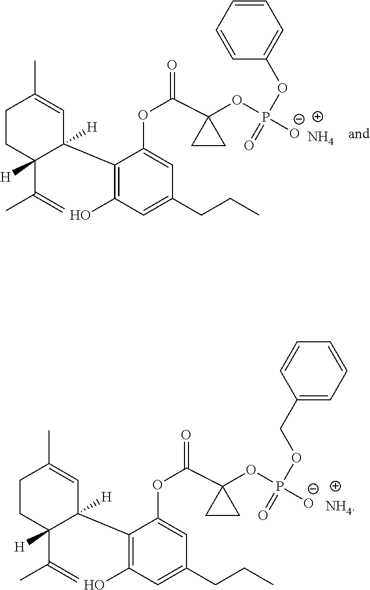

In some embodiments, the compound of Formula (I) is chosen from compounds having the following formulae:

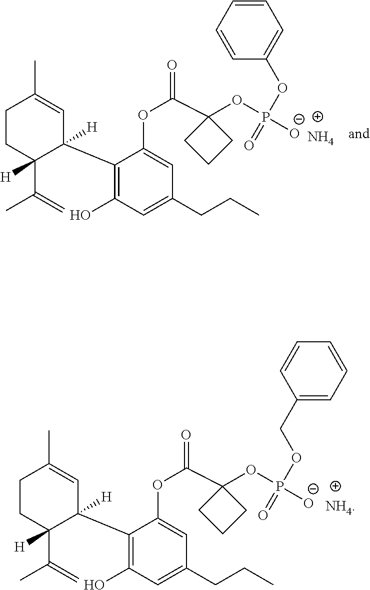

In some embodiments, the compound of Formula (I) is chosen from compounds having the following formulae:

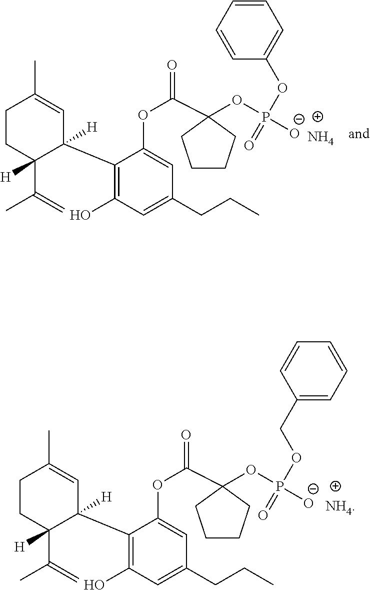

In some embodiments, the compound of Formula (I) is chosen from compounds having the following formulae:

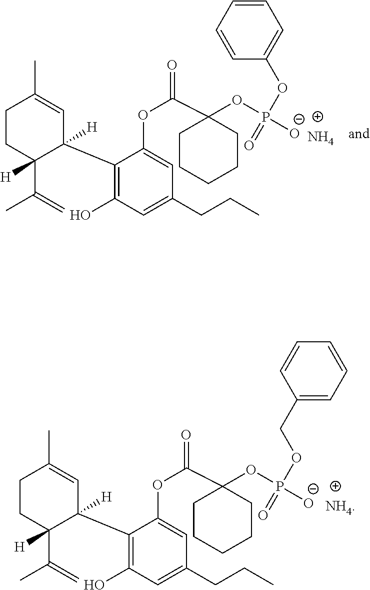

In some embodiments, the compound of Formula (I) is chosen from compounds having the following formulae:

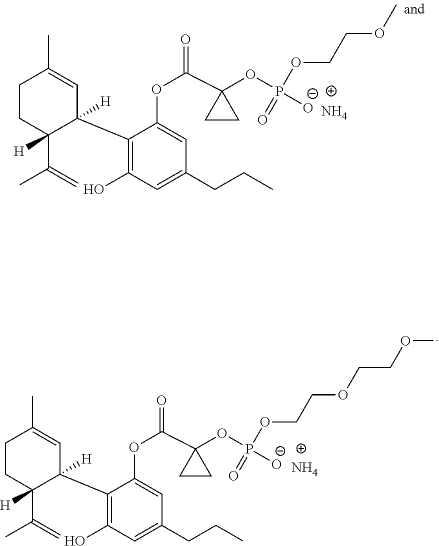

In some embodiments, the compound of Formula (I) is chosen from compounds having the following formulae:

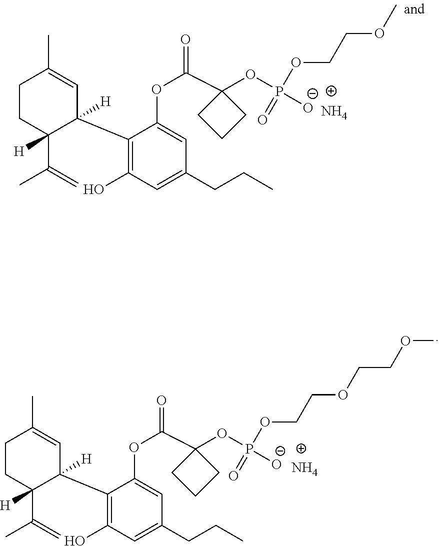

In some embodiments, the compound of Formula (I) is chosen from compounds having the following formulae:

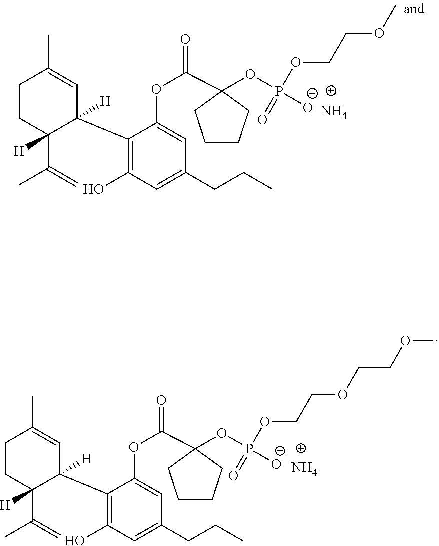

In some embodiments, the compound of Formula (I) is chosen from compounds having the following formulae:

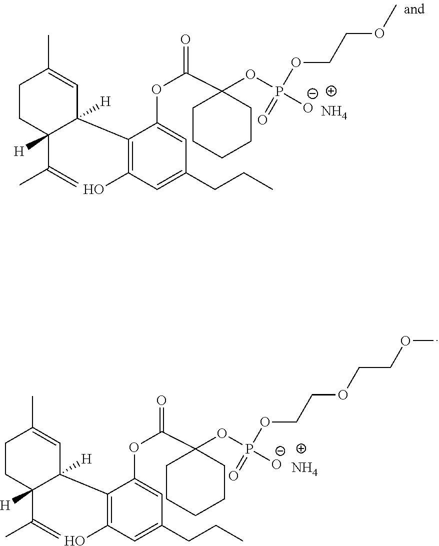

In some embodiments, the compound of Formula (I) is chosen from compounds having the following formulae:

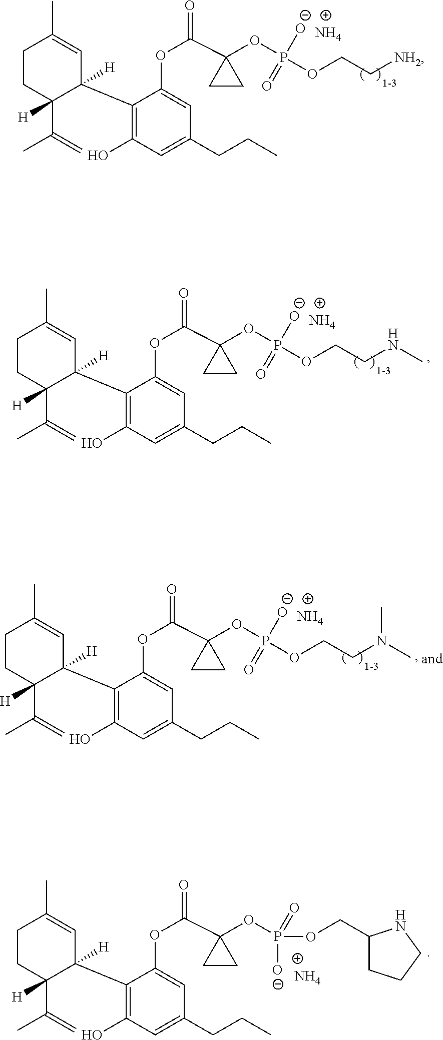

In some embodiments, the compound of Formula (I) is chosen from compounds having the following formulae:

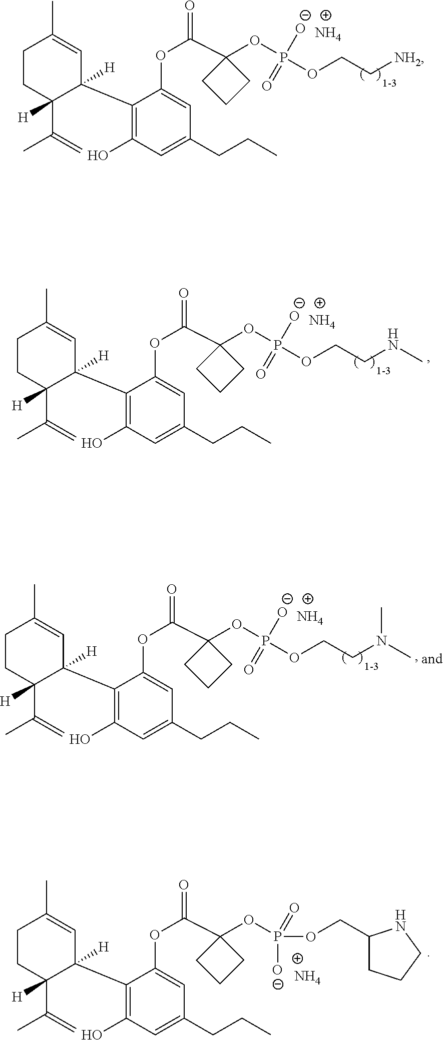

In some embodiments, the compound of Formula (I) is chosen from compounds having the following formulae:

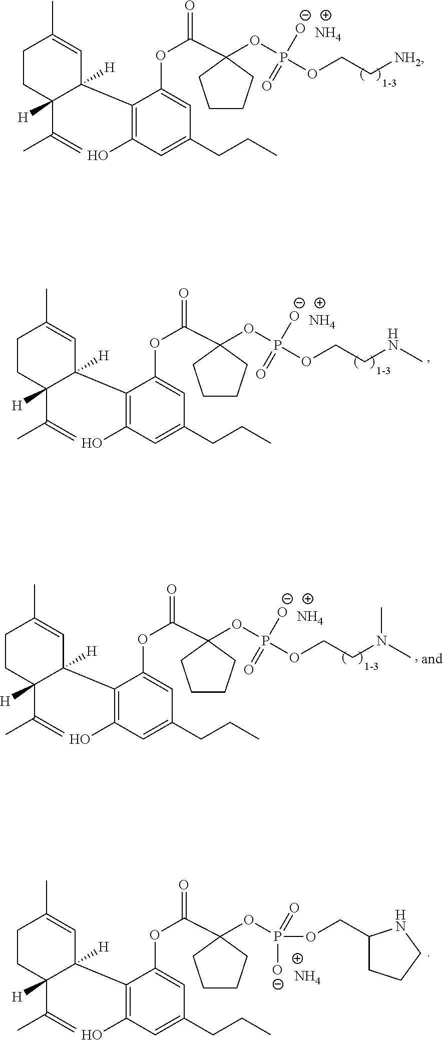

In some embodiments, the compound of Formula (I) is chosen from compounds having the following formulae:

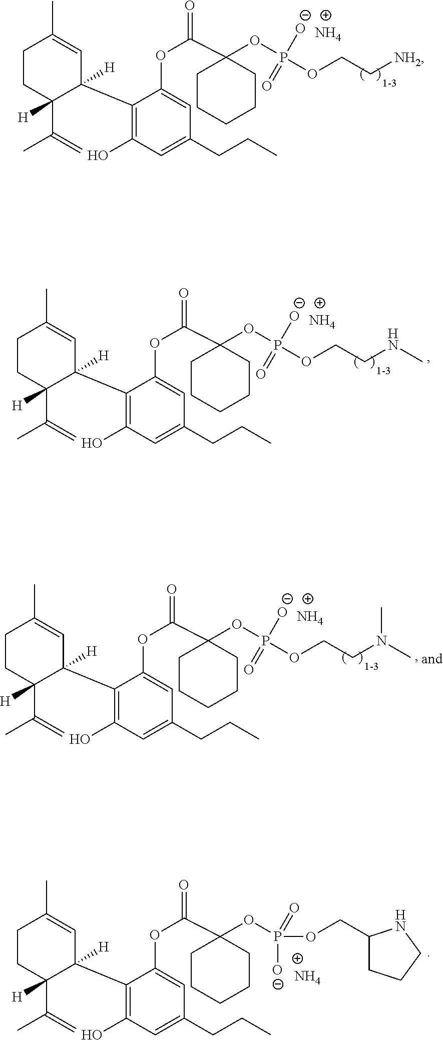

In some embodiments, the compound of Formula (I) is chosen from compounds having the following formula:

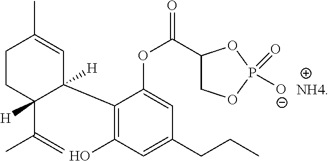

In some embodiments, the compound of Formula (I) has a human plasma stability, T_{1/2}, of no less than about 15 min. For example, the compound of Formula (I) may have a human plasma stability, T_{1/2}, of between about 15 min and about 15 hours. In some embodiments, the compound of Formula (I) has a human plasma stability, T_{1/2}, of between about 15 min and about 30 min. In some embodiments, the compound of Formula (I) has a human plasma stability, T_{1/2}, between about 30 min and about 45 min. In some embodiments, the compound of Formula (I) has a human plasma stability, T_{1/2}, between about 45 min and about 1 hour. In some embodiments, the compound of Formula (I) has a human plasma stability, T_{1/2}, between about 1 and about 2 hours. In some embodiments, the compound of Formula (I) has a human plasma stability, T_{1/2}, between about 2 and about 3 hours. In some embodiments, the compound of Formula (I) has a human plasma stability, T_{1/2}, between about 3 and about 5 hours. In some embodiments, the compound of Formula (I) has a human plasma stability, T_{1/2}, between about 5 and about 10 hours. In some embodiments, the compound of Formula (I) has a human plasma stability, T_{1/2}, of between about 10 and about 15 hours. In some embodiments, the compound of Formula (I) has a human plasma stability, T_{1/2}, of greater than about 1 hour. In some embodiments, the compound of Formula (I) has a human plasma stability, T_{1/2}, of greater than about 2 hours. In some embodiments, the compound of Formula (I) has a human plasma stability, T_{1/2}, of greater than about 4 hours. In some embodiments, the compound of Formula (I) has a human plasma stability, T_{1/2}, of greater than about 10 hours. In some embodiments, the compound of Formula (I) has a human plasma stability, T_{1/2}, of greater than about 15 hours.

As used herein, the “human plasma stability, T_{1/2},” of compounds of Formula (I) is determined according to the procedure described in Example 11.

In some embodiments, the compound of Formula (I) has a human liver microsomes stability, T_{1/2}, of no less than about 15 min. For example, the compound of Formula (I) may have a human liver microsomes stability, T_{1/2}, of between about 15 min and about 2 hours. In some embodiments, the compound of Formula (I) has a human liver microsomes stability, T_{1/2}, of between about 15 and about 30 min. In some embodiments, the compound of Formula (I) has a human liver microsomes stability, T_{1/2}, of between about 30 and about 45 min. In some embodiments, the compound of Formula (I) has a human liver microsomes stability, T_{1/2}, of between about 45 min and about 1 hour. In some embodiments, the compound of Formula (I) has a human liver microsomes stability, T_{1/2}, of between about 1 and about 1.25 hours. In some embodiments, the compound of Formula (I) has a human liver microsomes stability, T_{1/2}, of between about 1.25 and about 1.5 hours. In some embodiments, the compound of Formula (I) has a human liver microsomes stability, T_{1/2}, of between about 1.5 and about 1.75 hours. In some embodiments, the compound of Formula (I) has a human liver microsomes stability, T_{1/2}, of between about 1.75 and about 2 hours. In some embodiments, the compound of Formula (I) has a human liver microsomes stability, T_{1/2}, of greater than about 2 hours.

In some embodiments, the compound of Formula (I) has a human liver microsomes stability, T_{1/2}, greater than the stability of CBD, measured under the same experimental conditions. In some embodiments, the compound of Formula (I) has a human liver microsomes stability, T_{1/2}, of at least 1.5 times the stability of CBD. In some embodiments, the compound of Formula (I) has a human liver microsomes stability, T_{1/2}, of at least 2 times the stability of CBD. In some embodiments, the compound of Formula (I) has a human liver microsomes stability, T_{1/2}, of at least 2.5 times the stability of CBD. In some embodiments, the compound of Formula (I) has a human liver microsomes stability, T_{1/2}, of at least 3 times the stability of CBD. In some embodiments, the compound of Formula (I) has a human liver microsomes stability, T_{1/2}, of at least 3.5 times the stability of CBD. In some embodiments, the compound of Formula (I) has a human liver microsomes stability, T_{1/2}, of at least 4 times the stability of CBD. In some embodiments, the compound of Formula (I) has a human liver microsomes stability, T_{1/2}, of at least 4.5 times the stability of CBD. In some embodiments, the compound of Formula (I) has a human liver microsomes stability, T_{1/2}, of at least 5 times the stability of CBD.

As used herein, the “human liver microsomes stability, T_{1/2},” of compounds of Formula (I) is determined according to the procedure described in Example 10.

In some embodiments, the compound of Formula (I) has a solubility in 1% DMSO of no less than about 50 μM at 25° C. For example, the compound of Formula (I) may have a solubility in 1% DMSO of between about 50 and about 500 μM at 25° C. In some embodiments, the compound of Formula (I) has a solubility in 1% DMSO between about 50 and about 75 μM at 25° C. In some embodiments, the compound of Formula (I) has a solubility in 1% DMSO between about 75 and about 100 μM at 25° C. In some embodiments, the compound of Formula (I) has a solubility in 1% DMSO between about 100 and about 125 μM at 25° C. In some embodiments, the compound of Formula (I) has a solubility in 1% DMSO between about 125 and 150 μM at 25° C. In some embodiments, the compound of Formula (I) has a solubility in 1% DMSO of greater than about 150 μM at 25° C. In some embodiments, the compound of Formula (I) has a solubility in 1% DMSO of greater than about 200 μM at 25° C. In some embodiments, the compound of Formula (I) has a solubility in 1% DMSO of greater than about 300 μM at 25° C. In some embodiments, the compound of Formula (I) has a solubility in 1% DMSO of greater than about 400 μM at 25° C. In some embodiments, the compound of Formula (I) has a solubility in 1% DMSO of greater than about 500 μM at 25° C.

In some embodiments, the compound of Formula (I) has a solubility in 1% DMSO greater than CBD, measured under the same experimental conditions. In some embodiments, the compound of Formula (I) has a solubility in 1% DMSO of at least 2 times the solubility of CBD. In some embodiments, the compound of Formula (I) has a solubility in 1% DMSO of at least 3 times the solubility of CBD. In some embodiments, the compound of Formula (I) has a solubility in 1% DMSO of at least 5 times the solubility of CBD. In some embodiments, the compound of Formula (I) has a solubility in 1% DMSO of at least 10 times the solubility of CBD. In some embodiments, the compound of Formula (I) has a solubility in 1% DMSO at least 20 times the solubility of CBD. In some embodiments, the compound of Formula (I) has a solubility in 1% DMSO at least 30 times the solubility of CBD.

As used herein, the “solubility in 1% DMSO” of compounds of Formula (I) is determined by performing nephelometry experiments according to the procedure described in Example 8.

Whenever a term in the specification is identified as a range (e.g., C₁₋₄ alkyl) or “ranging from”, the range independently discloses and includes each element of the range. As a non-limiting example, C₁₋₄ alkyl groups includes, independently, C₁ alkyl groups, C₂ alkyl groups, C₃ alkyl groups, and C₄ alkyl groups. As another non-limiting example, “n is an integer ranging from 0 to 2” includes, independently, 0, 1, and 2.

The term “at least one” refers to one or more, such as one, two, etc. For example, the term “at least one group” refers to one or more groups, such as one group, two groups, etc.

The term “alkyl” includes saturated straight, branched, and cyclic (also identified as cycloalkyl) hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “aryl” includes hydrocarbon ring system groups comprising at least 6 carbon atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be monocyclic, bicyclic, tricyclic or tetracyclic ring systems, which may include fused, spiro, or bridged ring systems and combinations thereof, and may be partially or fully saturated; any nitrogen, carbon or sulfur atom(s) in the heterocyclyl group may be optionally oxidized; any nitrogen atom in the heterocyclyl group may be optionally quaternized. Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

The term “pharmaceutically acceptable salts” includes both acid and base addition salts. Non-limiting examples of pharmaceutically acceptable acid addition salts include acetates, adipates, ascorbates, aspartates, benzoates, besylates, bicarbonates/carbonates, bisulphates/sulphates, borates, camsylates, citrates, cyclamates, edisylates, esylates, formates, fumarates, gluceptates, gluconates, glucuronates, hexafluorophosphates, hibenzates, hydrochlorides/chlorides, hydrobromides/bromides, hydroiodides/iodides, isethionates, lactates, malates, maleates, malonates, mesylates, sulphates, sulfonates, naphthylates, 2-napsylates, nicotinates, nitrates, orotates, oxalates, palmitates, pamoates, phosphates/hydrogen phosphates/dihydrogen phosphates, pyroglutamates, saccharates, salicylates, stearates, succinates, tannates, tartrates, tosylates, trifluoroacetates, and xinofoates. Non-limiting examples of pharmaceutically acceptable base addition salts include aluminium, arginine, benzathine, calcium, choline, copper, diethylamine, diolamine, glycine, iron, lithium, lysine, magnesium, manganese, meglumine, olamine, potassium, sodium, tromethamine and zinc salts. Suitable base salts also include both unsubstituted and substituted ammonium salts. Hemisalts of acids and bases may also be formed, for example, hemisulphate and hemicalcium salts. Pharmaceutically acceptable salts may, for example, be obtained using standard procedures well known in the field of pharmaceuticals. For a review on suitable salts, see Handbook of Pharmaceutical Salts: Properties, Selection and Use by Stahl and Wermuth (Wiley-VCH, 2002), incorporated herein by reference.

When the compounds of the present disclosure contain an acidic group as well as a basic group, the compounds may also form internal salts and such compounds are within the scope of the disclosure. When a compound contains a hydrogen-donating heteroatom (e.g., NH), salts are contemplated to covers isomers formed by transfer of said hydrogen atom to a basic group or atom within the molecule.

The term “substituted” includes the situation where, in any of the groups above, at least one hydrogen atom is replaced by a non-hydrogen atom such as, for example, a halogen atom such as F, Cl, Br, and I; a carbon atom in groups such as alkyl, aryl, heterocyclyl, heteroaryl, arylalkyl, heterocyclylalkyl, and heteroarylalkyl; an oxygen atom in groups such as hydroxyl, alkoxy groups, and ester groups; a sulfur atom in groups such as thiol, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the groups above, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

The present disclosure includes within its scope all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof, conventional resolution methods, e.g., fractional crystallization and chiral chromatography, may be used.

The present disclosure includes within its scope all possible tautomers. Furthermore, the present disclosure includes in its scope both the individual tautomers and any mixtures thereof.

Biological activity of a compound described herein may be determined, for example, by performing at least one in vitro and/or in vivo study routinely practiced in the art and described herein or in the art. In vitro assays include without limitation binding assays, immunoassays, competitive binding assays, and cell-based activity assays.

Conditions for a particular assay include temperature, buffers (including salts, cations, and media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

Also provided are pharmaceutical compositions comprising at least one compound of Formula (I). Such pharmaceutical compositions are described in greater detail herein. These compounds and compositions may be used in the methods described herein.

In some embodiments, a method for treating and/or preventing at least one disease, disorder, and/or condition where treatment with a cannabinoid may be useful is disclosed, the method comprising administering to a subject in need thereof an effective amount of at least one compound of Formula (I) and/or a pharmaceutical composition comprising same.

In some embodiments, a method for treating and/or preventing at least one disease, disorder, and/or condition where treatment with an antagonist of cannabinoid 1 receptors (CB₁Rs) and/or cannabinoid 2 receptors (CB₂Rs) may be useful is disclosed, the method comprising administering to a subject in need thereof an effective amount of at least one compound of Formula (I) and/or a pharmaceutical composition comprising same.

In some embodiments, a method for treating and/or preventing at least one disease, disorder, and/or condition where treatment with an anxiolytic, analgesic, antiemetic, mood-stabilizing agent, and/or antipsychotic agent may be useful is disclosed, the method comprising administering to a subject in need thereof an effective amount of at least one compound of Formula (I) and/or a pharmaceutical composition comprising same.

In some embodiments, a method for treating and/or preventing at least one psychiatric disease, disorder, and/or condition is disclosed, the method comprising administering to a subject in need thereof an effective amount of at least one compound of Formula (I) and/or a pharmaceutical composition comprising same.

In some embodiments, the at least one psychiatric disease, disorder, and/or condition is chosen from depression, anxiety, dementia, bipolar disorder, schizophrenia, addiction, and nausea. In some embodiments, the at least one psychiatric disease, disorder, and/or condition is chosen from depression and anxiety.

In some embodiments, the at least one psychiatric disease, disorder, and/or condition is chosen from depression, anxiety, dementia, schizophrenia, addiction, and nausea. In some embodiments, the at least one psychiatric disease, disorder, and/or condition is chosen from depression and anxiety.

In some embodiments, a method for treating and/or preventing at least one disease, disorder, and/or condition where treatment with an anti-inflammatory, anti-oxidative, and/or neuroprotective agent may be useful is disclosed, the method comprising administering to a subject in need thereof an effective amount of at least one compound of Formula (I) and/or a pharmaceutical composition comprising same.

In some embodiments, a method for treating and/or preventing pain is disclosed, the method comprising administering to a subject in need thereof an effective amount of at least one compound of Formula (I) and/or a pharmaceutical composition comprising same. In some embodiments, the pain is neuropathic pain. In some embodiments, the pain is chronic pain.

In some embodiments, a method for treating and/or preventing diabetes is disclosed, the method comprising administering to a subject in need thereof an effective amount of at least one compound of Formula (I) and/or a pharmaceutical composition comprising same.

In some embodiments, a method for treating and/or preventing epilepsy or similar seizure disorder is disclosed, the method comprising administering to a subject in need thereof an effective amount of at least one compound of Formula (I) and/or a pharmaceutical composition comprising same. Patients can include those with epilepsy that are inadequately controlled by existing medications, individuals with developmental epileptic encephalopathy, or individuals with rare diseases or genetic conditions that produce epilepsy, seizures, spasms, abnormally hypersynchronous brain activity, or other conditions associated with enhanced neuronal synchrony. In some embodiments, the patients may be paediatric patients with epilepsy.

In some embodiments, a method for treating and/or preventing at least one neurological disease, disorder, and/or condition is disclosed, the method comprising administering to a subject in need thereof an effective amount of at least one compound of Formula (I) and/or a pharmaceutical composition comprising same.

In some embodiments, the at least one neurological disease, disorder, and/or condition is chosen from major mental disorders, conditions that involve basal ganglia or altered dopamine, movement disorders, substance abuse/addiction or predisposition to substance abuse/addiction, pain disorders, developmental delay or situations with impaired learning, intellectual disability, memory, and/or cognition, multiple sclerosis, and circuit disorder.

In some embodiments, the at least one neurological disease, disorder, and/or condition is chosen from depression, autism, postpartum depression, attention-deficit disorder, schizophrenia, anxiety, various psychoses, and epilepsies.

In some embodiments, the at least one neurological disease, disorder, and/or condition is chosen from depression, postpartum depression, attention-deficit disorder, schizophrenia, anxiety, various psychoses, and epilepsies.

In some embodiments, the at least one neurological disease, disorder, and/or condition is chosen from dystonia and related motor disorders, Parkinson's disease, and L-DOPA-induced dyskinesias or dyskinesias that result from medication.

In some embodiments, the at least one neurological disease, disorder, and/or condition is chosen from neurodegenerative diseases.

In some embodiments, the at least one neurological disease, disorder, and/or condition is chosen from Alzheimer's disease, Parkinson's disease, Lewy body dementia, and frontal lobe dementia, and motor retraining after acute injury, spasticity, and spasticity due to brain or spinal cord injury.

In some embodiments, the at least one neurological disease, disorder, and/or condition is chosen from Alzheimer's disease, Parkinson's disease, and frontal lobe dementia, and motor retraining after acute injury, spasticity, and spasticity due to brain or spinal cord injury.

In some embodiments, the at least one neurological disease, disorder, and/or condition is multiple sclerosis.

In some embodiments, the at least one neurological disease, disorder, and/or condition is chosen from circuit disorders.

In some embodiments, a method for treating and/or preventing at least one disease, disorder, and/or condition associated with schizophrenia, Parkinson's disease, depression, anxiety, neuropsychiatric or mood disorders, motor dysfunction, spasticity, movement disorders, neuropathic pain, amyotrophic lateral sclerosis (ALS), epilepsy or other neurologic events, neurocognitive disorders, tardive dyskinesia, motor disorders, and/or mood disorders is disclosed, the method comprising administering to a subject in need thereof an effective amount of at least one compound of Formula (I) and/or a pharmaceutical composition comprising same. An exemplary movement disorder is Huntington's disease (Peres et al., Front Pharmacol, 2018, 9:482).

In some embodiments, a method for treating at least one symptom associated with epilepsies and/or similar seizure disorders is disclosed, the method comprising administering to a subject in need thereof an effective amount of at least one compound of Formula (I) and/or a pharmaceutical composition comprising same.

In some embodiments, a method for treating at least one symptom associated with Lennox-Gastaut syndrome, Dravet syndrome, developmental epileptic encephalopathy, and/or tuberous sclerosis complex is disclosed, the method comprising administering to a subject in need thereof an effective amount of at least one compound of Formula (I) and/or a pharmaceutical composition comprising same.

In some embodiments, a method for treating at least one symptom associated with Lennox-Gastaut syndrome, Dravet syndrome, and/or tuberous sclerosis complex is disclosed, the method comprising administering to a subject in need thereof an effective amount of at least one compound of Formula (I) and/or a pharmaceutical composition comprising same.

In some embodiments, a method for reducing the severity and/or intensity of seizures associated with epilepsies and/or rare genetic disorders/diseases (e.g., those that can cause occasional seizures or abnormal electroencephalography) is disclosed, the method comprising administering to a subject in need thereof an effective amount of at least one compound of Formula (I) and/or a pharmaceutical composition comprising same.

In some embodiments, a method for reducing the frequency of seizures associated with epilepsies and/or rare genetic disorders/diseases (e.g., those that can cause occasional seizures or abnormal electroencephalography) is disclosed, the method comprising administering to a subject in need thereof an effective amount of at least one compound of Formula (I) and/or a pharmaceutical composition comprising same.

In some embodiments, a method for treating Parkinson's disease is disclosed, the method comprising administering to a subject in need thereof an effective amount of at least one compound of Formula (I) and/or a pharmaceutical composition comprising same.

In some embodiments, a method for treating at least one symptom of Parkinson's disease is disclosed, the method comprising administering to a subject in need thereof an effective amount of at least one compound of Formula (I) and/or a pharmaceutical composition comprising same.

In some embodiments, the at least one symptom of Parkinson's disease is chosen from spasticity, rigidity, dystonia and movement disorders.

In some embodiments, a method for treating and/or preventing ischemic injury, stroke, or stroke associated damages is disclosed, the method comprising administering to a subject in need thereof an effective amount of at least one compound of Formula (I) and/or a pharmaceutical composition comprising same. In some embodiments, the ischemic injury is caused by coronary artery bypass graft (CABG) or subarachnoid hemorrhage (SAH).

In some embodiments, a method for treating and/or preventing stroke or stroke associated damages is disclosed, the method comprising administering to a subject in need thereof an effective amount of at least one compound of Formula (I) and/or a pharmaceutical composition comprising same.

In some embodiments, a method for treating and/or preventing ischemic injury, stroke, or stroke associated damages is disclosed, the method comprising administering to a subject in need thereof an effective amount of at least one compound of Formula (I) and/or a pharmaceutical composition comprising same, wherein the at least one compound of Formula (I) and/or pharmaceutical composition comprising same is administered under emergency care for an ischemic injury or stroke. In some embodiments, the ischemic injury is caused by CABG or SAH.

In some embodiments, a method for treating and/or preventing stroke or stroke associated damages is disclosed, the method comprising administering to a subject in need thereof an effective amount of at least one compound of Formula (I) and/or a pharmaceutical composition comprising same, wherein the at least one compound of Formula (I) and/or pharmaceutical composition comprising same is administered under emergency care for a stroke.

In some embodiments, a method for treating and/or preventing ischemic injury, stroke, or stroke associated damages is disclosed, the method comprising administering to a subject in need thereof an effective amount of at least one compound of Formula (I) and/or a pharmaceutical composition comprising same, wherein the at least one compound of Formula (I) and/or pharmaceutical composition comprising same is administered under maintenance treatment of ischemic injury or stroke. In some embodiments, the ischemic injury is caused by CABG or SAH.

In some embodiments, a method for treating and/or preventing stroke or stroke associated damages is disclosed, the method comprising administering to a subject in need thereof an effective amount of at least one compound of Formula (I) and/or a pharmaceutical composition comprising same, wherein the at least one compound of Formula (I) and/or pharmaceutical composition comprising same is administered under maintenance treatment of stroke.

In some embodiments, a method for treating and/or preventing ischemic injury, stroke or stroke associated damages is disclosed, the method comprising administering to a subject in need thereof an effective amount of at least one compound of Formula (I) and/or a pharmaceutical composition comprising same, wherein the at least one compound of Formula (I) and/or pharmaceutical composition comprising same is administered for rehabilitation of ischemic injury or stroke. In some embodiments, the ischemic injury is caused by CABG or SAH.

In some embodiments, a method for treating and/or preventing stroke or stroke associated damages is disclosed, the method comprising administering to a subject in need thereof an effective amount of at least one compound of Formula (I) and/or a pharmaceutical composition comprising same, wherein the at least one compound of Formula (I) and/or pharmaceutical composition comprising same is administered for rehabilitation of stroke.

In some embodiments, a method for treating traumatic brain injuries (TBIs) is disclosed, the method comprising administering to a subject in need thereof an effective amount of at least one compound of Formula (I) and/or a pharmaceutical composition comprising same. The efficacy of cannabidiol in treating TBIs is documented in the art (for example, Belardo et al., Front Pharmacol, 2019, 10:352; Friedman et al., Exp Neurol, 2021 346:113844).

In some embodiments, a method for treating TBIs is disclosed, the method comprising administering to a subject in need thereof an effective amount of at least one compound of Formula (I) and/or a pharmaceutical composition comprising same, wherein the at least one compound of Formula (I) and/or pharmaceutical composition comprising same is administered under emergency care for TBIs.

In some embodiments, a method for treating TBIs is disclosed, the method comprising administering to a subject in need thereof an effective amount of at least one compound of Formula (I) and/or a pharmaceutical composition comprising same, wherein the at least one compound of Formula (I) and/or pharmaceutical composition comprising same is administered under maintenance treatment of TBIs.

In some embodiments, a method for treating TBIs is disclosed, the method comprising administering to a subject in need thereof an effective amount of at least one compound of Formula (I) and/or a pharmaceutical composition comprising same, wherein the at least one compound of Formula (I) and/or pharmaceutical composition comprising same is administered for rehabilitation of TBIs.

In some embodiments, a method for treating sleep disorders is disclosed, the method comprising administering to a subject in need thereof an effective amount of at least one compound of Formula (I) and/or a pharmaceutical composition comprising same. The use of cannabidiol in treating sleep disorders is documented in the art (for example, Shannon et al., Perm J, 2019, 23:18-041; Babson et al., Curr Psychiatry Rep, 2017, 19(4):23; Suraev et al., Sleep Med Rev, 2020, 53:101339; de Almeida et al., Mov Disord, 2021, 36(7):1711-1715).

In some embodiments, a method for treating high blood pressure or hypertension is disclosed, the method comprising administering to a subject in need thereof an effective amount of at least one compound of Formula (I) and/or a pharmaceutical composition comprising same. The use of cannabidiol in reducing blood pressure is documented in the art (for example, Jadoon et al., JCI Insight, 2017, 2(12):e93760).

In some embodiments, a method for treating and/or preventing neuropathic pain is disclosed. In some embodiments, the neuropathic pain is chosen from peripheral diabetic neuropathy, postherpetic neuralgia, complex regional pain syndromes, peripheral neuropathies, rheumatoid arthritis, chemotherapy-induced neuropathic pain, cancer neuropathic pain, neuropathic low back pain, HIV neuropathic pain, trigeminal neuralgia and/or central post-stroke pain.

In some embodiments, the neuropathic pain results from peripheral or central nervous system pathologic events.

In some embodiments, the neuropathic pain that results from trauma, ischemia; cancer (such as the neuropathic pain caused by sensory fibers at the site of the tumor); infections or from ongoing metabolic or toxic diseases, infections or endocrinologic disorders, including, but not limited to, diabetes mellitus, diabetic neuropathy, amyloidosis, amyloid polyneuropathy (primary and familial), neuropathies with monoclonal proteins, vasculitic neuropathy, HIV infection; neuropathy associated with Guillain-Barre syndrome; neuropathy associated with Fabry's disease; trigeminal and other CNS neuralgias; inflammatory conditions or autoimmune disorders, including, but not limited to, demyelinating inflammatory disorders, rheumatoid arthritis, systemic lupus erythematosus; and cryptogenic causes, including, but not limited to idiopathic distal small-fibre neuropathy. Other causes of neuropathic pain that can be treated according to the methods and compositions described herein include, but are not limited to, exposure to toxins or drugs (such as arsenic, thallium, alcohol, vincristine, cisplatin and dideoxynucleosides), dietary or absorption abnormalities, immunoglobulinemias. Neuropathic pain can also result from compression of nerve fibres, such as radiculopathies and carpal tunnel syndrome.

In some embodiments, a method for treating and/or preventing at least one disease, disorder, and/or condition chosen from chronic nerve injury, chronic pain syndromes, seizures, spreading depression, restless leg syndrome, hypoxic-ischemic encephalopathy, spinal cord injury, status epilepticus, concussion, migraine, hyperventilation, and/or retinopathiesis disclosed, the method comprising administering to a subject in need thereof an effective amount of at least one compound of Formula (I) and/or a pharmaceutical composition comprising same.

In some embodiments, a method for treating and/or preventing ischemia following transient or permanent vessel occlusion is disclosed, the method comprising administering to a subject in need thereof an effective amount of at least one compound of Formula (I) and/or a pharmaceutical composition comprising same.

In some embodiments, a method for reducing at least one symptom of neuropathic pain, stroke, epilepsy, and/or other neurologic events or neurodegeneration is disclosed, the method comprising administering to a subject in need thereof an effective amount of at least one compound of Formula (I) and/or a pharmaceutical composition comprising same.

In some embodiments, a method for treating a subject being treated for cancer is disclosed, the method comprising administering to a subject in need thereof an effective amount of at least one compound of Formula (I) and/or a pharmaceutical composition comprising same.

In some embodiments, a method for reducing pain and/or nausea in a subject being treated for cancer is disclosed, the method comprising administering to a subject in need thereof an effective amount of at least one compound of Formula (I) and/or a pharmaceutical composition comprising same.

In some embodiments, a method for increasing appetite in a subject being treated for cancer is disclosed, the method comprising administering to a subject in need thereof an effective amount of at least one compound of Formula (I) and/or a pharmaceutical composition comprising same.

In some embodiments, a method for reducing cell viability in a subject being treated for cancer is disclosed, the method comprising administering to a subject in need thereof an effective amount of at least one compound of Formula (I) and/or a pharmaceutical composition comprising same.

In some embodiments, a method for increasing cancer cell death in a subject being treated for cancer is disclosed, the method comprising administering to a subject in need thereof an effective amount of at least one compound of Formula (I) and/or a pharmaceutical composition comprising same.

In some embodiments, a method for decreasing tumour growth in a subject being treated for cancer is disclosed, the method comprising administering to a subject in need thereof an effective amount of at least one compound of Formula (I) and/or a pharmaceutical composition comprising same.

In some embodiments, a method for inhibiting metastasis of at least one cancer is disclosed, the method comprising administering to a subject in need thereof an effective amount of at least one compound of Formula (I) and/or a pharmaceutical composition comprising same.

In some embodiments, the administration of at least one compound of the present disclosure or pharmaceutical composition comprising at least one such compound may be in conjunction with one or more other therapies. For example, at least one palliative agent to counteract (at least in part) a side effect of a therapy (e.g., anti-depression therapy, anti-epileptic therapy) may be administered. Agents (chemical or biological) that promote recovery, or counteract side effects of administration of antibiotics or corticosteroids, are examples of such palliative agents.

At least one compound described herein may be administered before, after, or concurrently with administration of at least one additional therapeutic agent or at least one palliative agent. When administration is concurrent, the combination may be administered from a single container or two (or more) separate containers.

The terms “treat” and “treatment” include medical management of a disease, disorder, and/or condition of a subject as would be understood by a person of ordinary skill in the art (see, e.g., Stedman's Medical Dictionary). In general, an appropriate dose and treatment regimen provide at least one of the compounds of the present disclosure in an amount sufficient to provide therapeutic benefit. Therapeutic benefit includes, for example, an improved clinical outcome, wherein the object is to slow or lessen an undesired physiological change or disorder, or to slow or lessen the expansion or severity of such disorder. As discussed herein, improved clinical outcomes from treating a subject include, but are not limited to, abatement, lessening, or alleviation of symptoms that result from or are associated with the disease, condition, and/or disorder to be treated; decreased occurrence of symptoms; improved quality of life; diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment.

The terms “prevent” and “preventing” include the reducing or decreasing the likelihood of occurrence, recurrence, spread, or onset in a statistically or clinically significant manner. It is not intended that the present disclosure be limited to complete prevention. In some embodiments, the onset is delayed, or the severity of the disease is reduced.

In some embodiments of the methods described herein, the subject is a human. In some embodiments of the methods described herein, the subject is a human under the age of 18. In some embodiments of the methods described herein, the subject is a non-human animal. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the pharmaceutical composition is in the form of a tablet, capsule, pill, gel, granules, aerosol, solution (such as aqueous solution, e.g., a saline or phosphate buffer), suspension, nanoparticle formulation (including liposomes), emulsion, etc. The pharmaceutical composition may also include one or more further active agents or may be administered in combination with one or more such active agent. In some embodiments, the pharmaceutical composition is an oral formulation. For example, the oral formulation may be in a solid form, such as tablet, capsule, pill, and granules. Alternatively, the oral formulation may be in a liquid form, such as solution, suspension, and emulsion.

Pharmaceutical compositions for use in the present disclosure comprise an effective amount of at least one compound of Formula (I) and optionally a suitable pharmaceutical acceptable carrier. The preparations may be prepared in a manner known per se, which usually involves mixing the at least one compound according to the disclosure with the one or more pharmaceutically acceptable carriers and, if desired, in combination with other pharmaceutical active compounds, when necessary, under aseptic conditions. Reference is again made to standard handbooks, such as the latest edition of Remington's Pharmaceutical Sciences.

In some embodiments, the compounds of the present disclosure may be formulated as a pharmaceutical composition comprising at least one compound of Formula (I) and at least one pharmaceutically acceptable carrier, diluent or excipient and/or adjuvant and optionally one or more further pharmaceutically active compounds.

In some embodiments, the pharmaceutical compositions of the present disclosure are in a unit dosage form and may be suitably packaged, for example in a box, blister, vial, bottle, sachet, ampoule or in any other suitable single-dose or multi-dose holder or container (which may be properly labelled); optionally with one or more leaflets containing product information and/or instructions for use. In some embodiments, such unit dosages comprise between 1 and 1000 mg, or between 5 and 500 mg, of the at least one compound of the disclosure, e.g., about 10, 25, 50, 100, 200, 300 or 400 mg per unit dosage. For human patients (including both adult and pediatric patients), the dose of the compound of Formula (I) may be lower than 20 mg/kg per day, lower than 15 mg/kg per day, lower than 12.5 mg/kg per day, lower than 10 mg/kg per day, lower than 5 mg/kg per day, or lower than 2.5 mg/kg per day. In some embodiments, the dose of the compound of Formula (I) is lower than 2.5 mg/kg per day.

The compounds and compositions of the present disclosure may be administered by a variety of routes including the oral, ocular, rectal, transdermal, subcutaneous, intravenous, intramuscular, or intranasal routes. In some embodiments, the at least one compound of Formula (I) or composition comprising the same is administered by inhalation through the lungs. In some embodiments, the at least one compound of Formula (I) or composition comprising the same is administered orally. In some embodiments, the at least one compound of Formula (I) or composition comprising the same is administered topically. In some embodiments, the at least one compound of Formula (I) or composition comprising the same is administered intravenously.

The effectiveness of the compounds of the present disclosure in treating and/or preventing diseases, disorders, and/or conditions can readily be determined by a person of ordinary skill in the relevant art. Determining and adjusting an appropriate dosing regimen (e.g., adjusting the amount of compound per dose and/or number of doses and frequency of dosing) can also readily be performed by a person of ordinary skill in the relevant art. One or any combination of diagnostic methods, including physical examination, assessment and monitoring of clinical symptoms, and performance of analytical tests and methods described herein, may be used for monitoring the health status of the subject.

Depending upon the manner of introduction, the compounds described herein may be formulated in a variety of ways. Formulations containing one or more compounds can be prepared in various pharmaceutical forms, such as granules, tablets, capsules, suppositories, powders, controlled release formulations, solutions (such as aqueous solutions, e.g., salines and buffered salines), suspensions, emulsions, creams, gels, ointments, salves, lotions, or aerosols and the like. In some embodiments, these formulations are employed in solid dosage forms suitable for simple and oral administration of precise dosages. Solid dosage forms for oral administration include, but are not limited to, tablets, soft or hard gelatin or non-gelatin capsules and caplets. However, liquid dosage forms, such as solutions, syrups, suspension, shakes, etc. can also be utilized. In another embodiment, the formulation is administered topically. Suitable topical formulations include, but are not limited to, lotions, ointments, creams and gels. In some embodiments, the topical formulation is a gel. In another embodiment, the formulation is administered intranasally.

In some embodiments, the pharmaceutical composition comprises at least one compound of Formula (I) and a propellant. In some embodiments, the propellant is an aerosolizing propellant. In some embodiments, the aerosolizing propellant is chosen from compressed air, ethanol, nitrogen, carbon dioxide, nitrous oxide, hydrofluoroalkanes (HFAs), 1,1,1,2,-tetrafluoroethane, 1,1,1,2,3,3,3-heptafluoropropane, and combinations thereof.

In some embodiments, the disclosure contemplates a pressurized or unpressurized container comprising at least one compound of Formula (I). In some embodiments, the container is a manual pump spray, inhaler, meter-dosed inhaler, dry powder inhaler, nebulizer, vibrating mesh nebulizer, jet nebulizer, or ultrasonic wave nebulizer.

Formulations containing at least one compound of Formula (I) may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which may include excipients, plasticizers, pigments, colorants, stabilizing agents, and glidants. Delayed release, extended release, and/or pulsatile release dosage formulations may be prepared as described in standard references, such as “Pharmaceutical dosage form tablets”, eds. Liberman et al. (New York, Marcel Dekker, Inc., 1989), “Remington—The science and practice of pharmacy”, 20th ed., Lippincott Williams & Wilkins, Baltimore, MD, 2000 and “Pharmaceutical dosage forms and drug delivery systems”, 6th Edition, Ansel et al., (Media, PA: Williams and Wilkins, 1995). These references provide information on carriers, materials, equipment and process for preparing tablets and capsules and delayed release dosage forms of tablets, capsules and granules.

Examples of suitable coating materials include, but are not limited to, cellulose polymers, such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers and methacrylic resins that are commercially available under the trade name Eudragit® (Roth Pharma, Westerstadt, Germany), zein, shellac and polysaccharides.

Additionally, the coating material may contain conventional carriers, such as plasticizers, pigments, colorants, glidants, stabilization agents, pore formers, and surfactants.

Optional pharmaceutically acceptable excipients present in the drug-containing tablets, beads, granules, or particles include, but are not limited to, diluents, binders, lubricants, disintegrants, colorants, stabilizers, and surfactants. Diluents, also referred to as “fillers,” may be desired to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, and powdered sugar.

Binders may be used to impart cohesive qualities to a solid dosage formulation and thus ensure that a tablet or bead or granule remains intact after the formation of the dosage forms. Suitable binder materials include, but are not limited to, starch, pregelatinized starch, gelatin, sugars (including sucrose, glucose, dextrose, lactose, and sorbitol), polyethylene glycol, waxes, natural and synthetic gums, such as acacia, tragacanth, sodium alginate, cellulose, including hydroxypropylmethylcellulose, hydroxypropylcellulose, ethylcellulose and veegum and synthetic polymers, such as acrylic acid and methacrylic acid copolymers, methacrylic acid copolymers, methyl methacrylate copolymers, aminoalkyl methacrylate copolymers, polyacrylic acid/polymethacrylic acid, and polyvinylpyrrolidone.

Lubricants may be used to facilitate tablet manufacture. Examples of suitable lubricants include, but are not limited to, magnesium stearate, calcium stearate, stearic acid, glycerol behenate, polyethylene glycol, talc, and mineral oil.

Disintegrants may be used to facilitate dosage form disintegration or “breakup” after administration. Exemplary disintegrants include, but are not limited to, starch, sodium starch glycolate, sodium carboxymethyl starch, sodium carboxymethylcellulose, hydroxypropyl cellulose, pregelatinized starch, clays, cellulose, alginine, gums, and cross linked polymers, such as cross-linked PVP (Polyplasdone XL from GAF Chemical Corp).

Stabilizers may be used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate, and sulfate ions. Examples of anionic surfactants include sodium, potassium, ammonium of long chain alkyl sulfonates and alkyl aryl sulfonates, such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates, such as sodium lauryl sulfate. Cationic surfactants include, but are not limited to, quaternary ammonium compounds, such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-o-alanine, sodium N-lauryl-p-iminodipropionate, myristoamphoacetate, lauryl betaine, and lauryl sulfobetaine.

If desired, the tablets, beads, granules, or particles may also contain nontoxic auxiliary substances, such as wetting or emulsifying agents, dyes, pH buffering agents, or preservatives.

The concentration of the at least one compound of Formula (I) to carrier and/or other substances may vary from about 0.5 to about 100 wt. % (weight percent). For oral use, the pharmaceutical formulation may comprise from about 5 to about 100% by weight of the active material. For other uses, the pharmaceutical formulation may comprise from about 0.5 to about 50 wt. % of the active material.

The pharmaceutical compositions described herein can be formulated for modified or controlled release. Examples of controlled release dosage forms include extended release dosage forms, delayed release dosage forms, pulsatile release dosage forms, and combinations thereof.

The extended release formulations may be prepared as diffusion or osmotic systems, for example, as described in “Remington—The science and practice of pharmacy” (20th ed., Lippincott Williams & Wilkins, Baltimore, MD, 2000). A diffusion system may consist of two types of devices, a reservoir and a matrix and is well known and described in the art. The matrix devices may be prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers, such as methyl and ethyl cellulose, and hydroxyalkylcelluloses, such as hydroxypropyl-cellulose, hydroxypropylmethylcellulose, sodium carboxymethylcellulose and carbopol CARBOPOL® 934, polyethylene oxides, and mixtures thereof. Fatty compounds include, but are not limited to, various waxes, such as carnauba wax and glyceryl tristearate and wax-type substances including hydrogenated castor oil or hydrogenated vegetable oil, and mixtures thereof.

In some embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In some embodiments, the acrylic polymer is comprised of one or more ammonio methacrylate copolymers. Ammonio methacrylate copolymers are well known in the art and are described in NF XVII as fully polymerized copolymers of acrylic and methacrylic acid esters with a low content of quaternary ammonium groups.

In some embodiments, the acrylic polymer is an acrylic resin lacquer such as that which is commercially available from Rohm Pharma under the tradename EUDRAGIT®. In further embodiments, the acrylic polymer comprises a mixture of two acrylic resin lacquers commercially available from Rohm Pharma under the Tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also contemplated. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above, such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT® RS and 10% EUDRAGIT® RL and 90%: EUDRAGIT® 90% RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and, capsules containing tablets, beads, or granules, etc.

An immediate release portion can be added to the extended release system by means of either applying an immediate release layer on top of the extended release core using a coating or compression process or in a multiple unit system, such as a capsule containing extended and immediate release beads.

Extended release tablets containing hydrophilic polymers are prepared by techniques commonly known in the art, such as direct compression, wet granulation, or dry granulation processes. Their formulations may incorporate polymers, diluents, binders, and lubricants as well as the active pharmaceutical ingredient. The usual diluents include inert powdered substances, such as starches, powdered cellulose, crystalline and microcrystalline cellulose, sugars, such as fructose, mannitol and sucrose, grain flours, and similar edible powders. Diluents include, for example, various types of starch, lactose, mannitol, kaolin, calcium phosphate or sulfate, inorganic salts, such as sodium chloride, and powdered sugar. Powdered cellulose derivatives may also be useful. Tablet binders may include substances, such as starch, gelatin and sugars, such as lactose, fructose, and glucose. Natural and synthetic gums, including acacia, alginates, methylcellulose and polyvinylpyrrolidone can also be used. Polyethylene glycol, hydrophilic polymers, ethylcellulose, and waxes can also serve as binders. A lubricant may be used in a tablet formulation to prevent the tablet and punches from sticking in the die. The lubricant may be chosen from such slippery solids as talc, magnesium and calcium stearate, stearic acid, and hydrogenated vegetable oils.

Extended release tablets containing wax materials may be prepared using methods known in the art, such as a direct blend method, a congealing method, and an aqueous dispersion method. In the congealing method, the drug is mixed with a wax material and either spray- congealed or congealed and screened and processed.

Delayed release formulations may be created by coating a solid dosage form with a polymer film, which is insoluble in the acidic environment of the stomach and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. In some embodiments, coating materials include bioerodible, gradually hydrolyzable, gradually water-soluble and/or enzymatically degradable polymers and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers, such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, formed from acrylic acid, methacrylic acid, methyl acrylate, ethyl acrylate, methyl methacrylate and/or ethyl methacrylate and other methacrylic resins that are commercially available under the tradename EUDRAGIT® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification) and EUDRAGIT® NE, RL and RS (water-insoluble polymers having different degrees of permeability and expandability); vinyl polymers and copolymers, such as polyvinyl pyrrolidone, vinyl acetate, vinylacetate phthalate, vinylacetate crotonic acid copolymer and ethylene-vinyl acetate copolymer; enzymatically degradable polymers, such as azo polymers, pectin, chitosan, amylose and guar gum; and zein and shellac. Combinations of different coating materials may also be used. Multi-layer coatings using different polymers may also be applied.

The coating weights for particular coating materials may be readily determined by those skilled in the art by evaluating individual release profiles for tablets, beads, and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer may be present to reduce the fragility of the coating. A plasticizer may represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides. A stabilizing agent may be used to stabilize particles in the dispersion. Stabilizing agents may be nonionic emulsifiers, such as sorbitan esters, polysorbates, and polyvinylpyrrolidone. Glidants may reduce sticking effects during film formation and drying. Glidants may represent approximately 25 wt. % to 100 wt. % of the polymer weight in the coating solution. One effective glidant is talc. Other glidants, such as magnesium stearate and glycerol monostearates may also be used. Pigments, such as titanium dioxide may also be used. Small quantities of an anti-foaming agent, such as a silicone (e.g., simethicone), may also be added to the coating composition.

The formulation can provide pulsatile delivery of the one or more compounds of the present disclosure. By “pulsatile” it is meant that a plurality of drug doses are released at spaced apart intervals of time. In some embodiments, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses may be designed. The duration of the nearly drug release-free time interval will vary depending upon the dosage form design e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In some embodiments, the pulsatile release profile is achieved with dosage forms that are closed and sealed capsules housing at least two drug-containing “dosage units” wherein each dosage unit within the capsule provides a different drug release profile. Control of the delayed release dosage unit(s) may be accomplished by a controlled release polymer coating on the dosage unit, or by incorporation of the active agent in a controlled release polymer matrix. Each dosage unit may comprise a compressed or molded tablet, wherein each tablet within the capsule provides a different drug release profile. For dosage forms mimicking a twice a day dosing profile, a first tablet releases drug substantially immediately following ingestion of the dosage form, while a second tablet releases drug approximately 3 hours to less than 14 hours following ingestion of the dosage form. For dosage forms mimicking a three times daily dosing profile, a first tablet releases drug substantially immediately following ingestion of the dosage form, a second tablet releases drug approximately 3 hours to less than 10 hours following ingestion of the dosage form and the third tablet releases drug at least 5 hours to approximately 18 hours following ingestion of the dosage form. It is possible that the dosage form includes more than three tablets. While the dosage form will not generally include more than a third tablet, dosage forms housing more than three tablets can be utilized.

Alternatively, each dosage unit in the capsule may comprise a plurality of drug-containing beads, granules, or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that may or may not include one or more additional excipients or polymers. In contrast to drug-containing beads, granules and particles do not contain an inert support. Granules may comprise drug particles and require further processing. In some embodiments, particles are smaller than granules and are not further processed. Although beads, granules, and particles may be formulated to provide immediate release, beads and granules may be employed to provide delayed release.

In some embodiments, the at least one compound Formula (I) is formulated for topical administration. Suitable topical dosage forms include lotions, creams, ointments and gels. A “gel” is a semisolid system containing a dispersion of the active agent, i.e., compound, in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because, for example, they do not contain a homogenized blend of immiscible components. Methods for preparing lotions, creams, ointments, and gels are well known in the art.

The at least one compound of Formula (I) can be administered adjunctively (i.e., in the same dosage form or in separate dosage forms with one or more other active agents) with other active compounds. These other active compounds include but are not limited to analgesics, antinociceptive agents, anti-inflammatory drugs, antipyretics, antidepressants, antiepileptics, antihistamines, antimigraine drugs, antimuscarinics, anxiolytics, sedatives, hypnotics, antipsychotics, bronchodilators, anti-asthma drugs, cardiovascular drugs (such as antihypertensive agents and antiarrhythmia agents), corticosteroids, dopaminergics, electrolytes, gastro-intestinal drugs, mood stabilizers (such as those for treating bipolar disorders), muscle relaxants, nutritional agents, vitamins, parasympathomimetics, stimulants, anorectics, and anti-narcoleptics.

Specific examples of other active compounds that can be adjunctively administered with the at least one compound of Formula (I) include, but are not limited to, acetazolamide, aceclofenac, acetaminophen, atomoxetine, almotriptan, alprazolam, amantadine, amcinonide, aminocyclopropane, amitriptyline, amlodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clobazam, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproex, dizatriptan, dolasetron, donepezil, dothiepin, doxepin, duloxetine, ergotamine, escitalopram, estazolam, ethosuximide, etodolac, felbamate, femoxetine, fenamates, fenoprofen, fentanyl, fludiazepam, fluoxetine, fluphenazine, flurazepam, flurbiprofen, flutazolam, fluvoxamine, frovatriptan, gabapentin, gabitril, galantamine, gepirone, Ginkgo biloba, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lacosamide, lamotrigine (an anticonvulsant and mood stabilizer), lesopitron, levodopa, levetiracetam, lipase, lithium (a mood stabilizer), lofepramine, lorazepam, loxapine, maprotiline, mazindol, mefenamic acid, melatonin, melitracen, memantine, meperidine, meprobamate, mesalamine, methsuximide, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nitrazepam, nomifensine, nortriptyline, olanzapine, olsalazine, ondansetron, opipramol, orphenadrine, oxaflozane, oxaprozin, oxazepam, oxitriptan, oxycodone, oxymorphone, pancrelipase, parecoxib, paroxetine, pemoline, pentazocine, pepsin, perphenazine, phenobarbital, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, primidone, propranolol, propizepine, propoxyphene, protriptyline, quazepam, quinupramine, reboxetine, reserpine, risperidone, ritanserin, rivastigmine, rizatriptan, rofecoxib, ropinirole, rotigotine, rufinamide, salsalate, sertraline, sibutramine, sildenafil, stiripentol, sulfasalazine, sulindac, sumatriptan, tacrine, temazepam, tetrabenazine, thiazides, thioridazine, thiothixene, tiapride, taziprinone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, vigabatrin, viloxazine, vitamin E, zimeldine, ziprasidone, zolmitriptan, zolpidem, zonisamide, zopiclone, and isomers, salts and combinations of any of the foregoing.

The additional active agent(s) can be formulated for immediate release, controlled release, or combinations thereof, either together with or separate from the compound of the present disclosure.

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound of Formula (I) and/or pharmaceutical composition comprising the same.

### EXAMPLES

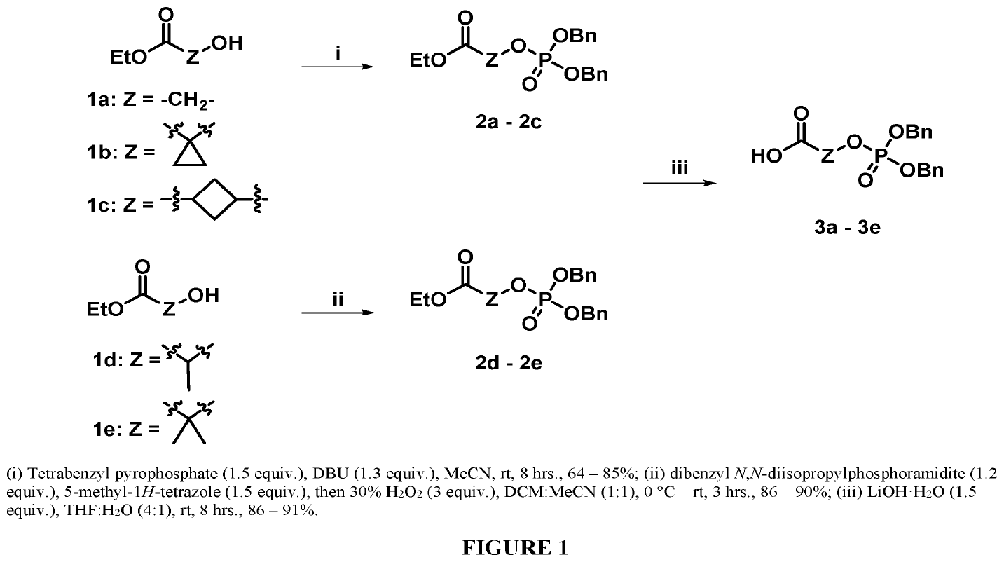

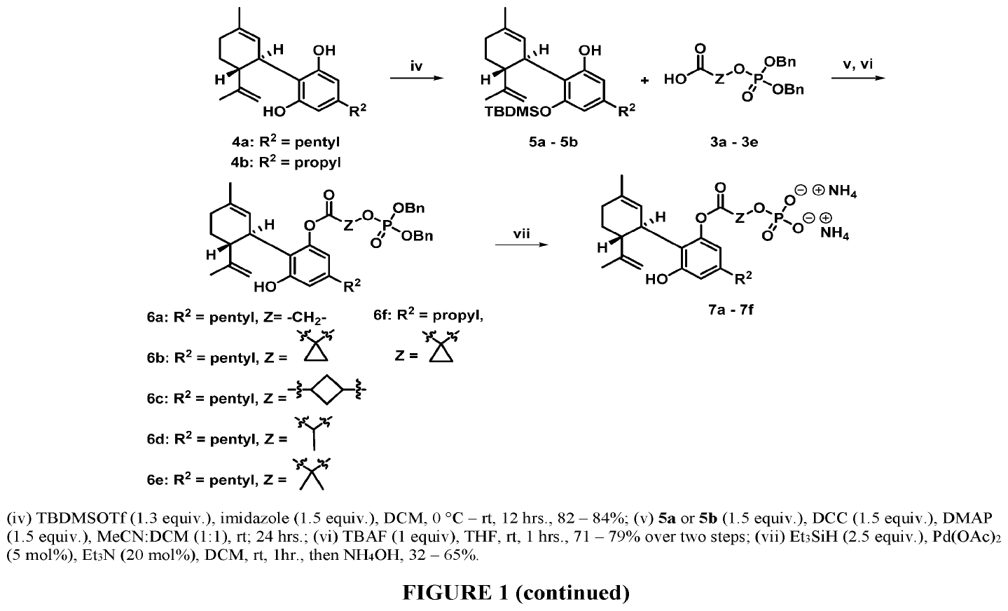

Compounds of Formula (I) may be prepared as shown in, for example, in FIGS. 1 to 7. It is understood that one of ordinary skill in the art may be able to make these compounds by similar methods or by combining other methods known to one of ordinary skill in the art. It is also understood that one of ordinary skill in the art would be able to make other compounds of Formula (I) not specifically illustrated herein by using appropriate starting components and modifying the parameters of the synthesis as needed. In general, starting components may be obtained from sources such as Sigma-Aldrich, Alfa Aesar, Maybridge, Matrix Scientific, TCI, and Fluorochem USA, etc. and/or synthesized according to sources known to those of ordinary skill in the art (see, for example, Advanced Organic Chemistry: Reactions, Mechanisms, and Structure, 5th edition (Wiley, December 2000)) and/or prepared as described herein.

It will also be appreciated by those skilled in the art that in the processes described herein the functional groups of intermediate compounds may need to be protected by suitable protecting groups, even if not specifically described. Such functional groups include hydroxy, amino, mercapto, and carboxylic acid. Suitable protecting groups for hydroxy include but are not limited to trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include but are not limited to t-butoxycarbonyl, benzyloxycarbonyl, and the like. Suitable protecting groups for mercapto include but are not limited to —C(O)R″ (where R″ is alkyl, aryl or arylalkyl), p-methoxybenzyl, trityl and the like. Suitable protecting groups for carboxylic acid include but are not limited to alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry,” 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin, G., “Organic Synthesis: Concepts, Methods, Starting Materials,” Second, Revised and Enlarged Edition, John Wiley & Sons ISBN: 3-527-29074-5, 1994; Hoffman, R. V., “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J., “Advanced Organic Chemistry: Reactions, Mechanisms, and Structure,” 4th Edition (1992) John Wiley & Sons, ISBN: 0-471-60180-2; Otera, J. (editor), “Modern Carbonyl Chemistry” (2000) Wiley-VCH, ISBN: 3-527-29871-1; Patai, S., “Patai's 1992 Guide to the Chemistry of Functional Groups” (1992) Interscience ISBN: 0-471-93022-9; Quin, L. D., et al., “A Guide to Organophosphorus Chemistry” (2000) Wiley-Interscience, ISBN: 0-471-31824-8; Solomons, T. W. G., “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopaedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

The present disclosure will now be described in more detail with reference to the following non-limiting examples. It should be noted that the particular assays used in the examples section are designed to provide an indication of activity.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

The examples below described studies to generate phosphate-based prodrugs of CBD with increased aqueous solubility, enhanced cellular absorption, improved metabolic stability, and/or more favorable tissue distribution.

### General Chemistry Synthesis and Characterization

Automated flash column chromatography was performed using a Teledyne ISCO CombiFlash Companion system with silica gel-packed columns (SiliCycle Inc. or RediSep® Rf). Analytical thin-layer chromatography (TLC, commercially available from Sigma-Aldrich) was carried out on aluminum-supported silica gel plates (thickness: 200 m) or glass-supported (thickness: 240 μm) silica gel plates with fluorescent indicator (F-254). Visualization of compounds on TLC plates was accomplished with UV light (254 nm) and/or with phosphomolybdic acid (PMA), potassium permanganate (KMnO₄) or ceric ammonium molybdate (CAM) stains. NMR spectra (¹H, ³¹P) were obtained using either a Varian INOVA 600 MHz spectrometer, a Varian INOVA 500 MHz spectrometer, a Varian INOVA 400 MHz spectrometer, a Varian VNMR 400 MHz spectrometer, a Bruker AVIIIHD 600 MHz spectrometer or a Mercury 300 MHz spectrometer. NMR samples were prepared in deuterated dimethylsulfoxide (DMSO-d₆) or deuterated methanol (CD₃OD) using the residual solvent peak (DMSO-d₆: ¹H=2.54 ppm, CD₃O D: ¹H=3.31 ppm) as an internal reference. Alternatively, the residual dimethylsulfoxide or methanol peak in ¹H NMR was used as an absolute reference for ³¹P NMR. In some cases, phosphoric acid (³¹P=40.48 ppm) was used as an external reference for ³¹P NMR. MestReNova software was used to process all NMR spectra. NMR data are reported to include chemical shifts (δ) reported in ppm, multiplicities indicated as s (singlet), d (doublet), t (triplet), q (quartet), m (multiplet), br (broad), or app (apparent), coupling constants (J) reported in Hz, and integration normalized to 1 atom (H, P). Final compound purity was assessed using ¹H NMR and LC-MS.

### General Bioanalytical Experimental, Instrumentation and Materials

All solvents for the LC-MS/MS analysis are UHPLC grade. Methanol and water were purchased from Thermo Fischer, and acetonitrile was obtained from Sigma-Aldrich. The HPLC grade formic acid was obtained from Thermo Fischer. The LC-MS vials with embedded inserts and screw caps were obtained from VWR.

An Agilent 1260 Infinity II HPLC system which includes a micro vacuum degasser, quaternary pump, high-performance autosampler with thermostat, and a thermostatted column compartment coupled to an Agilent 6460C Triple Quadrupole Mass Spectrometer was utilised. The mass spectrometer operated in the ESI mode with Agilent's Jet Stream Technology. The Agilent MassHunter Workstation (version B.02.00) was used for data acquisition, and the MassHunter Quantitative analysis software (version B.01.04) was used for data analysis. We employed the Agilent MassHunter Optimizer software (B.02.00) to optimize two important MS parameters: the fragmentor voltage and collision energies. The optimizer also provided the most abundant MRM transitions used in this study. Individual methods were developed for each compound in the presence of an internal standard (ISTD) d5-7-ethoxy coumarin in a positive mode. All compounds were analyzed using multiple reaction monitoring (MRM) with quantifying and qualifying ions for increased reliability. Reverse-phase HPLC separation for each compound was achieved on an Agilent Infinity Poroshell 120 EC C18 or C8 column or Eclipse XDB C18 column (2.1×50 mm, 2.7 microns). Mobile phases consisted of water (0.1% formic acid) and ACN (0.1% formic acid) at a 0.5 mL/min flow rate. The column temperature was maintained at 40° C. for most of the samples unless otherwise noted. Other MS conditions were as follows: dwell time 100 ms; gas flow 10 L/min; nebulizer pressure 45 psi; delta EMV 200 V.

### Example 1 (Compounds 7A-F)

### Synthesis of Alpha-Substituted Ester Linked Mono-Ester Phosphate Prodrug Derivatives

### Synthetic Procedure:

Compounds with a mono-ester phosphate prodrug moiety attached by an alpha substituted ester-linker to CBD were designed and synthesized using the procedures described below. The synthesis of the mono-ester phosphate prodrugs 7a-f is shown in FIG. 1.

The glycolic acid derivatives 3a-e were accessed via two synthetic pathways. First, the ethyl glycolate ester derivatives 1a-c were reacted with tetrabenzyl pyrophosphate by the slow addition of DBU, which served as base, to afford the dibenzyl phosphate analogues 2a-c. In contrast, the methyl substituted glycolate ester analogues 1d-e were first reacted with dibenzyl N,N-diisopropylphosphoramidite in the presence of 5-methyl-1H-tetrazole, followed by oxidation to the phosphate via hydrogen peroxide to afford the dibenzyl phosphate analogues 2d-e. The glycolic ester derivatives 2a-e were then hydrolyzed using lithium hydroxide monohydrate to afford the glycolic acid derivatives 3a-e. To avoid the formation of the di-ester intermediates, a silyl protecting group was installed on CBD and cannabidivarin (CBDV). Commercially available cannabidiol or cannabidivarin isolate (4a or 4b, respectively) and imidazole were treated with TBDMSOTf and stirred at r.t. overnight to generate the TBDMS-protected CBD or CBDV intermediate 5a-b. Steglich esterification between 5a-b and the previously synthesized carboxylic acids 3a-e proceeded smoothly to afford the silyl protected intermediates, which were isolated by aqueous workup and column chromatography, and deprotected using a 1M solution of TBAF in THF. This furnished the penultimate dibenzyl phosphate compounds 6a-f, which underwent palladium acetate catalyzed and triethylsilane facilitated debenzylation of the phosphate moiety.

Compounds were subjected to NH₄OH prior to purification to afford the desired prodrugs 7a-f as bis-ammonium salts.

### Chemical Characterization:

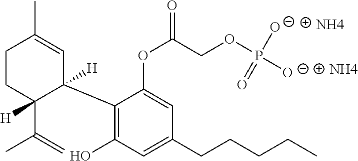 [SMILES: C=C(C)[C@@H]1CCC(C)=C[C@H]1c1c(O)cc(CCCCC)cc1OC(=O)COP(=O)([O-])[O-] ; [NH4+] ; [NH4+]]

Compound 7a: 2-(((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)-2-oxoethyl phosphate di-ammonium salt.

¹H NMR (600 MHz, DMSO-d₆) δ 7.36 (s, 1H), 6.49 (s, 1H), 6.24 (s, 1H), 4.98 (s, 1H), 4.46-4.37 (m, 3H), 4.30 (s, 1H), 3.81 (br s, 1H), 2.62 (br s, 1H), 2.43-2.36 (m, 2H), 2.16 (s, 1H), 1.96-1.88 (m, 1H), 1.70-1.62 (m, 2H), 1.60 (s, 3H), 1.58 (s, 3H), 1.53-1.45 (m, 2H), 1.33-1.20 (m, 4H), 0.85 (t, J=7.1 Hz, 3H); ³¹P NMR (243 MHz, DMSO-d₆) δ-1.14.

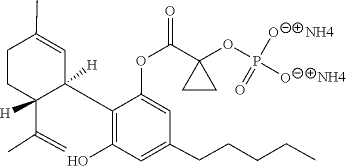 [SMILES: C=C(C)[C@@H]1CCC(C)=C[C@H]1c1c(O)cc(CCCCC)cc1OC(=O)C1(OP(=O)([O-])[O-])CC1 ; [NH4+] ; [NH4+]]

Compound 7b: 1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl phosphate di-ammonium salt.

¹H NMR (400 MHz, DMSO-d₆) δ 7.32 (s, 1H), 6.45 (s, 1H), 6.24 (s, 1H), 5.02 (s, 1H), 4.51 (s, 1H), 4.41 (s, 1H), 3.80 (br s, 1H), 2.74 (br s, 1H), 2.41-2.33 (m, 2H), 2.14 (s, 1H), 1.97-1.87 (m, 1H), 1.74-1.60 (m, 2H), 1.59 (s, 3H), 1.56 (s, 3H), 1.53-1.44 (m, 2H), 1.38-1.08 (m, 8H), 0.86 (t, J=6.9 Hz, 3H); ³¹P NMR (162 MHz, DMSO-d₆) δ-1.73.

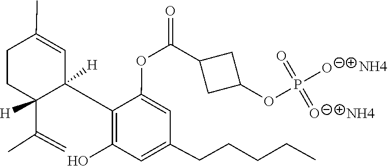

Compound 7c: 3-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclobutyl phosphate di-ammonium salt.

¹H NMR (400 MHz, DMSO-d₆) δ 7.51 (s, 1H), 6.47 (s, 1H), 6.19 (s, 1H), 5.00 (s, 1H), 4.51-4.39 (m, 3H), 3.82 (br s, 1H), 2.94 (q, J=7.2 Hz, 1H), 2.75 (br s, 1H), 2.63-2.54 (m, 2H), 2.39 (t, J=7.7 Hz, 2H), 2.26-2.15 (m, 2H), 2.11 (s, 1H), 1.98-1.88 (m, 1H), 1.72-1.60 (m, 2H), 1.57 (s, 6H), 1.53-1.44 (m, 2H), 1.33-1.20 (m, 4H), 0.85 (t, J=7.0 Hz, 3H); ³¹P NMR (162 MHz, DMSO-d₆) δ-2.49.

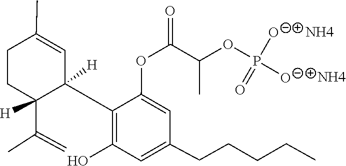 [SMILES: C=C(C)[C@@H]1CCC(C)=C[C@H]1c1c(O)cc(CCCCC)cc1OC(=O)C(C)OP(=O)([O-])[O-] ; [NH4+] ; [NH4+]]

Compound 7d: 1-(((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)-1-oxopropan-2-yl phosphate di-ammonium salt.

¹H NMR (400 MHz, DMSO-d₆) δ 7.32 (s, 1H), 6.48 (s, 1H), 6.25 (s, 1H), 5.04 (s, 1H), 4.77-4.65 (m, 1H), 4.48 (s, 1H), 4.41 (s, 1H), 3.82 (br s, 1H), 2.70 (br s, 1H), 2.43-2.34 (m, 2H), 2.13 (s, 1H), 1.96-1.87 (m, 1H), 1.66 (dq, J=12.5, 7.4, 5.2 Hz, 2H), 1.57 (s, 6H), 1.53-1.45 (m, 2H), 1.40 (d, J=6.7 Hz, 2H), 1.33-1.20 (m, 4H), 0.86 (t, J=6.9 Hz, 4H); ³¹P NMR (162 MHz, DMSO-d₆) δ-1.23.

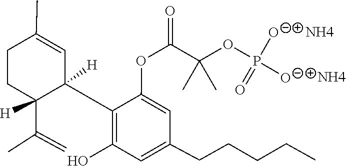 [SMILES: C=C(C)[C@@H]1CCC(C)=C[C@H]1c1c(O)cc(CCCCC)cc1OC(=O)C(C)(C)OP(=O)([O-])[O-] ; [NH4+] ; [NH4+]]

Compound 7e: 1-(((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)-2-methyl-1-oxopropan-2-yl phosphate di-ammonium salt.

¹H NMR (600 MHz, CD₃OD) δ 6.47 (d, J=1.7 Hz, 1H), 6.41 (d, J=1.7 Hz, 1H), 5.21 (s, 1H), 4.55-4.52 (m, 1H), 4.45 (s, 1H), 3.35 (s, 1H), 2.46 (t, J=7.68 Hz, 2H), 2.22-2.11 (m, 1H), 2.04-1.97 (m, 1H), 1.79-1.71 (m, 8H), 1.65-1.53 (m, 8H), 1.39-1.26 (m, 5H), 0.90 (t, J=7.1 Hz, 3H). ³¹P NMR (243 MHz, CD₃O D) δ-4.93.

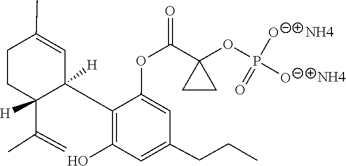 [SMILES: C=C(C)[C@@H]1CCC(C)=C[C@H]1c1c(O)cc(CCC)cc1OC(=O)C1(OP(=O)([O-])[O-])CC1 ; [NH4+] ; [NH4+]]

Compound 7f: 1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl phosphate di-ammonium salt.

¹H NMR (400 MHz, DMSO-d₆) δ 6.46 (d, J=1.7 Hz, 1H), 6.23 (d, J=1.7 Hz, 1H), 5.04-4.99 (m, 1H), 4.53-4.48 (m, 1H), 4.43-4.37 (m, 1H), 3.69 (br s, 1H), 2.85-2.67 (m, 1H), 2.36 (t, J=7.7 Hz, 2H), 2.20-2.08 (m, 1H), 1.97-1.87 (m, 1H), 1.72-1.60 (m, 1H), 1.58 (s, 3H), 1.56 (s, 3H), 1.54-1.44 (m, 2H), 1.25-1.17 (m, 2H), 1.14 (t, J=7.3 Hz, 4H), 0.86 (t, J=7.3 Hz, 3H). ³¹P NMR (162 MHz, DMSO-d₆) δ-1.41.

### Example 2 (Compounds 8 and 14A-K)

### Synthesis of Ester Linked Di-Ester Phosphate Prodrug Derivatives

### Synthetic Procedure:

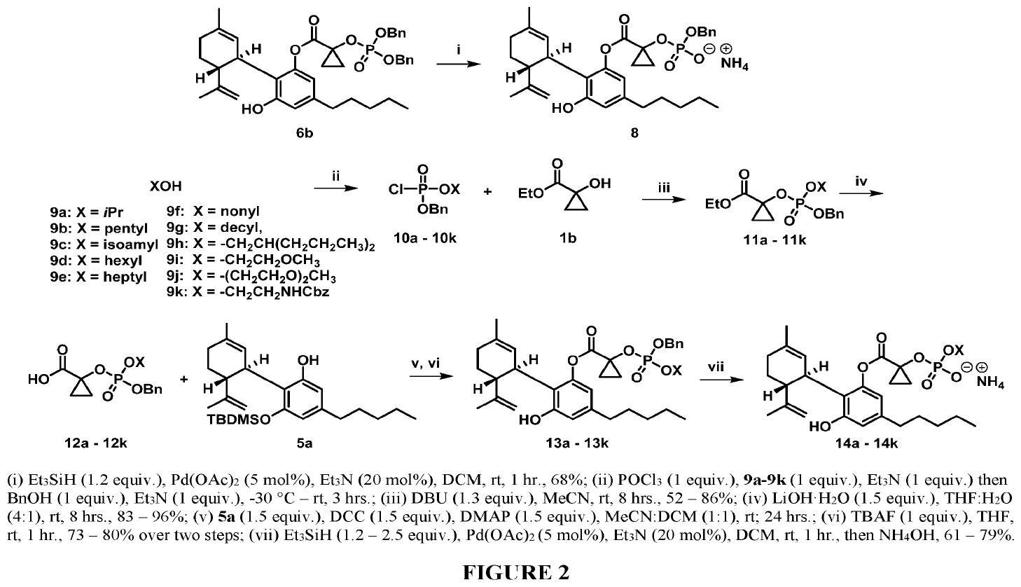

Compounds with a di-ester phosphate prodrug moiety attached by a cyclopropyl-glycolic ester linker to CBD were designed and synthesized using the procedures described below. The synthesis of the di-ester phosphate prodrugs 8 and 14a-k is shown in FIG. 2.

Firstly, the stoichiometric amount of triethylsilane added in the palladium acetate catalyzed benzyl phosphate deprotection of 6b was reduced to 1.2 equivalents. This facilitated mono-debenzylation of 6b and the mono-benzyl di-ester phosphate prodrug 8, which was furnished as the ammonium salt by treatment with NH₄OH.

To access the remaining di-ester phosphate prodrugs 14a-k, the corresponding substituted phosphorochloridates were first synthesized. A cooled solution of phosphoryl chloride was sequentially treated by dropwise addition of one equivalent of triethylamine and the respective alcohol (9a-k), followed by one equivalent of triethylamine and benzyl alcohol. Following aqueous workup with 1M citric acid, the pure phosphorochloridates 10a-k were isolated by column chromatography. Acylation of 2b with 10a-k was carried out utilizing DBU as base, followed by subsequent ester hydrolysis using lithium hydroxide monohydrate in a THF/H₂O mixture to afford the carboxylic acid derivatives 12a-k. As previously described, Steglich esterification between 5a and 12a-k afforded the silyl protected intermediates, which were isolated by aqueous workup and column chromatography, and deprotected using a 1M solution of TBAF in THE to afford the respectively substituted phosphate compounds 13a-k. Debenzylation using 1.2 equivalents of triethylsilane and palladium acetate afforded the desired di-ester phosphate prodrugs 14a-k as ammonium salts following treatment with NH₄OH.

### Chemical Characterization:

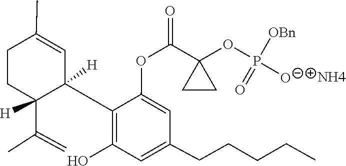 [SMILES: C=C(C)[C@@H]1CCC(C)=C[C@H]1c1c(O)cc(CCCCC)cc1OC(=O)C1(OP(=O)([O-])OCc2ccccc2)CC1 ; [NH4+]]

Compound 8: benzyl (1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl) phosphate ammonium salt.

¹H NMR (400 MHz DMSO-d₆) δ 9.39 (br s, 1H), 7.35-7.19 (m, 5H), 6.44 (s, 1H), 6.19 (s, 1H), 5.01 (s, 1H), 4.80 (s, 2H), 4.46 (s, 1H), 4.37 (s, 1H), 3.73 (br s, 1H), 2.74 (br s, 1H), 2.34 (t, J=7.8 Hz, 2H), 2.21-2.08 (m, 1H), 1.97-1.87 (m, 1H), 1.70-1.59 (m, 2H), 1.57 (s, 3H), 1.54 (s, 3H), 1.48-1.40 (m, 2H), 1.31-1.18 (m, 4H), 1.15 (t, J=7.3 Hz, 4H), 0.84 (t, J=6.9 Hz, 3H); ³¹P NMR (162 MHz, DMSO-d₆) δ-2.48.

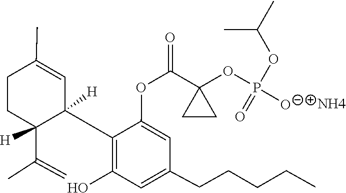 [SMILES: C=C(C)[C@@H]1CCC(C)=C[C@H]1c1c(O)cc(CCCCC)cc1OC(=O)C1(OP(=O)([O-])OC(C)C)CC1 ; [NH4+]]

Compound 14a: 1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl isopropyl phosphate ammonium salt.

¹H NMR (400 MHz, DMSO-d₆) δ 9.44 (br s, 1H), 6.45 (s, 1H), 6.25 (s, 1H), 5.01 (s, 1H), 4.47 (s, 1H), 4.40 (s, 1H), 4.39-4.31 (m, 1H), 3.72 (br s, 1H), 2.74 (br s, 1H), 2.38 (t, J=7.8 Hz, 2H), 2.21-2.08 (m, 1H), 1.97-1.87 (m, 1H), 1.71-1.60 (m, 2H), 1.58 (s, 3H), 1.56 (s, 3H), 1.52-1.40 (m, 2H), 1.32-1.20 (m, 4H), 1.17 (t, J=7.3 Hz, 4H), 1.11 (dd, J=6.2, 2.5 Hz, 6H), 0.85 (t, J=6.9 Hz, 3H); ³¹P NMR (162 MHz, DMSO-d₆) δ-2.86.

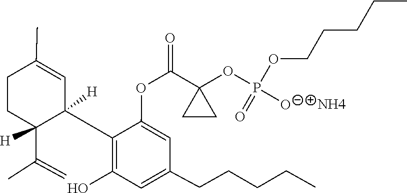 [SMILES: C=C(C)[C@@H]1CCC(C)=C[C@H]1c1c(O)cc(CCCCC)cc1OC(=O)C1(OP(=O)([O-])OCCCCC)CC1 ; [NH4+]]

Compound 14b: 1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl pentyl phosphate ammonium salt.

¹H NMR (400 MHz, DMSO-d₆) δ 9.49 (br s, 1H), 6.46 (s, 1H), 6.23 (s, 1H), 5.01 (s, 1H), 4.46 (s, 1H), 4.40 (s, 1H), 3.73 (br s, 3H), 2.70 (br s, 1H), 2.38 (t, J=7.7 Hz, 2H), 2.20-2.08 (m, 1H), 1.97-1.87 (m, 1H), 1.72-1.61 (m, 2H), 1.58 (s, 3H), 1.56 (s, 3H), 1.54-1.41 (m, 5H), 1.32-1.19 (m, 9H), 1.17 (t, J=7.3 Hz, 2H), 0.88-0.79 (m, 6H); ³¹P NMR (162 MHz, DMSO-d₆) δ-2.65.

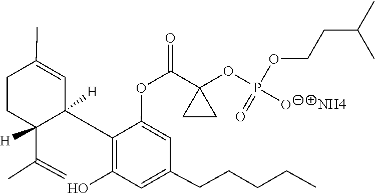 [SMILES: C=C(C)[C@@H]1CCC(C)=C[C@H]1c1c(O)cc(CCCCC)cc1OC(=O)C1(OP(=O)([O-])OCCC(C)C)CC1 ; [NH4+]]

Compound 14c: 1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl isopentyl phosphate ammonium salt.

¹H NMR (400 MHz, DMSO-d₆) δ 9.44 (br s, 1H), 6.45 (s, 1H), 6.24 (s, 1H), 4.47 (s, 1H), 4.40 (s, 1H), 3.80-3.72 (m, 3H), 2.74 (br s, 1H), 2.38 (t, J=7.7 Hz, 2H), 2.21-2.10 (m, 1H), 1.97-1.87 (m, 1H), 1.71-1.60 (m, 2H), 1.58 (s, 3H), 1.56 (s, 3H), 1.53-1.43 (m, 4H), 1.40-1.33 (m, 2H), 1.31-1.20 (m, 5H), 1.19-1.13 (m, 3H), 0.88-0.78 (m, 9H); ³¹P NMR (162 MHz, DMSO-d₆) δ-2.38.

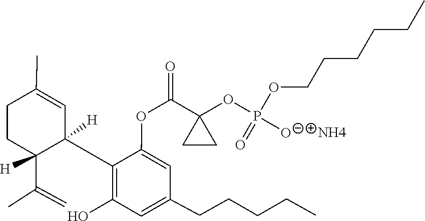 [SMILES: C=C(C)[C@@H]1CCC(C)=C[C@H]1c1c(O)cc(CCCCC)cc1OC(=O)C1(OP(=O)([O-])OCCCCCC)CC1 ; [NH4+]]

Compound 14d: hexyl (1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl) phosphate ammonium salt.

¹H NMR (400 MHz, DMSO-d₆) δ 9.55 (br s, 1H), 6.47 (s, 1H), 6.22 (s, 1H), 5.01 (s, 1H), 4.49-4.44 (m, 1H), 4.42-4.36 (m, 1H), 3.78-3.68 (m, 2H), 2.70 (br s, 1H), 2.37 (t, J=7.7 Hz, 2H), 2.21-2.09 (m, 1H), 1.97-1.87 (m, 1H), 1.71-1.60 (m, 2H), 1.58 (s, 3H), 1.56 (s, 3H), 1.52-1.41 (m, 5H), 1.34-1.12 (m, 16H), 0.87-0.81 (m, 6H); ³¹P NMR (162 MHz, DMSO-d₆) δ-2.53.

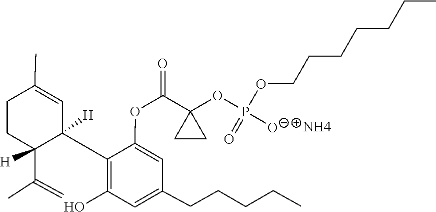 [SMILES: C=C(C)[C@@H]1CCC(C)=C[C@H]1c1c(O)cc(CCCCC)cc1OC(=O)C1(OP(=O)([O-])OCCCCCCC)CC1 ; [NH4+]]

Compound 14e: heptyl (1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl) phosphate ammonium salt.

¹H NMR (400 MHz, DMSO-d₆) δ 9.55 (br s, 1H), 6.47 (s, 1H), 6.22 (s, 1H), 5.01 (s, 1H), 4.49-4.44 (m, 1H), 4.42-4.36 (m, 1H), 3.78-3.68 (m, 2H), 2.70 (br s, 1H), 2.37 (t, J=7.7 Hz, 2H), 2.21-2.09 (m, 1H), 1.97-1.87 (m, 1H), 1.71-1.60 (m, 2H), 1.58 (s, 3H), 1.56 (s, 3H), 1.52-1.41 (m, 5H), 1.34-1.12 (m, 16H), 0.87-0.81 (m, 6H); ³¹P NMR (162 MHz, DMSO-d₆) δ-2.53.

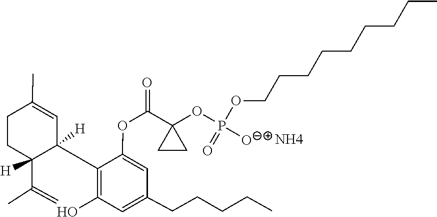 [SMILES: C=C(C)[C@@H]1CCC(C)=C[C@H]1c1c(O)cc(CCCCC)cc1OC(=O)C1(OP(=O)([O-])OCCCCCCCCC)CC1 ; [NH4+]]

Compound 14f: 1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl nonyl phosphate ammonium salt.

¹H NMR (400 MHz, DMSO-d₆) δ 9.47 (br s, 1H), 6.45 (s, 1H), 6.23 (s, 1H), 5.01 (s, 1H), 4.47 (s, 1H), 4.39 (s, 1H), 3.75-3.66 (m, 3H), 2.74 (br s, 1H), 2.38 (t, J=7.7 Hz, 2H), 2.22-2.08 (m, 1H), 1.97-1.86 (m, 1H), 1.72-1.61 (m, 2H), 1.58 (s, 3H), 1.56 (s, 3H), 1.52-1.40 (m, 5H), 1.32-1.12 (m, 20H), 0.90-0.80 (m, 6H); ³¹P NMR (162 MHz, DMSO-d₆) δ-2.18.

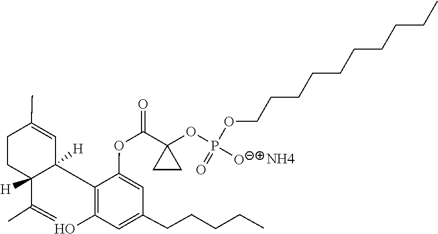 [SMILES: C=C(C)[C@@H]1CCC(C)=C[C@H]1c1c(O)cc(CCCCC)cc1OC(=O)C1(OP(=O)([O-])OCCCCCCCCCC)CC1 ; [NH4+]]

Compound 14g: decyl (1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl) phosphate ammonium salt.

¹H NMR (400 MHz, DMSO-d₆) δ 9.46 (br s, 1H), 6.45 (s, 1H), 6.23 (s, 1H), 5.01 (s, 1H), 4.49-4.44 (m, 1H), 4.42-4.36 (m, 1H), 3.79-3.69 (m, 2H), 2.73 (br s, 1H), 2.38 (t, J=7.7 Hz, 2H), 2.21-2.09 (m, 1H), 1.97-1.86 (m, 1H), 1.74-1.60 (m, 2H), 1.58 (s, 3H), 1.56 (s, 3H), 1.53-1.40 (m, 6H), 1.32-1.13 (m, 21H), 0.88-0.80 (m, 6H); ³¹P NMR (162 MHz, DMSO-d₆) δ-2.47.

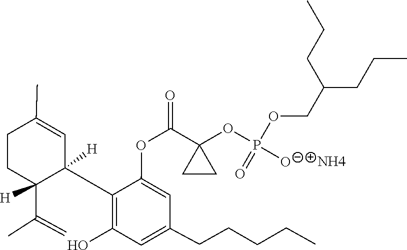 [SMILES: C=C(C)[C@@H]1CCC(C)=C[C@H]1c1c(O)cc(CCCCC)cc1OC(=O)C1(OP(=O)([O-])OCC(CCC)CCC)CC1 ; [NH4+]]

Compound 14h: 1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl (2-propylpentyl) phosphate ammonium salt.

¹H NMR (400 MHz, DMSO-d₆) δ 9.44 (br s, 1H), 6.45 (s, 1H), 6.24 (s, 1H), 5.01 (s, 1H), 4.49-4.44 (m, 1H), 4.42-4.36 (m, 1H), 3.69 (br s, 1H), 3.66-3.56 (m, 2H), 2.79 (br s, 1H), 2.37 (t, J=7.7 Hz, 2H), 2.22-2.09 (m, 1H), 1.97-1.85 (m, 1H), 1.71-1.60 (m, 2H), 1.58 (s, 3H), 1.56 (s, 3H), 1.53-1.38 (m, 4H), 1.32-1.07 (m, 15H), 0.90-0.76 (m, 9H); ³¹P NMR (162 MHz, DMSO-d₆) δ-2.03.

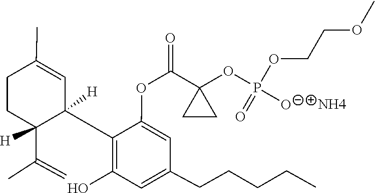 [SMILES: C=C(C)[C@@H]1CCC(C)=C[C@H]1c1c(O)cc(CCCCC)cc1OC(=O)C1(OP(=O)([O-])OCCOC)CC1 ; [NH4+]]

Compound 14i: 1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl (2-methoxyethyl) phosphate ammonium salt.

¹H NMR (400 MHz, DMSO-d₆) δ 9.44 (br s, 1H), 6.49-6.44 (m, 1H), 6.28-6.23 (m, 1H), 5.02 (s, 1H), 4.49-4.39 (m, 2H), 3.87-3.76 (m, 2H), 3.22 (s, 3H), 3.06-2.98 (m, 2H), 2.73 (br s, 1H), 2.42-2.35 (m, 2H), 2.15 (s, 1H), 1.93 (s, 1H), 1.71-1.62 (m, 2H), 1.59 (s, 3H), 1.56 (s, 3H), 1.53-1.44 (m, 2H), 1.34-1.22 (m, 4H), 1.21-1.12 (m, 4H), 0.86 (t, J=6.9 Hz, 3H); ³¹P NMR (162 MHz, DMSO-d₆) δ-2.30.

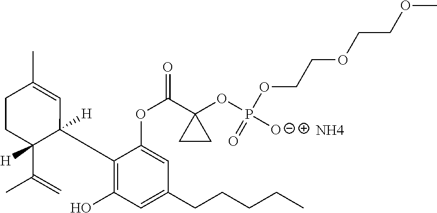 [SMILES: C=C(C)[C@@H]1CCC(C)=C[C@H]1c1c(O)cc(CCCCC)cc1OC(=O)C1(OP(=O)([O-])OCCOCCOC)CC1 ; [NH4+]]

Compound 14j: 1-((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)cyclopropyl (2-(2-methoxyethoxy)ethyl) phosphate ammonium salt.

¹H NMR (400 MHz, DMSO-d₆) δ 9.48 (br s, 1H), 6.51-6.41 (m, 1H), 6.30-6.23 (m, 1H), 5.07-4.99 (m, 1H), 4.53-4.37 (m, 2H), 3.82 (s, 2H), 3.52-3.43 (m, 6H), 3.23 (s, 3H), 2.74 (br s, 1H), 2.42-2.36 (m, 2H), 2.14 (s, 1H), 1.99-1.88 (m, 1H), 1.74-1.61 (m, 2H), 1.59 (s, 3H), 1.56 (s, 3H), 1.54-1.44 (m, 2H), 1.34-1.21 (m, 4H), 1.22-1.13 (m, 4H), 0.86 (t, J=6.9 Hz, 3H); ³¹P NMR (162 MHz, DMSO-d₆) δ-2.58.

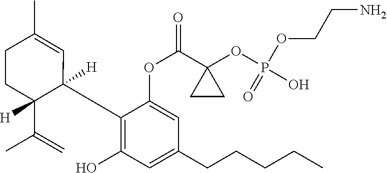 [SMILES: C=C(C)[C@@H]1CCC(C)=C[C@H]1c1c(O)cc(CCCCC)cc1OC(=O)C1(OP(=O)(O)OCCN)CC1]

Compound 14k: (1′R,2′R)-6-hydroxy-5′-methyl-4-pentyl-2′-(prop-1-en-2-yl)-1′,2′,3′,4′-tetrahydro-[1,1′-biphenyl]-2-yl 1-(((2-aminoethoxy(hydroxy)phosphoryl)oxy)-cyclopropane-1-carboxylate.

¹H NMR (400 MHz, DMSO) δ 9.40 (br s, 1H), 8.36 (br s, 2H), 6.45 (d, J=1.7 Hz, 1H), 6.21 (d, J=1.6 Hz, 1H), 5.03 (s, 1H), 4.50-4.45 (m, 1H), 4.43-4.38 (m, 1H), 3.97-3.87 (m, 2H), 3.75 (br s, 1H), 3.00-2.88 (m, 2H), 2.69 (br s, 1H), 2.39 (t, J=7.7 Hz, 2H), 2.21-2.08 (m, 1H), 1.98-1.88 (m, 2H), 1.72-1.64 (m, 2H), 1.59 (s, 3H), 1.56 (s, 3H), 1.53-1.42 (m, 4H), 1.34-1.18 (m, 6H), 0.85 (t, J=6.9 Hz, 3H). ³¹P NMR (162 MHz, DMSO) δ-1.29.

### Example 3 (Compound 24)

### Synthesis of Carbonate Linked Mono-Ester Phosphate Prodrug Derivatives

### Synthetic Procedure:

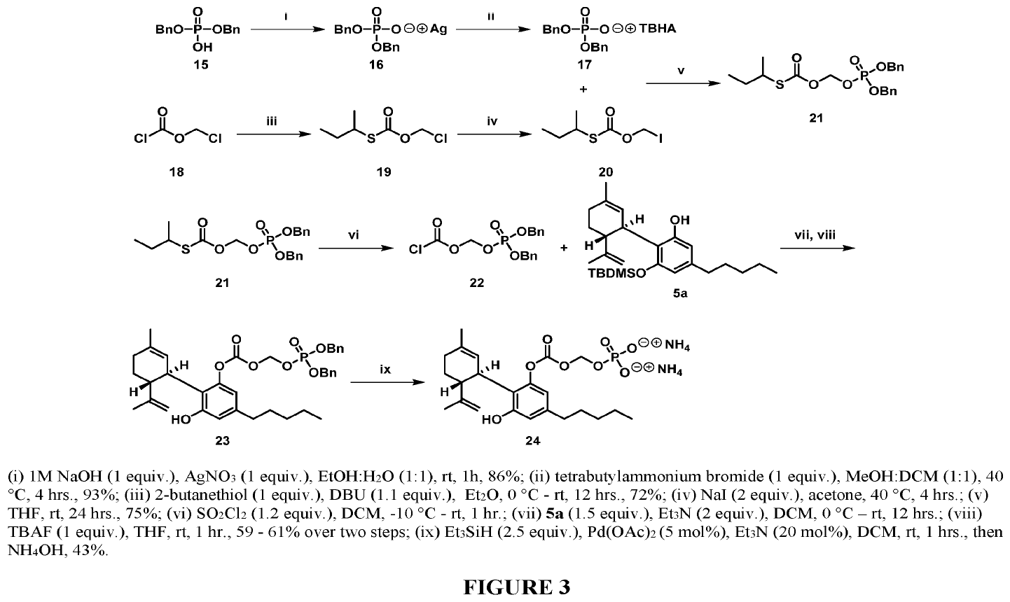

Compounds with a mono-ester phosphate prodrug moiety attached by carbonate linker to CBD were designed and synthesized using the procedures described below. The synthesis of the methylene carbonate linked mono-ester phosphate prodrug 24 is shown in FIG. 3.

To access intermediate 21, the two component fragments 17 and 20 first had to be synthesized. The stepwise conversion of dibenzyl phosphate 15 to its silver salt using silver nitrate, followed by treatment with tetrabutylammonium bromide afforded the tetrabutylammonium salt 17. For fragment 20, acylation between 2-butanethiol and chloromethyl chloroformate yielded intermediate 19, which underwent a Finkelstein reaction to furnish 20. Fragments 17 and 20 were then reacted in THE for 24 hr to afford intermediate 21. Conversion to the acid chloride 22 was facilitated by sulfuryl chloride, which was immediately followed by acylation with TBDMS protected CBD (5a) and TBAF mediated deprotection, to afford the dibenzyl phosphate compound 23. Lastly, palladium acetate and triethylsilane mediated debenzylation and treatment with NH₄OH afforded the target methylene carbonate linked phosphate prodrug 24 as the di-ammonium salt.

### Chemical Characterization:

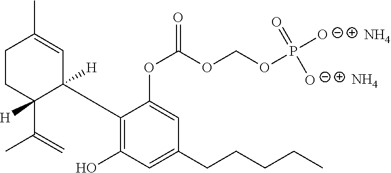 [SMILES: C=C(C)[C@@H]1CCC(C)=C[C@H]1c1c(O)cc(CCCCC)cc1OC(=O)OCOP(=O)([O-])[O-] ; [NH4+] ; [NH4+]]

Compound 24: (1′R,2′R)-6-hydroxy-5′-methyl-4-pentyl-2′-(prop-1-en-2-yl)-1′,2′,3′,4′-tetrahydro-[1,1′-biphenyl]-2-yl ((phosphonooxy)methyl) carbonate di-ammonium salt.

¹H NMR (400 MHz, DMSO-d₆) δ 8.84 (s, 1H), 6.56-6.49 (m, 1H), 6.33 (d, J=1.6 Hz, 1H), 5.49-5.41 (m, 1H), 5.37-5.31 (m, 1H), 5.10-4.97 (m, 1H), 4.50-4.38 (m, 2H), 3.19-3.14 (m, 1H), 2.72 (s, 1H), 2.45-2.37 (m, 2H), 2.13 (s, 1H), 1.98-1.86 (m, 1H), 1.71-1.62 (m, 2H), 1.62-1.55 (m, 6H), 1.53-1.44 (m, 2H), 1.36-1.21 (m, 4H), 0.86 (t, J=6.9 Hz, 3H); ³¹P NMR (162 MHz, DMSO) δ-2.27.

### Example 4 (Compounds 29A-C)

### Synthetic Procedure:

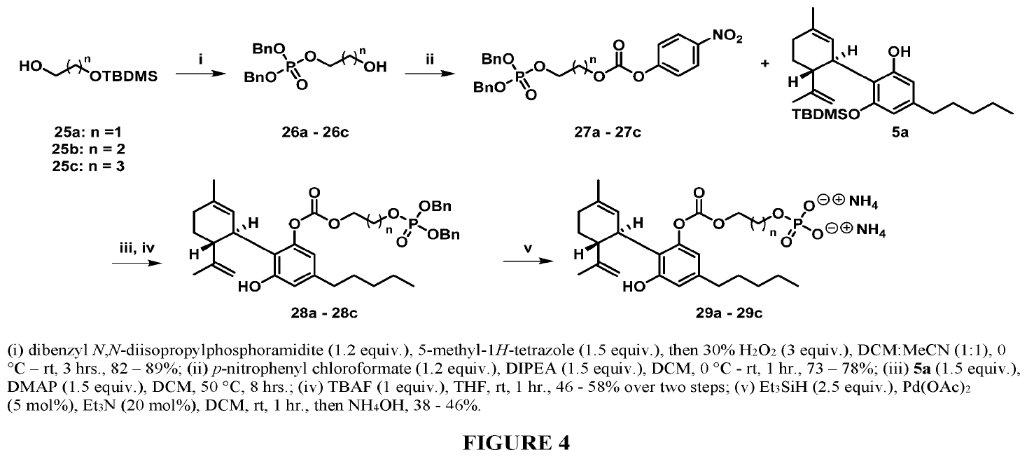

The synthesis of the longer chain carbonate linked mono-ester phosphate prodrug 29a-c is shown in FIG. 4.

For the synthesis of longer chain carbonate linked phosphate prodrugs, the commercially available TBDMS protected alcohols 25a-c were reacted with dibenzyl N,N-diisopropylphosphoramidite and subsequently oxidized with hydrogen peroxide to furnish the dibenzyl phosphate intermediates 26a-c. Acylation of the aliphatic hydroxyl with p-nitrophenyl chloroformate afforded the p-nitrophenyl carbonate compounds 27a-c, which were reacted with 5a and 4-dimethylaminopyridine to yield the penultimate intermediates 28a-c. Finally, debenzylation utilizing triethylsilane and palladium acetate, and treatment with NH₄OH, afforded the desired longer chain carbonate linked phosphate CBD prodrugs 29a-c as the di-ammonium salts.

### Chemical Characterization:

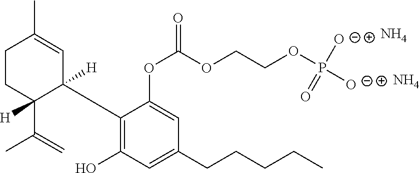 [SMILES: C=C(C)[C@@H]1CCC(C)=C[C@H]1c1c(O)cc(CCCCC)cc1OC(=O)OCCOP(=O)([O-])[O-] ; [NH4+] ; [NH4+]]

Compound 29a: (1′R,2′R)-6-hydroxy-5′-methyl-4-pentyl-2′-(prop-1-en-2-yl)-1′,2′,3′,4′-tetrahydro-[1,1′-biphenyl]-2-yl(2-(phosphonooxy)ethyl) carbonate di-ammonium salt.

¹H NMR (400 MHz, DMSO-d₆) δ 9.50 (br s, 1H), 6.49 (s, 1H), 6.33 (s, 1H), 5.01 (s, 1H), 4.49-4.38 (m, 2H), 4.36-4.10 (m, 2H), 3.99 (s, 2H), 3.01 (br s, 1H), 2.72 (br s, 1H), 2.44-2.36 (m, 2H), 2.13 (s, 1H), 1.97-1.85 (m, 1H), 1.71-1.62 (m, 2H), 1.58 (s, 6H), 1.53-1.44 (m, 2H), 1.32-1.18 (m, 4H), 0.85 (t, J=6.8 Hz, 3H); ³¹P NMR (162 MHz, DMSO-d₆) δ-2.14.

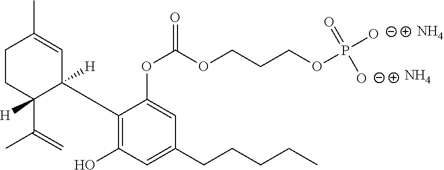 [SMILES: C=C(C)[C@@H]1CCC(C)=C[C@H]1c1c(O)cc(CCCCC)cc1OC(=O)OCCCOP(=O)([O-])[O-] ; [NH4+] ; [NH4+]]

Compound 29b: (1′R,2′R)-6-hydroxy-5′-methyl-4-pentyl-2′-(prop-1-en-2-yl)-1′,2′,3′,4′-tetrahydro-[1,1′-biphenyl]-2-yl(3-(phosphonooxy)propyl) carbonate di-ammonium salt.

¹H NMR (400 MHz, DMSO-d₆) δ 6.52 (s, 1H), 6.32 (s, 1H), 5.02 (s, 1H), 4.43 (s, 2H), 4.26-4.16 (m, 2H), 4.16-4.07 (m, 2H), 3.80-3.72 (m, 1H), 2.70 (br s, 1H), 2.45-2.36 (m, 2H), 2.10 (s, 1H), 1.97-1.90 (m, 1H), 1.93-1.83 (m, 2H), 1.72-1.60 (m, 2H), 1.62-1.55 (m, 6H), 1.56-1.44 (m, 2H), 1.35-1.18 (m, 4H), 0.85 (t, J=6.9 Hz, 3H); ³¹P NMR (162 MHz, DMSO-d₆) δ-0.08.

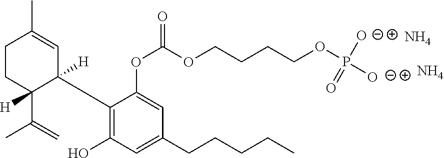 [SMILES: C=C(C)[C@@H]1CCC(C)=C[C@H]1c1c(O)cc(CCCCC)cc1OC(=O)OCCCCOP(=O)([O-])[O-] ; [NH4+] ; [NH4+]]

Compound 29c: (1′R,2′R)-6-hydroxy-5′-methyl-4-pentyl-2′-(prop-1-en-2-yl)-1′,2′,3′,4′-tetrahydro-[1,1′-biphenyl]-2-yl(4-(phosphonooxy)butyl) carbonate di-ammonium salt.

¹H NMR (400 MHz, DMSO-d₆) δ 6.52 (s, 1H), 6.31 (s, 1H), 5.02 (s, 1H), 4.42 (s, 2H), 4.21-4.11 (m, 2H), 4.08-4.01 (m, 2H), 3.75-3.66 (m, 1H), 2.71 (br s, 1H), 2.45-2.36 (m, 2H), 2.10 (s, 1H), 1.98-1.88 (m, 1H), 1.75-1.62 (m, 2H), 1.64-1.52 (m, 10H), 1.54-1.44 (m, 2H), 1.35-1.20 (m, 4H), 0.85 (t, J=6.9 Hz, 3H); ³¹P NMR (162 MHz, DMSO-d₆) δ-0.16.

### Example 5 (Compound 32)

### Synthesis of Long Chain Ester Linked Mono-Ester Phosphate Prodrug Derivatives

### Synthetic Procedure:

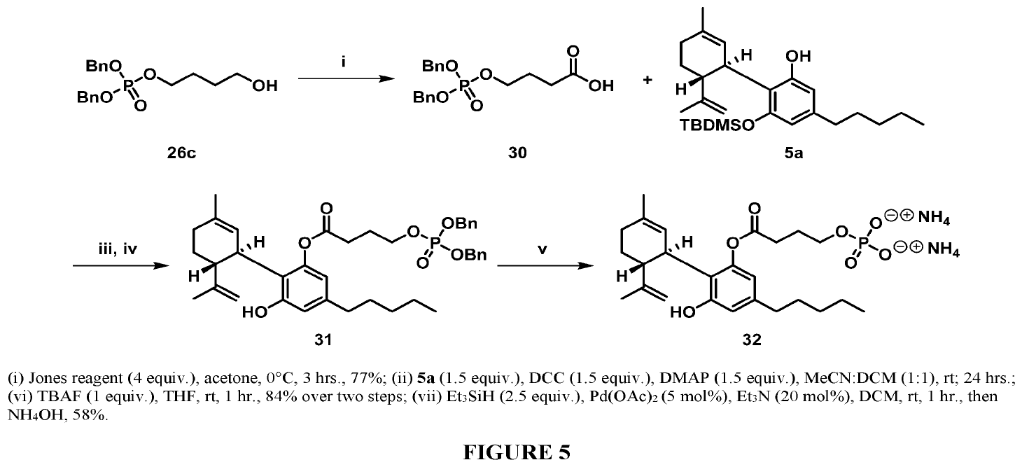

Compounds with a mono-ester phosphate prodrug moiety attached by a long chain ester linker to CBD were designed and synthesized using the procedures described below. The synthesis of the long chain ester linked mono-ester phosphate prodrug 32 is shown in FIG. 5.

For the longer chain ester linked phosphate CBD prodrugs, the previously synthesized analogue 26c underwent a Jones oxidation to afford the carboxylic acid 30. Subsequent Steglich esterification, followed by TBAF mediated silyl deprotection, afforded the dibenzyl phosphate intermediate 31. Lastly, debenzylation via triethylsilane and palladium acetate furnished the desired prodrug 32 as the di-ammonium salt following treatment with NH₄OH.

### Chemical Characterization:

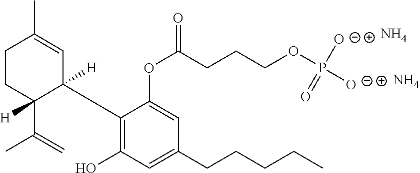 [SMILES: C=C(C)[C@@H]1CCC(C)=C[C@H]1c1c(O)cc(CCCCC)cc1OC(=O)CCCOP(=O)([O-])[O-] ; [NH4+] ; [NH4+]]

Compound 32: (1′R,2′R)-6-hydroxy-5′-methyl-4-pentyl-2′-(prop-1-en-2-yl)-1′,2′,3′,4′-tetrahydro-[1,1′-biphenyl]-2-yl 4-(phosphonooxy)butanoate di-ammonium salt.

¹H NMR (400 MHz, DMSO) δ 6.47 (s, 1H), 6.21 (s, 1H), 4.99 (s, 1H), 4.42 (dd, J=6.6, 2.3 Hz, 1H), 3.80-1.73 (m, 1H), 3.72 (br s, 1H), 2.71-2.53 (br s, 1H), 2.38 (t, J=7.7 Hz, 2H), 2.19-2.06 (m, 1H), 1.98-1.88 (m, 1H), 1.88-1.75 (m, 2H), 1.71-1.61 (m, 2H), 1.58 (s, 6H), 1.48 (p, J=7.4 Hz, 2H), 1.35-1.12 (m, 5H), 0.84 (t, J=6.9 Hz, 3H). ³¹P NMR (162 MHz, DMSO) δ-0.53.

### Example 6 (Compound 38A-C)

### Synthesis of Carbamate Linked Mono-Ester Phosphate Prodrug Derivatives

### Synthetic Procedure:

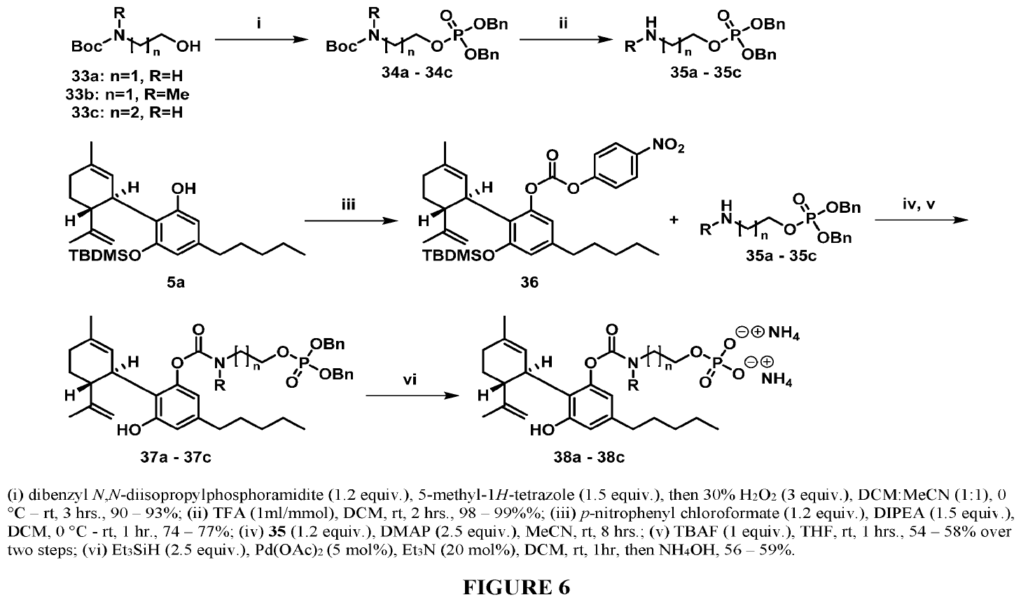

Compounds with a mono-ester phosphate prodrug moiety attached by a carbamate linker to CBD were designed and synthesized using the procedures described below. The synthesis of the carbamate linked mono-ester phosphate prodrug 38a-c is shown in FIG. 6.

The dibenzyl phosphate moiety was introduced to commercially available N-boc-ethanolamine through reaction with dibenzyl N,N-diisopropylphosphoramidite and subsequent oxidation with hydrogen peroxide, affording intermediate 34a-c in a good yield. Deprotection by stirring in a solution of trifluoroacetic acid in DCM resulted in formation of amine 35a-c, which was dried under vacuum and used without further purification. The activated carbonate 36, was accessed by acylation of TBDMS protected CBD (5a) with p-nitrophenyl chloroformate. This compound was purified by directly loading onto silica after removal of the solvent in vacuo and carrying out column chromatography. Reaction between 35a-c and 36 was undertaken in DCM with DMAP serving as base, followed by immediate TBAF mediated deprotection to afford dibenzyl phosphate intermediate 37a-c. Lastly, this compound was submitted to debenzylation conditions with triethylsilane and palladium acetate, yielding the desired carbamate linked phosphate prodrug 38a-c as the di-ammonium salt following treatment with NH₄OH.

### Chemical Characterization:

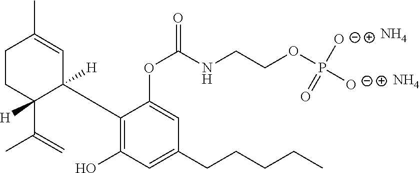 [SMILES: C=C(C)[C@@H]1CCC(C)=C[C@H]1c1c(O)cc(CCCCC)cc1OC(=O)NCCOP(=O)([O-])[O-] ; [NH4+] ; [NH4+]]

Compound 38a: (1′R,2′R)-6-hydroxy-5′-methyl-4-pentyl-2′-(prop-1-en-2-yl)-1′,2′,3′,4′-tetrahydro-[1,1′-biphenyl]-2-yl(2-(phosphonooxy)ethyl)carbamate di-ammonium salt.

¹H NMR (400 MHz, DMSO) δ 7.64 (br s, 1H), 6.40 (s, 1H), 6.20 (s, 1H), 5.00 (s, 1H), 4.44 (s, 1H), 4.39 (s, 1H), 3.32-3.03 (m, 3H), 2.84 (br s, 1H), 2.38 (t, J=7.7 Hz, 2H), 2.30-2.17 (m, 1H), 1.90-1.83 (m, 1H), 1.68-1.40 (m, 11H), 1.35-1.18 (m, 5H), 0.85 (t, J=6.9 Hz, 3H). ³¹P NMR (162 MHz, DMSO) δ-0.42.

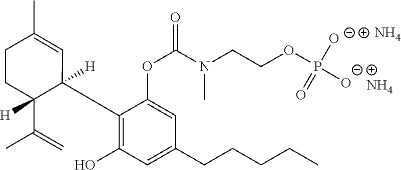 [SMILES: C=C(C)[C@@H]1CCC(C)=C[C@H]1c1c(O)cc(CCCCC)cc1OC(=O)N(C)CCOP(=O)([O-])[O-] ; [NH4+] ; [NH4+]]

Compound 38b: (1′R,2′R)-6-hydroxy-5′-methyl-4-pentyl-2′-(prop-1-en-2-yl)-1′,2′,3′,4′-tetrahydro-[1,1′-biphenyl]-2-yl methyl(2-(phosphonooxy)ethyl)carbamate di-ammonium salt.

¹H NMR (500 MHz, DMSO) δ 6.42 (s, 1H), 6.20 (s, 1H), 5.09 (s, 1H), 4.41 (d, J=9.4 Hz, 2H), 3.88-3.75 (m, 3H), 3.03-2.89 (m, 7H), 2.42-2.35 (m, 2H), 2.08 (s, 1H), 1.94 (d, J=16.7 Hz, 1H), 1.72-1.54 (m, 8H), 1.53-1.44 (m, 2H), 1.34-1.20 (m, 4H), 0.88-0.82 (m, 3H). ³¹P NMR (162 MHz, DMSO) δ-0.19.

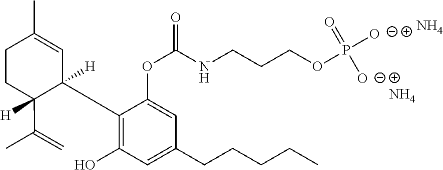 [SMILES: C=C(C)[C@@H]1CCC(C)=C[C@H]1c1c(O)cc(CCCCC)cc1OC(=O)NCCCOP(=O)([O-])[O-] ; [NH4+] ; [NH4+]]

Compound 38c. (1′R,2′R)-6-hydroxy-5′-methyl-4-pentyl-2′-(prop-1-en-2-yl)-1′,2′,3′,4′-tetrahydro-[1,1′-biphenyl]-2-yl (3-(phosphonooxy)propyl)carbamate di-ammonium salt.

¹H NMR (500 MHz, DMSO) δ 7.48 (s, 1H), 6.40 (s, 1H), 6.18 (s, 1H), 5.01 (s, 1H), 4.47-4.42 (m, 1H), 4.42-4.38 (m, 1H), 3.77-3.68 (m, 3H), 3.11-2.83 (m, 4H), 2.38 (t, J=7.8 Hz, 2H), 1.92-1.84 (m, 1H), 1.73-1.54 (m, 11H), 1.53-1.44 (m, 2H), 1.35-1.20 (m, 4H), 0.88-0.79 (m, 3H). ³¹P NMR (162 MHz, DMSO) δ-0.03.

### Example 7 (Prophetic Compound 43)

### Synthesis of Ester Linked Cyclical-Diester Phosphate Prodrug Derivatives

### Synthetic Procedure:

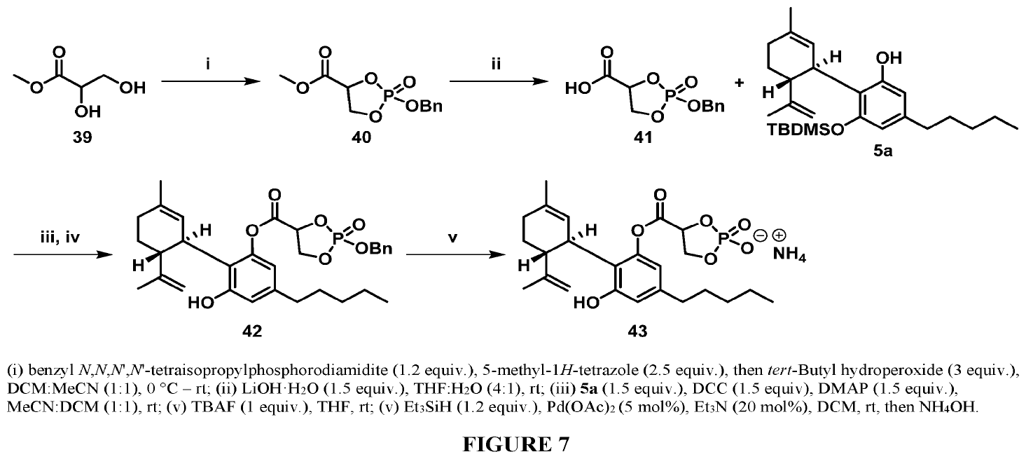

Compounds with a cyclical di-ester phosphate prodrug moiety attached by an ester linker to CBD are designed and can be synthesized using the procedures described below. The prophetic synthesis of the ester linked cyclical di-ester phosphate prodrug 43 is shown in FIG. 7.

For the prophetic synthesis of the cyclical di-ester phosphate prodrugs, the steps will be like that undertaken for the previously synthesized prodrugs. Reaction of commercially available 39 with benzyl N,N,N′,N′-tetraisopropylphosphorodiamidite using 5-methyl-1H-tetrazole, followed by oxidation to the desired phosphate can yield intermediate 40. This will be followed by lithium hydroxide mediate hydrolysis, and Steglich esterification with the silyl protected CBD analogue 5a. Subsequent silyl deprotection will then yield the mono-benzyl protected cyclical phosphate intermediate 42. Lastly, debenzylation via triethylsilane and palladium acetate and treatment with ammonium hydroxide will afford the desired prodrug 43 as the ammonium salt.

### Example 8

### Nephelometry

General Experimental: Nephelometry experiments were performed using untreated Corning® Costar® 96-well black polystyrene plates with clear flat bottoms. Sample stock solutions and serial dilutions were prepared with DriSolv® DMSO purchased from MilliporeSigma. All 100-fold dilutions and replicate experiments were prepared using Gibco® Dulbecco's phosphate-buffered saline (DPBS, no calcium, no magnesium) with a pH range of 7.0-7.3 as aqueous media. Incubation of the 96-well plates was achieved with a Benchmark Incu-Shaker™ Mini shaking incubator. Nephelometry data was obtained using a NEPHELOstar® microplate reader and processed with MARS data analysis software from BMG LabTech.

Procedure for Nephelometry Experiments: Tested compounds were dissolved in 100% DMSO to make stock solutions of specified concentrations, ranging from 10 mM minimum up to 50 mM maximum. The sample then underwent serial dilution in a 96-well plate (Corning® Costar®). Well A1 of the plate contained 100% DMSO. Wells A2-A12 possessed the test compound in DMSO with concentration factors as follows: X mM for A2, (0.8)X mM for A3, (0.6)X mM for A4, (0.4)X mM for A5, (0.2)X mM for A6, (0.1)X mM for A7, (0.05)X mM for A8, (0.025)X mM for A9, (0.0125)X mM for A10, (0.00625)X mM for A11, and (0.003125)X mM for A12. Using a 12 channel multichannel pipette, 2.5 μL of sample from row A was transferred to each well in row B through row H of the plate. Next, 30 μL of PBS (pH=7.0-7.3) was added to row B through row H, providing each well with 32.5 μL. The plate was then incubated for 30 sec with shaking. Finally, 217.5 μL of PBS buffer was added to row B through row H, and the entire plate was incubated with shaking at 25° C. for 90 min. Note: The final volume of DMSO in actual experiments with PBS buffer was 1% throughout the plate. After 90 min, the 96-well plate was analyzed with a NEPHELOstar® instrument and the data was processed with MARS data analysis software. The solubility data determined by the nephelometry experiments are summarized in Tables 1-3.

### Example 9

### Simulated Gastric Fluid Stability Assay

General Experimental: Simulated Gastric Fluid (SGF) is a pH 1.2 HCl solution with purified pepsin (800-2500 units of activity) (Test Solutions, The United States Pharmacopoeia (USP), 2008). SGF was prepared according to a procedure described in Test Solution—USP and other published articles (Bioorganic Chemistry, 49 (2013), 40-48). Sodium chloride 0.20 g and purified pepsin 0.32 g (Sigma), derived from porcine stomach mucosa with an activity of 800 to 2500 units per mg of protein, were dissolved in 0.70 mL hydrochloric acid and sufficient water to make 100 mL. This test solution had a pH of about 1.2. The SGF solution prepared was used for in vitro studies to evaluate the prodrug stability.

Procedure for Simulated Gastric Fluid Stability Assay: 20 μL from the 100 mM stock solution of the test compound (CBD prodrug) in acetonitrile was added to 980 μL SGF and incubated at 37° C. At specified time intervals, aliquots (100 μL) were withdrawn and added to pre-cooled 100% acetonitrile (200 μL spiked with ISTD) and mixed well by vortex mixing for 2 min. The mixture was centrifuged at 13000 rpm for 15 min at 4° C. The supernatant was transferred to an HPLC vial and analyzed using LC-MS/MS to check the amounts of the remaining intact CBD prodrug to obtain its half-life. The results of the SGF stability assay are summarized in Table 1.

### Example 10

### Liver Microsome Stability Assay

General Experimental: Human and mouse liver microsomes were purchased from Xenotech at 20 mg/mL. NADPH (Sigma-Aldrich) 10 mM stocks were prepared in deionized water.

Procedure for Liver Microsome Stability Assay: Test compounds and positive controls were dissolved in 100% DMSO to make 10 mM stock solutions. Verapamil (Sigma-Aldrich) and diphenhydramine (Sigma-Aldrich) were used as positive controls for both human liver microsomes (HLM) and mouse liver microsomes (MLM). The 10 mM stock solutions of test and control compounds were further diluted in potassium phosphate buffer (100 mM, pH 7.4) to 500 μM to ensure that the organic solvent content was <0.2%. The liver microsome (HLM or MLM) assay was prepared in a 1.5 mL Eppendorf tube with a final volume of 1100 μL for duplicate runs. Each reaction contained phosphate buffer (928.4 μL), liver microsomes (55 μL), and test compound (6.6 μL of 500 μM), resulting in a final concentration of 3 μM for the test compound. The reaction was initiated with 110 μL of 10 mM NADPH. Aliquots (100 μL) were removed in duplicate at 0, 5, 10, 15, and 30 min and quenched in 100 mL of 100% cold methanol which contained internal standard (ISTD: d5-7-ethoxy coumarin 2 μM). The aliquots were centrifuged at 12,000 g for 5 min, and the supernatant was transferred and placed in an LC-MS vial. Each time point was assessed by LC-MS/MS, and the area under the m/z curve (AUC), based on the MRM transition, was integrated w.r.t the ISTD. Positive controls were conducted at a final volume of 550 μL to enable a single run for each time point. Negative controls in the absence of NADPH were performed with the test and control compounds, respectively, at a final volume of 150 μL and measured at the most prolonged time point. Control compounds were processed and analyzed like test compounds. Each time point was run in triplicates on LC-MS/MS followed by in-between blank washes to avoid the carryover and to equilibrate the column. Half-life (T_{1/2}) was calculated using the data obtained from LC-MS/MS and by plotting ln (% remaining of the parent prodrug) versus time and performing linear regression to determine slope. Slope=−k and T_{1/2}=0.693/k for first-order kinetics. The results of the HLM and MLM stability assays are summarized in Tables 1-3.

### Example 11

### Plasma Stability Assay

General Experimental: Procaine and Procainamide were purchased from Sigma-Aldrich. Human plasma was obtained from BioIVT (Cat. No. HUMANPLLHP2N) and 1X Dulbecco's PBS (pH 7.4, Gibco) was obtained from Thermos Fischer. Stock solutions were prepared in 100% DMSO, and suitable dilutions of target analytes were prepared in methanol for method development.

Procedure for Plasma Stability Assay: Test compounds were dissolved in DMSO to make a stock solution of 10 mM and then diluted to 500 μM in buffer (or 70% MeOH). Human plasma was thawed at ambient temperature and is aliquoted (994.0 L) to a 1.5 mL Eppendorf tube in duplicates (A and B) for each compound. Plasma was incubated at 37° C. for 10 minutes in an incubator shaker at 150 rpm; the reaction was initiated by the addition of the test compound (6.0 μL) and vortex mixing. The total reaction volume was 1000 μL where the final organic solvent concentrations were 0.6% MeOH and 0.03% DMSO. The spiked plasma samples were incubated at 37° C. for 4 hrs. The reactions were terminated at time points 0, 15, 30, 60, 120, 180, and 240 min by taking a 100 μL aliquot from the test incubation mixture and immediately quenching it in ice-cold acetonitrile containing internal standard (150 μL with 2 μM ISTD), followed by a quick vortex mixing. In addition, matrix blank was prepared by adding acetonitrile solutions containing ISTD to plasma samples without any of the analytes or control compounds. Also, an additional tube was made to measure compound degradation in PBS buffer. The samples were centrifuged at 15000 rpm for 25 min at 4° C. The supernatant was transferred to an LC-MS vial for analysis by LC/MS-MS. Each time point was run in duplicates followed by in-between blank washes to avoid the carryover and to equilibrate the column. Half-life (T_{1/2}) was calculated using the data obtained from LC-MS/MS and by plotting ln (% remaining of the parent prodrug) versus time and performing linear regression to determine slope. Slope=−k and T_{1/2}=0.693/k for first-order kinetics. The results of the human plasma stability assays are summarized in Tables 1-3.

Procaine (poor plasma stability) and procainamide (good plasma stability) were used as positive controls at a final concentration of 3 μM. These positive controls were run in parallel to test the systems for competency.

1.5 mL conical polypropylene microcentrifuge tubes were labelled in duplicate for 0, 15, 30, 60, 120, 180, 240 min for each compound being tested. **

Example: No. Of Tubes Prepared Per Test Compound (TC):

Test compound 1: 994 μL human plasma+6.0 μL TC1 (Vial A)
994 μL human plasma+6.0 μL TC1 (Vial B)

Positive control: 596 μL human plasma+3.6 μL (procaine+pracainamide)

Matrix blank: 500 μL PBS buffer+100 μL human plasma

Negative control: 142 μL PBS buffer+0.9 μL TC1

Quenching mixture: 150 μL acetonitrile with ISTD (2 μM) or 70% MeOH:H₂O (with ISTD)

Final volume after quenching: 250 μL (100 μL from the incubation mixture and 150 μL from the quenching mixture; ISTD conc. 1.2 μM)

### Example 12

### Hepatocyte Cellular Uptake and Stability Assay

General Experimental: Fresh mouse hepatocytes (seeded on 24-well culture plates with Matrigel) were obtained from BioIVT. INVITROGRO HI medium and Torpedo antibiotic mix were obtained from BioIVT.

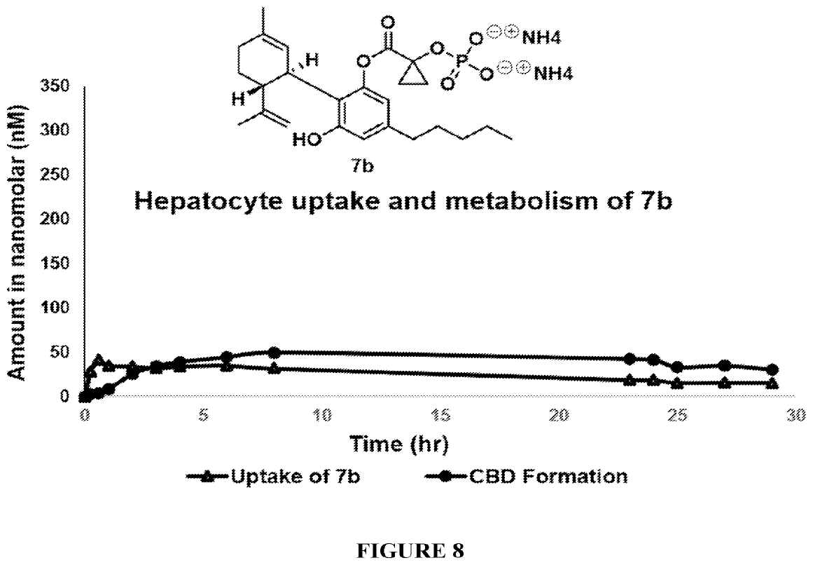

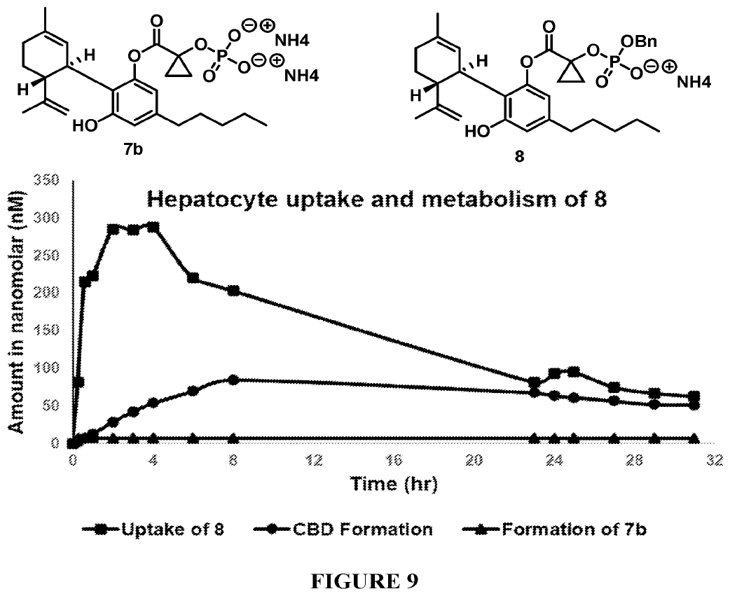

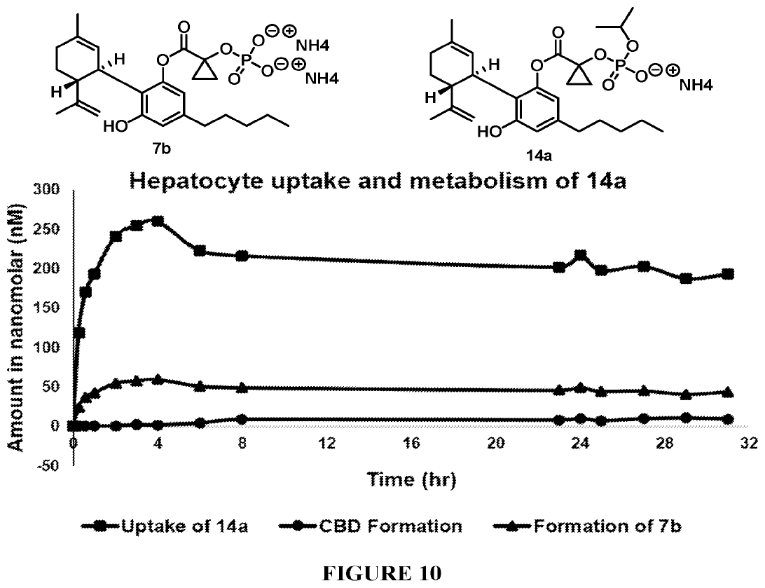

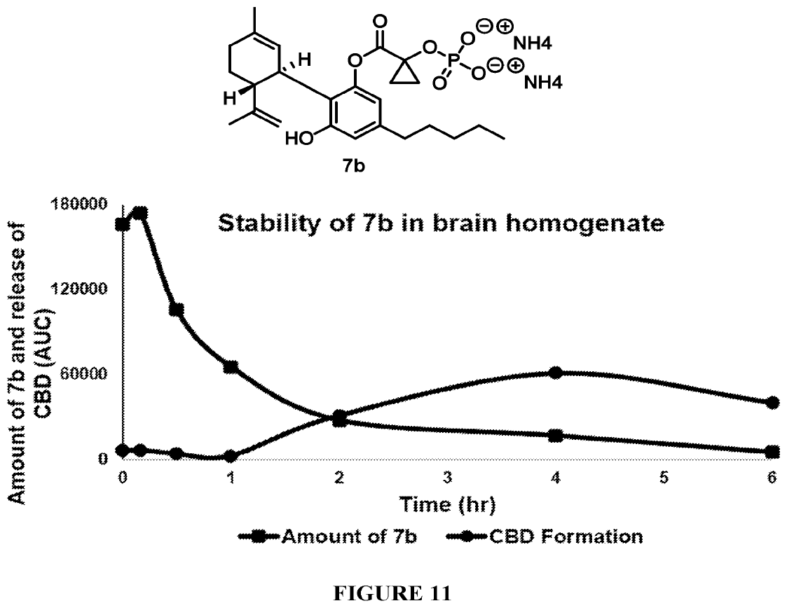

Procedure for Hepatocyte Cellular Uptake and Stability Assay: Complete INVITROGRO HI medium (i.e., the growth medium) was prepared by adding 1.0 mL TORPEDO antibiotic mix to 45 mL INVITROGEN HI medium. The shipping medium was removed from the 24-well plates of fresh mouse hepatocytes seeded at 175K viable cells and replaced by the growth medium described above (1.0 mL/well). The plates were incubated at 37° C. at 5% CO₂ overnight. 40 mM stock solutions of test compounds were prepared in 100% DMSO. The 40 mM stock solutions were then diluted to 20 μM in 50 mL of the growth media. For compound-treatment plates, the blank growth medium was removed and replaced with the compound-containing medium described above (compound concentration at 20 μM). A separate plate of cells in the blank growth medium (with no addition of the test compounds) was used as a control and TO. The compound-treatment plates were incubated at 37° C. at 5% CO₂ for the following time points: 0, 0.5, 1, 2, 3, 4, 6, and 24 hrs. The non-treatment plate was sampled at 0 hrs. After incubation at the desired time points, the cells were washed two times with 1.0 mL of DPBS. The cells were then extracted by adding 500 L of 70% MeOH:H₂O spiked with the internal standard (ISTD). The ISTD was d5-ethoxy coumarin at 2 μM concentration. After mixing, the samples were transferred to pre-labelled microcentrifuge tubes and centrifuged at 15000 for 10 mins at 4° C. After centrifugation, 300 L of the supernatant was transferred to labelled HPLC vials and measured by LC-MS/MS. Each time points were run in duplicates. Standard curves for each test compound and the metabolites thereof were built separately to quantify their concentrations in the samples. The results of the hepatocyte cellular uptake assay for compounds 7b, 8, and 14a are shown in FIGS. 8-10.

### Example 13

### Brain Homogenate Stability Assay

General Experimental: Purchases were made for DPBS (1X) (Gibco) and Pierce BCA Assay kit for protein quantification (Thermo Fischer). Frozen mouse brain homogenate was provided by Dr. Scott Myers at Emory University School of Medicine (C57BL/6J adult male mice (Jackson Laboratory), 239 days (34 weeks) old). Briefly, each mouse was euthanized via isoflurane overdose and immediately upon cessation of breathing the chest cavity was opened and the left ventricle punctured with a 21G needle, the vena cava was then clipped and the body perfused with cold PBS (pH 7.35) for 2 min. Following, the brain was removed (on ice) and the forebrain separated from the cerebellum. The forebrain wet weight was recorded and placed into 3 mL ice cold HBSS (Hank's Balanced Salt Solution, pH 7.4), and then homogenized for 10 secs at 3/4 max setting. The brain homogenate tubes were then capped and quickly frozen on dry ice. The amount of protein in the mouse brain homogenate was quantified using the Pierce BCA assay kit before running the stability assay.

Procedure for Brain Homogenate Stability Assay: 10 mM stock solution was prepared in 100% DMSO for each test compound. The stock solutions were then diluted to 500 μM in DPBS or a 50% MeOH:DPBS solution (for compounds with poor aqueous solubility). Each test sample was prepared in duplicates—in two 1.5 mL Eppendorf tubes labelled as A and B. Each Eppendorf tube contained the following components: 580 μL DPBS+20 μL test compound solution (from the 500 μM batch described above)+400 μL brain homogenate. The negative control was run in parallel in the absence of brain homogenate, i.e., only DPBS with test compound (TC). The assay was initiated by adding the test compound to each Eppendorf tube, followed by vortex mixing and incubation at 37° C. on a shaker at 185 rpm. A small aliquot (100 L) was taken from the Eppendorf tubes at different time points (0, 10, 30, 60, 120, 240, 360, and 1440 min) and quenched by 200 μL cold ACN spiked with 2 μM ISTD. The t=0 sample was taken immediately after adding the TC solution to its corresponding Eppendorf tube. Each aliquoted sample was vortexed thoroughly and centrifuged for 5 min at 12000 rpm at 4° C. The supernatant was transferred into the HPLC vials, and measured by LC-MS/MS. Half-life (T_{1/2}) was calculated using the data obtained from LC-MS/MS and by plotting ln (% remaining of the parent prodrug) versus time and performing linear regression to determine slope. Slope=−k and T_{1/2}=0.693/k for first-order kinetics. The results of the brain homogenate stability assay for compounds 7b are shown in FIG. 11.

### Example 14

### Cyp Inhibition Panel Results

General Experimental: Cytochrome P450 enzymes (CYP) inhibition assays were carried out by SAI Life Sciences Limited. The objective of the study was to evaluate the potential of CBD and compound 7b to inhibit CYP1A2, CYP2A6, CYP2B6, CYP2C₈, CYP2C₉, CYP2C₁₉, CYP2D6, and CYP3A4 respectively. This was accomplished by determining the inhibitory effect of test compounds on phenacetin, coumarin, bupropion, amodiaquin, diclofenac, S-mephenytoin, bufuralol, and midazolam metabolism to acetaminophen, 7-OH coumarin, OH-bupropion, N-desethylamodiaquine, 4-OH diclofenac, 4-OH mephenytoin, OH-bufuralol maleate, and 1-OH Midazolam, respectively, in human liver microsomes. All the metabolites were analysed using a Waters ACQUITYTM, ultra-performance LC-MS/MS. Substrates and inhibitors were purchased from Sigma Aldrich (Germany), pooled human liver microsomes (50 donors) were purchased from Gibco (USA) and NADPH from SRL (India).

Preparation of Reagents: 50 mM potassium phosphate buffer (pH 7.4) was prepared by adding 0.647 g potassium phosphate monobasic (KH₂PO₄) and 3.527 g potassium phosphate dibasic (K₂HPO₄) to 400 mL of Milli-Q water and volume was made up to 500 mL. Microsomes (20 mg/mL) were diluted 50 mM potassium phosphate buffer (pH 7.4) buffer to prepare a concentration of 0.2 mg/mL. For CYP2C19, a concentration of 1 mg/mL was prepared. Stock solutions of test compounds were prepared in DMSO at a concentration of 10 mM and 1 mM. A stock solution of 35 mM phenacetin was prepared in DMSO. The stock was diluted in incubation buffer to prepare a working stock of 140 μM. A stock solution of 20 mM bufuralol was prepared in DMSO. The stock was diluted in incubation buffer to prepare a working stock of 20 μM. A stock solution of 10 mM midazolam was prepared in DMSO. The stock was diluted in incubation buffer to prepare a working stock of 10 μM. A stock solution of 20 mM diclofenac was prepared in DMSO. The stock was diluted in incubation buffer to prepare a working stock of 40 μM. A stock solution of 50 mM S-mephenytoin was prepared in DMSO. The stock was diluted in incubation buffer to prepare a working stock of 120 μM. A stock solution of 50 mM bupropion was prepared in DMSO. The stock was diluted in incubation buffer to prepare a working stock of 200 μM. A stock solution of 2 mM Coumarin was prepared in DMSO. The stock was diluted in incubation buffer to prepare a working stock of 8 μM. A stock solution of 10 mM Amodiaquine was prepared in DMSO. The stock was diluted in incubation buffer to prepare a working stock of 8 μM. Stock solutions of 10 mM fluvoxamine, sulfaphenazole, tranylcypromine, and ticlopidine were prepared in DMSO. Stock solutions of 1 mM ketoconazole and quinidine was prepared in DMSO. Stock solutions of 30 mM Quercetin was prepared in DMSO. Stock solutions of 3 mM Ticlopidine was prepared in DMSO. A stock solution of 4 mM was prepared by dissolving appropriate amount of NADPH in 50 mM potassium phosphate buffer pH 7.4.

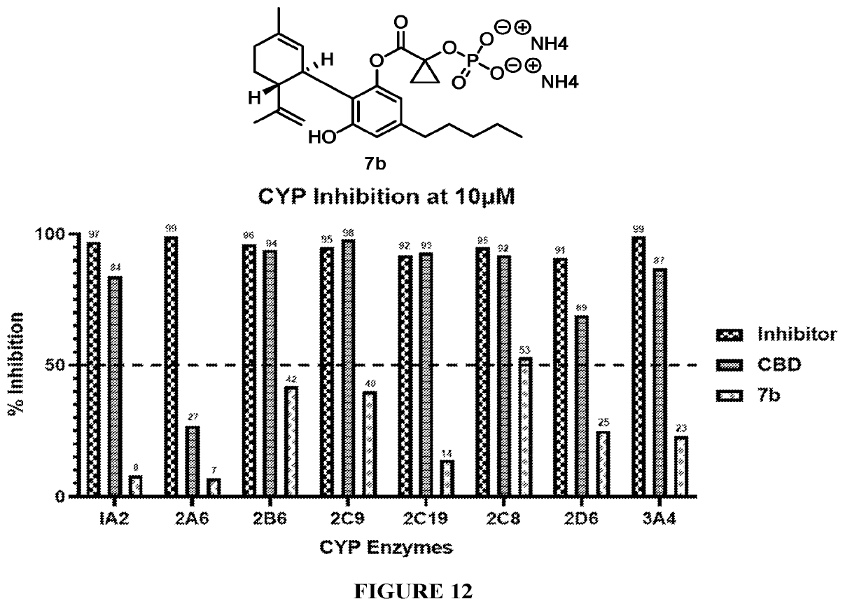

Procedure for CYP Inhibition Assay: Human liver microsomes were taken from −80° C. Microsomes were then thawed on ice bath for 30 min. A microsomal working solution of 0.2 mg/mL was prepared in the 50 mM potassium phosphate buffer (pH 7.4) for 3A4, 2D6, 2B6, 1A2, 2A6, 2C8, and 2C9. A microsomal working solution of 1 mg/mL was prepared in the 50 mM potassium phosphate buffer (pH 7.4) for 2C19. Spike 0.3 μL of test compounds/positive controls and vehicle controls (DMSO) into 150 μL of microsomal working solution for each isoform. From the above microsomal mixture, aliquot 50 μL (n=2) was transferred in a separate plate and 25 μL of marker substrate (isoform specific) was added and pre-incubated for 5 min at 37° C. The reaction was initiated by adding 25 μL of 4 mM NADPH (Note: In final reaction, NADPH concentration was 1 mM and protein concentration was 0.1 mg/mL for 3A4, 2D6, 2B6, 1A2, 2A6, 2C8, 2C9, and 1 mg/ml for CYP 2C19). The reaction mixture was incubated in 37° C. for between 5-20 minutes depending on CYP enzyme. The reaction mixture was terminated by adding 100 μL of stop solution. All samples were vortexed for 10 min and centrifuged at 4000 rpm for 10 min. Post centrifugation, a 100 μL of supernatant was transferred in LCMS loading plate for analysis. Samples were monitored by identifying the substrate metabolites in MRM mode using LC-MS/MS. The peak area ratios-PAR (metabolites to internal standard) should be used to calculate % inhibition. % Activity=PA ratios in presence of test compound/PA ratios of DMSO control*100. % Inhibition=100−% Activity. % Inhibition for the selected positive controls (against specific CYP isoforms) and test compounds are shown graphically in FIG. 12.

### Example 15 In Vivo Pharmacokinetic Studies of Prodrugs 7C, 29a, 32, and 38C

General Experimental: In vivo pharmacokinetic studies of 7c, 29a, 32, and 38c were carried out by SAI Life Sciences Limited. The objective of this study was to determine the pharmacokinetics and brain tissue distribution of CBD released from 7c, 29a, 32, and 38c in male C57BL/6 mice following a single oral administration of prodrugs at 50 mg/kg equivalent dose to CBD. Plasma and brain sampling was performed in triplicate at 0.5, 2, and 4 hr timepoints. Prodrugs were formulated as 10% v/v PEG-300, 10% v/v Solutol HS-15 and 80% v/v Normal saline. Doses for 7c, 29a, 32, and 38c were 84 mg/kg, 82 mg/kg, 82 mg/kg and 84 mg/kg, respectively, at dose volume of 10 mL/kg.

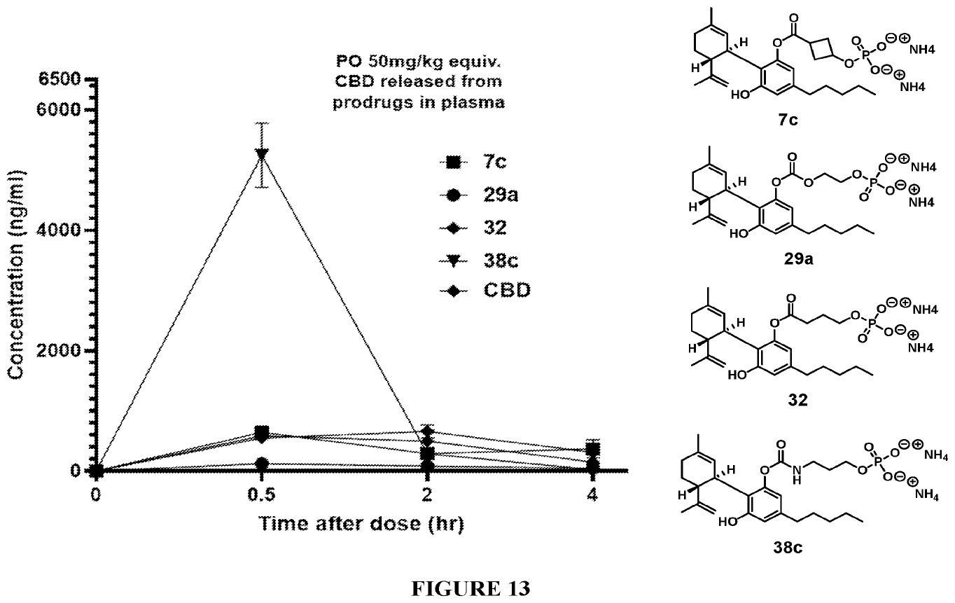

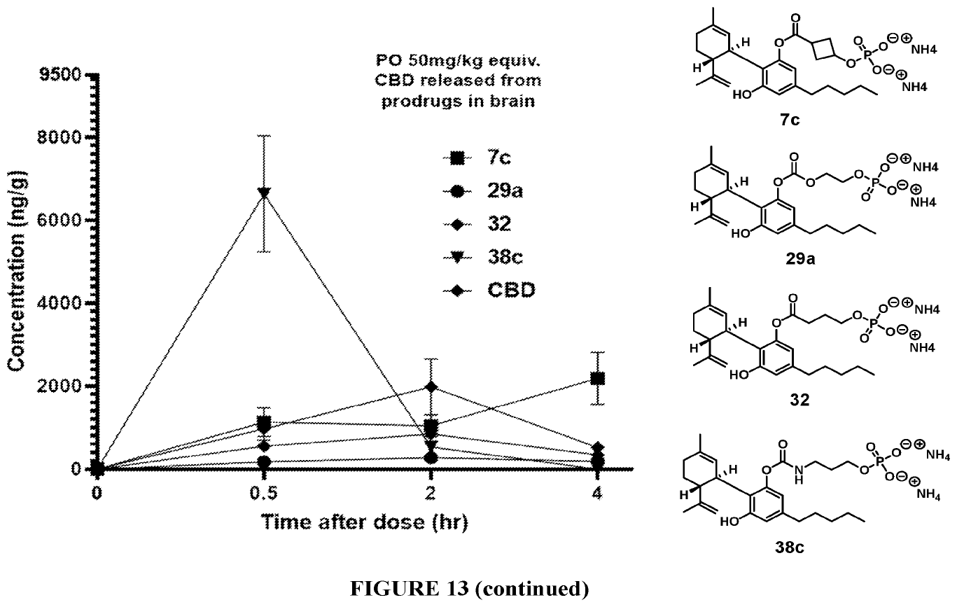

Procedure for In Vivo Pharmacokinetic Studies: Nine male C57BL/6 mice were administered orally with solution formulation of each prodrug at dosage described above. Blood samples (approximately 60 μL) were collected under light isoflurane anaesthesia from a set of three mice at 0.5 hrs, 2 hrs, and 4 hrs. The blood samples were collected at each time point in labelled micro centrifuge tube containing 6 μL of stabilizer (5×Halt (5 μL)+1000 μM PMSF+1000 μM paraoxon) and K₂EDTA as anticoagulant. Plasma was harvested by centrifugation of blood and stored at −70±10° C. until analysis. Immediately after collection of blood, animals were anesthetized, and brain samples were collected from each mouse at respective time points. Brain samples were homogenized using ice-cold phosphate buffer saline (pH 7.4) containing stabilizer (5× Halt+1000 μM PMSF+1000 μM paraoxon) and homogenates were stored below −70±10° C. until analysis. Total homogenate volume was three times the brain weight. Brain samples were diluted (1-part of tissue: 2-part of buffer) and homogenized. The homogenate was submitted for bioanalysis and the concentrations (ng/mL) received were corrected with dilution factor (3×) and the final reported concentrations were represented in ng/g. The plasma and brain concentration-time data of CBD and prodrugs were used for calculation. Plasma and brain samples were quantified by fit-for-purpose LC-MS/MS method. The extraction procedure for plasma samples and the spiked plasma calibration standards were carried out on ice bath: (PMSF+HALT Inhibitor Stabilized Plasma and Brain used for preparation of calibration standards). A 20 μL of study sample plasma or spiked plasma calibration standard was added to individual pre-labelled microcentrifuge tubes followed by 200 μL of internal standard prepared in 0.1% Formic acid in methanol (Cetrizine+Bicalcutamide, 50 ng/mL) was added except for blank, where 200 μL of in 0.1% Formic acid in methanol was added. Samples were vortexed for 5 minutes. Samples were centrifuged for 10 minutes at a speed of 4000 rpm at 4° C. Following centrifugation, 200 μL of clear supernatant was transferred in 96 well plates and analysed using LC-MS/MS. The results of these studies are shown graphically in FIG. 13.

### Example 16

### In Vivo Efficacy Antiseizure Studies of CBD, 7a, and 7B

General Experimental: CBD and prodrugs 7a and 7b were screened for their ability to block psychomotor seizures induced by a low-frequency (6 Hz), long-duration (3 s) stimulus delivered through corneal electrodes. Stimulus was delivered to mice 2 hours post IP injection dosage of CBD and prodrugs 7a and 7b. Dosages ranged from 25-200 mg/kg for compounds. CBD and prodrugs were formulated as 10% v/v Ethanol, 10% v/v Cremaphor and 80% v/v Normal saline. Testing results were quantified using a modified Racine score to determine the degree of seizure and seizure protection in the epilepsy electroshock model.

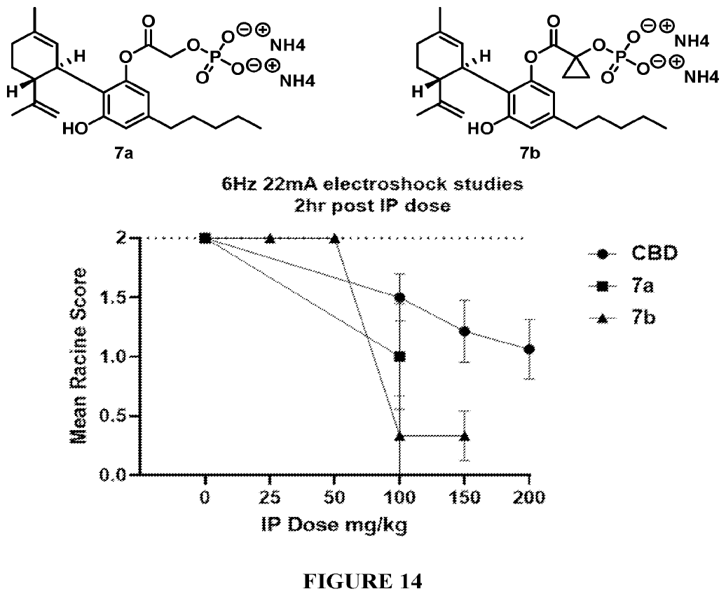

Procedure for In Vivo Efficacy Antiseizure Studies: Male albino CF-1 mice (18-25 g; Charles River, Kingston, NY) were used as experimental animals. All animals were allowed free access to both food (Prolab RMH 3000) and water except when they were removed from their cages for the experimental procedure. All mice were housed, fed, and handled in a manner consistent with the recommendations in the National Research Council publication, “Guide for the Care and Use of Laboratory Animals” and approved by the University of Utah Institutional Animal Care and Use Committee (IACUC). Mice were ˜12 weeks old and were visually inspected to be healthy with no signs of skin problems or fighting. CBD was administered to mice by intraperitoneal (IP) route at various dosage concentrations in the formulation stated above at a dose volume of 10 ml/kg. 2 hours post IP injection, a 6 Hz stimulus was delivered through corneal electrodes. An electrolyte solution containing an anaesthetic agent was applied to the eyes before stimulation (0.5% tetracaine HCl). Mice were challenged with a 22-mA current (convulsive current that elicits seizures in 97% of CF1 mice). The seizure was characterized by an initial momentary stun followed immediately by forelimb clonus, twitching of the vibrissae, and Straub tail; absence of these behaviors were the criteria for a “protected mouse. Mice were evaluated by a modified Racine score, with a score of 2 being no seizure protection and 0 being fully seizure protected. The results of these studies are shown graphically in FIG. 14.

### Example 17

### Bioanalytical Results

The following tables and FIGS. 8-11 detail the bioanalytical results of exemplary compounds of the present disclosure.

TABLE 1
Bioanalytical data of ester linked mono-ester phosphate prodrugs of CBD.

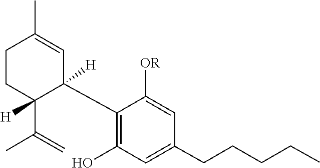

| | | Solubility | T½ | T½ | T½ in | T 1/2 | T½ in |
|---|---|---|---|---|---|---|---|
| | | in 1% | in | in | human | in | mouse |
| | | DMSO | SGF | HLM | plasma | MLM | plasma |
| Code | Prodrug (R) | (uM) | (h) | (min) | (h) | (min) | (h) |
| CBD | H | 16.93 ± 0.55 | >3 | 5.8 | 24 ± 6 | 2.9 | 9.6 |
| 7a | 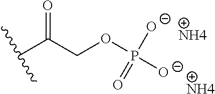 [SMILES: *C(=O)COP(=O)([O-])[O-] ; [NH4+] ; [NH4+]] | >500 | 5.25 | 59 | 20 min | 6.1 | 4.5 min |
| 7b | 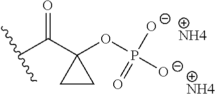 [SMILES: *C(=O)C1(OP(=O)([O-])[O-])CC1 ; [NH4+] ; [NH4+]] | >500 | >3 | 157 | 5.3 | 33 | 3.7 |
| 7c | 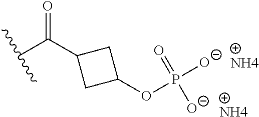 [SMILES: *C(=O)C1CC(OP(=O)([O-])[O-])C1 ; [NH4+] ; [NH4+]] | >500 | — | 53 | 7.2 | — | — |
| 7d | 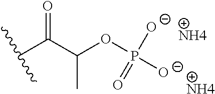 | >500 | — | 50 | 21 min | — | — |
| 7e | 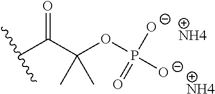 [SMILES: *C(=O)C(C)(C)OP(=O)([O-])[O-] ; [NH4+] ; [NH4+]] | >500 | — | — | 24 min | — | — |

TABLE 2
Bioanalytical data of CBD and alpha substituted ester linked di-ester
phosphate prodrugs of CBD.

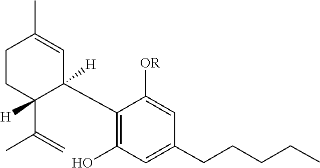

| | | Solubility in | T½ in | T½ in |
|---|---|---|---|---|
| | | 1% DMSO | HLM | human |
| Code | Prodrug (R) | (uM) | (min) | plasma (h) |
| CBD | H | 16.93 ± 0.55 | 5.8 | 24 ± 6 |
| 8 | 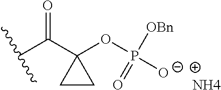 [SMILES: *C(=O)C1(OP(=O)([O-])OCc2ccccc2)CC1 ; [NH4+]] | 287 ± 13 | 23 | 5.3 |
| 14a | 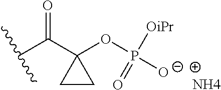 [SMILES: *C(=O)C1(OP(=O)([O-])OC(C)C)CC1 ; [NH4+]] | >500 | 13 | >3 |
| 14b | 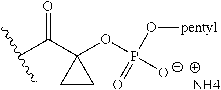 | 276 ± 4.52 | 24 | 9.6 |
| 14c | 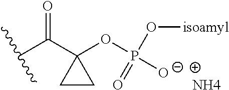 | 345 ± 4.51 | 24 | >4 |
| 14d | 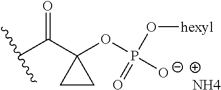 | 98 ± 1.24 | 32 | >4 |
| 14e | 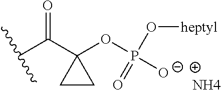 | 57 ± 1.24 | 45 | >4 |
| 14f | 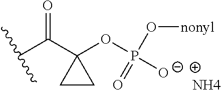 | 67 ± 5.98 | >30 | >4 |
| 14g | 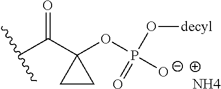 | 75 ± 2.14 | >30 | >4 |
| 14h | 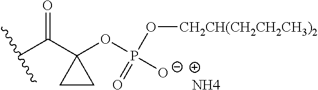 | 66 ± 3.17 | 26 | >4 |
| 14i | 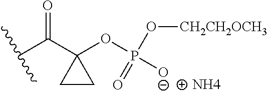 | >500 | 19 | >4 |
| 14j | 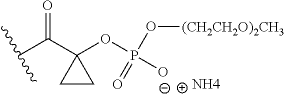 | >500 | 24 | >4 |

TABLE 3
Bioanalytical data of CBD and carbonate linked mono-ester phosphate
prodrugs of CBD.

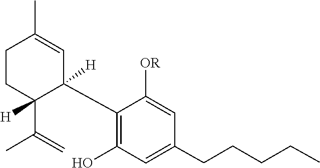

| | | Solubility in | T½ in | T½ in |
|---|---|---|---|---|
| | | 1% DMSO | HLM | human |
| Code | Prodrug (R) | (uM) | (min) | plasma (h) |
| CBD | H | 16.93 ± 0.55 | 5.8 | 24 ± 6 |
| 24 | 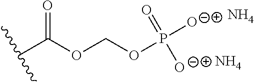 [SMILES: *C(=O)OCOP(=O)([O-])[O-] ; [NH4+] ; [NH4+]] | >500 | — | >4 |
| 29a | 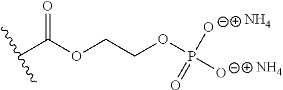 [SMILES: *C(=O)OCCOP(=O)([O-])[O-] ; [NH4+] ; [NH4+]] | >500 | 42 | 16.5 |
| 29b | 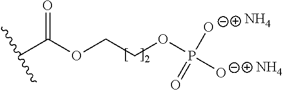 | >500 | 19 | >4 |
| 29c | 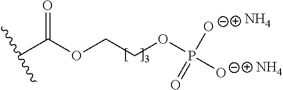 | >500 | 25 | >4 |

TABLE 4
Bioanalytical data of CBD and carbamate linked mono-ester phosphate
prodrugs of CBD

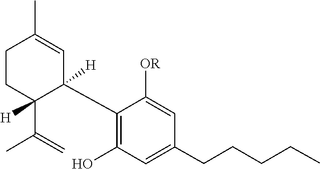

| | | Solubility in | T½ in | T½ in |
|---|---|---|---|---|
| | | 1% DMSO | HLM | human |
| Code | Prodrug (R) | (uM) | (min) | plasma (h) |
| CBD | H | 16.93 ± 0.55 | 5.8 | 24 ± 6 |
| 38a | 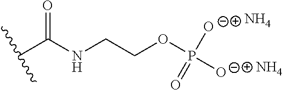 [SMILES: *C(=O)NCCOP(=O)([O-])[O-] ; [NH4+] ; [NH4+]] | >500 | 121 | >4 |
| 38b | 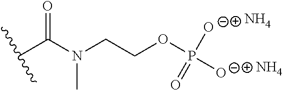 [SMILES: *C(=O)N(C)CCOP(=O)([O-])[O-] ; [NH4+] ; [NH4+]] | >500 | 230 | >4 |
| 38c | 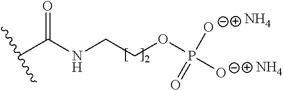 | >500 | 73 | >4 |

1. At least one compound chosen from cannabinoid prodrugs of Formula (I): 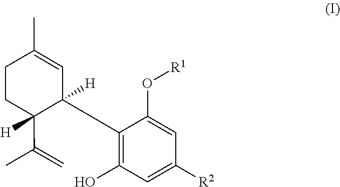
and pharmaceutically acceptable salts thereof, wherein
R¹ is chosen from 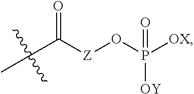
groups, wherein
Z is chosen from divalent C₁₋₁₈ alkyl and divalent C₁₋₁₈ haloalkyl groups, wherein the divalent C₁₋₁₈ alkyl and divalent C₁₋₁₈ haloalkyl groups are optionally substituted with one or more groups independently chosen from C₆₋₈ aryl, C₁₋₁₃ heteroaryl, C₂₋₁₂ heterocyclyl, —OC₁₋₁₈ alkyl, —SC₁₋₁₈ is alkyl, —N(T²)C₁₋₁₈ alkyl, and —N(T²)AA groups, wherein T² is chosen from H and C₁₋₈ alkyl groups and AA is chosen from amino acid residues and is attached to the nitrogen via its C-terminus carbonyl group;
X and Y, which may be identical or different, are independently chosen from H, Q, C₁₋₁₈ alkyl, C₁₋₁₈ haloalkyl, C₆₋₈ aryl, C₁₋₁₃ heteroaryl, C₇₋₁₉ arylalkyl, C₂₋₁₄ heteroarylalkyl, —(CH₂CH₂O)ₙCH₃, 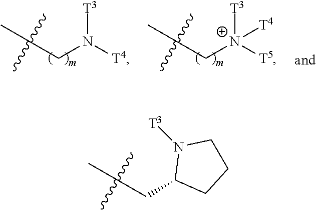
groups, wherein
each T³, T⁴, and T⁵, which may be the same or different, are independently chosen from H and C₁₋₈ alkyl groups,
each Q is independently chosen from pharmaceutically acceptable cations,
each n is independently chosen from integers ranging from 1 to 12, and
each m is independently chosen from integers ranging from 1 to 8; and
R² is chosen from C₁₋₈ alkyl groups.

2-6. (canceled)

7. The at least one compound according to claim 1, wherein Z is chosen from divalent C₁₋₁₈ alkyl groups.

8. The at least one compound according to claim 7, wherein Z is chosen from —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—,

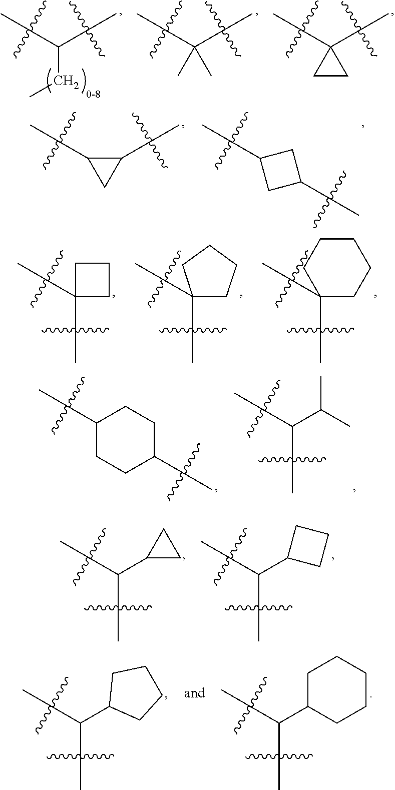

9. The at least one compound according to claim 8, wherein Z is —(CH₂)₂—.

10. The at least one compound according to claim 8, wherein Z is —(CH₂)₃—.

11. The at least one compound according to claim 8, wherein Z is

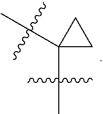 [SMILES: *C1(*)CC1]

12. The at least one compound according to claim 8, wherein Z is

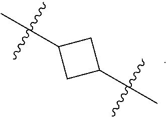 [SMILES: *C1CC(*)C1]

13. The at least one compound according to claim 1, wherein Z is chosen from divalent C₁₋₁₈ haloalkyl groups.

14. The at least one compound according to claim 13, wherein Z is

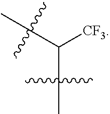 [SMILES: *C(*)C(F)(F)F]

15. The at least one compound according to claim 1, wherein Z is chosen from divalent C₁₋₁₈ alkyl groups substituted with at least one group chosen from C₆₋₁₈ aryl groups.

16. The at least one compound according to claim 15, wherein Z is

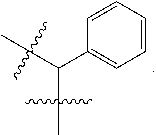 [SMILES: *C(*)c1ccccc1]

17. The at least one compound according to claim 15, wherein Z is

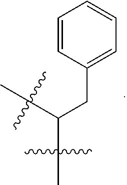 [SMILES: *C(*)Cc1ccccc1]

18. The at least one compound according to claim 1, wherein Z is chosen from divalent C₁₋₁₈ alkyl groups substituted with at least one group chosen from C₁₋₁₃ heteroaryl groups.

19. The at least one compound according to claim 18, wherein Z is chosen from

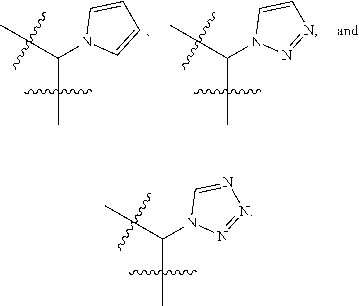 [SMILES: *C(*)n1cccc1 ; *C(*)n1ccnn1 ; *C(*)n1cnnn1]

20. The at least one compound according to claim 1, wherein Z is chosen from divalent C₁₋₁₈ alkyl groups substituted with at least one group chosen from C₂₋₁₂ heterocyclyl groups.

21. The at least one compound according to claim 20, wherein Z is chosen from

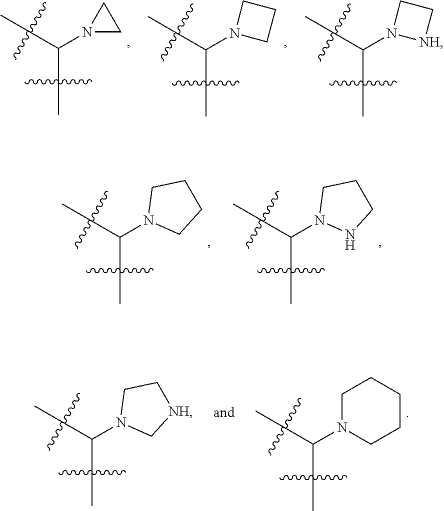

22. The at least one compound according to claim 1, wherein Z is chosen from divalent C₁₋₁₈ alkyl groups substituted with at least one group chosen from —OC₁₋₁₈ alkyl groups.

23. The at least one compound according to claim 22, wherein Z is chosen from

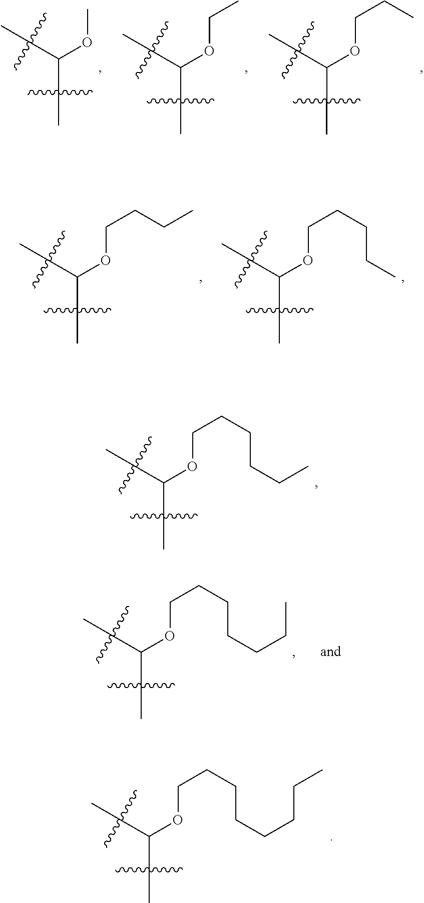

24. The at least one compound according to claim 1, wherein Z is chosen from divalent C₁₋₁₈ alkyl groups substituted with at least one group chosen from —SC₁₋₁₈ alkyl groups.

25. The at least one compound according to claim 24, wherein Z is chosen from

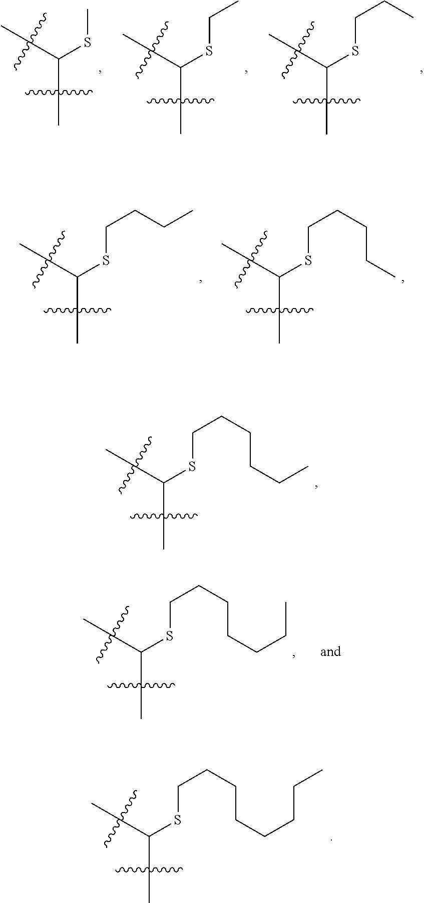

26. The at least one compound according to claim 1, wherein Z is chosen from divalent C₁₋₁₈ alkyl groups substituted with at least one group chosen from —N(T²)C₁₋₁₈ alkyl groups.

27. The at least one compound according to claim 26, wherein Z is chosen from

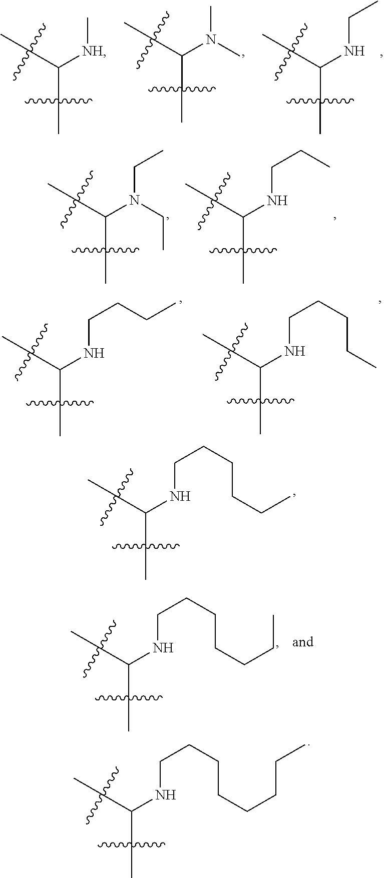

28. The at least one compound according to claim 1, wherein Z is chosen from divalent C₁₋₁₈ alkyl groups substituted with at least one group chosen from —N(T²)AA groups.

29. The at least one compound according to claim 28, wherein Z is chosen from 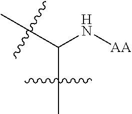
groups.

30. The at least one compound according to claim 28, wherein AA is chosen from natural L-amino acid residues.

31-46. (canceled)

47. The at least one compound according to claim 1, wherein X is H.

48. The at least one compound according to claim 1, wherein X is Q.

49-64. (canceled)

65. The at least one compound according to claim 48, wherein Y is H.

66. The at least one compound according to claim 48, wherein Y is Q.

67. The at least one compound according to claim 66, wherein each Q is independently chosen from ammonium (substituted and unsubstituted) cations.

68. The at least one compound according to claim 67, wherein each Q is unsubstituted ammonium cation.

69-73. (canceled)

74. The at least one compound according to claim 1, wherein R² is chosen from C₂₋₆ alkyl groups.

75. The at least one compound according to claim 1, wherein the carbon in Z that is next to the carbonyl group has no hydrogen atoms.

76. The at least one compound according to claim 1, wherein the carbon in Z that is next to the carbonyl group has one hydrogen atom.

77. The at least one compound according to claim 1, wherein the carbon in Z that is next to the carbonyl group is substituted.

78-80. (canceled)

81. The at least one compound according to claim 75, wherein X and Y are each unsubstituted ammonium cations.

82. The at least one compound according to claim 76, wherein X and Y are each unsubstituted ammonium cation.

83. The at least one compound according to claim 81, wherein R² is chosen from n-pentyl and n-propyl.

84. The at least one compound according to claim 82, wherein R² is chosen from n-pentyl and n-propyl.

85. A composition comprising at least one compound according to claim 1 and at least one pharmaceutically acceptable carrier.

86. The composition according to claim 85, wherein the composition is an oral formulation.

87. The composition of claim 86, wherein the oral formulation is a solid form chosen from tablets, capsules, pills, and granules.

88. The composition of claim 86, wherein the oral formulation is a liquid form chosen from solutions, suspensions, and emulsions.

89. A method for treatment and/or prevention of at least one disease, disorder, and/or condition where treatment with an anxiolytic, analgesic, antiemetic, mood-stabilizing agent, and/or antipsychotic agent is useful, the method comprising administering to a subject in need thereof an effective amount of the at least one compound of claim 1.

90. A method for treatment and/or prevention of at least one psychiatric disease, disorder, and/or condition, the method comprising administering to a subject in need thereof an effective amount of the at least one compound of claim 1.

91. The method according to claim 90, wherein the at least one psychiatric disease, disorder, and/or condition is chosen from depression, anxiety, dementia, bipolar disorder, schizophrenia, addiction, and nausea.

92. The method according to claim 91, wherein the at least one psychiatric disease, disorder, and/or condition is chosen from depression and anxiety.

93. A method for treatment and/or prevention of pain, the method comprising administering to a subject in need thereof an effective amount of at least one compound of claim 1.

94. The method according to claim 93, wherein the pain is chronic pain.

95. The method according to claim 93, wherein the pain is neuropathic pain.

96. The method according to claim 95, wherein the neuropathic pain results from peripheral and/or central nervous system pathologic events.

97. The method according to claim 95, wherein the neuropathic pain is chosen from peripheral diabetic neuropathy, postherpetic neuralgia, complex regional pain syndromes, peripheral neuropathies, rheumatoid arthritis, chemotherapy-induced neuropathic pain, cancer neuropathic pain, neuropathic low back pain, HIV neuropathic pain, trigeminal neuralgia and/or central post-stroke pain.

98. A method for treatment and/or prevention of at least one neurological disease, disorder, and/or condition, the method comprising administering to a subject in need thereof an effective amount of at least one compound of claim 1.

99. The method according to claim 98, wherein the at least one neurological disease, disorder, and/or condition is chosen from depression, autism, postpartum depression, attention-deficit disorder, schizophrenia, anxiety, various psychoses, and epilepsies.

100. A method for treatment and/or prevention of at least one symptom associated with epilepsies and/or similar seizure disorders, the method comprising administering to a subject in need thereof an effective amount of at least one compound of claim 1.

101. A method for treatment and/or prevention of at least one symptom associated with Lennox-Gastaut syndrome, Dravet syndrome, developmental epileptic encephalopathy, and/or tuberous sclerosis complex, the method comprising administering to a subject in need thereof an effective amount of at least one compound of claim 1.

102. A method for reducing the frequency of seizures associated with epilepsies and/or rare genetic disorders/diseases, the method comprising administering to a subject in need thereof an effective amount of at least one compound of claim 1.

103. A method for reducing the severity and/or intensity of seizures associated with epilepsies and/or rare genetic disorders/diseases, the method comprising administering to a subject in need thereof an effective amount of at least one compound of claim 1.

104. A method for treating traumatic brain injuries, the method comprising administering to a subject in need thereof an effective amount of at least one compound of claim 1.

105. A method for treating sleep disorders, the method comprising administering to a subject in need thereof an effective amount of at least one compound of claim 1.

106. A method for treating high blood pressure and/or hypertension, the method comprising administering to a subject in need thereof an effective amount of at least one compound of claim 1.

107. The method according to any one of claims 89-106, wherein the subject is human.

108. The method according to claim 107, wherein the human is under the age of 18.

109. The method according to claim 107, wherein the at least one compound of claim 1 is administered orally.

110. The at least one compound according to claim 1, wherein the at least one compound is chosen from:

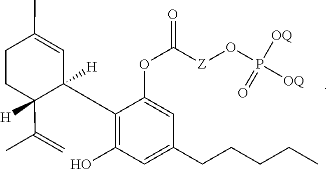

111. The at least one compound according to claim 110, wherein Z is chosen from divalent C₁₋₁₈ alkyl groups.

112. The at least one compound according to claim 111, wherein Z is chosen from —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—,

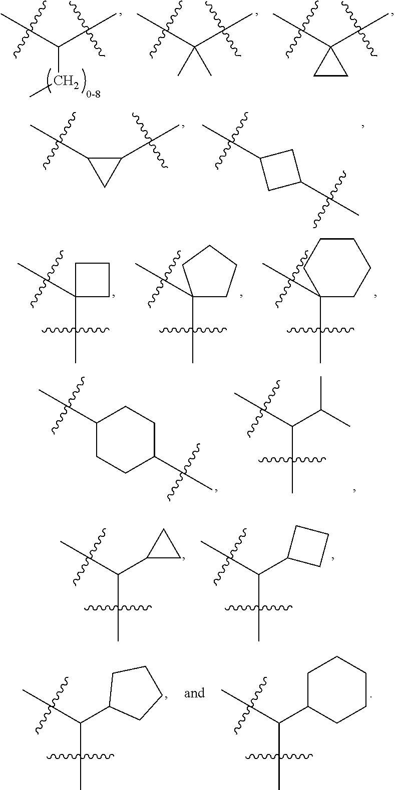

113. The at least one compound according to claim 112, wherein each Q is independently chosen from ammonium (substituted and unsubstituted) cations.

114. The at least one compound according to claim 112, wherein each Q is unsubstituted ammonium cation.

115. The at least one compound according to claim 1, wherein the at least one compound is chosen from: 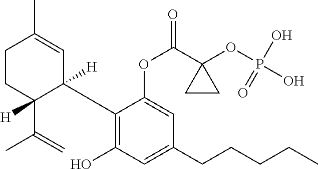 [SMILES: C=C(C)[C@@H]1CCC(C)=C[C@H]1c1c(O)cc(CCCCC)cc1OC(=O)C1(OP(=O)(O)O)CC1]
and pharmaceutically acceptable salts thereof.

116. The at least one compound according to claim 1, wherein the at least one compound is chosen from: 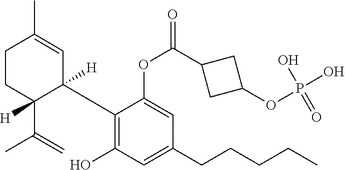 [SMILES: C=C(C)[C@@H]1CCC(C)=C[C@H]1c1c(O)cc(CCCCC)cc1OC(=O)C1CC(OP(=O)(O)O)C1]
and pharmaceutically acceptable salts thereof.

117. The at least one compound according to claim 1, wherein the at least one compound is:

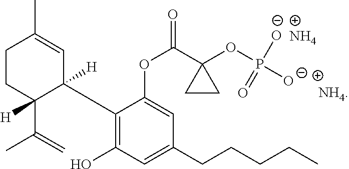

118. The at least one compound according to claim 1, wherein the at least one compound is: [chemical structure]
